SEQUENCE LISTING (236 sequences)

SEQ ID NO: 1 - protein, 106 aa, Mus musculus
DIVLTQSPVSLAVSLGQRATISCRASESVDDYGISFMHWYQQKPGQPPKLLIYRASDLESGIPARFSGSGSGTDFTLTINPVETDDVAIYYCQQTSKDPWTFGGGT

SEQ ID NO: 2 - protein, 122 aa, Mus musculus
EVQLQESGPSLVKPSQTLSLTCSVTGDSITRGYWNWIRKFPGNKLEFMGYINYSGTTYYNPSLKSRISITRDTSKNQYYLQLNSVTTEDTATYYCATPHTYGAMDYWGQGTTLTVSAASTKG

SEQ ID NO: 3 - protein, 112 aa, Artificial Sequence
  Synthetic polypeptide
DIQMTQSPSSLSASVGDRVTITCRASESVDDYGISFMHWYQQKPGKAPKLLIYRASDLESGVPSRFSGSGSGTDFTLTISSLQPEDFATYYCQQTSKDPWTFGQGTKVEIKR

SEQ ID NO: 4 - protein, 117 aa, Artificial Sequence
  Synthetic polypeptide
EVQLVESGGGLVQPGGSLRLSCAASGDSITRGYWNWVRQAPGKGLEWVGYINYSGTTYYNPSLKSRFTISADTSKNTAYLQMNSLRAEDTAVYYCATPHTYGAMDYWGQGTLVTVSS

SEQ ID NO: 5 - protein, 117 aa, Artificial Sequence
  Synthetic polypeptide
EVQLVESGGGLVQPGGSLRLSCAASGDSITRGYWNWVRQAPGKGLEWVGYINYSGTTYYNPSLKSRFTISRDTSKNTLYLQMNSLRAEDTAVYYCATPHTYGAMDYWGQGTLVTVSS

SEQ ID NO: 6 - protein, 117 aa, Artificial Sequence
  Synthetic polypeptide
EVQLVESGGGLVQPGGSLRLSCAASGDSITRGYWNWVRQAPGKGLEWVGYINYSGTTYYNPSLKSRFTISRDTSKNTFYLQMNSLRAEDTAVYYCATPHTYGAMDYWGQGTLVTVSS

SEQ ID NO: 7 - protein, 117 aa, Artificial Sequence
  Synthetic polypeptide
EVQLVESGGGLVQPGGSLRLSCAASGDSITRGYWNWVRQAPGKGLEWVGYINYSGTTYYNPSLKSRFTISRDTSKNTFYLQMNSLRAEDTAVYYCATTLPYGAMDYWGQGTLVTVSS

SEQ ID NO: 8 - protein, 117 aa, Artificial Sequence
  Synthetic polypeptide
EVQLVESGGGLVQPGGSLRLSCAASGDSITRGYWNWVRQAPGKGLEWVGYINYSGTTYYNPSLKSRFTISRDTSKNTFYLQMNSLRAEDTAVYYCANSNFYGAMDYWGQGTLVTVSS

SEQ ID NO: 9 - protein, 117 aa, Artificial Sequence
  Synthetic polypeptide
EVQLVESGGGLVQPGGSLRLSCAASGDSITRGYWNWVRQAPGKGLEWVGYINYSGTTYYNPSLKSRFTISRDTSKNTFYLQMNSLRAEDTAVYYCANLNYYGAMDYWGQGTLVTVSS

SEQ ID NO: 10 - protein, 117 aa, Artificial Sequence
  Synthetic polypeptide
EVQLVESGGGLVQPGGSLRLSCAASGDSITRGYWNWVRQAPGKGLEWVGYINYSGTTYYNPSLKSRFTISRDTSKNTLYLQMNSLRAEDTAVYYCANANYYGAMDYWGQGTLVTVSS

SEQ ID NO: 11 - protein, 117 aa, Artificial Sequence
  Synthetic polypeptide
EVQLVESGGGLVQPGGSLRLSCAASGDSITRGYWNWVRQAPGKGLEWVGYINYSGTTYYNPSLKSRFTISRDTSKNTLYLQMNSLRAEDTAVYYCATSHNTGEMDYWGQGTLVTVSS

SEQ ID NO: 12 - protein, 117 aa, Artificial Sequence
  Synthetic polypeptide
EVQLVESGGGLVQPGGSLRLSCAASGDSITRGYWNWVRQAPGKGLEWVGYINYSGTTYYNPSLKSRFTISRDTSKNTLYLQMNSLRAEDTAVYYCATTLPYGAMDYWGQGTLVTVSS

SEQ ID NO: 13 - protein, 117 aa, Artificial Sequence
  Synthetic polypeptide
EVQLVESGGGLVQPGGSLRLSCAASGDSITRGYWNWVRQAPGKGLEWVGYINYSGTTYYNPSLKSRFTISRDTSKNTLYLQMNSLRAEDTAVYYCANSNFYGAMDYWGQGTLVTVSS

SEQ ID NO: 14 - protein, 237 aa, Artificial Sequence
  Synthetic polypeptide
MGWSCIILFLVATATGVHSDIQMTQSPSSLSASVGDRVTITCRASESVDDYGISFMHWYQQKPGKAPKLLIYRASDLESGVPSRFSGSGSGTDFTLTISSLQPEDFATYYCQQTSKDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 441 aa, Artificial Sequence
  Synthetic polypeptide
ESGGGLVQPGGSLRLSCAASGDSITRGYWNWVRQAPGKGLEWVGYINYSGTTYYNPSLKSRFTISRDTSKNTFYLQMNSLRAEDTAVYYCANSNFYGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 117 aa, Artificial Sequence
  Synthetic polypeptide
EVQLVESGGGLVQPGGSLRLSCAASGDSITRGYWNWVRQAPGKGLEWVGYINYSGTTYYNPSLKSRFTISRDTSKNTFYLQMNSLRAEDTAVYYCATNHLYGAMDYWGQGTLVTVSS

SEQ ID NO: 17 - protein, 117 aa, Artificial Sequence
  Synthetic polypeptide
EVQLVESGGGLVQPGGSLRLSCAASGDSITRGYWNWVRQAPGKGLEWVGYINYSGTTYYNPSLKSRFTISRDTSKNTFYLQMNSLRAEDTAVYYCARPHNYGAMDYWGQGTLVTVSS

SEQ ID NO: 18 - protein, 117 aa, Artificial Sequence; identical to SEQ ID NO: 17
  Synthetic polypeptide

SEQ ID NO: 19 - protein, 108 aa, Mus musculus
DIVMSQSPSSLAVSVGEKVTMSCKSSQSLLYSSNQNNYLAWYQQKPGQSPKLLIYWASTRESGVPDRFTGSGSGTDFTLTISSVKAEDLAVYYCQQYYTYPYTFGGGT

SEQ ID NO: 20 - protein, 123 aa, Mus musculus
EVKLVESGGGLVQPGGSLRLSCAISGFTVTAYYMSWVRQPPGKALEWLGFIRDKANGYTTEYNPSVKGRFTISRDNSQSIFYLQMNTLRAEDSATYYCAQVRRALDYWGQGTTVTVSAASTKG

SEQ ID NO: 21 - protein, 114 aa, Artificial Sequence
  Synthetic polypeptide
DIQMTQSPSSLSASVGDRVTITCKSSQSLLYSSNQNNYLAWYQQKPGKAPKLLIYWASTRESGVPSRFSGSGSGTDFTLTISSLQPEDFATYYCQQYYTYPYTFGQGTKVEIKR

SEQ ID NO: 22 - protein, 118 aa, Artificial Sequence
  Synthetic polypeptide
EVQLVESGGGLVQPGGSLRLSCAASGFTVTAYYMSWVRQAPGKGLEWVGFIRDKANGYTTEYNPSVKGRFTISADTSKNTAYLQMNSLRAEDTAVYYCAQVRRALDYWGQGTLVTVSS

SEQ ID NO: 23 - protein, 114 aa, Artificial Sequence
  Synthetic polypeptide
DIQMTQSPSSLSASVGDRVTITCKSSQSLLYSSNQNNYLAWYQQKPGKAPKLLIYWAQHLDSGVPSRFSGSGSGTDFTLTISSLQPEDFATYYCQQYYTYPYTFGQGTKVEIKR

SEQ ID NO: 24 - protein, 118 aa, Artificial Sequence
  Synthetic polypeptide
EVQLVESGGGLVQPGGSLRLSCAASGLPMAGFYTSWVRQAPGKGLEWVGFIRDKANGYTTEYNPSVKGRFTISADTSKNTAYLQMNSLRAEDTAVYYCAQVRRALDYWGQGTLVTVSS

SEQ ID NO: 25 - protein, 114 aa, Artificial Sequence
  Synthetic polypeptide
DIQMTQSPSSLSASVGDRVTITCKSSQSLLYSSNQNNYLAWYQQKPGKAPKLLIYWASIRDSGVPSRFSGSGSGTDFTLTISSLQPEDFATYYCQQYYTYPYTFGQGTKVEIKR

SEQ ID NO: 26 - protein, 118 aa, Artificial Sequence
  Synthetic polypeptide
EVQLVESGGGLVQPGGSLRLSCAASDSPRSGYYISWVRQAPGKGLEWVGFIRDKANGYTTEYNPSVKGRFTISADTSKNTAYLQMNSLRAEDTAVYYCAQVRRALDYWGQGTLVTVSS

SEQ ID NO: 27 - protein, 118 aa, Artificial Sequence
  Synthetic polypeptide
EVQLVESGGGLVQPGGSLRLSCAASAWPVTGYYMSWVRQAPGKGLEWVGFIRDKANGYTTEYNPSVKGRFTISADTSKNTAYLQMNSLRAEDTAVYYCAQVRRALDYWGQGTLVTVSS

SEQ ID NO: 28 - protein, 118 aa, Artificial Sequence
  Synthetic polypeptide
EVQLVESGGGLVQPGGSLRLSCAASGSTVSSYYFSWVRQAPGKGLEWVGFIRDKANGYTTEYNPSVKGRFTISADTSKNTAYLQMNSLRAEDTAVYYCAQVRRALDYWGQGTLVTVSS

SEQ ID NO: 29 - protein, 118 aa, Artificial Sequence
  Synthetic polypeptide
EVQLVESGGGLVQPGGSLRLSCAASSPAVAPHYWSWVRQAPGKGLEWVGFIRDKANGYTTEYNPSVKGRFTISADTSKNTAYLQMNSLRAEDTAVYYCAQVRRALDYWGQGTLVTVSS

SEQ ID NO: 30 - protein, 118 aa, Artificial Sequence
  Synthetic polypeptide
EVQLVESGGGLVQPGGSLRLSCAASGFPYTSYYITWVRQAPGKGLEWVGFIRDKANGYTTEYNPSVKGRFTISADTSKNTAYLQMNSLRAEDTAVYYCAQVRRALDYWGQGTLVTVSS

SEQ ID NO: 31 - protein, 118 aa, Artificial Sequence
  Synthetic polypeptide
EVQLVESGGGLVQPGGSLRLSCAASGFTGGSYYIGWVRQAPGKGLEWVGFIRDKANGYTTEYNPSVKGRFTISADTSKNTAYLQMNSLRAEDTAVYYCAQVRRALDYWGQGTLVTVSS

SEQ ID NO: 32 - protein, 118 aa, Artificial Sequence
  Synthetic polypeptide
EVQLVESGGGLVQPGGSLRLSCAASGFTESAYYISWVRQAPGKGLEWVGFIRDKANGYTTEYNPSVKGRFTISADTSKNTAYLQMNSLRAEDTAVYYCAQVRRALDYWGQGTLVTVSS

SEQ ID NO: 33 - protein, 118 aa, Artificial Sequence
  Synthetic polypeptide
EVQLVESGGGLVQPGGSLRLSCAASGATAAAYYISWVRQAPGKGLEWVGFIRDKANGYTTEYNPSVKGRFTISADTSKNTAYLQMNSLRAEDTAVYYCAQVRRALDYWGQGTLVTVSS

SEQ ID NO: 34 - protein, 118 aa, Artificial Sequence
  Synthetic polypeptide
EVQLVESGGGLVQPGGSLRLSCAASGATGIGYYISWVRQAPGKGLEWVGFIRDKANGYTTEYNPSVKGRFTISADTSKNTAYLQMNSLRAEDTAVYYCAQVRRALDYWGQGTLVTVSS

SEQ ID NO: 35 - protein, 118 aa, Artificial Sequence
  Synthetic polypeptide
EVQLVESGGGLVQPGGSLRLSCAASGFTVTAYYMSWVRQAPGKGLEWVGFIRDKANGYTTEYNPSVKGRFTISRDTSKNTFYLQMNSLRAEDTAVYYCAQVRRALDYWGQGTLVTVSS

SEQ ID NO: 36 - protein, 118 aa, Artificial Sequence
  Synthetic polypeptide
EVQLVESGGGLVQPGGSLRLSCAASGWTEHGHYMSWVRQAPGKGLEWVGFIRDKANGYTTEYNPSVKGRFTISRDTSKNTFYLQMNSLRAEDTAVYYCAQVRRALDYWGQGTLVTVSS

SEQ ID NO: 37 - protein, 118 aa, Artificial Sequence
  Synthetic polypeptide
EVQLVESGGGLVQPGGSLRLSCAASGFTVTAYYMSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 38 - protein, 118 aa, Artificial Sequence
  Synthetic polypeptide
EVQLVESGGGLVQPGGSLRLSCAASGRPRRGYYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 39 - protein, 118 aa, Artificial Sequence
  Synthetic polypeptide
EVQLVESGGGLVQPGGSLRLSCAASGVTGGSFYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 40 - protein, 118 aa, Artificial Sequence
  Synthetic polypeptide
EVQLVESGGGLVQPGGSLRLSCAASGLTGTGYYTSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 41 - protein, 118 aa, Artificial Sequence
  Synthetic polypeptide
EVQLVESGGGLVQPGGSLRLSCAASGFPVTGSYVSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 42 - protein, 118 aa, Artificial Sequence
  Synthetic polypeptide
EVQLVESGGGLVQPGGSLRLSCAASGFTTTARYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 43 - protein, 118 aa, Artificial Sequence
  Synthetic polypeptide
EVQLVESGGGLVQPGGSLRLSCAASGFTVTASYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 44 - protein, 118 aa, Artificial Sequence
  Synthetic polypeptide
EVQLVESGGGLVQPGGSLRLSCAASGITVTASYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 45 - protein, 118 aa, Artificial Sequence
  Synthetic polypeptide
EVQLVESGGGLVQPGGSLRLSCAASGFALRGSYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 46 - protein, 118 aa, Artificial Sequence
  Synthetic polypeptide
EVQLVESGGGLVQPGGSLRLSCAASEFAVTGSYIAWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 47 - protein, 118 aa, Artificial Sequence
  Synthetic polypeptide
EVQLVESGGGLVQPGGSLRLSCAASTRAVTGYYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 48 - protein, 118 aa, Artificial Sequence
  Synthetic polypeptide
EVQLVESGGGLVQPGGSLRLSCAASGYIATGHYIAWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 49 - protein, 118 aa, Artificial Sequence
  Synthetic polypeptide
EVQLVESGGGLVQPGGSLRLSCAASVDKLTGSYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 50 - protein, 118 aa, Artificial Sequence
  Synthetic polypeptide
EVQLVESGGGLVQPGGSLRLSCAASGFLGPGRYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 51 - protein, 118 aa, Artificial Sequence
  Synthetic polypeptide
EVQLVESGGGLVQPGGSLRLSCAASGLQATGSYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 52 - protein, 118 aa, Artificial Sequence
  Synthetic polypeptide
EVQLVESGGGLVQPGGSLRLSCAASGLSMTGVYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 53 - protein, 118 aa, Artificial Sequence
  Synthetic polypeptide
EVQLVESGGGLVQPGGSLRLSCAASGSSLTGYYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 54 - protein, 118 aa, Artificial Sequence
  Synthetic polypeptide
EVQLVESGGGLVQPGGSLRLSCAASGFTLAGYYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 55 - protein, 118 aa, Artificial Sequence
  Synthetic polypeptide
EVQLVESGGGLVQPGGSLRLSCAASGFTVNGRYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 56 - protein, 118 aa, Artificial Sequence; identical to SEQ ID NO: 55
  Synthetic polypeptide

SEQ ID NO: 57 - protein, 118 aa, Artificial Sequence
  Synthetic polypeptide
EVQLVESGGGLVQPGGSLRLSCAASGFTWTGRYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 58 - protein, 118 aa, Artificial Sequence
  Synthetic polypeptide
EVQLVESGGGLVQPGGSLRLSCAASGLTVTGSYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 59 - protein, 118 aa, Artificial Sequence
  Synthetic polypeptide
EVQLVESGGGLVQPGGSLRLSCAASGFTVSPYYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 60 - protein, 118 aa, Artificial Sequence
  Synthetic polypeptide
EVQLVESGGGLVQPGGSLRLSCAASGDSLDTSYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 61 - protein, 118 aa, Artificial Sequence
  Synthetic polypeptide
EVQLVESGGGLVQPGGSLRLSCAASGDTDGTYYISWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 62 - protein, 118 aa, Artificial Sequence
  Synthetic polypeptide
EVQLVESGGGLVQPGGSLRLSCAASGFPVTGSYLSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 63 - protein, 118 aa, Artificial Sequence
  Synthetic polypeptide
EVQLVESGGGLVQPGGSLRLSCAASGFSDTGHYLSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 64 - protein, 118 aa, Artificial Sequence
  Synthetic polypeptide
EVQLVESGGGLVQPGGSLRLSCAASGISLNGYYLSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 65 - protein, 118 aa, Artificial Sequence
  Synthetic polypeptide
EVQLVESGGGLVQPGGSLRLSCAASGFTDYGNYLSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 66 - protein, 118 aa, Artificial Sequence
  Synthetic polypeptide
EVQLVESGGGLVQPGGSLRLSCAASGFPGTGSYMSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 67 - protein, 118 aa, Artificial Sequence
  Synthetic polypeptide
EVQLVESGGGLVQPGGSLRLSCAASGFPLTGSYMSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 68 - protein, 118 aa, Artificial Sequence
  Synthetic polypeptide
EVQLVESGGGLVQPGGSLRLSCAASGFSLIGSYMSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 69 - protein, 118 aa, Artificial Sequence
  Synthetic polypeptide
EVQLVESGGGLVQPGGSLRLSCAASGFTLSAHYMSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 70 - protein, 118 aa, Artificial Sequence
  Synthetic polypeptide
EVQLVESGGGLVQPGGSLRLSCAASSYTENGYYMSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 71 - protein, 118 aa, Artificial Sequence
  Synthetic polypeptide
EVQLVESGGGLVQPGGSLRLSCAASGFTEGGFYVSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 72 - protein, 118 aa, Artificial Sequence
  Synthetic polypeptide
EVQLVESGGGLVQPGGSLRLSCAASGFIEDSYYVSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 73 - protein, 118 aa, Artificial Sequence
  Synthetic polypeptide
EVQLVESGGGLVQPGGSLRLSCAASGFNGGTFYVSWVRQAPGKGLEWVGFIRDKANGYTTEYNPSVKGRFTISRDTSKNTLYLQMNSLRAEDTAVYYCAQVRRALDYWGQGTLVTVSS

SEQ ID NO: 74 - protein, 220 aa, Artificial Sequence
  Synthetic polypeptide
DIQMTQSPSSLSASVGDRVTITCKSSQSLLYSSNQNNYLAWYQQKPGKAPKLLIYWASTRESGVPSRFSGSGSGTDFTLTISSLQPEDFATYYCQQYYTYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 447 aa, Artificial Sequence
  Synthetic polypeptide
EVQLVESGGGLVQPGGSLRLSCAASGFTVTAYYMSWVRQAPGKGLEWVGFIRDKANGYTTEYNPSVKGRFTISRDTSKNTFYLQMNSLRAEDTAVYYCAQVRRALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 447 aa, Artificial Sequence
  Synthetic polypeptide
EVQLVESGGGLVQPGGSLRLSCAASGFTVTAYYMSWVRQAPGKGLEWVGFIRDKANGYTTEYNPSVKGRFTISRDTSKNTFYLQMNSLRAEDTAVYYCAQVRRALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHXHYTQKSLSLSPG

SEQ ID NO: 77 - protein, 108 aa, Mus musculus
DIVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPPFTFGSGT

SEQ ID NO: 78 - protein, 123 aa, Mus musculus
DVQLQESGPSLVKPSQTLSLTCSVTGDSITSGYWNWIRKFPGNKLEYMGYISYSGSTYYNPSLKSRISITRDTSKNQFYLQLLSVTTEDTATYYCAGLDGLYWYFDVWGAGTLVTVSSASTKG

SEQ ID NO: 79 - protein, 114 aa, Artificial Sequence
  Synthetic polypeptide
DIQMTQSPSSLSASVGDRVTITCRSSQSLVHSNGNTYLHWYQQKPGKAPKLLIYKVSNRFSGVPSRFSGSGSGTDFTLTISSLQPEDFATYYCSQSTHVPPFTFGQGTKVEIKR

SEQ ID NO: 80 - protein, 118 aa, Artificial Sequence
  Synthetic polypeptide
EVQLVESGGGLVQPGGSLRLSCAASGDSITSGYWNWVRQAPGKGLEWVGYISYSGSTYYNPSLKSRFTISADTSKNTAYLQMNSLRAEDTAVYYCAGLDGLYWYFDVWGQGTLVTVSS

SEQ ID NO: 81 - protein, 118 aa, Artificial Sequence
  Synthetic polypeptide
EVQLVESGGGLVQPGGSLRLSCAASGDSITSGYWNWVRQAPGKGLEWVGYISYSGSTYYNPSLKSRFTISRDTSKNTFYLQMNSLRAEDTAVYYCAGLDGLYWYFDVWGQGTLVTVSS

SEQ ID NO: 82 - protein, 118 aa, Artificial Sequence
  Synthetic polypeptide
EVQLVESGGGLVQPGGSLRLSCAASGDSINFGYWNWVRQAPGKGLEWVGYISYSGSTYYNPSLKSRFTISRDTSKNTFYLQMNSLRAEDTAVYYCAALNDLFLYFDVWGQGTLVTVSS

SEQ ID NO: 83 - protein, 118 aa, Artificial Sequence
  Synthetic polypeptide
EVQLVESGGGLVQPGGSLRLSCAASGDSITSGYWNWVRQAPGKGLEWVGYISYSGSTYYNPSLKSRFTISRDTSKNTFYLQMNSLRAEDTAVYYCAGLNDLYLYFDVWGQGTLVTVSS

SEQ ID NO: 84 - protein, 118 aa, Artificial Sequence
  Synthetic polypeptide
EVQLVESGGGLVQPGGSLRLSCAASGDSITSGYWNWVRQAPGKGLEWVGNISYSGSTYYNPSLKSRFTISRDTSKNTFYLQMNSLRAEDTAVYYCAGLDGLYWYFDVWGQGTLVTVSS

SEQ ID NO: 85 - protein, 118 aa, Artificial Sequence
  Synthetic polypeptide
EVQLVESGGGLVQPGGSLRLSCAASGDSITSGYWNWVRQAPGKGLEWVGNIGYSGSTYYNPSLKSRFTISRDTSKNTFYLQMNSLRAEDTAVYYCAGLDGLYWYFDVWGQGTLVTVSS

SEQ ID NO: 86 - protein, 214 aa, Artificial Sequence
  Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 232 aa, Artificial Sequence
  Synthetic polypeptide
EVQLVESGGGLVQPGGSLRLSCAASGFTISSNSIHWVRQAPGKGLEWVAWITPSDGNTDYADSVKGRFTISADTSKNTAYLQMNSLRAEDTAVYYCARRVCYSSVRGCAGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 88 - protein, 119 aa, Artificial Sequence
  Synthetic polypeptide
EVQLVESGGGLVQPGGSLRLSCAASGFTISGSSIHWVRQAPGKGLEWVGTIYPYGGNTYYADSVKGRFTISADTSKNTAYLQMNSLRAEDTAVYYCARRAFVMSGMDYWGQGTLVTVSS

SEQ ID NO: 89 - protein, 117 aa, Artificial Sequence
  Synthetic polypeptide
EVQLVESGGGLVQPGGSLRLSCAASGFTITGSYIHWVRQAPGKGLEWVAWIYPDGGYTDYADSVKGRFTISADTSKNTAYLQMNSLRAEDTAVYYCASKPAGPFGYWGQGTLVTVSS

SEQ ID NO: 90 - protein, 119 aa, Artificial Sequence
  Synthetic polypeptide
EVQLVESGGGLVQPGGSLRLSCAASGFTITGYGIHWVRQAPGKGLEWVAGITPANGYTDYADSVKGRFTISADTSKNTAYLQMNSLRAEDTAVYYCAKSFPFHYNFDYWGQGTLVTVSS

SEQ ID NO: 91 - protein, 120 aa, Artificial Sequence
  Synthetic polypeptide
EVQLVESGGGLVQPGGSLRLSCAASGFTINSSAIHWVRQAPGKGLEWVGYITPASGYTDYADSVKGRFTISADTSKNTAYLQMNSLRAEDTAVYYCAKGFHWYRGFFDYWGQGTLVTVSS

SEQ ID NO: 92 - protein, 116 aa, Artificial Sequence
  Synthetic polypeptide
EVQLVESGGGLVQPGGSLRLSCAASGFTITGSYIHWVRQAPGKGLEWVAWIYPDGGYTDYADSVKGRFTISADTSKNTAYLQMNSLRAEDTAVYYCASKPAGFGYWGQGTLVTVSS

SEQ ID NO: 93 - protein, 125 aa, Artificial Sequence
  Synthetic polypeptide
EVQLVESGGGLVQPGGSLRLSCAASGFTISSTGIHWVRQAPGKGLEWVGGISPSSGSTNYADSVKGRFTISADTSKNTAYLQMNSLRAEDTAVYYCARRKVVSSHVTNKYVMDYWGQGTLVTVSS

SEQ ID NO: 94 - protein, 121 aa, Artificial Sequence
  Synthetic polypeptide
EVQLVESGGGLVQPGGSLRLSCAASGFTINGSWIHWVRQAPGKGLEWVAWITPSNGSTDYADSVKGRFTISADTSKNTAYLQMNSLRAEDTAVYYCARLSRRPWLWGMDYWGQGTLVTVSS

SEQ ID NO: 95 - protein, 120 aa, Artificial Sequence
  Synthetic polypeptide
EVQLVESGGGLVQPGGSLRLSCAASGFTIXXXXIHWVRQAPGKGLEWVGWISPXXGNTXYADSVKGRFTISADTSKNTAYLQMNSLRAEDTAVYYCARXXXXXXAAMDYWGQGTLVTVSS

SEQ ID NO: 96 - protein, 120 aa, Artificial Sequence
  Synthetic polypeptide
EVQLVESGGGLVQPGGSLRLSCAASGFTIXXXXIHWVRQAPGKGLEWVGXISPXXGDTYYADSVKGRFTISADTSKNTAYLQMNSLRAEDTAVYYCARALCAPXXAMDYWGQGTLVTVSS

SEQ ID NO: 97 - protein, 108 aa, Artificial Sequence
  Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSRITPPTFGQGTKVEIKR

SEQ ID NO: 98 - protein, 125 aa, Artificial Sequence
  Synthetic polypeptide
EVQLVESGGGLVQPGGSLRLSCAASGFTISSNSIHWVRQAPGKGLEWVAWITPSDGNTDYADSVKGRFTISADTSKNTAYLQMNSLRAEDTAVYYCARRVCYNRLGVCAGGMDYWGQGTLVTVSS

SEQ ID NO: 99 - protein, 108 aa, Artificial Sequence
  Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQTSTSPPTFGQGTKVEIKR

SEQ ID NO: 100 - protein, 125 aa, Artificial Sequence
  Synthetic polypeptide
EVQLVESGGGLVQPGGSLRLSCAASGFTISSNSIHWVRQAPGKGLEWVAWITPSDGNTDYADSVKGRFTISADTSKNTAYLQMNSLRAEDTAVYYCARRVCYNNLGVCAGAMDYWGQGTLVTVSS

SEQ ID NO: 101 - protein, 108 aa, Artificial Sequence
  Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSYSTPPTFGQGTKVEIKR

SEQ ID NO: 102 - protein, 125 aa, Artificial Sequence
  Synthetic polypeptide
EVQLVESGGGLVQPGGSLRLSCAASGFTISSNSIHWVRQAPGKGLEWVAWITPSDGNTDYADSVKGRFTISADTSKNTAYLQMNSLRAEDTAVYYCARRVCYDRARVCAGAMDYWGQGTLVTVSS

SEQ ID NO: 103 - protein, 108 aa, Artificial Sequence
  Synthetic polypeptide
DIQMTQSPSSLSASVGDRVTITCRASQDVSTAVAWYQQKPGKAPKLLIYGASNLYSGVPSRFSGSGSGTDFTLTISSLQPEDFATYYCQQSHASPPTFGQGTKVEIKR

SEQ ID NO: 104 - protein, 125 aa, Artificial Sequence
  Synthetic polypeptide
EVQLVESGGGLVQPGGSLRLSCAASGFTISRRSIHWVRQAPGKGLEWVAWITPSDGNTDYADSVKGRFTISADTSKNTAYLQMNSLRAEDTAVYYCARRVCYSSVRGCAGAMDYWGQGTLVTVSS

SEQ ID NO: 105 - protein, 108 aa, Artificial Sequence
  Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSRISPPTFGQGTKVEIKR

SEQ ID NO: 106 - protein, 125 aa, Artificial Sequence
  Synthetic polypeptide
EVQLVESGGGLVQPGGSLRLSCAASGFTISSNSIHWVRQAPGKGLEWVAWVTPSGGSTDYADSVKGRFTISADTSKNTAYLQMNSLRAEDTAVYYCARRVCYNRLGVCAGGMDYWGQGTLVTVSS

SEQ ID NO: 107 - protein, 125 aa, Artificial Sequence
  Synthetic polypeptide
EVQLVESGGGLVQPGGSLRLSCAASGFTISSSSIHWVRQAPGKGLEWVAWITPGHGSTDYADSVKGRFTISADTSKNTAYLQMNSLRAEDTAVYYCARRVCYNRLGVCAGGMDYWGQGTLVTVSS

SEQ ID NO: 108 - protein, 108 aa, Artificial Sequence
  Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSHNTPPTFGQGTKVEIKR

SEQ ID NO: 109 - protein, 125 aa, Artificial Sequence
  Synthetic polypeptide
EVQLVESGGGLVQPGGSLRLSCAASGFSISSNSIHWVRQAPGKGLEWVAWITPTHGSTDYADSVKGRFTISADTSKNTAYLQMNSLRAEDTAVYYCARRVCYNRLGVCAGGMDYWGQGTLVTVSS

SEQ ID NO: 110 - protein, 125 aa, Artificial Sequence
  Synthetic polypeptide
EVQLVESGGGLVQPGGSLRLSCAASGFSIARSSIHWVRQAPGKGLEWVAWILPSAGSTDYADSVKGRFTISADTSKNTAYLQMNSLRAEDTAVYYCARRVCYNRLGVCAGGMDYWGQGTLVTVSS

SEQ ID NO: 111 - protein, 108 aa, Artificial Sequence
  Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSLITPPTFGQGTKVEIKR

SEQ ID NO: 112 - protein, 125 aa, Artificial Sequence
  Synthetic polypeptide
EVQLVESGGGLVQPGGSLRLSCAASGFSIRSISIHWVRQAPGKGLEWVAWITPFNGTTDYADSVKGRFTISADTSKNTAYLQMNSLRAEDTAVYYCARRVCYNRLGVCAGGMDYWGQGTLVTVSS

SEQ ID NO: 113 - protein, 108 aa, Artificial Sequence
  Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSRMSPPTFGQGTKVEIKR

SEQ ID NO: 114 - protein, 125 aa, Artificial Sequence
  Synthetic polypeptide
EVQLVESGGGLVQPGGSLRLSCAASGFTISSNSIHWVRQAPGKGLEWVAWITPSDGNTDYADSVKGRFTISADTSKNTAYLQMNSLRAEDTAVYYCARRVCYNHLGVCAGGMDYWGQGTLVTVSS

SEQ ID NO: 115 - protein, 108 aa, Artificial Sequence
  Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSRTTPPTFGQGTKVEIKR

SEQ ID NO: 116 - protein, 125 aa, Artificial Sequence
  Synthetic polypeptide
EVQLVESGGGLVQPGGSLRLSCAASGFSISNHSIHWVRQAPGKGLEWVAWVTPSYGITDYADSVKGRFTISADTSKNTAYLQMNSLRAEDTAVYYCARRVCYNRLGVCAGGMDYWGQGTLVTVSS

SEQ ID NO: 117 - protein, 108 aa, Artificial Sequence
  Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSLMTPPTFGQGTKVEIKR

SEQ ID NO: 118 - protein, 125 aa, Artificial Sequence
  Synthetic polypeptide
EVQLVESGGGLVQPGGSLRLSCAASGFTISSNSIHWVRQAPGKGLEWVAWVTPGVGSTDYADSVKGRFTISADTSKNTAYLQMNSLRAEDTAVYYCARRVCYNRLGVCAGGMDYWGQGTLVTVSS

SEQ ID NO: 119 - protein, 108 aa, Artificial Sequence; identical to SEQ ID NO: 105
  Synthetic polypeptide

SEQ ID NO: 120 - protein, 125 aa, Artificial Sequence
  Synthetic polypeptide
EVQLVESGGGLVQPGGSLRLSCAASGFSISRRSIHWVRQAPGKGLEWVAWITPLYGSTHYADSVKGRFTISADTSKNTAYLQMNSLRAEDTAVYYCARRVCYNRLGVCAGGMDYWGQGTLVTVSS

SEQ ID NO: 121 - protein, 108 aa, Artificial Sequence
  Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSGISPPTFGQGTKVEIKR

SEQ ID NO: 122 - protein, 125 aa, Artificial Sequence
  Synthetic polypeptide
EVQLVESGGGLVQPGGSLRLSCAASGFSIRNNSIHWVRQAPGKGLEWVAWVLPSNGVTDYADSVKGRFTISADTSKNTAYLQMNSLRAEDTAVYYCARRVCYNRLGVCAGGMDYWGQGTLVTVSS

SEQ ID NO: 123 - protein, 108 aa, Artificial Sequence
  Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSQISPPTFGQGTKVEIKR

SEQ ID NO: 124 - protein, 125 aa, Artificial Sequence
  Synthetic polypeptide
EVQLVESGGGLVQPGGSLRLSCAASGFTISNSSIHWVRQAPGKGLEWVAWVLPSVGFTDYADSVKGRFTISADTSKNTAYLQMNSLRAEDTAVYYCARRVCYNRLGVCAGGMDYWGQGTLVTVSS

SEQ ID NO: 125 - protein, 125 aa, Artificial Sequence
  Synthetic polypeptide
EVQLVESGGGLVQPGGSLRLSCAASGFTISASSIHWVRQAPGKGLEWVAWVLPSVGFTDYADSVKGRFTISADTSKNTAYLQMNSLRAEDTAVYYCARRVCYNRLGVCAGGMDYWGQGTLVTVSS

SEQ ID NO: 126 - protein, 125 aa, Artificial Sequence
  Synthetic polypeptide
EVQLVESGGGLVQPGGSLRLSCAASGFTISQSSIHWVRQAPGKGLEWVAWVLPSVGFTDYADSVKGRFTISADTSKNTAYLQMNSLRAEDTAVYYCARRVCYNRLGVCAGGMDYWGQGTLVTVSS

SEQ ID NO: 127 - protein, 125 aa, Artificial Sequence
  Synthetic polypeptide
EVQLVESGGGLVQPGGSLRLSCAASGFTISSSSIHWVRQAPGKGLEWVAWVLPSVGFTDYADSVKGRFTISADTSKNTAYLQMNSLRAEDTAVYYCARRVCYNRLGVCAGGMDYWGQGTLVTVSS

SEQ ID NO: 128 - protein, 214 aa, Artificial Sequence [sequence omitted]

SEQ ID NO: 129 - protein, 232 aa, Artificial Sequence [sequence omitted]

SEQ ID NO: 130 - protein, 454 aa, Artificial Sequence
  Synthetic polypeptide
EVQLVESGGGLVQPGGSLRLSCAASGFTISSSSIHWVRQAPGKGLEWVAWVLPSVGFTDYADSVKGRFTISADTSKNTAYLQMNSLRAEDTAVYYCARRVCYNRLGVCAGGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 131 - protein, 454 aa, Artificial Sequence
  Synthetic polypeptide
EVQLVESGGGLVQPGGSLRLSCAASGFTISSSSIHWVRQAPGKGLEWVAWVLPSVGFTDYADSVKGRFTISADTSKNTAYLQMNSLRAEDTAVYYCARRVCYNRLGVCAGGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHXHYTQKSLSLSPG

SEQ ID NO: 132 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 218 aa, Homo sapiens
PAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 217 aa, Artificial Sequence
  Synthetic polypeptide
PAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALXNHYTQKSLSLSPG

SEQ ID NO: 135 - protein, 218 aa, Homo sapiens
PAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 217 aa, Homo sapiens
PAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 137 - protein, 218 aa, Homo sapiens
PAPELLGGPSVFLFPPKPKDTLMISRTPEVTCVVVDVSHEDPEVQFKWYVDGVEVHNAKTKPREEQFNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 138 - protein, 218 aa, Homo sapiens
PAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWZSNGQPENNYKTTPPVLDSDGSFFLYSRLTVDKSRWQEGNVFSCSVMHEALHNHYTQKSLSLSLGK

SEQ ID NO: 139 - protein, 215 aa, Mus musculus
TVPEVSSVFIFPPKPKDVLTITLTPKVTCVVVDISKDDPEVQFSWFVDDVEVHTAQTQPREEQFNSTFRSVSELPIMHQDCLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 140 - protein, 218 aa, Mus musculus
PAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 141 - protein, 218 aa, Mus musculus
PAPNLEGGPSVFIFPPNIKDVLMISLTPKVTCVVVDVSEDDPDVQISWFVNNVEVHTAQTQTHREDYNSTIRVVSHLPIQHQDWMSGKEFKCKVNNKDLPSPIERTISKPKGLVRAPQVYTLPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 142 - protein, 217 aa, Mus musculus
PPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPG

SEQ ID NO: 143 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 144 - protein, 309 aa, Mus musculus
MDESAKTLPPPCLCFCSEKGEDMKVGYDPITPQKEEGAWFGICRDGRLLAATLLLALLSSSFTAMSLYQLAALQADLMNLRMELQSYRGSATPAAAGAPELTAGVKLLTPAAPRPHNSSRGHRNRRAFQGPEETEQDVDLSAPPAPCLPGCRHSQHDDNGMNLRNIIQDCLQLIADSDTPTIRKGTYTFVPWLLSFKRGNALEEKENKIVVRQTGYFFIYSQVLYTDPIFAMGHVIQRKKVHVFGDELSLVTLFRCIQNMPKTLPNNSCYSAGIARLEEGDEIQLAIPRENAQISRNGDDTFFGALKLL

SEQ ID NO: 145 - protein, 184 aa, Homo sapiens
MRRGPRSLRGRDAPAPTPCVPAECFDLLVRHCVACGLLRTPRPKPAGASSPAPRTALQPQESVGAGAGEAALPLPGLLFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 146 - protein, 185 aa, Homo sapiens
MRRGPRSLRGRDAPAPTPCVPAECFDLLVRHCVACGLLRTPRPKPAGAASSPAPRTALQPQESVGAGAGEAALPLPGLLFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 147 - protein, 175 aa, Mus musculus
MGARRLRVRSQRSRDSSVPTQCNQTECFDPLVRNCVSCELFHTPDTGHTSSLEPGTALQPQEGSALRPDVALLVGAPALLGLILALTLVGLVSLVSWRWRQQLRTASPDTSEGVQQESLENVFVPSSETPHASAPTWPPLKEDADSALPRHSVPVPATELGSTELVTTKTAGPEQ

SEQ ID NO: 148 - protein, 175 aa, Rattus norvegicus
MGVRRLRVRSRRSRDSPVSTQCNQTECFDPLVRNCVSCELFYTPETRHASSLEPGTALQPQEGSGLRPDVALLFGAPALLGLVLALTLVGLVSLVGWRWRQQRRTASLDTSEGVQQESLENVFVPPSETLHASAPNWPPFKEDADNILSCHSIPVPATELGSTELVTTKTAGPEQ

SEQ ID NO: 149 - protein, 183 aa, Macaca mulatta
MKRGPRSLRGRDAPAPTPCVPAECFDLLVRHCVACGLLRTPRPKPAAPASSPAPRTALQPQESVGAGAGEAALSLPGLLFGAPALLGLALVLALVLVGLVSWRRRQRRLRGASSAEAPDGDKDKDEPLDKVIILSPGISDAAAPAWPPPGEDPGTTPPGHSVPVPATELGSTELVTTKTAGPE

SEQ ID NO: 150 - protein, 23 aa, Artificial Sequence
  Synthetic polypeptide
TPCVPAECFDLLVRHCVACGLLR

SEQ ID NO: 151 - protein, 61 aa, Homo sapiens
MRRGPRSLRGRDAPAPTPCVPAECFDLLVRHCVACGLLRTPRPKPAGASSPAPRTALQPQE

SEQ ID NO: 152 - protein, 64 aa, Mus musculus
MGARRLRVRSQRSRDSSVPTQCNQTECFDPLVRNCVSCELFHTPDTGHTSSLEPGTALQPQEGS

SEQ ID NO: 153 - protein, 314 aa, Artificial Sequence
  Synthetic polypeptide
MSALLILALVGAAVASTGARRLRVRSQRSRDSSVPTQCNQTECFDPLVRNCVSCELFHTPDTGHTSSLEPGTALQPQEGQVTGDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 154 - DNA, 36 bp, Artificial Sequence
  Synthetic Construct
tcttgtgacaaaactcacagtggcggtggctctggt

SEQ ID NO: 155 - DNA, 48 bp, Artificial Sequence
  Synthetic Construct
tattactgtcagcaacattaataaaggccttaacctcccacgttcgga

SEQ ID NO: 156 - DNA, 55 bp, Artificial Sequence
  Synthetic Construct
acctgccgtgccagtcagrdtrktrvwanwthtgtagcctggtatcaacagaaac

SEQ ID NO: 157 - DNA, 55 bp, Artificial Sequence
  Synthetic Construct
acctgccgtgccagtcagrdtrktrvwanwthtctggcctggtatcaacagaaac

SEQ ID NO: 158 - DNA, 48 bp, Artificial Sequence
  Synthetic Construct
ccgaagcctctgatttackbggcatccavcctctactctggagtccct

SEQ ID NO: 159 - DNA, 54 bp, Artificial Sequence
  Synthetic Construct
ccgaagcttctgatttackbggcatccavcctcgmatctggagtcccttctcgc

SEQ ID NO: 160 - DNA, 57 bp, Artificial Sequence
  Synthetic Construct
gcaacttattactgtcagcaatmtdmcrvtnhtcctykgacgttcggacagggtacc

SEQ ID NO: 161 - DNA, 57 bp, Artificial Sequence
  Synthetic Construct
gcaacttattactgtcagcaatmtdmcrvtnhtccttwtacgttcggacagggtacc

SEQ ID NO: 162 - DNA, 57 bp, Artificial Sequence
  Synthetic Construct
gcaacttattactgtcagcaasrtdmcrvtnhtcctykgacgttcggacagggtacc

SEQ ID NO: 163 - DNA, 57 bp, Artificial Sequence
  Synthetic Construct
gcaacttattactgtcagcaasrtdmcrvtnhtccttwtacgttcggacagggtacc

SEQ ID NO: 164 - DNA, 57 bp, Artificial Sequence
  Synthetic Construct
gcaacttattactgtcagcaannnnnnnnnnnnccgnnnacgttcggacagggtacc

SEQ ID NO: 165 - DNA, 57 bp, Artificial Sequence
  Synthetic Construct
tgtgcagcttctggcttcwccnttnnnnnnnnnnnnnnnnnntgggtgcgtcaggcc

SEQ ID NO: 166 - DNA, 69 bp, Artificial Sequence
  Synthetic Construct
aagggcctggaatgggttgstnnnatcnnnnnnnnnnnnnnnnnnnnnnnntatgccgatagcgtcaag

SEQ ID NO: 167 - DNA, 79 bp, Artificial Sequence
  Synthetic Construct
gccgtctattattgtgctcgtnnnnnntgcnnnnnnnnnnnnnnnnnntgcnnnnnnnnnatggactactggggtcaag

SEQ ID NO: 168 - DNA, 79 bp, Artificial Sequence
  Synthetic Construct
gccgtctattattgtgctcgtnnnnnnnnnnnnnnnnnnnnnnnnnnnnnnnnnnnnnnnatggactactggggtcaag

SEQ ID NO: 169 - DNA, 63 bp, Artificial Sequence
  Synthetic Construct
gccgtctattattgtgctnnnnnnnnntgcnnnnnnnnnnnnnnnggctgcgcgggggcaatg

SEQ ID NO: 170 - DNA, 63 bp, Artificial Sequence
  Synthetic Construct
gctcgtcgggtctgctacnnnnnnnnnnnnnnntgcnnnnnnnnnatggactactggggtcaa

SEQ ID NO: 171 - DNA, 28 bp, Artificial Sequence
  Synthetic Construct
gctcggttgccgccgggcgttttttatg

SEQ ID NO: 172 - DNA, 51 bp, Artificial Sequence
  Synthetic Construct
acttattactgtcagcaannnnnnnnnnnnccgnnnacgttcggacagggt

SEQ ID NO: 173 - DNA, 54 bp, Artificial Sequence
  Synthetic Construct
acttattactgtcagcaannnnnnnnnnnnnnnccgnnnacgttcggacagggt

SEQ ID NO: 174 - DNA, 48 bp, Artificial Sequence
  Synthetic Construct
acttattactgtcagcaannknnknnkccgcccacgttcggacagggt

SEQ ID NO: 175 - DNA, 49 bp, Artificial Sequence
  Synthetic Construct
gcagcttctggcttcwccattnnnnnnnnnnnnatacactgggtgcgtc

SEQ ID NO: 176 - DNA, 63 bp, Artificial Sequence
  Synthetic Construct
ctggaatgggttgcttggrttnnncctnnnnnnggtnnnactnnntatgccgatagcgtcaag

SEQ ID NO: 177 - DNA, 60 bp, Artificial Sequence
  Synthetic Construct
gtctattattgtgctcgtnnnnnntgcnnnnnnnnnnnnnnnnnntgcgctggtgggatg

SEQ ID NO: 178 - DNA, 75 bp, Artificial Sequence
  Synthetic Construct
gtctattattgtgctcgtnnnnnntgcnnnnnnnnncttggtgtttgcnnnnnnnnnatggactactggggtcaa

SEQ ID NO: 179 - DNA, 72 bp, Artificial Sequence
  Synthetic Construct
gtctattattgtgctcgtnnnnnnrstnnnnnnnnnnnnnnnnnnrstgstgstgsgatggactactggggt

SEQ ID NO: 180 - DNA, 70 bp, Artificial Sequence
  Synthetic Construct
tattattgtgctcgtcggnnnrstnnnnnnnnnnnnnnnnnnrstnnnnnnnnnatggactactggggtc

SEQ ID NO: 181 - DNA, 55 bp, Artificial Sequence
  Synthetic Construct
acctgccgtgccagtsaagamrttkccasckctgtagcctggtatcaacagaaac

SEQ ID NO: 182 - DNA, 54 bp, Artificial Sequence
  Synthetic Construct
ccgaagcttctgatttwckccgcatcctwcctctwctctggagtcccttctcgc

SEQ ID NO: 183 - DNA, 57 bp, Artificial Sequence
  Synthetic Construct
gcaacttattactgtcagcaskccsaarttkccccgscaacgttcggacagggtacc

SEQ ID NO: 184 - DNA, 51 bp, Artificial Sequence
  Synthetic Construct
gcagcttctggcttcaccattkcckcckcckccatacactgggtgcgtcag

SEQ ID NO: 185 - DNA, 51 bp, Artificial Sequence
  Synthetic Construct
gcagcttctggcttcaccattagtkccagckccatacactgggtgcgtcag

SEQ ID NO: 186 - DNA, 51 bp, Artificial Sequence
  Synthetic Construct
gcagcttctggcttcaccattkccagckcctctatacactgggtgcgtcag

SEQ ID NO: 187 - DNA, 72 bp, Artificial Sequence
  Synthetic Construct
aagggcctggaatgggttgcatkgrttmtcscakccrttgsttwcascgamtatgccgatagcgtcaagggc

SEQ ID NO: 188 - DNA, 72 bp, Artificial Sequence
  Synthetic Construct
aagggcctggaatgggttgcttggrttcttscatctrttggttwcactgamtatgccgatagcgtcaagggc

SEQ ID NO: 189 - DNA, 72 bp, Artificial Sequence
  Synthetic Construct
aagggcctggaatgggttgcttkggttmtccctkccgtggsttttascgactatgccgatagcgtcaagggc

SEQ ID NO: 190 - DNA, 81 bp, Artificial Sequence
  Synthetic Construct
actgccgtctattattgtgcaaraarartttgctwcracaramtcgstrtttgckctgstgstatggactactggggtcaa

SEQ ID NO: 191 - DNA, 81 bp, Artificial Sequence
  Synthetic Construct
actgccgtctattattgtgctcgtaragtctgctwcaacaracttgstgtttgckctggtgstatggactactggggtcaa

SEQ ID NO: 192 - DNA, 81 bp, Artificial Sequence
  Synthetic Construct
actgccgtctattattgtgctaracggrtttgctacraccgcmtcggtrtttgcgctgstggtatggactactggggtcaa

SEQ ID NO: 193 - protein, 125 aa, Artificial Sequence
  Synthetic polypeptide
EVQLVESGGGLVQPGGSLRLSCAASGFTIASSSIHWVRQAPGKGLEWVAWVLPSVGFTDYADSVKGRFTISADTSKNTAYLQMNSLRAEDTAVYYCARRVCYNRLGVCAGGMDYWGQGTLVTVSS

SEQ ID NO: 194 - protein, 107 aa, Artificial Sequence
  Synthetic polypeptide
DIQMTQSPSSLSASVGDRVTITCRASEDIATSVAWYQQKPGKAPKLLIFAASFLYSGVPSRFSGSGSGTDFTLTISSLQPEDFATYYCQQSQISPPTFGQGTKVEIK

SEQ ID NO: 195 - protein, 107 aa, Artificial Sequence
  Synthetic polypeptide
DIQMTQSPSSLSASVGDRVTITCRASEDISSSVAWYQQKPGKAPKLLIFSASFLFSGVPSRFSGSGSGTDFTLTISSLQPEDFATYYCQQSEVSPPTFGQGTKVEIK

SEQ ID NO: 196 - protein, 107 aa, Artificial Sequence
  Synthetic polypeptide
DIQMTQSPSSLSASVGDRVTITCRASEDISTAVAWYQQKPGKAPKLLIFAASYLYSGVPSRFSGSGSGTDFTLTISSLQPEDFATYYCQQSQISPPTFGQGTKVEIK

SEQ ID NO: 197 - protein, 107 aa, Artificial Sequence
  Synthetic polypeptide
DIQMTQSPSSLSASVGDRVTITCRASEDISSAVAWYQQKPGKAPKLLIFAASYLFSGVPSRFSGSGSGTDFTLTISSLQPEDFATYYCQQSQVSPPTFGQGTKVEIK

SEQ ID NO: 198 - protein, 107 aa, Artificial Sequence
  Synthetic polypeptide
DIQMTQSPSSLSASVGDRVTITCRASEDISSAVAWYQQKPGKAPKLLIFAASFLYSGVPSRFSGSGSGTDFTLTISSLQPEDFATYYCQQSQISPPTFGQGTKVEIK

SEQ ID NO: 199 - protein, 107 aa, Artificial Sequence
  Synthetic polypeptide
DIQMTQSPSSLSASVGDRVTITCRASEEIATAVAWYQQKPGKAPKLLIFAASYLFSGVPSRFSGSGSGTDFTLTISSLQPEDFATYYCQQSQVAPPTFGQGTKVEIK

SEQ ID NO: 200 - protein, 107 aa, Artificial Sequence
  Synthetic polypeptide
DIQMTQSPSSLSASVGDRVTITCRASEEIATAVAWYQQKPGKAPKLLIFAASFLYSGVPSRFSGSGSGTDFTLTISSLQPEDFATYYCQQSEVSPPTFGQGTKVEIK

SEQ ID NO: 201 - protein, 107 aa, Artificial Sequence
  Synthetic polypeptide
DIQMTQSPSSLSASVGDRVTITCRASQDIATSVAWYQQKPGKAPKLLIFAASYLFSGVPSRFSGSGSGTDFTLTISSLQPEDFATYYCQQSQVSPPTFGQGTKVEIK

SEQ ID NO: 202 - protein, 107 aa, Artificial Sequence
  Synthetic polypeptide
DIQMTQSPSSLSASVGDRVTITCRASEEISTAVAWYQQKPGKAPKLLIFSASYLYSGVPSRFSGSGSGTDFTLTISSLQPEDFATYYCQQSEVSPPTFGQGTKVEIK

SEQ ID NO: 203 - protein, 107 aa, Artificial Sequence
  Synthetic polypeptide
DIQMTQSPSSLSASVGDRVTITCRASEDISSAVAWYQQKPGKAPKLLIFSASFLFSGVPSRFSGSGSGTDFTLTISSLQPEDFATYYCQQSQVSPPTFGQGTKVEIK

SEQ ID NO: 204 - protein, 107 aa, Artificial Sequence
  Synthetic polypeptide
DIQMTQSPSSLSASVGDRVTITCRASQDVATAVAWYQQKPGKAPKLLIFAASFLYSGVPSRFSGSGSGTDFTLTISSLQPEDFATYYCQQSQISPPTFGQGTKVEIK

SEQ ID NO: 205 - protein, 107 aa, Artificial Sequence
  Synthetic polypeptide
DIQMTQSPSSLSASVGDRVTITCRASEEIATSVAWYQQKPGKAPKLLIFSASYLYSGVPSRFSGSGSGTDFTLTISSLQPEDFATYYCQQSQVSPPTFGQGTKVEIK

SEQ ID NO: 206 - protein, 107 aa, Artificial Sequence
  Synthetic polypeptide
DIQMTQSPSSLSASVGDRVTITCRASEDISTAVAWYQQKPGKAPKLLIYAASFLYSGVPSRFSGSGSGTDFTLTISSLQPEDFATYYCQQSQISPPTFGQGTKVEIK

SEQ ID NO: 207 - protein, 107 aa, Artificial Sequence
  Synthetic polypeptide
DIQMTQSPSSLSASVGDRVTITCRASEDVSSAVAWYQQKPGKAPKLLIFAASYLFSGVPSRFSGSGSGTDFTLTISSLQPEDFATYYCQHSQVSPPTFGQGTKVEIK

SEQ ID NO: 208 - protein, 8 aa, Artificial Sequence
  Synthetic polypeptide
RDNSKNTL

SEQ ID NO: 209 - protein, 8 aa, Artificial Sequence
  Synthetic polypeptide
RDTSKNTA

SEQ ID NO: 210 - protein, 8 aa, Artificial Sequence
  Synthetic polypeptide
RDTSKNTF

SEQ ID NO: 211 - protein, 8 aa, Artificial Sequence
  Synthetic polypeptide
RDTSKNTL

SEQ ID NO: 212 - protein, 8 aa, Mus musculus
QVRRALDY

SEQ ID NO: 213 - protein, 20 aa, Mus musculus
GFIRDKANGYTTEYNPSVKG

SEQ ID NO: 214 - protein, 10 aa, Mus musculus
GFTVTAYYMS

SEQ ID NO: 215 - protein, 7 aa, Artificial Sequence
  Synthetic polypeptide
WAXXXXS

SEQ ID NO: 216 - protein, 10 aa, Artificial Sequence
  Synthetic polypeptide
XXXXXXXYXX

SEQ ID NO: 217 - protein, 10 aa, Artificial Sequence
  Synthetic polypeptide
XXXXXGAMDY

SEQ ID NO: 218 - protein, 10 aa, Artificial Sequence
  Synthetic polypeptide
XXXXXGXMDY

SEQ ID NO: 219 - protein, 10 aa, Artificial Sequence
  Synthetic polypeptide
NSNFYGAMDY

SEQ ID NO: 220 - protein, 16 aa, Artificial Sequence
  Synthetic polypeptide
RVCYNXLGVCAGGMDY

SEQ ID NO: 221 - protein, 16 aa, Artificial Sequence
  Synthetic polypeptide
RVCYNRLGVCAGGMDY

SEQ ID NO: 222 - protein, 10 aa, Artificial Sequence
  Synthetic polypeptide
GFTISSNSIH

SEQ ID NO: 223 - protein, 11 aa, Artificial Sequence
  Synthetic polypeptide
AWITPSDGNTD

SEQ ID NO: 224 - protein, 10 aa, Artificial Sequence
  Synthetic polypeptide
GFTISSSSIH

SEQ ID NO: 225 - protein, 11 aa, Artificial Sequence
  Synthetic polypeptide
AWVLPSVGFTD

SEQ ID NO: 226 - protein, 11 aa, Artificial Sequence
  Synthetic polypeptide
RASXXXXXXVA

SEQ ID NO: 227 - protein, 8 aa, Artificial Sequence
  Synthetic polypeptide
XXASXLXS

SEQ ID NO: 228 - protein, 9 aa, Artificial Sequence
  Synthetic polypeptide
QXSXXXPPT

SEQ ID NO: 229 - protein, 11 aa, Artificial Sequence
  Synthetic polypeptide
RASEDISTAVA

SEQ ID NO: 230 - protein, 8 aa, Artificial Sequence
  Synthetic polypeptide
YAASFLYS

SEQ ID NO: 231 - protein, 9 aa, Artificial Sequence
  Synthetic polypeptide
QQSQISPPT

SEQ ID NO: 232 - protein, 17 aa, Mus musculus
KSSQSLLYSSNQNNYLA

SEQ ID NO: 233 - protein, 7 aa, Mus musculus
WASTRES

SEQ ID NO: 234 - protein, 9 aa, Mus musculus
QQYYTYPYT

SEQ ID NO: 235 - protein, 10 aa, Mus musculus
TPHTYGAMDY

SEQ ID NO: 236 - protein, 17 aa, Mus musculus
KSSQSLLYSSNQNNYLA

(19) United States
(12) Patent Application Publication
(10) Pub. No.: US 2010/0166741 A1
(43) Pub. Date: Jul. 1, 2010

(54) ALTERED BR-3 BINDING POLYPEPTIDES

(75) Inventors: Robert F. Kelley, San Bruno, CA (US); Amy Yijuan Shen, San Mateo, CA (US); Dorothea Reilly, San Francisco, CA (US); Mark S. Dennis, San Carlos, CA (US); Germaine Fuh, Pacifica, CA (US); Chingwei V. Lee, Foster City, CA (US); Christine M. Ambrose, Reading, MA (US); Jeffrey S. Thompson, Stoneham, MA (US)

Correspondence Address: CLARK & ELBING LLP, 101 FEDERAL STREET, BOSTON, MA, 02110, US

(73) Assignees: Genentech , Inc., South San Francisco, CA (US); Biogen Idec Ma Inc., Cambridge, MA (US)

(21) Appl. No.: 12/373,662

(22) PCT Filed: Jul. 12, 2007
(86) PCT No.: PCT/US2007/015975

§ 371 Date: Dec. 4, 2009

Related U.S. Application Data
(60) Provisional application No. 60/830,969, filed on Jul. 13, 2006.

(51) Int. Cl. A61K 39/395; C12N 5/00; C12N 5/02; C12N 5/10; C12N 1/19; C12N 15/63; C07K 16/00; C07H 21/04; A61P 35/04

(52) U.S. Cl. 424/133.1; 435/325; 435/375; 435/419; 435/254.2; 435/320.1; 530/387.3; 530/391.3; 530/391.7; 536/23.1

(57) ABSTRACT

The present invention relates to novel BR3 binding antibodies having altered Fc effector function and/or having a mature core carbohydrate structure in the Fc region which lacks fiicose. The present invention also relates to the use of those BR3 binding antibodies and polypeptides in, e.g., methods of treatment, screening methods, diagnostic methods, assays and protein purification methods.

### FIELD OF THE INVENTION

The invention relates to novel compositions comprising antibodies that bind BR3, wherein the antibodies have altered Fc sequences and/or wherein the antibodies in the composition are underfucosylated, and uses thereof.

### BACKGROUND OF THE INVENTION

BAFF (also known as BLyS, TALL-1, THANK, TNFSF13B, or zTNF4) is a member of the TNF ligand superfamily that is essential for B cell survival and maturation (reviewed in Mackay & Browning (2002) Nature Rev. Immunol. 2, 465-475). BAFF overexpression in transgenic mice leads to B cell hyperplasia and development of severe autoimmune disease (Mackay, et al. (1999) J. Exp. Med. 190, 1697-1710; Gross, et al. (2000) Nature 404, 995-999; Clare, et al. (2000) Proc. Natl. Acad. Sci. U.S.A. 97, 3370-33752-4). BAFF levels are elevated in human patients with a variety of autoimmune disorders, such as systemic lupus erythematosus, rheumatoid arthritis, Wegener's granulomatosis and Sjögren's syndrome (Cheema, G. S, et al., (2001) Arthritis Rheum. 44, 1313-1319; Groom, J., et al. (2002) J. Clin. Invest. 109, 59-68; Zhang, J., et al., (2001) J. Immunol. 166, 6-10; Krumbholz et al., ANCA Workshop, Prague, Czech Republic, 2003). Furthermore, BAFF levels correlate with disease severity, suggesting that BAFF may play a direct role in the pathogenesis of these illnesses. BAFF blockade in animal models of collagen-induced arthritis (CIA), lupus (e.g., BWF1 mice), multiple sclerosis (e.g., experimental autoimmune encephalomyelitis (EAE)) resulted in an alleviation of the disease. BR3:Fc treatment in a chronic graft-versus-host disease (cGVHD) model significantly inhibited splenomegaly associated with cGVHD, not by preventing B cell activation, but by inhibiting B cell survival (Kalled, S L et al. (2005) Curr Dir Autoimmun. 8:206-42). Thus, it is likely that BAFF blockade will provide efficacy in other animal models of autoimmunity with a strong B cell component.

In addition, there have been reports that both CD4⁺ and CD8⁺T cells can be costimulated by recombinant BAFF to produce Type I and Type II cytokines and increase CD25 expression (Ng, L G, et al. 2004. J Immunol 173:807). Further, BAFF-R:Fc reportedly blocked BAFF-mediated human T cell proliferation (Huard, B, et al., (2000) J Immunol 167:6225). Still further, some patients with B-lymphoid malignancies have elevated levels of BAFF (Kern, C et al., (2004) Blood 103(2):679-88). According to one report, adding soluble BAFF or APRIL protected B-CLL cells against spontaneous and drug-induced apoptosis and stimulated NF-kappaB activation. Conversely, adding soluble BCMA-Fc or anti-BAFF and anti-APRIL antibodies enhanced B-CLL apoptosis (Kern, C et al., supra). BAFF may act as an essential autocrine survival factor for malignant B cells (Mackay F, et al., (2004) Curr Opin Pharmacol. 4(4):347-54). Thus, BAFF has been linked to a variety of disease states.

BAFF binds to three members of the TNF receptor superfamily, TACI, BCMA, and BR3 (also known as BAFF-R) (Gross, et al., supra; 8. Thompson, J. S., et al., (2001) Science 293, 2108-2111. Yan, M., et al.; (2001) Curr. Biol. 11, 1547-1552; Yan, M., et al., (2000) Nat. Immunol. 1, 37-41. Schiemann, B., et al., (2001) Science 293, 2111-2114). Of the three, only BR3 is specific for BAFF; the other two also bind the related TNF family member, APRIL. Comparison of the phenotypes of BAFF and receptor knockout or mutant mice indicates that signaling through BR3 mediates the B cell survival functions of BAFF (Thompson, et al., supra; Yan, (2002), supra; Schiemann, supra). In contrast, TACI appears to act as an inhibitory receptor (Yan, M., (2001) Nat. Immunol. 2, 638-643), while the role of BCMA is less clear (Schiemann, supra).

BR3 is a 184-residue type III transmembrane protein expressed on the surface of B cells (Thompson, et al., supra; Yan, (2002), supra). The intracellular region bears no sequence similarity to known structural domains or protein-protein interaction motifs. Several lines of investigation have provided strong evidence that BR3 is the primary receptor through which B cells receive a BAFF-mediated survival signal (reviewed in Kalled, S., et al., Curr Dir Autoimmun. 2005; 8:206-42). This has been confirmed by the recent generation of BAFF-R knockout mice (Shulga-Morkskaya, S. et al., (2004) J Immunol. 15; 173(4):2331-41). BR3 is expressed in a variety of disease tissue including multiple myeloma and non-Hodgkin's Lymphoma (Novak, A J (2004) Blood 104:2247-2253; Novak, A J (2004) Blood 103:689-694).

### SUMMARY OF THE INVENTION

The present invention provides anti-BR3 antibodies and anti-BR3 antibody compositions comprising novel antibody sequences (described generically and specifically below) having altered Fc sequences compared to a wild-type sequence. The anti-BR3 antibodies of the invention can be afucosylated (i.e., where the antibody includes a mature core carbohydrate structure which lacks fucose). A composition of anti-BR3 antibodies can include anywhere from 1 to 100% afucosylated antibodies. In preferred embodiments, a composition of anti-BR3 antibodies includes 2%, 4%, 6%, 10%, 19%, 20%, or anywhere up to 100% afucosylated antibodies. In one embodiment, the percent afucosylation includes only the measurement of the G0-F (also known as G0-Fuc) content. In another embodiment, the percent afucoslyation includes the measurement of the G0-F and the G1-F or G2-F content or both. In additional preferred embodiments, the invention includes an underfucosylated composition of anti-BR3 antibodies, where 20-100% of the N-linked oligosaccharide molecules released from the anti-BR3 antibody composition comprise a mature core carbohydrate structure which lacks fucose, attached to the Fc region of the antibody. Such compositions were demonstrated herein to exhibit an improvement in binding inter alia to FcγRIIIA(F158), which is not as effective as FcγRIIIA(V158) in interacting with human IgG.

Afucosylated antibodies or underfucosylated antibody compositions of this invention can be derived from a variety of methods, e.g., including expression of the antibodies from a cell line (e.g., CHO cells) having a gene involved in the fucosyl synthesis or transfer pathway removed from its genome (e.g., a FUT8 gene) or having such gene silenced by RNAi-mediated gene silencing or having such gene inhibited from expression using an inhibitor of such fucosyl synthase or transferase. Antibody compositions of this invention are useful as potent agents for therapeutic, diagnostic or research use. The present invention further provides variant BR3-binding polypeptides and compositions having altered Fc sequences useful for therapeutic diagnostic or research use. The present invention includes novel anti-BR3 antibody compositions, which compositions have potent ADCC activity against B cells while lacking neutrophil killing activity compared to other B cell depleting therapeutics, such as anti-CD20 antibodies.

According to one embodiment, the anti-BR3 binding antibodies of this invention comprise amino acid substitutions in their Fc region at 239 and 332 (EU numbering). According to another embodiment, the anti-BR3 binding polypeptides of this invention comprise amino acid substitutions in their Fc region at 239, 298 and 332 (EU numbering). According to another embodiment, the altered Fc sequence region comprises amino acid substitution(s) that increase ADCC activity relative to wild type sequence selected from the group consisting of (EU numbering): 268D, 326D, 333A/334A, 298A/333A, 298A/334A, 239D/332E, 239D/298A/332E, 239D/268D/298A/332E, 239D/268D/298A/326A/332A, 239D/268D/298A/326A/332E, 239D/268D/283L/298A/332E, 239D/268D/283L/298A/326A/332E and 239D/330L/332E of the Fc region. According to one specific embodiment, the anti-BR3 antibody further comprises altered Fc sequences that increase FcRn binding relative to a wild-type Fc sequence selected from the group consisting of N434A, N434F, N4343Y, N434W, N434H, 272Y/254T/256E and T250Q/M428L. According to another embodiment, the substitutions are to decrease ADCC activity such as D265A (in the absence of N297A) or N297A (in the absence of D265A). According to another embodiment, the substitutions are S298A/E333A/K334A or S298A/K326A/E333A/K334A. According to another embodiment, the substitution is K322A. According one preferred embodiment, the Fc variant increases the ADCC activity of the anti-BR3 antibody compared to 9.1RF. According to another embodiment, the Fc-mutations are as described elsewhere herein. According to one embodiment, the anti-BR3 binding antibody has an IgG Fc sequence of human IgG1.

According to another embodiment, the anti-BR3 antibodies of this invention comprise variant Fc sequences that increase ADCC activity and are underfucosylated. According to one embodiment, the underfucosylated compositions comprise anti-BR3 antibodies with variant Fc sequences at positions selected from the group consisting of 434, 298/326/333/334 and 239/332. In another embodiment the afucosylated anti-BR3 is Hu9.1RF which comprises the VH and VL sequences of SEQ ID NOs: 35 and 21, respectively (see Table 2).

According to another embodiment, the anti-BR3 antibodies include any one, any combination, or all of the following additional properties: (1) binds to a human BR3 extracellular domain sequence with an apparent Kd value of 500 nM or less, 100 nM or less, 50 nM or less, 10 nM or less, 5 nM or less or 1 nM or less; (2) binds to a human BR3 extracellular domain sequence and binds to a mouse BR3 extracellular domain sequence with an apparent Kd value of 500 nM or less, 100nM or less, 50 nM or less, 10 nM or less, 5 nM or less or 1 nM or less; (3) has a functional epitope on human BR3 comprising a specific residue(s); (4) inhibits the binding of human BR3 to human BAFF; (5) has increased ADCC in the presence of human effector cells compared to wild-type IgG; (6) is derived from any one of the antibodies disclosed herein; (7) binds the human Fc neonatal receptor (FcRn) with a higher affinity than a polypeptide or parent polypeptide having wild type or native sequence IgG Fc; and (8) kills or depletes B cells in vitro or in vivo, preferably by at least 20% when compared to the baseline level or appropriate negative control which is not treated with such antibody. BR3 binding polypeptides include peptides that bind BR3 (e.g., derived from phage display) that are fused to Fc domains (e.g., peptibodies).

In one embodiment, compared to treatment with a control antibody that does not bind a B cell surface antigen or as compared to the baseline level before treatment, an anti-BR3 antibody of this invention can deplete at least 20% of the B cells in any one, any combination or all of following population of cells in mice: (1) B cells in blood, (2) B cells in the lymph nodes, (3) follicular B cells in the spleen and (4) marginal zone B cells in the spleen. In other embodiments, B cell depletion is 25%, 30%, 40%, 50%, 60%, 70%, 80% or greater.

According to one embodiment, the anti-BR3 antibodies of this invention have a functional epitope on human BR3 comprising residues F25, V33 and A34, wherein the monoclonal antibody is not the 9.1 antibody or the 2.1 antibody. According to a further embodiment, the functional epitope further comprises residue R30. According to one embodiment, the BR3 binding antibodies of this invention have a functional epitope on human BR3 comprising residues P21 and A22. According to one embodiment, the BR3 binding antibodies of this invention have a functional epitope on human BR3 comprising residues L38 and R39, wherein the antibody is not the 9.1 antibody. According to another embodiment, the BR3 binding antibodies have a functional epitope on human BR3 comprising residue G36, wherein the antibody is not the 2.1 antibody. According to a further embodiment, the BR3 binding antibodies of this invention have a functional epitope on human BR3 comprising residues V29 and L28. According to yet another embodiment, the functional epitope further comprises L28 and V29. According to one embodiment, the anti-BR3 antibody that has a functional epitope on human BR3 that comprises any one, any combination or all of L38, R39, P21 and A22 is an antagonistic BR3 binding antibody.

The present invention includes anti-BR3 antibodies of Table 2, anti-BR3 antibodies derived from those antibodies and antibodies that bind BR3 and have an H1, H2, 1-13, L1, L2 or L3 region with at least 70% homology to any one of the underlined portions of the antibody sequences described in the Figures or to the CDRs or hypervariable regions described in the Sequence Listing. According to one embodiment, an antibody of this invention binds BR3 and has H1, H2 and H3 regions with at least 70% homology to the H1, H2 and H3 region, respectively, of any one of the antibodies of Table 2. According to one embodiment, an antibody of this invention binds BR3 and has L1, L2 and L3 regions with at least 70% homology to the L1, L2 and L3 region, respectively, of any one of the antibodies of Table 2. According to another embodiment, the antibodies bind BR3 and have a VH domain with at least 70% homology to a VH domain of any one of the antibodies of Table 2.

The present invention provides humanized anti-BR3 antibodies comprising an H3 hypervariable region (HVR3) comprising the residues QVRRALDY (SEQ ID NO:212). According to another embodiment, an anti-BR3 antibody comprises: (1) an H3 hypervariable region (HVR3) comprising the residues QVRRALDY (SEQ ID NO:212); and (2) a heavy chain framework 3 region (HC-FR3) comprising the residues RDTSKNTF (SEQ ID NO:210). In one embodiment, the BR3 binding antibody further comprises an HVR1 comprising residues numbered 26-35 and an HVR2 comprising residues 49-65 (Kabat numbering) of an antibody sequence of any one of SEQ ID NOs: 35-36. In another embodiment, the anti-BR3 antibody further comprises residues GFTVTAYYMS (SEQ ID NO:214) in the H1 hypervariable region (HVR1) and residues GFIRDKANGYTTEYNPSVKG (SEQ ID NO: 213) in the H2 hypervariable region (HVR2). According to one embodiment, the antibody further comprises residues KSSQSLLYSSNQNNYLA (SEQ ID NO:232) in the LVR1, residues WASTRES (SEQ ID NO:233) in the LVR2 and residues QQSQISPPT (SEQ ID NO:231) in the LVR3.

According to another embodiment, an anti-BR3 binding antibody of the invention comprises: (1) an H3 hypervariable region (HVR3) comprising QVRRALDY (SEQ ID NO:212); and (2) a heavy chain framework 3 region (HC-FR3) comprising RDTSKNTL (SEQ ID NO:211). In one embodiment, the BR3 binding antibody comprises residues numbered 26-35 and 49-65 (Kabat numbering) of any one of the antibody sequences of SEQ ID NOs:37-73. According to one embodiment, the antibody further comprises residues KSSQSLLYSSNQNNYLA (SEQ ID NO:232) in the LVR1, residues WASTRES (SEQ ID NO:233) in the LVR2 and residues QQSQISPPT (SEQ ID NO:231) in the LVR3.

According to another embodiment, an anti-BR3 binding antibody of the invention comprises an L2 hypervariable region (LVR2) comprising Formula I:

W-A-X3-X3-X4-X3X5-X6-S (SEQ ID NO:215) (Formula I),

wherein X3 is Q or S; X4 is H, I or T; X5 is L or R and X6 is D or E and wherein Formula I is not WASTRES (SEQ ID NO:233). According to one embodiment, the anti-BR3 antibody further comprises an H3 hypervariable region (HVR3) comprising QVRRALDY (SEQ ID NO:212). According to one embodiment, the LVR2 comprises residues numbered 50-56 (Kabat numbering) of the antibody sequence selected from the group consisting of SEQ ID NOs:23 and 25. According to another embodiment, the antibody further comprises residues GFTVTAYYMS (SEQ ID NO:214) in the HVR1 and residues GFIRDKANGYTTEYNPSVKG (SEQ ID NO:213) in the HVR2. According to yet another embodiment, the antibody further comprises residues KSSQSLLYSSNQNNYLA (SEQ ID NO:232) in the LVR1 and residues QQSQISPPT (SEQ ID NO:231) in the LVR3.

According to another embodiment, an anti-BR3 binding antibody of the invention comprises: an H1 hypervariable region (HVR1) comprising Formula II:

X1-X2-X3-X4-X5-X6-X7-Y-X9-X10 (SEQ ID NO:216) (Formula II),

wherein X1 is G or D, S, A, V, E or T; X2 is L, S, W, P, F, A, V, I, R, Y or D; X3 is P, T, A, N, S, I, K, L or Q; X4 is M, R, V, Y, G, E, A, T, L, W or D; X5 is A, S, T, G, I, R, P, N, D, Y or H; X6 is G, A, S, P or T; X7 is F, H, Y, R, S, V or N; X9 is T, I, M, F, W or V; X10 is T, G, S or A and wherein Formula II is not GFTVTAYYMS (SEQ ID NO:214). According to one embodiment, the antibody further comprises an H3 hypervariable region (HVR3) comprising QVRRALDY (SEQ ID NO:212). According to one embodiment, the HVR1 comprises residues numbered 26-35 (Kabat numbering) of the antibody sequence selected from the group consisting of SEQ ID NOs:24, 26-34, 36 and 38-73. According to one embodiment, the antibody further comprises residues WASTRES (SEQ ID NO:233) in the LVR2. According to one embodiment, the antibody further comprises residues KSSQSLLYSSNQNNYLA (SEQ ID NO:232) in the LVR1, residues WASTRES (SEQ ID NO:233) in the LVR2 and residues QQSQISPPT (SEQ ID NO:231) in the LVR3. According to one embodiment, the antibody further comprises residues GFIRDKANGYTTEYNPSVKG (SEQ ID NO:213) in the HVR2.

According to another embodiment, an anti-BR3 binding antibody of this invention is an antibody that comprises: (1) an H3 hypervariable region (HVR3) comprising QVRRALDY (SEQ ID, NO:212) and (2) residues numbered 50-56 of the LVR2 and residues numbered 26-35 of the HVR1 of an antibody selected from the group consisting of Hu9.1-73, Hu9.1-70, Hu9.1-56, Hu9.1-51, Hu9.1-59, Hu9.1-61, Hu9.1-A, Hu9.1-B and Hu9.1-C. According to one embodiment, the antibody further comprises residues GFIRDKANGYTTEYNPSVKG (SEQ ID NO:213) in the HVR2. According to one embodiment, the antibody further comprises residues KSSQSLLYSSNQNNYLA (SEQ ID NO:232) in the LVR1 and residues QQSQISPPT (SEQ ID NO:231) in the LVR3.

The present invention also provides anti-BR3 antibodies comprising an HVR3 comprising residues numbered 94-102 (Kabat numbering) of the antibody sequence selected from the group consisting of SEQ ID NOS:7-13 and 16-18. According to one embodiment, the antibody further comprises an HVR1 and HVR2 comprising residues 26-35 and residues 49-65 (Kabat numbering), respectively, of the antibody sequence of any one of SEQ ID NOS:7-13 and 16-18. According to one embodiment, the LVR1, LVR2 and LVR3 of the antibody comprises residues 24-34, residues 50-56 and residues 89-97 (Kabat numbering), respectively, of the antibody sequence of SEQ ID NO:3.

According to one embodiment, the anti-BR3 comprises a variable heavy chain domain comprising the variable heavy chain sequence of any one of SEQ ID NOs:22, 24 and 26-73. According to one embodiment, the anti-BR3 comprises a variable light chain domain comprising the variable light chain sequence of any one of SEQ ID NOs:21, 23 and 25. According to another embodiment, the antibody comprises the sequence of SEQ ID NO:74. According to another embodiment, the antibody comprises the sequence of SEQ ID NO:76, wherein X is N, A, W, H, Y, S or F. According to one specific embodiment, the antibody comprises the sequence of SEQ ID NO:75.

The present invention also provides an anti-BR3 antibody comprising an HVR3 comprising Formula III:

X1-X2-X3-X4-X5-G-X7-MDY (SEQ ID NO:218) (Formula III),

wherein X1 is N, T or R; X2 is A, S, T, L, N or P; X3 is N, H or L; X4 is P, Y, F, N, T or L; X5 is Y, T or D; and X7 is A or E. According to one embodiment, Formula III is not TPHTYGAMDY (SEQ ID NO:235). According to one embodiment, Formula III is NSNFYGAMDY (SEQ ID NO:219). According to one embodiment, the antibody further comprises an HC-FR3 comprising residues RDTSKNTF (SEQ ID NO:210) or RDTSKNTL (SEQ ID NO:211). According to one embodiment, the LVR1, LVR2 and LVR3 of the antibody comprise residues 24-34, residues 50-56 and residues 89-97 (Kabat numbering), respectively, of the antibody sequence of SEQ ID NO:3. According to one embodiment, the HVR1 and HVR2 of the antibody comprise residues 26-35 and residues 49-65 (Kabat numbering), respectively, of the antibody sequence of SEQ ID NO:4.

Alternatively, the present invention provides an anti-BR3 antibody comprising an HVR3 comprising Formula III:

X1-X2-X3-X4-X5-G-X7-MDY (SEQ ID NO:218) (Formula III),

wherein X1 is N, T or R; X2 is A, S, T, L, N or P; X3 is N, 1-1 or L; X4 is P, Y, F, N, T or L; X5 is Y, T or D; and X7 is A or E and wherein the antibody further comprises an HC-FR3 comprising residues RDTSKNTF (SEQ ID NO:210) or RDTSKNTL (SEQ ID NO:211). According to one embodiment, when the HC-FR3 comprises RDTSKNTF (SEQ ID NO:210), then HVR3 of the antibody comprises residues 94-102 (Kabat numbering) of the antibody sequence of any one of SEQ ID NOs:6-9 and 16-17. According to one embodiment, when the HC-FR3 comprises RDTSKNTL (SEQ ID NO:211), then the HVR3 of the antibody comprises residues 94-102 (Kabat numbering) of the antibody sequence of any one of SEQ ID NOs:5 and 10-13. According to one embodiment, the LVR1, LVR2 and LVR3 of the antibody comprise residues 24-34, residues 50-56 and residues 89-97 (Kabat numbering), respectively, of the antibody sequence of SEQ ID NO:3. According to one embodiment, the HVR1 and HVR2 of the antibody comprise residues 26-35 and residues 49-65 (Kabat numbering) of the antibody sequence of SEQ ID NO:4, respectively.

In one embodiment, the sequence of Formula III is Formula IV:

X1-X2-X3-X4-X5-GAMDY (SEQ ID NO:217) (Formula IV),

wherein X1 is N, T or R; X2 is S, T, L, N or P; X3 is N or L; X4 is P, Y, F, N or L; X5 is Y or D.

According to one embodiment, the anti-BR3 antibody of the invention comprises an HVR3 comprising the sequence of Formula IV and an HC-FR3 comprising the sequence of SEQ ID NO:210. In a further embodiment, the antibody comprises the light chain sequence of SEQ ID NO:14. In a further embodiment, the antibody comprises an Fc region having D265A/N297A (EU numbering) mutations or another Fc mutation that decreases ADCC activity of the antibody.

According to one embodiment, the anti-BR3 antibody of the invention comprises a variable heavy chain domain comprising the variable heavy chain sequence of any one of SEQ ID NOs:4-13 and 16-18. According to one embodiment, the anti-BR3 comprises a variable light chain domain comprising the variable light chain sequence of SEQ ID NO:3. According to another embodiment, the antibody comprises the sequence of SEQ ID NO:14. According to another embodiment, the antibody comprises the sequence of SEQ ID NO:15.

The present invention further provides an anti-BR3 antibody of the invention comprises the variable light chain sequence SEQ ID NO:77 and the variable heavy chain sequence SEQ ID NO:78, and variants thereof. According to one embodiment, an anti-BR3 antibody comprises the variable light chain sequence of SEQ ID NO:79. According to another embodiment, an anti-BR3 antibody comprises the variable heavy chain sequence of any one of SEQ ID NOs:80-85. According to one embodiment, an anti-BR3 antibody comprises an HVR1 comprising residues numbered 26-35 (Kabat numbering) of the antibody sequence of any one of SEQ ID NOs:80 or 82. According to one embodiment, an anti-BR3 antibody comprises an HVR2 comprising residues numbered 49-65 (Kabat numbering) of the antibody sequence of any one of SEQ ID NOs:80, 84 or 85. According to one embodiment, an anti-BR3 antibody comprises an HVR3 comprising residues numbered 94-102 (Kabat numbering) of the antibody sequence of any one of SEQ ID NOs:80, 82 or 83. In another embodiment, the anti-BR3 antibody comprises (1) an HVR3 comprising residues 94-102 (Kabat numbering) of the antibody sequence of any one of SEQ ID NOs: 81-85 and (2) a heavy chain framework 3 region (HC-FR3) comprising RDTSKNTF (SEQ ID NO:210). According to one embodiment, an anti-BR3 antibody comprises residues numbered 26-35, 49-65 and 94-102 of the antibody sequence of any one of SEQ ID NOs:80-85. According to one embodiment, the anti-BR3 antibody comprises an LVR1 comprising residues numbered 24-34 (Kabat numbering) of the antibody sequence SEQ ID NO:79. According to one embodiment, the anti-BR3 antibody comprises an LVR2 comprising residues numbered 50-56 (Kabat numbering) of the antibody sequence SEQ ID NO:79. According to one embodiment, the anti-BR3 antibody comprises an LVR3 comprising residues numbered 89-97 (Kabat numbering) of the antibody sequence SEQ ID NO:79. According to another embodiment, the LVR1, LVR2 and LVR3 of an anti-BR3 antibody comprises residues numbered 24-34, 50-56 and 89-97 (Kabat numbering), respectively, of SEQ ID NO:79.

According to one embodiment, the anti-BR3 comprises a variable heavy chain domain comprising the variable heavy chain sequence of any one of SEQ ID NOs:78 and 80-85. According to one embodiment, the anti-BR3 antibody comprises a variable light chain domain comprising the variable light chain sequence of SEQ ID NO:77 and 79.

The present invention also provides an anti-BR3 antibody comprising an HVR3 comprising residues numbered 95-102 of the antibody sequence of any one of SEQ ID NOs:87-94. The present invention provides an anti-BR3 antibody comprising an HVR2 comprising residues numbered 49-58 of the antibody sequence of any one of SEQ ID NOs:87-94, 98, 100, 102, 104, 106, 107, 109-110, 112, 114, 116, 118, 120, 122, 124-127, 129 and 193. The present invention provides an anti-BR3 antibody comprising an HVR1 comprising residues numbered 24-34 of the antibody sequence of any one of SEQ ID NOs:87-94, 98, 100, 102, 104, 106, 107, 109-110, 112, 114, 116, 118, 120, 122, 124-127, 129 and 193. The present invention provides an anti-BR3 antibody comprising an LVR1 comprising residues numbered 24-34 of the antibody sequence of any one of SEQ ID NOs:86, 97, 99, 101, 103, 105, 108, 111, 113, 115, 117, 119, 121, 123, 128 and 194-207. The present invention provides an anti-BR3 antibody comprising an LVR2 comprising residues numbered 50-56 of the antibody sequence of any one of SEQ ID NOs:86, 97, 99, 101, 103, 105, 108, 111, 113, 115, 117, 119, 121, 123, 128 and 194-207. The present invention provides an anti-BR3 antibody comprising an LVR3 comprising residues numbered 89-97 of the antibody sequence of any one of SEQ ID NOs:86, 97, 99, 101, 103, 105, 108, 111, 113, 115, 117, 119, 121, 123, 128 and 194-207. According to one embodiment, the LVR1, LVR2 and LVR3 comprise residues numbered 24-34, 50-56 and 89-97 of the antibody sequence of any one of SEQ ID NOs:86, 97, 99, 101, 103, 105, 108, 111, 113, 115, 117, 119, 121, 123, 128 and 194-207. According to one embodiment, the HVR1, HVR2 and HVR3 comprise residues numbered 24-34, 49-58 and 95-102 of the antibody sequence of any one of SEQ ID NOs:87-94, 98, 100, 102, 104, 106, 107, 109-110, 112, 114, 116, 118, 120, 122, 124-127, 129 and 193. In one embodiment, the anti-BR3 antibody comprises a variable heavy chain domain comprising the VH sequence of any one of SEQ ID NOs:87-94, 98, 100, 102, 104, 106, 107, 109-110, 112, 114, 116, 118, 120, 122, 124-127, 129 and 193. In another embodiment, the anti-BR3 antibody comprises a variable light chain domain comprising the VL sequence of any one of SEQ ID NOs:86, 97, 99, 101, 103, 105, 108, 111, 113, 115, 117, 119, 121, 123, 128 and 194-207.

The present invention further provides an anti-BR3 antibody comprising HVR3 comprising RVCYN-X6-LGVCAGGMDY (SEQ ID NO:220) (Formula V), wherein X6 is R or H.

The present invention provides an anti-BR3 antibody comprising an LVR1 comprising the Formula VI:

RAS-X4-X5-X6-X7-X8-X9-VA (Formula VI) (SEQ ID NO: 226),

wherein X4 is Q or E; X5 is D or E; X6 is I or E; X7 is S or A, X8 is S or T and X9 is A or S.

The present invention provides an anti-BR3 antibody comprising an LVR2 comprising the Formula VII:

X1-X2-A-S—X5-L-X7-S (Formula VII) (SEQ ID NO: 227),

### Wherein X1 is Y or F; X2 is S, A or G; X5 is N, F or Y; and X7 is F or Y.

The present invention provides an anti-BR3 antibody comprising an LVR3 comprising the Formula VIII:

Q-X2-S—X4-X5-X6-PPT (Formula VIII) (SEQ ID NO: 228),

wherein X2 is Q or H; X4 is G, L, R, H, Y, Q or E; X5 is N, T, M, S, A, T, I or V; and X6 is T or S. According to one embodiment, the anti-BR3 antibody comprises a light chain comprising the sequences of Formula I, II and III. According to another embodiment, the anti-BR3 antibody comprises a light chain comprising the sequences of Formula I, II and III and comprises a HVR3 comprising the sequence of Formula V or SEQ ID NO:220.

The present invention also provides an anti-BR3 binding antibody that comprises an H3 comprising RVCYNRLGVCAGGMDY (SEQ ID NO:221); an H1 comprising residues SGFTISSNSIH (SEQ ID NO:222) and an H2 comprising AWITPSDGNTD (SEQ ID NO: 223). In another embodiment, the anti-BR3 binding antibody comprises an H3 comprising RVCYNRLGVCAGGMDY (SEQ ID NO:221); an H1 comprising residues SGFTISSSSIH (SEQ ID NO:224) and an H2 comprising AWVLPSVGFTD (SEQ ID NO: 225).

According to one embodiment, the anti-BR3 comprises a variable heavy chain comprising the variable heavy chain sequence of any one of SEQ ID NOs:87-96, 98, 100, 102, 104, 106, 107, 109-110, 112, 114, 116, 118, 120, 122, 124-127, 129 and 193. According to one embodiment, the anti-BR3 antibody comprises a variable light chain comprising the variable light chain sequence of any one of SEQ ID NOs:86, 97, 99, 101, 103, 105, 108, 111, 113, 115, 117, 119, 121, 123, 128 and 194-207.

In one embodiment, the anti-BR3 antibody can competitively inhibit the binding of an antibody produced by the hybridoma deposited as 3.1 (ATCC Deposit PTA-6622) or 12B12.1 (ATCC Deposit PTA-6624) to the human BR3 extracellular domain. In a further embodiment, the antibody comprises the variable region sequence of the antibody produced by the hybridoma deposited as 3.1 (ATCC Deposit PTA-6622) or 12B12.1 (ATCC Deposit PTA-6624) to the human BR3 extracellular domain. In another embodiment, the antibody comprises the hypervariable region sequence of the antibody produced by the hybridoma deposited as 3.1 (ATCC Deposit PTA-6622) or 12B12.1 (ATCC Deposit PTA-6624). In another embodiment, the antibody is a humanized form of the antibody produced by the hybridoma deposited as 3.1 (ATCC Deposit PTA-6622) or 12B12.1 (ATCC Deposit PTA-6624).

In one embodiment, the anti-BR3 antibody can competitively inhibit the binding of an antibody produced by the hybridoma deposited as 3.1 (ATCC Deposit PTA-6622) or 12B12.1 (ATCC Deposit PTA-6624) to human BR3. In a further embodiment, the antibody comprises the variable region sequence of the antibody produced by the hybridoma deposited as 3.1 (ATCC Deposit PTA-6622) or 12B12.1 (ATCC Deposit PTA-6624) to human BR3. In another embodiment, the antibody comprises the hypervariable region sequence of the antibody produced by the hybridoma deposited as 3.1 (ATCC Deposit PTA-6622) or 12B12.1 (ATCC Deposit PTA-6624). In another embodiment, antibody is a humanized form of the antibody produced by the hybridoma deposited as 3.1 (ATCC Deposit PTA-6622) or 12B12.1 (ATCC Deposit PTA-6624).

In one embodiment, the antibody of this invention binds to the same epitope as any one of the antibodies specifically described herein. In another embodiment, the antibody of this invention comprises the sequence of the deposited antibodies.

According to one embodiment of the invention, the anti-BR3 antibody is conjugated to a cytotoxic agent or a chemotherapeutic agent.

According to another embodiment, the antibody is a monoclonal antibody. According to another embodiment, the antibody is a humanized antibody. According to another embodiment, the antibody is a human antibody. According to another embodiment, the antibody is a chimeric antibody. According to another embodiment, the antibody is selected from the group consisting of a Fab, Fab′, a F(ab)′₂, single-chain Fv (scFv), an Fv fragment; a antiabody and a linear antibody. According to another embodiment, the antibody is a multi-specific antibody such as a bispecific antibody.

Also provided is a composition comprising an antibody of any one of the preceding embodiments, and a carrier. In one embodiment, the carrier is a pharmaceutically acceptable carrier. These compositions can be provided in an article of manufacture or a kit.

The invention also provided a liquid formulation comprising an anti-BR3 antibody in a histidine buffer. According to one embodiment, the buffer is a histidine sulfate buffer. According to another embodiment, a formulation or composition of this invention is packaged as a pre-filled syringe.

The invention also provides an isolated nucleic acid that encodes any of the antibody sequences disclosed herein, including an expression vector for expressing the antibody.

Another aspect of the invention are host cells comprising the preceding nucleic acids, and host cells that produce the antibody. In one preferred embodiment of the latter, the host cell is a CHO cell. A method of producing these antibodies is provided, the method comprising culturing the host cell that produces the antibody and recovering the antibody from the cell culture.

Yet another aspect of the invention is an article of manufacture comprising a container and a composition contained therein and a package insert, wherein the composition comprises an antibody of any of the preceding embodiments. According to one embodiment, the article of manufacture is a diagnostic kit comprising a BR3-binding antibody of this invention.

The invention also provides methods of treating the diseases disclosed herein by administration of an anti-BR3 antibody of the invention, polypeptide or functional fragment thereof, to a mammal such as a human patient having a bone marrow transplant and a human patient suffering from the disease such as an autoimmune disease, a cancer, a B cell neoplasm, a BR3 positive cancer or an immunodeficiency disease. According to one preferred embodiment for treating an autoimmune disease, B cell neoplasm or a BR3 positive cancer, the BR3 binding polypeptide or antibody to be administered is preferably an antagonist BR3-binding antibody or polypeptide or is not an agonist BR3 binding antibody or polypeptide. According to one embodiment, the cancer to be treated according to this invention is selected from the group consisting of non-Hodgkin's lymphoma, chronic lymphocytic leukemia, multiple myeloma, (including follicular lymphoma, diffuse large B cell lymphoma, marginal zone lymphoma and mantle cell lymphoma).

In one embodiment of the methods for treating an autoimmune disease, cancer, B cell neoplasm or a BR3 positive cancer, the antibody is a BR3-binding antibody that has increased ability to bind FcRn at pH 6.0 compared to a 9.1RF antibody of this invention. In one embodiment of the methods for treating an autoimmune disease, B cell neoplasm or a BR3 positive cancer, the BR3 binding antibody is a BR3-binding antibody that has increased ADCC effector function in the presence of human effector cells compared to a 9.1RF antibody.

In one embodiment, the BR3 positive cancer is a B cell lymphoma or leukemia including non-Hodgkin's lymphoma (NHL) or lymphocyte predominant Hodgkin's disease (LPHD), chronic lymphocytic leukemia (CLL), acute lymphocytic leukemia (ALL) or small lymphocytic lymphoma (SLL). According to another embodiment, the BR3 positive cancer is multiple myeloma. In additional embodiments, the treatment method further comprises administering to the patient at least one chemotherapeutic agent, wherein for non-Hodgkin's lymphoma (NHL), the chemotherapeutic agent is selected from the group consisting of doxorubicin, cyclophosphamide, vincristine and prednisolone.

Also provided is a method of treating an autoimmune disease, comprising administering to a patient suffering from the autoimmune disease, a therapeutically effective amount of a BR3 binding antibody or polypeptide of this invention. According to one embodiment, the autoimmune disease is selected from the group consisting of rheumatoid arthritis, juvenile rheumatoid arthritis, lupus including systemic lupus erythematosus (SLE), Wegener's disease, inflammatory bowel disease including Crohn's Disease and ulcerative colitis, idiopathic thrombocytopenic purpura (ITP), thrombotic thrombocytopenic purpura (TTP), autoimmune thrombocytopenia, multiple sclerosis, psoriasis, Ig neuropathies including IgA nephropathy, IgM polyneuropathies, and IgG neuropathy, myasthenia gravis, vasculitis including ANCA-associated vasculitis, diabetes mellitus, Reynaud's syndrome, Sjorgen's syndrome, neuromyelitis optica (NMO), pemphigus including paraneoplastic pemphigus, pemphigus vulgaris and pemphigus foliaceus, polymyositis/dermatomyositis and glomerulonephritis. Where the autoimmune disease is rheumatoid arthritis, the antibody can be administered in conjunction with a second therapeutic agent. According to one embodiment, the second therapeutic agent is methotrexate.

In these treatment methods for autoimmune diseases, B cell neoplasms, BR3 positive cancers, the BR3 binding antibodies can be administered alone or in conjunction with a second therapeutic agent such as a second antibody, another B cell depleting agent, a chemotherapeutic agent, an immunosuppressive agent or another biologic that modulates human immune responses (e.g., a biologic response modifier). The second antibody can be one that binds CD20 or a different B cell antigen, or an NK or T cell antigen. In one embodiment, the anti-CD20 antibody is selected from the group consisting of rituximab (Rituxan®), m2H7 (murine 2H7), hu2H7 (humanized 2H7) and all its functional variants, hu2H7.v16 (v stands for version), v31, v96, v114 and v115, (e.g., see, WO 2004/056312). In one embodiment, the second antibody is a radiolabeled anti-CD20 antibody. In other embodiments, the CD20 binding antibody is conjugated to a cytotoxic agent including a toxin or a radioactive isotope. In another embodiment, the second therapeutic agent is selected from the group consisting of an interleukin (e.g., IL-2, IL-12), an interferon, fludarabine, cyclophosphamide, an antibody that targets TNF-alpha (e.g., Enbrel®, Remicade®, and Humira®), or a colony-stimulating factor (e.g., CSF, GM-CSF, G-CSF). In another embodiment, the second antibody or biologic can be another BAFF antagonist (e.g., a BR3 antibody, anti-BAFF antibody, TACI-Fc, BCMA-Fc and BR3-Fc). According to one embodiment, the BAFF antagonist that is being administered as a second therapeutic for autoimmune diseases or cancer does not have ADCC activity. In another embodiment, the second therapeutic is selected from the group consisting of an anti-VEGF antibody (e.g., the Avastin™ antibody), anti-CD64 antibody, an anti-C32a antibody, an anti-CD16 antibody, anti-INFalpha antibody, anti-CD79a antibody, an anti-CD70b antibody, an anti-CD52 antibody, anti-CD40 antibody, CTLA4-Ig, anti-CD22 antibody, anti-CD23 antibody, anti-CD80 antibody, anti-HLA-DR antibody, anti-MHCII (IA) antibody, anti-IL-7 antibody, anti-IL-2 antibody, anti-IL-4 antibody, an anti-IL-21 antibody and anti-IL-10 antibody. Specific examples of B cell depletion agents include, but are not limited to, the aforementioned anti-CD20 antibodies, Alemtuzumab (anti-CD52 antibody), and Epratuzumab or CMC-544 (Wyeth) (anti-CD22 antibodies). In another embodiment, the second therapeutic is a small molecule that depletes B cells or an IAP inhibitor.

In another aspect, the invention provides a method of treating an autoimmune disease selected from the group consisting of Dermatomyositis, Wegner's granulomatosis, ANCA-associated vasculitis (AAV), Aplastic anemia, Autoimmune hemolytic anemia (AIHA), factor VIII deficiency, hemophilia A, Autoimmune neutropenia, Castleman's syndrome, Goodpasture's syndrome, solid organ transplant rejection, graft versus host disease (GVHD), IgM mediated, thrombotic thrombocytopenic purpura (TTP), Hashimoto's Thyroiditis, autoimmune hepatitis, lymphoid interstitial pneumonitis (LIP), bronchiolitis obliterans (non-transplant) vs. NSIP, Guillain-Barre Syndrome, large vessel vasculitis, giant cell (Takayasu's) arteritis, medium vessel vasculitis, Kawasaki's Disease, polyarteritis nodosa, comprising administering to a patient suffering from the disease, a therapeutically effective amount of a BR3 binding antibody.

The present invention also provides a method for diagnosing an autoimmune disease or a cancer to be treated with a BR3 binding therapy antagonist which comprises: (a) contacting a biological sample from a test subject with an anti-BR3 antibody of this invention; (b) assaying the level of BR3 polypeptide in the biological sample; and (c) comparing the level of BR3 polypeptide in the biological sample in the biological sample with a standard level of BR3 protein; whereby the presence or an increase in the level of BR3 protein compared to the standard level of BR3 protein is indicative of an autoimmune disease or cancer to be treated with a BR3 binding therapy.

The present invention also provides a method of detecting BR3 polypeptide comprising the steps of binding the anti-BR3 antibody or immunoadhesin of this invention in a test sample or a subject and comparing the antibody or immunoadhesin bound compared to a control antibody or immunoadhesin. In one embodiment, the antibody or immunoadhesin is used in an assay selected from the group consisting of a FACS analysis, an immunohistochemistry assay (IHC) and an ELISA assay. Non-BAFF blocking anti-BR3 antibodies have the advantage of detecting BR3 whether it is bound to ligand or not and can be useful in measuring free and bound BR3.

### BRIEF DESCRIPTION OF THE DRAWINGS

FIG. 1 graphically depicts the results of ADCC assays with 9.1RF Fc variants and BJAB cells.

FIGS. 2A and 2B graphically depict the results of ADCC assays with 9.1 RF Fc variants and (A) BJAB or (B) WIL2-S cells.

FIG. 3 graphically depicts fold change in binding of V3-46s Fc variants to mouse or human FcγR relative to a control.

FIGS. 4A and 4B graphically depict the ADCC activity of V3-46s Fc variants on BJAB cells.

FIG. 5 is a schematic describing the experimental design of B cell depletion assays in mice using V3-46s Fc variants.

FIG. 6 graphically depicts the results of blood B cell depletion after treatment with V3-46s Fc variants.

FIG. 7 shows examples of oligosaccharide structures present on IgG, their numerical assignments in m/z via MALDI-TOF analyses.

FIG. 8 is a schematic of the configuration of the RNAi plasmids. Abbreviations: CMV, cytomegalovirus promoter and enhancer sequence; PD, PUR-DHFR; HC, heavy chain; LC, light chain.

FIG. 9 shows the RNAi probes developed to decrease fucosyltransferase (FUT8) in cells.

FIG. 10 graphically depicts the percentage of non-fucosylated antibody in samples of 9.1RF or 9.1(5) Fc variant produced from cells transiently transfected with or without RNAi2 and RNAi4.

FIG. 11 graphically depicts the ADCC activity of 9.1RF Fc variants expressed from cells transiently transfected with RNAi2 and RNAi4 or not.

FIGS. 12A and 12B graphically depict the fold increased binding of 9.1 RF Fc variants to (12A) human or (12B) cyno FcγRIIIa relative to a control antibody (the Herceptin® antibody).

FIGS. 13A and 13B graphically depict the direct and competitive binding of control and afucosylated anti-BR3 mAbs to BJAB cells. FIG. 13A graphically depicts the direct binding assay where the indicated concentrations of mAbs were added to BJAB cells and bound mAb was detected with anti-human IgG Fc-HRP. FIG. 13B graphically depicts the competitive binding assay where the indicated concentrations of mAbs were added to cells in the presence of 25 nM FLAG-BAFF followed by detection of bound BAFF using anti-FLAG-HRP.

FIGS. 14A and 14B graphically depict the evaluation of the antagonistic effects of anti-BR3 (Hu9.1 RF) and control antibodies on primary human B cell proliferation. B cells were incubated with a dilution curve of anti-BR3 mAbs in the presence of anti-IgM (4 μg/ml) and BAFF (20 ng/ml). In FIG. 14A, the control and afucosylated anti-BR3 antibodies caused an identical dose-dependent inhibition of B cell proliferation. In FIG. 14B, neither the agonistic positive control (“Control”-Hu9.1RF with an N434W mutation) nor the isotype negative control (Herceptin®) antibodies inhibited B cell proliferation.

FIGS. 15A and 15B graphically depict the evaluation of potential agonistic effects of anti-BR3 and control antibodies. B cells were incubated with a dilution curve of antibodies in the presence of anti-IgM (4 μg/ml) alone. In FIG. 15A, neither the control nor the afucosylated (AF) Hu9.1RF IgG1 antibody stimulated B cell proliferation above the level elicited by treatment with anti-IgM. In FIG. 15B, the IgG1 isotype control had no effect, whereas Hu9.1RF IgG1 with an N434W mutation (“Control”) caused a dose-dependent increase in B cell proliferation.

FIG. 16 graphically depicts the results of an ELISA showing the binding of antibodies to human FcRN at pH 6.0. Various concentrations of control and test material were incubated with human FcRn immobilized on the assay plate at pH 6.0. All data points were collected in duplicate, and the mean absorbance values were plotted versus the antibody concentration

FIG. 17 graphically depicts the results of an ELISA shown the dissociation of bound IgG at pH 6.0 or 7.4. Various concentrations of control and test material were incubated with human FcRn immobilized on the assay plate at pH 6.0. The dissociation of bound IgG from FcRn was evaluated after incubation in pH 6.0 or 7.4 assay buffer. All data points were collected in singlet, and the absorbance values were plotted versus the antibody concentration.

FIGS. 18A and 18B graphically depict the CDC activity of AF HU9.1RF IgG1 negative control and positive control monoclonal antibodies on BJAB and WIL2 cells.

FIGS. 19A-19C graphically depict the ADCC activity of AF HU9.1RF IgG1 with 2% afucosylated HU9.1RF IgG1 using normal human NK cells from three normal donors.

FIGS. 20A-20C graphically depict the ADCC activity of HU9.1RF IgG1 monoclonal antibodies differing in percent afucosylation using normal human NK cells from three normal donors.

FIGS. 21A-21C graphically depict the induction of apoptosis by AF Hu9.1RF IgG1 in BR3 positive BJAB cells. AF Hu9.1RF IgG1 at concentrations ranging from 0.1 to 100 nM in the presence of anti IgG crosslinker did not affect the viability of the cells (FIG. 21A), the level of annexin staining (FIG. 21B), or the level of propidium iodide staining (FIG. 21C) as compared to untreated cells.

### DETAILED DESCRIPTION OF THE INVENTION

The carbohydrate moieties of the present invention will be described with reference to commonly used nomenclature for the description of oligosaccharides. A review of carbohydrate chemistry which uses this nomenclature is found in Hubbard et al. Ann. Rev. Biochem. 50:555-583 (1981). This nomenclature includes, for instance, Man, which represents mannose; GlcNAc, which represents 2-N-acetylglucosamine; Gal which represents galactose; Fuc for fucose; and Glc, which represents glucose. Sialic acids are described by the shorthand notation NeuNAc, for 5-N-acetylneuraminic acid, and NeuNGc for 5-glycolylneuraminic.

The term “glycosylation” means the attachment of oligosaccharides (carbohydrates containing two or more simple sugars linked together e.g. from two to about twelve simple sugars linked together) to a protein to form a glycoprotein. The oligosaccharide side chains are typically linked to the backbone of the glycoprotein through either N- or O-linkages. The oligosaccharides of the present invention occur generally are attached to a CH2 domain of an Fc region as N-linked oligosaccharides.

“N-linked glycosylation” refers to the attachment of the carbohydrate moiety to an asparagine residue in a glycoprotein chain. The skilled artisan will recognize that, for example, each of murine IgG1, IgG2a, IgG2b and IgG3 as well as human IgG1, IgG2, IgG3, IgG4, IgA and IgD CH2 domains have a single site for N-linked glycosylation at amino acid residue 297 (Kabat et al. Sequences of Proteins of Immunological Interest, 1991).

“Glycoproteins” are polypeptides having one or more oligosaccharide side chains attached thereto.

For the purposes herein, a “mature core carbohydrate structure” refers to a processed core carbohydrate structure attached to an Fc region which generally comprises the following carbohydrate structure GlcNAc(Fucose)-GlcNAc-Man-(Man-GleNAc)₂ typical of biantennary oligosaccharides represented schematically below:

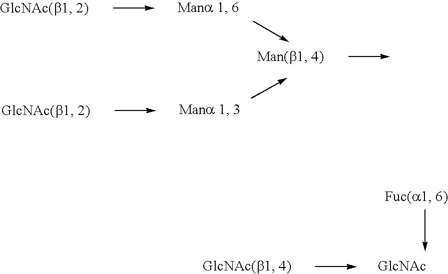

However, this term specifically includes G-1 forms of the core mature carbohydrate structure lacking a β1,2 GlcNAc residue. Preferably, however, the core carbohydrate structure includes both β1,2 GlcNAc residues. The mature core carbohydrate structure herein generally is not hypermannosylated.

The mature core carbohydrate structure is attached to the Fc region of the glycoprotein, generally via N-linkage to Asn297 of a CH2 domain of the Fc region.

The term “underfucosylated” means that 20-100% of the N-linked oligosaccharide molecules released from the Fc region of the anti-BR3 antibody composition comprise a mature core carbohydrate structure which lacks fucose (“non-fucosylated”), attached to the Fc region of the antibody. Thus, it should be understood that the term underfucosylated includes anti-BR3 antibody compositions having N-linked oligosaccharides with no fucose entirely. According to one preferred embodiment, the percentage of non-fucosylated oligosaccharides obtained from the compositions is determined by using MALDI-TOF analysis (e.g., see Table 6, below). According to one embodiment, the percentage of non-fucosylated oligosaccharides is determined by measuring the percentage of G0-Fuc (m/z 2000) released from the anti-BR3 antibody composition. According to another embodiment, the underfucosylated composition is selected from the group consisting of 21-100%, 22-100%, 23-100%, 24-100%, 25-100%, 26-100%, 27-100%, 28-100%, 29-100%, 30-100%, 31-100%, 32-100%, 33-100%, 34-100%, 35-100%, 36-100%, 37-100%, 38-100%, 39-100%, 40-100%, 41-100%, 42-100%, 43-100%, 44-100%, 45-100%, 46-100%, 47-100%, 48-100%, 49-100%, 50-100%, 51-100%, 52-100%, 53-100%, 54-100%, 55-100%, 55-100%, 56-100%, 57-100%, 58-100%, 59-100%, 60-100%, 61-100%, 62-100%, 63-100%, 64-100%, 65-100%, 66-100%, 67-100%, 68-100%, 69-100%, 70-100%, 71-100%, 72-100%, 73-100%, 74-100%, 75-100%, 76-100%, 77-100%, 78-100%, 79-100%, 80-100%, 81-100%, 82-100%, 83-100%, 84-100%, 85-100%, 86-100%, 87-100%, 88-100%, 89-100%, 90-100%, 91-100%, 92-100%, 93-100%, 94-100%, 95-100%, 96-100%, 97-100%, 98-100% and 99-100% of the N-linked oligosaccharide molecules released from the Fc region of the anti-BR3 antibody composition comprise a mature core carbohydrate structure which lacks fucose.

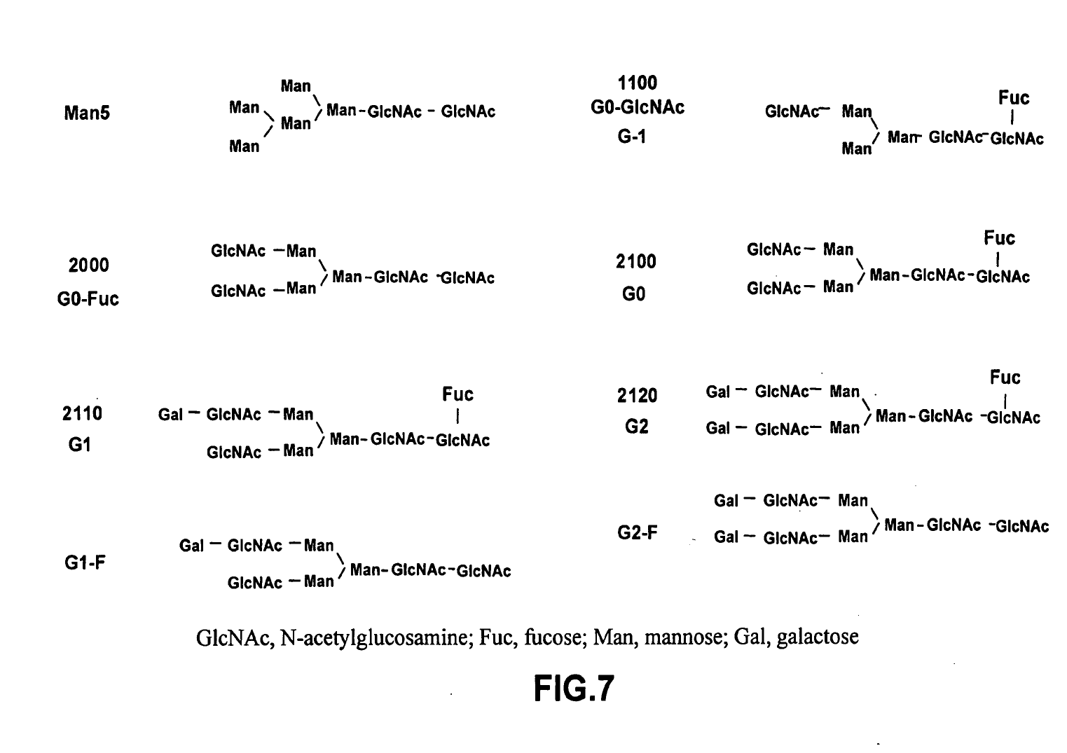

By “afucosylated” (AF) or “non-fucosylated” is meant the state of a molecule wherein the molecule includes a mature core carbohydrate structure which lacks a fucose. Examples of an afucosylated state include G0-F (also known as G0-Fuc), G1-F, or G2-F (see FIG. 7). For example, an afucosylated antibody may have a fucose lacking from a carbohydrate on one heavy chain or both heavy chains of the antibody molecule. Methods for determining the fucosylation state of a molecule include capillary electrophoresis (see for example, Ragu, Analytical Biochemistry 283:125-132 (2000)) and mass spectrometry (e.g., MALDI-TOF). A composition can include antibodies that are anywhere from 0 to 100% afucosylated. In one embodiment, a composition can include from 1%, 2%, 3%, 4%, 5%, 6%, 7%, 8%, 9%, 10%, 11%, 12%, 13%, 14%, 15%, 16%, 17%, 18%, or 19% of the antibodies in an afucosylated state. In another embodiment, a composition can include up to 100% of the antibodies in an afucosylated state.

A “bisecting GlcNAc” is a GlcNAc residue attached to the β1,4 mannose of the mature core carbohydrate structure. The bisecting GlcNAc can be enzymatically attached to the mature core carbohydrate structure by a β(1,4)-N-acetylglucosaminyltransferase III enzyme (GnTIII). CHO cells do not normally express GnTIII (Stanley et al. J. Biol. Chem. 261:13370-13378 (1984)), but may be engineered to do so (Umana et al. Nature Biotech. 17:176-180 (1999)).

A glycoprotein that is “essentially free” of one or more selected sugar groups (e.g. bisecting GlcNAc, one or more galactose residues, or one or more sialic acid residues) is generally produced in a host cell that is defective in the addition of the selected sugar group(s) to the mature core carbohydrate structure, such that about 90-100% of the glycoprotein in a composition will lack the selected sugar group(s) attached to the mature core carbohydrate structure.

A “glycosidase” is an enzyme involved in the biosynthesis of asparagine-linked (N-linked) glycoproteins. A “trimming” enzyme is one which removes oligosaccharide(s), whereas a “transferase” adds oligosaccharide(s). Examples of glycosidases include trimming glucosidases such as glucosidase I and glucosidase II; trimming mannosidases such as rough endoplasmic reticulum mannosidase (rER mannosidase), mannosidase IA, mannosidase IB and mannosidase II; as well as transferases such as glycosyl transferases, e.g. β(1,4)-N-acetylglucosaminyltransferase III (GnT III), Gal-transferases, sialic-acid-transferases and fuc-transferases.

A “glycosidase inhibitor” refers to a compound or composition which reduces or prevents N-linked oligosaccharide processing by one or more glycosidase(s). Examples include, nojirimycin, 1-deoxynojirimycin (dNM), N-Methyl-1-deoxy-nojirimycin (M-dNM), castanospermine, bromoconduritol, 1-deoxymannojirimycin (dMM), australine, MDL, lentiginosine, and Swainsonine (Sw). Glycosidase inhibitors are reviewed in Fuhrmann et al. Biochim. Biophys. Acta 825:95-110 (1985); Kaushal and Elbein, Methods in Enzym. 230:316-329 (1994); and Elbein, A. FASEB 5:3055-3063 (1991).

“Lec13” refers to the lectin-resistant Chinese Hamster Ovary (CHO) mutant cell line which displays a defective fucose metabolism and therefore has a diminished ability to add fucose to complex carbohydrates. That cell line is described in Ripka and Stanley, Somatic Cell & Molec. Gen. 12(1):51-62 (1986); and Ripka et al. Arch. Biochem. Biophys. 249(2):533-545 (1986) and is available from the Albert Einstein College of Medicine of Yeshiva University, Bronx, N.Y. Lec13 cells are believed lack the transcript for GDP-D-mannose-4,6-dehydratase, a key enzyme for fucose metabolism. Ohyama et al. J. Biol. Chem. 273(23):14582-14587 (1988). GDP-D-mannose-4,6-dehydratase generates GDP-mannose-4-keto-6-D-deoxymannose from GDP-mannose, which is then converted by the FX protein to GDP-L-fucose. Expression of fucosylated oligosaccharides is dependent on the GDP-L-fucose donor substrates and fucosyltransferase(s).

A “fucosyltransferase” is an enzyme that adds one or more fucose(s) to a glycoprotein. Examples include α1,6-fucosyltransferase, FucTI, FucTII, FucTIII, FucTIV, FucTV, FucTVI and FucTVII. Porcine and human α1,6-fucosyltransferases are described in Uozumi et al. J. Biol. Chem. 271:27810-27817 (1996), and Yanagidani et al. J. Biochem. 121:626-632 (1997), respectively.

A “sialyltransferase” is an enzyme that adds one or more sialic acid residue(s) to a glycoprotein. An α2,3 sialytransferase can add sialic acid residue(s) to galactose residue(s) attached to a mature core carbohydrate structure.

A “galactotransferase” is an enzyme that adds one or more galactose residue(s) to a glycoprotein. A β1,4-galactosyltransferase can add galactose residue(s) to the mature core carbohydrate structure.

The term “Fc region-containing glycoprotein” refers to a glycoprotein, such as an antibody or immunoadhesin, which comprises an Fc region.

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The Fc region of an immunoglobulin generally comprises two constant domains, CH2 and CH3.

The terms “BAFF,” “BAFF polypeptide,” “TALL-1,” “TALL-1 polypeptide,” or “BLyS” when used herein encompass “native sequence BAFF polypeptides” and “BAFF variants”. “BAFF” is a designation given to those polypeptides which are encoded by any one of the amino acid sequences of SEQ ID NO:143 or SEQ ID NO:144 and homologs and fragments and variants thereof, which have the biological activity of the native sequence BAFF. A biological activity of BAFF can be selected from the group consisting of promoting B cell survival, promoting B cell maturation and binding to BR3, BCMA or TACI. Variants of BAFF will preferably have at least 80% or any successive integer up to 100% including, more preferably, at least 90%, and even more preferably, at least 95% amino acid sequence identity with a native sequence of a BAFF polypeptide. A “native sequence” BAFF polypeptide comprises a polypeptide having the same amino acid sequence as the corresponding BAFF polypeptide derived from nature. For example, BAFF exists in a soluble form following cleavage from the cell surface by furin-type proteases. Such native sequence BAFF polypeptides can be isolated from nature or can be produced by recombinant and/or synthetic means. The term “native sequence BAFF polypeptide” specifically encompasses naturally-occurring truncated or secreted forms (e.g., an extracellular domain sequence), naturally-occurring variant forms (e.g., alternatively spliced forms) and naturally-occurring allelic variants of the polypeptide. The term “BAFF” includes those polypeptides described in Shu et al., J. Leukocyte Biol., 65:680 (1999); GenBank Accession No. AF136293; WO98/18921 published May 7, 1998; EP 869,180 published Oct. 7, 1998; WO98/27114 published Jun. 25, 1998; WO99/12964 published Mar. 18, 1999; WO99/33980 published Jul. 8, 1999; Moore et al., Science, 285:260-263 (1999); Schneider et al., J. Exp. Med., 189:1747-1756 (1999); Mukhopadhyay et al., J. Biol. Chem., 274:15978-15981 (1999).

The term “BAFF antagonist” as used herein is used in the broadest sense, and includes any molecule that (1) binds a native sequence BAFF polypeptide or binds a native sequence BR3 polypeptide to partially or fully block BR3 interaction with BAFF polypeptide, and (2) partially or fully blocks, inhibits, or neutralizes native sequence BAFF signaling. Native sequence BAFF polypeptide signaling promotes, among other things, B cell survival and B cell maturation. The inhibition, blockage or neutralization of BAFF signaling results in, among other things, a reduction in the number of B cells. A BAFF antagonist according to this invention will partially or fully block, inhibit, or neutralize one or more biological activities of a BAFF polypeptide, in vitro or in vivo. In one embodiment, a biologically active BAFF potentiates any one or any combination of the following events in vitro or in vivo: an increased survival of B cells, an increased level of IgG and/or IgM production, or stimulated B cell proliferation.

The term “TACI antagonist” as used herein is used in the broadest sense, and includes any molecule that (1) binds a native sequence BAFF polypeptide or binds a native sequence TACI polypeptide to partially or fully block TACI interaction with BAFF polypeptide, and (2) partially or fully blocks, inhibits, or neutralizes native sequence BAFF signaling.

The term “BCMA antagonist” as used herein is used in the broadest sense, and includes any molecule that (1) binds a native sequence BAFF polypeptide or binds a native sequence BCMA polypeptide to partially or fully block BCMA interaction with BAFF polypeptide, and (2) partially or fully blocks, inhibits, or neutralizes native sequence BAFF signaling.

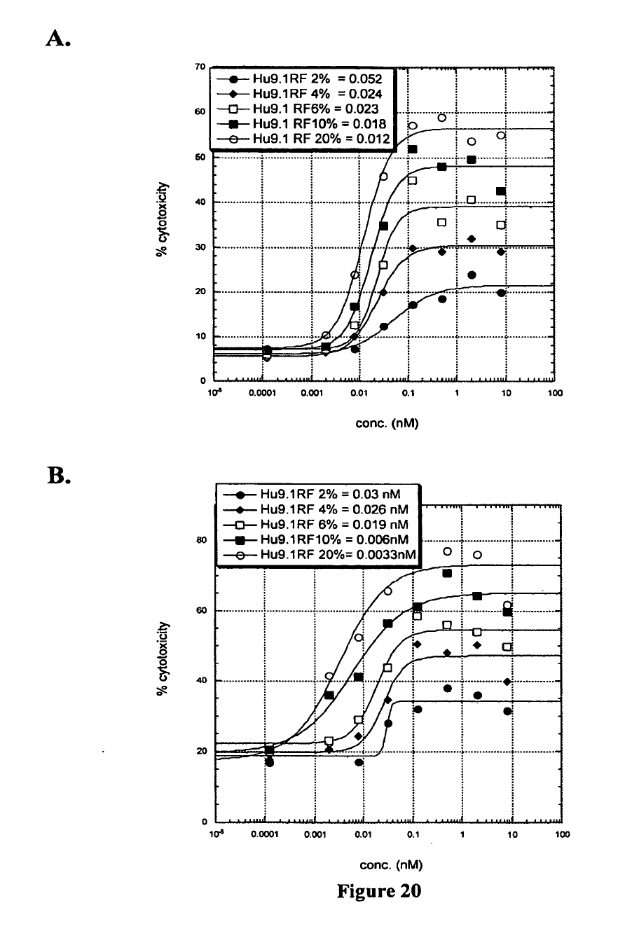

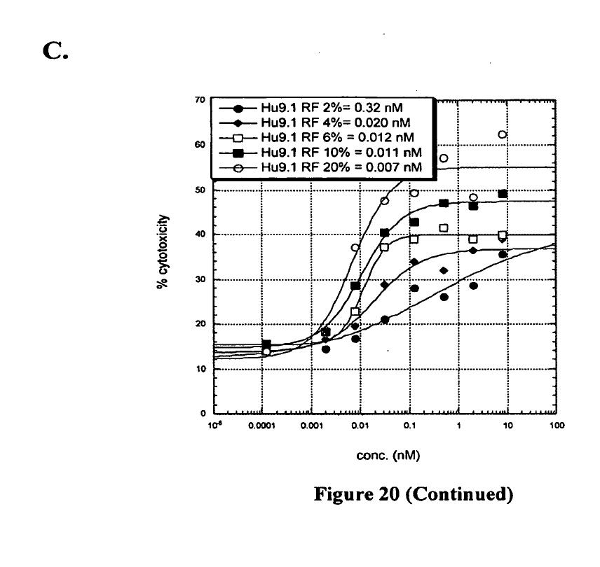

As mentioned above, a BAFF antagonist can function in a direct or indirect manner to partially or fully block, inhibit or neutralize BAFF signaling, in vitro or in vivo. For instance, the BAFF antagonist can directly bind BAFF. For example, anti-BAFF antibodies that bind within a region of human BAFF comprising residues 162-275 and/or a neighboring residue of a residue selected from the group consisting of 162, 163, 206, 211, 231, 233, 264 and 265 of human BAFF such that the antibody sterically hinders BAFF binding to BR3 is contemplated. In another example, a direct binder is a polypeptide comprising the extracellular domain of a BAFF receptor such as TACI, BR3 and BCMA, or comprising the boxed minimal region of the ECDs (corresponding to residues 19-35 of human BR3). Alternatively, the BAFF antagonist can bind an extracellular domain of a native sequence BR3 at its BAFF binding region to partially or fully block, inhibit or neutralize BAFF binding to BR3 in vitro, in situ, or in vivo. For example, such indirect antagonist is an anti-BR3 antibody that binds in a region of BR3 comprising residues 23-38 of human BR3 or a neighboring region of those residues such that binding of human BR3 to BAFF is sterically hindered. Other examples of BAFF binding Fc proteins that can be BAFF antagonists can be found in WO 02/66516, WO 00/40716, WO 01/87979, WO 03/024991, WO 02/16412, WO 02/38766, WO 02/092620 and WO 01/12812. BAFF antagonists include BAFF-binding sequences listed in FIG. 20 of WO 02/24909 and those described in WO 2003/024991, WO 02/092620, fragments of those sequences that bind BAFF, and fusion proteins comprising those sequences (e.g., Fc fusion proteins).

The terms “BR3”, “BR3 polypeptide” or “BR3 receptor” when used herein encompass “native sequence BR3 polypeptides” and “BR3 variants” (which are further defined herein). “BR3” is a designation given to those polypeptides comprising any one of SEQ ID NOs:145-149 and variants or fragments thereof. The BR3 polypeptides of the invention can be isolated from a variety of sources, such as from human tissue types or from another source, or prepared by recombinant and/or synthetic methods. The term BR3, includes the BR3 polypeptides described in WO 02/24909 and WO 03/14294.

A “native sequence” BR3 polypeptide comprises a polypeptide having the same amino acid sequence as the corresponding BR3 polypeptide derived from nature. Such native sequence BR3 polypeptides can be isolated from nature or can be produced by recombinant and/or synthetic means. The term “native sequence BR3 polypeptide” specifically encompasses naturally-occurring truncated, soluble or secreted forms (e.g., an extracellular domain sequence), naturally-occurring variant forms (e.g., alternatively spliced forms) and naturally-occurring allelic variants of the polypeptide. The BR3 polypeptides of the invention include the BR3 polypeptide comprising or consisting of the contiguous sequence of amino acid residues 1 to 184 of a human BR3.

A BR3 “extracellular domain” or “ECD” refers to a form of the BR3 polypeptide which is essentially free of the transmembrane and cytoplasmic domains. ECD forms of BR3 include those comprising any one of amino acids 1 to 77, 2 to 62, 2-71, 1-61, 8-71, 17-42, 19-35 or 2-63 of BR3.

“BR3 variant” means a BR3 polypeptide having at least about 60% amino acid sequence identity with the residues 19-35 of BR3ECD and binds a native sequence BAFF polypeptide. See Gordon, N. C., et al., (2003) Biochemistry 42:5977-5983). Optionally, the BR3 variant includes a single cysteine rich domain. Such BR3 variant polypeptides include, for instance, BR3 polypeptides wherein one or more amino acid residues are added, or deleted, at the N- and/or C-terminus, as well as within one or more internal domains, of the full-length amino acid sequence. Fragments of the BR3 ECD that bind a native sequence BAFF polypeptide are also contemplated. According to an embodiment, a BR3 variant polypeptide will have at least about 65% amino acid sequence identity, at least about 70% amino acid sequence identity, at least about 75% amino acid sequence identity, at least about 80% amino acid sequence identity, at least about 80% amino acid sequence identity, at least about 85% amino acid sequence identity, at least about 90% amino acid sequence identity, at least about 95% amino acid sequence identity, at least about 98% amino acid sequence identity or at least about 99% amino acid sequence identity in that portion corresponding to residues 19-35 of human BR3.

The term “antibody” is used in the broadest sense and specifically covers, for example, monoclonal antibodies, polyclonal antibodies, antibodies with polyepitopic specificity, single chain antibodies, multi-specific antibodies and fragments of antibodies. According to some embodiments, a polypeptide of this invention is fused into an antibody framework, for example, in the variable domain or in a CDR such that the antibody can bind to and inhibit BAFF binding to BR3 or BAFF signaling. The antibodies comprising a polypeptide of this invention can be chimeric, humanized, or human. The antibodies comprising a polypeptide of this invention can be an antibody fragment. Such antibodies and methods of generating them are described in more detail below. Alternatively, an antibody of this invention can be produced by immunizing an animal with a polypeptide of this invention. Thus, an antibody directed against a polypeptide of this invention is contemplated.

As used herein, the terms “anti-BR3” and “BR3 binding” are used interchangeably and indicate that the antibody or polypeptide binds a BR3 polypeptide. Preferably, the anti-BR3 antibody binds to an epitope on a BR3 polypeptide having an amino acid sequence selected from the group consisting of SEQ ID NO:145-149 and does not bind to human TACI or human BCMA. Preferably, the anti-BR3 antibody binds a human BR3 extracellular domain sequence with an apparent Kd value of 500 nM or less, 100 nM or less, 50 nM or less, 10 nM or less, 5 nM or less or 1 nM or less as a Fab in a BIAcore Assay at 25° C. According to one embodiment, the antibody or polypeptide binds to BR3 with an apparent Kd between 0.001 pM and 500 nM.

“Antagonistic anti-BR3 antibodies” according to this invention refer to antibodies that bind a BR3 polypeptide and inhibit BR3 signalling (e.g, inhibit BR3 related B cell proliferation, B cell survival or both B cell proliferation and survival).

“Agonistic anti-BR3 antibodies” according to this invention refer to antibodies that bind a BR3 polypeptide and stimulate BR3 signalling (e.g., BR3-related B cell proliferation, B cell survival or both B cell proliferation and survival).

The “CD20” antigen is a non-glycosylated, transmembrane phosphoprotein with a molecular weight of approximately 35 kD that is found on the surface of greater than 90% of B cells from peripheral blood or lymphoid organs. CD20 is expressed during early pre-B cell development and remains until plasma cell differentiation; it is not found on human stem cells, lymphoid progenitor cells or normal plasma cells. CD20 is present on both normal B cells as well as malignant B cells. Other names for CD20 in the literature include “B-lymphocyte-restricted differentiation antigen” and “Bp35.” The CD20 antigen is described in, for example, Clark and Ledbetter, Adv. Can. Res. 52:81-149 (1989) and Valentine et al. J. Biol. Chem. 264(19):11282-11287 (1989).

CD20 binding antibody and anti-CD20 antibody are used interchangeably herein and encompass all antibodies that bind CD20 with sufficient affinity such that the antibody is useful as a therapeutic agent in targeting a cell expressing the antigen, and do not significantly cross-react with other proteins such as a negative control protein in the assays described below. Bispecific antibodies wherein one arm of the antibody binds CD20 are also contemplated. Also encompassed by this definition of CD20 binding antibody are functional fragments of the preceding antibodies. The CD20 binding antibody will bind CD20 with a Kd of <10 nM. In preferred embodiments, the binding is at a Kd of <7.5 nM, more preferably <5 nM, even more preferably at between 1-5 nM, most preferably, <1 nM.

Examples of antibodies which bind the CD20 antigen include: “C2B8” which is now called “Rituximab” (“Rituxane®”) (U.S. Pat. No. 5,736,137, expressly incorporated herein by reference); the yttrium-[90]-labeled 2B8 murine antibody designated “Y2B8” or “Ibritumomab Tiuxetan” ZEVALIN® (U.S. Pat. No. 5,736,137, expressly incorporated herein by reference); murine IgG2a “B1,” also called “Tositumomab,” (Beckman Coulter) optionally labeled with ¹³¹I to generate the “131I-B1” antibody (iodine I131 tositumomab, BEXXAR™) (U.S. Pat. No. 5,595,721, expressly incorporated herein by reference); murine monoclonal antibody “1F5” (Press et al. Blood 69(2):584-591 (1987) and variants thereof including “framework patched” or humanized IFS (WO03/002607, Leung, S.); ATCC deposit HB-96450); murine 2H7 and chimeric 2H7 antibody (U.S. Pat. No. 5,677,180, expressly incorporated herein by reference); humanized 2H7; huMax-CD20 (Genmab, Denmark); AME-133 (Applied Molecular Evolution); A20 antibody or variants thereof such as chimeric or humanized A20 antibody (cA20, hA20, respectively) (US 2003/0219433, Immunomedics); and monoclonal antibodies L27, G28-2, 93-1B3, B-C1 or NU-B2 available from the International Leukocyte Typing Workshop (Valentine et al., In: Leukocyte Typing III (McMichael, Ed., p. 440, Oxford University Press (1987)).

The terms “rituximab” or “Rituxan®” herein refer to the genetically engineered chimeric murine/human monoclonal antibody directed against the CD20 antigen and designated “C2B8” in U.S. Pat. No. 5,736,137 expressly incorporated herein by reference, including fragments thereof which retain the ability to bind CD20 .

In a specific embodiment, the anti-CD20 antibodies bind human and primate CD20. In specific embodiments, the antibodies that bind CD20 are humanized or chimeric. CD20 binding antibodies include rituximab (Rituxan®), m2H7 (murine 2H7), hu2H7 (humanized 2H7) and all its functional variants, including without limitation, hu2H7.v16 (v stands for version), v31, v73, v75, as well as fucose deficient variants, and other 2H7 variants described in WO2004/056312. Unless indicated, the sequences disclosed herein of the humanized 2H7v.16 and variants thereof are of the mature polypeptide, i.e., without the leader sequence.

Patents and patent publications concerning CD20 antibodies include U.S. Pat. Nos. 5,776,456, 5,736,137, 5,843,439, 6,399,061, and 6,682,734, as well as US patent appln nos. US 2002/0197255A1, US 2003/0021781A1, US 2003/0082172 A1, US 2003/0095963 A1, US 2003/0147885 A1 (Anderson et al.); U.S. Pat. No. 6,455,043B1 and WO00/09160 (Grillo-Lopez, A.); WO00/27428 (Grillo-Lopez and White); WO00/27433 (Grillo-Lopez and Leonard); WO00/44788 (Braslawsky et al.); WO01/10462 (Rastetter, W.); WO01/10461 (Rastetter and White); WO01/10460 (White and Grillo-Lopez); US2001/0018041A1, US2003/0180292A1, WO01/34194 (Hanna and Hariharan); US appln no. US2002/0006404 and WO02/04021 (Hanna and Hariharan); US appln no. US2002/0012665 A1 and WO01/74388 (Hanna, N.); US appln no. US 2002/0058029 A1 (Hanna, N.); US appln no. US 2003/0103971 A1 (Hariharan and Hanna); US appln no. US2002/0009444A1, and WO01/80884 (Grillo-Lopez, A.); WO01/97858 (White, C.); US appln no. US2002/0128488A1 and WO02/34790 (Reff, M.); WO02/060955 (Braslawsky et al.); WO2/096948 (Braslawsky et al.); WO02/079255 (Reff and Davies); U.S. Pat. No. 6,171,586B1, and WO98/56418 (Lam et al.); WO98/58964 (Raju, S.); WO99/22764 (Raju, S.); WO99/51642, U.S. Pat. No. 6,194,551B1, U.S. Pat. No. 6,242,195B1, U.S. Pat. No. 6,528,624B1 and U.S. Pat. No. 6,538,124 (Idusogie et al.); WO00/42072 (Presta, L.); WO00/67796 (Curd et al.); WO01/03734 (Grillo-Lopez et al.); US appln no US 2002/0004587A1 and WO01/77342 (Miller and Presta); US appln no. US2002/0197256 (Grewal, I.); US Appln no. US 2003/0157108 A1 (Presta, L.); U.S. Pat. Nos. 6,565,827B1, 6,090,365B1, 6,287,537B1, 6,015,542, 5,843,398, and 5,595,721, (Kaminski et al.); U.S. Pat. Nos. 5,500,362, 5,677,180, 5,721,108, 6,120,767, 6,652,852B1 (Robinson et al.); U.S. Pat. No. 6,410,391B1 (Raubitschek et al.); U.S. Pat. No. 6,224,866B1 and WO00/20864 (Barbera-Guillem, E.); WO01/13945 (Barbera-Guillem, E.); WO00/67795 (Goldenberg); US Appl No. US 2003/0133930 A1 and WO00/74718 (Goldenberg and Hansen); WO00/76542 (Golay et al.); WO01/72333 (Wolin and Rosenblatt); U.S. Pat. No. 6,368,596B1 (Ghetie et al.); U.S. Pat. No. 6,306,393 and US Appln no. US2002/0041847 A1, (Goldenberg, D.); US Appln No. US2003/0026801A1 (Weiner and Hartmann); WO02/102312 (Engleman, E.); US Patent Application No. 2003/0068664 (Albitar et al.); WO03/002607 (Leung, S.); WO 03/049694, US2002/0009427A1, and US 2003/0185796 A1 (Wolin et al.); WO03/061694 (Sing and Siegall); US 2003/0219818 A1 (Bohen et al.); US 2003/0219433 A1 and WO 03/068821 (Hansen et al.); US2003/0219818A1 (Bohen et al.); US2002/0136719A1 (Shenoy et al.); WO2004/032828 (Wahl et al.), each of which is expressly incorporated herein by reference. See, also, U.S. Pat. No. 5,849,898 and EP appln no. 330,191 (Seed et al.); U.S. Pat. No. 4,861,579 and EP332,865A2 (Meyer and Weiss); U.S. Pat. No. 4,861,579 (Meyer et al.); WO95/03770 (Bhat et al.); US 2003/0219433 A1 (Hansen et al.).

The CD20 antibodies can be naked antibody or conjugated to a cytotoxic compound such as a radioisotope, or a toxin. Such antibodies include the antibody Zevalin™ which is linked to the radioisotope, Yttrium-90 (IDEC Pharmaceuticals, San Diego, Calif.), and Bexxar™ which is conjugated to I-131 (Corixa, Wash.). The humanized 2H7 variants include those that have amino acid substitutions in the FR and affinity maturation variants with changes in the grafted CDRs. The substituted amino acids in the CDR or FR are not limited to those present in the donor or acceptor antibody. In other embodiments, the anti-CD20 antibodies of the invention further comprise changes in amino acid residues in the Fc region that lead to improved effector function including enhanced CDC and/or ADCC function and B-cell killing (also referred to herein as B-cell depletion). In particular, three mutations have been identified for improving CDC and ADCC activity: S298A/E333A/K334A (also referred to herein as a triple Ala mutant or variant; numbering in the Fc region is according to the EU numbering system; Kabat et al., supra) as described (Idusogie et al., supra (2001); Shields et al., supra).

Other anti-CD20 antibodies of the invention include those having specific changes that improve stability. In one embodiment, the chimeric anti-CD20 antibody has murine V regions and human C region. One such specific chimeric anti-CD20 antibody is Rituxan® (Rituximab®; Genentech, Inc.). Rituximab and hu2H7 can mediate lysis of B-cells through both complement-dependent cytotoxicity (CDC) and antibody-dependent cellular cytotoxicity (ADCC). Antibody variants with altered Fc region amino acid sequences and increased or decreased C1q binding capability are described in U.S. Pat. No. 6,194,551B1 and WO99/51642. The contents of those patent publications are specifically incorporated herein by reference. See, also, Idusogie et al. J. Immunol. 164: 4178-4184 (2000).

Inhibitors of Apoptosis (IAP) refers to a family of proteins that inhibit apoptosis (Deveraux, et al., (1999) Genes Dev 13(3):239-252). Examples of IAPs includes melanoma IAP (ML-IAP) and human X-chromosome linked IAP (XIAP) cellular IAP 1 (cIAP-1), and cellular IAP 2 (cIAP-2), which inhibit caspase 3, caspase 7 and caspase 9 activity (Deveraux et al., J Clin Immunol (1999), 19:388-398; Deveraux et al., (1998) EMBO J. 17, 2215-2223; Vucic et al., (2000) Current Bio 10:1359-1366).

Examples of inhibitors of IAP (IAP inhibitors) includes antisense oligonucleotides directed against XIAP, clAP-1, cIAP-2 or ML-IAP, Smac/DIABLO-derived peptides or other molecules that block the interaction between IAPs and their caspases, and molecules that inhibit IAP-mediated suppression of caspase activity (Sasaki et al, Cancer Res., 2000, 60(20):5659; Lin et al, Biochem J., 2001, 353:299; Hu et al, Clin. Cancer Res., 2003, 9(7):2826; Arnt et al, 1. Biol. Chem., 2002, 277(46):44236; Fulda et al, Nature Med., 2002, 8(8):808; Guo et al, Blood, 2002, 99(9):3419; Vucic et al, J. Biol. Chem., 2002, 277(14):12275; Yang et al, Cancer Res., 2003, 63(4):831); WO 2005/097791, WO 2005/094818, US 2005/0197403 and U.S. Pat. No. 6,673,917).

A “B cell surface marker” or “B cell surface antigen” herein is an antigen expressed on the surface of a B cell which can be targeted with an antagonist which binds thereto. Exemplary B cell surface markers include, but are not limited to, CD10, CD19, CD20, CD21, CD22, CD23, CD24, CD37, CD40, CD52, D53, CD72, CD73, CD74, CDw75, CDw76, CD77, CDw78, CD79a, CD79b, CD80, CD81, CD82, CD83, CDw84, CD85, CD86, CD180 (RP105), FcRH2 (IRTA4), CD79A, C79B, CR2, CCR6, CD72, P2×5, HLA-DOB, CXCR5 (BLR1), FCER2, BR3 (aka BAFF-R), TACI, BTLA, NAG14 (aka LRRC4), SLGC16270 (ala LOC283663), FcRH1 (IRTA5), FcRH5 (IRTA2), ATWD578 (aka MGC15619), FcRH3 (IRTA3), FcRH4 (IRTA1), FcRH6 (aka LOC343413) and BCMA (aka TNFRSFI7), HLA-DO, HLA-Dr10 and MHC ClassII.

According to a preferred embodiment, the antibodies of this invention do not include the 9A antibody and the 2.1 antibody deposited and described in WO 02/24909.

According to one preferred embodiment, the “apparent Kd” or “apparent Kd value” as used herein is in one preferred embodiment is measured by surface plasmon resonance such as by performing a BIAcore® assay. In one preferred embodiment, an apparent Kd value for a BR3-binding antibody of this invention is measured by performing surface plasmon resonance wherein either a BR3 ECD is immobilized on a sensor chip and an anti-BR3 antibody in Fab form is flowed over the BR3 ECD-immobilized chip or an anti-BR3 antibody in IgG form is immobilized on a sensor chip and a BR3 ECD is flowed over the IgG-immobilized sensor chip, e.g., as described in Example 8 herein. According to one preferred embodiment, the sensor chips are immobilized with protein such that there is approximately 10 response units (RU) of coupled protein on a chip. In another preferred embodiment, an apparent Kd value for an FeRn-binding antibody of this invention is measured by performing surface plasmon resonance wherein a FcRn polypeptide is immobilized to a sensor chip and an antibody is flowed over the chip, e.g., as described in Example 16.

A “functional epitope” according to this invention refers to amino acid residues of an antigen that contribute energetically to the binding of an antibody. Mutation of any one of the energetically contributing residues of the antigen (for example, mutation of wild-type BR3 by alanine or homolog mutation) will disrupt the binding of the antibody to the antigen. In one preferred embodiment of this invention, a residue that is comprised within the functional epitope on an anti-BR3 antibody can be determined by shot-gun alanine scanning using phage displaying ala mutants of BR3 or a portion thereof (e.g, the extracellular domain or residues 17-42 if desired region of study). According to one preferred embodiment, the functional epitope is determined according to the procedure described in Example 9.

The term “variable” refers to the fact that certain segments of the variable domains differ extensively in sequence among antibodies. The V regions mediate antigen binding and define specificity of a particular antibody for its particular antigen. However, the variability is not evenly distributed across the 110-amino acid span of the variable domains. Instead, the V domains consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g. around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the V_{L}, and 26-32 (H1), 52A-55 (H2) and 96-101 (H3) in the V_{H} (Chothia and Lesk J. Mol. Biol. 196:901-917 (1987)).

Hypervariable regions may comprise “extended hypervariable regions” as follows: 24-36 or 24-34 (L1), 46-56 or 50-56 (L2) and 89-97 (L3) in the VL and 26-35 (H1), 50-65 or 49-65 (H2) and 93-102, 94-102 or 95-102 (H3) in the VH. The variable domain residues are numbered according to Kabat et al., supra for each of these definitions.

“Framework” or “FR” residues are those variable domain residues other than the hypervariable region residues as herein defined. For example, light chain framework 1 (LC-FR1), framework 2 (LC-FR2), framework 3 (LC-FR3) and framework 4 (LC-FR4) region comprise residues numbered 1-23, 35-49, 57-88 and 98-107 of an antibody (Kabat numbering system), respectively. In another example, heavy chain framework 1 (HC-FR1), heavy chain framework 2 (HC-FR2), heavy chain framework 3 (HC-FR3) and heavy chain framework 4 (HC-FR4) comprise residues 1-25, 36-48, 66-92 and 103-113, respectively, of an antibody (Kabat numbering system).

As referred to herein, the “consensus sequence” or consensus V domain sequence is an artificial sequence derived from a comparison of the amino acid sequences of known human immunoglobulin variable region sequences. Based on these comparisons, recombinant nucleic acid sequences encoding the V domain amino acids that are a consensus of the sequences derived from the human and the human H chain subgroup III V domains were prepared. The consensus V sequence does not have any known antibody binding specificity or affinity.

The term “monoclonal antibody” as used herein refers to an antibody from a population of substantially homogeneous antibodies, i.e., the individual antibodies comprising the population are identical and/or bind the same epitope(s), except for possible variants that may arise during production of the monoclonal antibody, such variants generally being present in minor amounts. Such monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones or recombinant DNA clones. It should be understood that the selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations which typically include different antibodies directed against different determinants (epitopes), each monoclonal antibody of a monoclonal antibody preparation is directed against a single determinant on an antigen. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including the hybridoma method (e.g., Kohler et al., Nature, 256:495 (1975); Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681, (Elsevier, N.Y., 1981), recombinant DNA methods (see, e.g., U.S. Pat. No. 4,816,567), phage display technologies (see, e.g., Clackson et al., Nature, 352:624-628 (1991); Marks et al., J. Mol. Biol., 222:581-597 (1991); Sidhu et al., J. Mol. Biol. 338(2):299-310 (2004); Lee et al., J. Mol. Biol. 340(5):1073-1093 (2004); Fellouse, Proc. Nat. Acad. Sci. USA 101(34):12467-12472 (2004); and Lee et al. J. Immunol. Methods 284(1-2):119-132 (2004) and technologies for producing human or human-like antibodies from animals that have parts or all of the human immunoglobulin loci or genes encoding human immunoglobulin sequences (see, e.g., WO98/24893, WO/9634096, WO/9633735, and WO/91 10741, Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806, 5,569,825, 5,591,669 (all of GenPharm); 5,545,807; WO 97/17852, U.S. Pat. Nos. 5,545,807; 5,545,806; 5,569,825; 5,625,126; 5,633,425; and 5,661,016, and Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); and Lonberg and Huszar, Intern. Rev. Immunol., 13: 65-93 (1995).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while portions of the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Methods of making chimeric antibodies are known in the art.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. In some embodiments, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies may comprise residues which are found neither in the recipient antibody nor in the imported CDR or framework sequences. These modifications are generally made to further refine and maximize antibody performance. Typically, the humanized antibody will comprise substantially all of at least one variable domain, in which all or substantially all of the hypervariable loops derived from a non-human immunoglobulin and all or substantially all of the FR regions are derived from a human immunoglobulin sequence although the FR regions may include one or more amino acid substitutions to, e.g., improve binding affinity. In some embodiments, the number of these amino acid substitutions in the FR are typically no more than 6 in the H chain, and in the L chain, no more than 3. In one preferred embodiment, the humanized antibody will also comprise at least a portion of an immunoglobulin constant region (Fc), typically that of a human immunoglobulin or a human consensus constant sequence. For further details, see Jones et al., Nature, 321:522-525 (1986); Reichmann et al., Nature, 332:323-329 (1988); and Presta, Curr. Op. Struct. Biol., 2:593-596 (1992). The humanized antibody includes a PRIMATIZED® antibody wherein the antigen-binding region of the antibody is derived from an antibody produced by, e.g., immunizing macaque monkeys with the antigen of interest. Methods of making humanized antibodies are known in the art.

Human antibodies can also be produced using various techniques known in the art, including phage-display libraries. Hoogenboom and Winter, J. Mol. Biol., 227:381 (1991); Marks et al., J. Mol. Biol., 222:581 (1991). The techniques of Cole et al. and Boerner et al. are also available for the preparation of human monoclonal antibodies. Cole et al., Monoclonal Antibodies and Cancer Therapy, Alan R. Liss, p. 77 (1985); Boerner et al., J. Immunol., 147(1):86-95 (1991). See also, Lonberg and Huszar, Int. Rev. Immunol. 13:65-93 (1995). PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598.

“Antibody fragments” comprise a portion of a full length antibody, generally the antigen binding or variable region thereof. Examples of antibody fragments include Fab, Fab′, F(ab′)2, and Fv fragments; diabodies; linear antibodies; single-chain antibody molecules; and multispecific antibodies formed from antibody fragments.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Functional fragments” of the BR3 binding antibodies of the invention are those fragments that retain binding to BR3 with substantially the same affinity as the intact full chain molecule from which they are derived and are active in at least one assay selected from the group consisting of depletion of B cells, inhibition of B cell proliferation or inhibition of BAFF binding to BR3 as measured by in vitro or in vivo assays such as those described herein.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g. B cell receptor); and B cell activation. A “native sequence Fc region” comprises an amino acid sequence identical to the amino acid sequence of an Fc region found in nature. Examples of Fc sequences are described in SEQ ID NOs:.133, 135-141 and include a native sequence human IgG1 Fc region (non-A and A allotypes, SEQ ID NO:133 and 135, respectively); native sequence human IgG2 Fc region (SEQ ID NO:136); native sequence human IgG3 Fc region (SEQ ID NO:137); and native sequence human IgG4 Fc region (SEQ ID NO:138) as well as naturally occurring variants thereof. Examples of native sequence murine Fc regions are described in SEQ ID NOs:139-142 (IgG1, IgG2a, IgG2b, IgG3, respectively).

A “variant Fc region” comprises an amino acid sequence which differs from that of a native sequence Fc region by virtue of at least one “amino acid modification” as herein defined. Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. In one embodiment, the variant Fc region herein will possess at least about 80% homology, at least about 85% homology, at least about 90% homology, at least about 95% homology or at least about 99% homology with a native sequence Fc region (e.g., SEQ ID NO: 133). According to another embodiment, the variant Fc region herein will possess at least about 80% homology, at least about 85% homology, at least about 90% homology, at least about 95% homology or at least about 99% homology with an Fc region of a parent polypeptide.

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

The term “Fc region-comprising polypeptide” refers to a polypeptide, such as an antibody or immunoadhesin (see definitions below), which comprises an Fc region. The C-terminal lysine (residue 447 according to the EU numbering system) of the Fc region may be removed, for example, during purification of the polypeptide or by recombinantly engineering the nucleic acid encoding the polypeptide. Accordingly, a composition comprising polypeptides, including antibodies, having an Fc region according to this invention can comprise polypeptides populations with all K447 residues removed, polypeptide populations with no K447 residues removed or polypeptide populations having a mixture of polypeptides with and without the K447 residue.

Throughout the present specification and claims, the Kabat numbering system is generally used when referring to a residue in the variable domain (approximately, residues 1-107 of the light chain and residues 1-113 of the heavy chain) (e.g, Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., Sequences of Proteins of Immunological Interest, 5th Ed. Public Health Service, National Institutes of Health, Bethesda, Md. (1991) expressly incorporated herein by reference). Unless stated otherwise herein, references to residues numbers in the variable domain of antibodies means residue numbering by the Kabat-numbering system. Unless stated otherwise herein, references to residue numbers in the constant domain of antibodies means residue numbering by the EU numbering system (e.g., see U.S. Provisional Application No. 60/640,323, Figures for EU numbering).

The terms “Fc receptor” or “FcR” are used to describe a receptor that binds to the Fc region of an antibody. In one embodiment, an FcR of this invention is one that binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see review M. in Daëron, Annu. Rev. Immunol. 15:203-234 (1997)). The term includes allotypes, such as FcγRIIIA allotypes: FcγRIIIA-Phe158, FcγRIIIA-Val158, FcγRIIA-R131 and/or FcγRIIA-H131. FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

The term “FcRn” refers to the neonatal Fc receptor (FcRn). FcRn is structurally similar to major histocompatibility complex (MHC) and consists of an α-chain noncovalently bound to β2-microglobulin. The multiple functions of the neonatal Fc receptor FcRn are reviewed in Ghetie and Ward (2000) Annu. Rev. Immunol. 18, 739-766. FcRn plays a role in the passive delivery of immunoglobulin IgGs from mother to young and the regulation of serum IgG levels. FcRn can act as a salvage receptor, binding and transporting pinocytosed IgGs in intact form both within and across cells, and rescuing them from a default degradative pathway.

WO00/42072 (Presta) and Shields et al. J. Biol. Chem. 9(2): 6591-6604 (2001) describe antibody variants with improved or diminished binding to FcRs. The contents of those publications are specifically incorporated herein by reference.

The “CH1 domain” of a human IgG Fc region (also referred to as “C1” of “H1” domain) usually extends from about amino acid 118 to about amino acid 215 (EU numbering system).

“Hinge region” is generally defined as stretching from Glu216 to Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions.

The “lower hinge region” of an Fc region is normally defined as the stretch of residues immediately C-terminal to the hinge region, i.e. residues 233 to 239 of the Fc region. In previous reports, FcR binding was generally attributed to amino acid residues in the lower hinge region of an IgG Fc region.

The “CH2 domain” of a human IgG Fc region (also referred to as “C2” of “H2” domain) usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec. Immunol. 22:161-206 (1985).

The “CH3 domain” (also referred to as “C2” or “113” domain) comprises the stretch of residues C-terminal to a CH2 domain in an Fc region (i.e. from about amino acid residue 341 to the C-terminal end of an antibody sequence, typically at amino acid residue 446 or 447 of an IgG)

A “functional Fc region” possesses an “effector function” of a native sequence Fc region. Exemplary “effector functions” include C1q binding; complement dependent cytotoxicity; Fc receptor binding; antibody-dependent cell-mediated cytotoxicity (ADCC); phagocytosis; down regulation of cell surface receptors (e.g. B cell receptor, BCR), etc. Such effector functions generally require the Fc region to be combined with a binding domain (e.g. an antibody variable domain) and can be assessed using various assays as herein disclosed, for example.

“C1q” is a polypeptide that includes a binding site for the Fc region of an immunoglobulin. C1q together with two serine proteases, C1r and C1s, forms the complex C1, the first component of the complement dependent cytotoxicity (CDC) pathway. Human C1q can be purchased commercially from, e.g. Quidel, San Diego, Calif.

The term “binding domain” refers to the region of a polypeptide that binds to another molecule. In the case of an FcR, the binding domain can comprise a portion of a polypeptide chain thereof (e.g. the alpha chain thereof) which is responsible for binding an Fc region. One useful binding domain is the extracellular domain of an FcR alpha chain.

A polypeptide with a variant IgG Fc with “altered” FcR binding affinity or ADCC activity is one which has either enhanced or diminished FcR binding activity (e.g, FcγR or FcRn) and/or ADCC activity compared to a parent polypeptide or to a polypeptide comprising a native sequence Fc region. The variant Fc which “exhibits increased binding” to an FcR binds at least one FcR with higher affinity (e.g., lower apparent Kd or IC50 value) than the parent polypeptide or a native sequence IgG Fc. According to some embodiments, the improvement in binding compared to a parent polypeptide is about 3 fold, preferably about 5, 10, 25, 50, 60, 100, 150, 200, up to 500 fold, or about 25% to 1000% improvement in binding. The polypeptide variant which “exhibits decreased binding” to an FcR, binds at least one FcR with lower affinity (e.g, higher apparent Kd or higher IC50 value) than a parent polypeptide. The decrease in binding compared to a parent polypeptide may be about 40% or more decrease in binding. In one embodiment, Fc variants which display decreased binding to an FcR possess little or no appreciable binding to an FcR, e.g., 0-20% binding to the FcR compared to a native sequence IgG Fc region, e.g. as determined in the Examples herein.

“Antibody-dependent cell-mediated cytotoxicity” or “ADCC” refers to a form of cytotoxicity in which secreted Ig bound to Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 or in the Examples below may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

The polypeptide comprising a variant Fc region which “exhibits increased ADCC” or mediates antibody-dependent cell-mediated cytotoxicity (ADCC) in the presence of human effector cells more effectively than a polypeptide having wild type IgG Fc or a parent polypeptide is one which in vitro or in vivo is substantially more effective at mediating ADCC, when the amounts of polypeptide with variant Fc region and the polypeptide with wild type Fc region (or the parent polypeptide) in the assay are essentially the same. Generally, such variants will be identified using the in vitro ADCC assay as herein disclosed, but other assays or methods for determining ADCC activity, e.g. in an animal model etc, are contemplated. In one embodiment, the preferred variant is from about 5 fold to about 100 fold, e.g. from about 25 to about 50 fold, more effective at mediating ADCC than the wild type Fc (or parent polypeptide).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g. as described in Gazzano-Santoro et al., J. Immunol. Methods 202:163 (1996), may be performed.

Polypeptide variants with altered Fc region amino acid sequences and increased or decreased C1q binding capability are described in U.S. Pat. No. 6,194,551B1 and WO99/51642. The contents of those patent publications are specifically incorporated herein by reference. See, also, Idusogie et al. J. Immunol. 164: 4178-4184 (2000).

“Human effector cells” are leukocytes which express one or more FcRs and perform effector functions. According to one embodiment, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g. from blood or PBMCs as described herein.

Methods of measuring binding to FcRn are known (see, e.g., Ghetie 1997, Hinton 2004) as well as described in the Examples below. Binding to human FcRn in vivo and serum half life of human FcRn high affinity binding polypeptides can be assayed, e.g, in transgenic mice or transfected human cell lines expressing human FcRn, or in primates administered with the Fc variant polypeptides. In one embodiment, the polypeptide and specifically the antibodies of the invention having a variant IgG Fc exhibits increased binding affinity for human FcRn over a polypeptide having wild-type IgG Fc, by at least 2 fold, at least 5 fold, at least 10 fold, at least 50 fold, at least 60 fold, at least 70 fold, at least 80 fold, at least 100 fold, at least 125 fold, at least 150 fold. In a specific embodiment, the binding affinity for human FcRn is increased about 170 fold.

For binding affinity to FcRn, in one embodiment, the EC50 or apparent Kd (at pH 6.0) of the polypeptide is less than 1 uM, more preferably less than or equal to 100 nM, more preferably less than or equal to 10 nM. In one embodiment, for increased binding affinity to FcγRIII (F158; i.e. low-affinity isotype) the EC50 or apparent Kd less is than or equal to 10 nM, and for FcγRIII (V158; high-affinity isotype) the EC50 or apparent Kd is less than or equal to 3 nM. According to another embodiment, a reduction in binding of an antibody to a Fc receptor relative to a control antibody (e.g., the Herceptin® antibody) may be considered significant relative to the control antibody if the ratio of the values of the absorbances at the midpoints of the test antibody and control antibody binding curves (e.g, A_{450 nm(antibody/A450 nm(control Ab)}) is less than or equal to 40%. According to another embodiment, an increase in binding of an antibody to a Fc receptor relative to a control antibody (e.g., the Herceptin® antibody) may be considered significant relative to the control antibody if the ratio of the values of the absorbances at the midpoints of the test antibody and control antibody binding curves (e.g, A_{450 nm(antibody/A450 nm(control Ab)}) is greater than or equal to 125%. See, e.g., Example 10.

A “parent polypeptide” or “parent antibody” is a polypeptide or antibody comprising an amino acid sequence from which the variant polypeptide or antibody arose and against which the variant polypeptide or antibody is being compared. Typically the parent polypeptide or parent antibody lacks one or more of the Fc region modifications disclosed herein and differs in effector function compared to a polypeptide variant as herein disclosed. The parent polypeptide may comprise a native sequence Fc region or an Fc region with pre-existing amino acid sequence modifications (such as additions, deletions and/or substitutions).

A “fusion protein” and a “fusion polypeptide” refer to a polypeptide having two portions of a polypeptide sequence covalently linked together. In most embodiments, each of the portions are polypeptide sequences not typically associated with each other in nature and/or have different properties. The property may be a biological property, such as activity in vitro or in vivo. The property may also be a simple chemical or physical property, such as binding to a target molecule, catalysis of a reaction, etc. The two portions may be linked directly by a single peptide bond or through a peptide linker containing one or more amino acid residues. Generally, the two portions will be linked in reading frame with each other.

An “isolated” antibody or polypeptide is one which has been identified and separated and/or recovered from a component of the environment from which it was produced. Contaminant components can be, e.g., materials which would interfere with diagnostic or therapeutic uses for the antibody, and may include enzymes, hormones, and other proteinaceous or nonproteinaceous solutes. In one preferred embodiment, the antibody or polypeptide will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody or polypeptide includes the antibody or polypeptide in situ within recombinant cells since at least one component of the antibody's natural environment will not be present. Ordinarily, however, isolated antibody or polypeptide will be prepared by at least one purification step.

An “isolated” polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide-adaptors or linkers are used in accordance with conventional practice.

“Vector” includes shuttle and expression vectors. Typically, the plasmid construct will also include an origin of replication (e.g., the ColE1 origin of replication) and a selectable marker (e.g., ampicillin or tetracycline resistance), for replication and selection, respectively, of the plasmids in bacteria. An “expression vector” refers to a vector that contains the necessary control sequences or regulatory elements for expression of the antibodies including antibody fragment of the invention, in bacterial or eukaryotic cells. Suitable vectors are disclosed below.

The cell that produces a BR3 binding antibody of the invention will include the bacterial and eukaryotic host cells into which nucleic acid encoding the antibodies have been introduced. Suitable host cells are disclosed below.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions,” as defined herein, can be identified by those that: (1) employ low ionic strength and high temperature for washing, for example 0.015 M sodium chloride/0.0015 M sodium citrate/0.1% sodium dodecyl sulfate at 50 C; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42 C; or (3) overnight hybridization in a solution that employs 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42 C, with a 10 minute wash at 42 C in 0.2×SSC (sodium chloride/sodium citrate) followed by a 10 minute high-stringency wash consisting of 0.1×SSC containing EDTA at 55 C.

“Moderately stringent conditions” can be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50 C. The skilled artisan will recognize how to adjust the temperature, ionic strength, etc. as necessary to accommodate factors such as probe length and the like.

The term “epitope tagged” when used herein refers to a chimeric polypeptide comprising a polypeptide fused to a “tag polypeptide.” The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues). Polypeptides and antibodies of this invention that are epitope-tagged are contemplated.

“Biologically active” and “biological activity” and “biological characteristics” with respect to an anti-BR3 polypeptide or antibody of this invention means the antibody or polypeptide binds BR3. According to one preferred embodiment, the antibody binds human BR3 polypeptide.

In a further embodiment, an anti-BR3 polypeptide or antibody of this invention also has any one, any combination or all of the following activities: (1) binds to a human BR3 extracellular domain sequence with an apparent Kd value of 500 nM or less, 100 nM or less, 50 nM or less, 10 nM or less, 5 nM or less or 1 nM or less; (2) binds to a human BR3 extracellular domain sequence and binds to a rodent BR3 extracellular domain sequence with an apparent Kd value of 500 nM or less, 100 nM or less, 50 nM or less, 10 nM or less, 5 nM or less or 1 nM or less; and (3) inhibits human BR3 binding to human BAFF. Depending on the desired use for the antibody, the antibody can further comprise the any one of the following activities (1) has antibody dependent cellular cytotoxicity (ADCC) in the presence of human effector cells compared to wild-type or native sequence IgG Fc; (2) has increased ADCC in the presence of human effector cells compared to wild-type or native sequence IgG Fc or (3) has decreased ADCC in the presence of human effector cells compared to wild-type or native sequence IgG Fc. According to another embodiment, an antibody of this invention binds the human Fc neonatal receptor (FcRn) with a higher affinity than a polypeptide or parent polypeptide having wild type or native sequence IgG Fc.

“Biologically active” and “biological activity” and “biological characteristics” with respect to an antagonist anti-BR3 polypeptide or antibody of this invention means the antibody or polypeptide has any one, any combination or all of the following activities: (1) inhibits B cell proliferation; (2) inhibits B cell survival; (3) kills or depletes B cells in vivo. According to one embodiment, the depletion of B cells when compared to the baseline level or appropriate negative control which is not treated with such anti-BR3 antibody or polypeptide is at least 20%. According to another embodiment, the antagonistic antibody has antibody dependent cellular cytotoxicity (ADCC) in the presence of human effector cells compared to wild-type or native sequence IgG Fc or has increased ADCC in the presence of human effector cells compared to wild-type or native sequence IgG Fc.

The amino acid sequences specifically disclosed herein are contiguous amino acid sequences unless otherwise specified.

Variations in polypeptides of this invention described herein, can be made, for example, using any of the techniques and guidelines for conservative and non-conservative mutations. Variations can be a substitution, deletion or insertion of one or more codons encoding the polypeptide that results in a change in the amino acid sequence of the polypeptide. Amino acid substitutions can be the result of replacing one amino acid with another amino acid having similar structural and/or chemical properties, such as the replacement of a leucine with a serine, i.e., conservative amino acid replacements. Insertions or deletions can optionally be in the range of about 1 to 5 amino acids. The variation allowed can be determined by systematically making insertions, deletions or substitutions of amino acids in the sequence and testing the resulting variants for activity exhibited by the full-length or mature native sequence.

The term “conservative” amino acid substitution as used within this invention is meant to refer to amino acid substitutions which substitute functionally equivalent amino acids. Conservative amino acid changes result in minimal change in the amino acid structure or function of the resulting peptide. For example, one or more amino acids of a similar polarity act as functional equivalents and result in a silent alteration within the amino acid sequence of the peptide. In general, substitutions within a group can be considered conservative with respect to structure and function. However, the skilled artisan will recognize that the role of a particular residue is determined by its context within the three-dimensional structure of the molecule in which it occurs. For example, Cys residues may occur in the oxidized (disulfide) form, which is less polar than the reduced (thiol) form. The long aliphatic portion of the Arg side chain can constitute a critical feature of its structural or functional role, and this may be best conserved by substitution of a nonpolar, rather than another basic residue. Also, it will be recognized that side chains containing aromatic groups (Trp, Tyr, and Phe) can participate in ionic-aromatic or “cation-pi” interactions. In these cases, substitution of one of these side chains with a member of the acidic or uncharged polar group may be conservative with respect to structure and function. Residues such as Pro, Gly, and Cys (disulfide form) can have direct effects on the main chain conformation, and often may not be substituted without structural distortions.

Conservative substitutions include the following specific substitutions based on the similarities in side chains and exemplary substitutions and preferred substitutions listed below. Amino acids may be grouped according to similarities in the properties of their side chains (in A. L. Lehninger, in Biochemistry, second ed., pp. 73-75, Worth Publishers, New York (1975)):

(1) non-polar: Ala (A), Val (V), Leu (L), Ile (I), Pro (P), Phe (F), Tip (W), Met (M)
(2) uncharged polar: Gly (G), Ser (S), Thr (T), Cys (C), Tyr (Y), Asn (N), Gln (Q)
(3) acidic: Asp (D), Glu (E)
(4) basic: Lys (K), Arg (R), His (H)

Alternatively, naturally occurring residues may be divided into groups based on common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Tip, Tyr, Phe.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

Non-conservative substitutions will entail exchanging a member of one of these classes for another class. Such substituted residues also may be introduced into the conservative substitution sites or, more preferably, into the remaining (non-conserved) sites.

The term “amino acid” within the scope of the present invention is used in its broadest sense and is meant to include the naturally occurring L alpha-amino acids or residues. The commonly used one and three letter abbreviations for naturally occurring amino acids are used herein (Lehninger, A. L., Biochemistry, 2d ed., pp. 71-92, (1975), Worth Publishers, New York). The term includes D-amino acids as well as chemically modified amino acids such as amino acid analogs, naturally occurring amino acids that are not usually incorporated into proteins such as norleucine, and chemically synthesized compounds having properties known in the art to be characteristic of an amino acid. For example, analogs or mimetics of phenylalanine or proline, which allow the same conformational restriction of the peptide compounds as natural Phe or Pro are included within the definition of amino acid. Such analogs and mimetics are referred to herein as “functional equivalents” of an amino acid. Other examples of amino acids are listed by Roberts and Vellaccio (The Peptides: Analysis, Synthesis, Biology,) Eds. Gross and Meiehofer, Vol. 5 p 341, Academic Press, Inc, N.Y. 1983, which is incorporated herein by reference.

Peptides synthesized by the standard solid phase synthesis techniques described here, for example, are not limited to amino acids encoded by genes for substitutions involving the amino acids. Commonly encountered amino acids which are not encoded by the genetic code, include, for example, those described in International Publication No. WO 90/01940, as well as, for example, 2-amino adipic acid (Aad) for Glu and Asp; 2-aminopimelic acid (Apm) for Glu and Asp; 2-aminobutyric (Abu) acid for Met, Leu, and other aliphatic amino acids; 2-aminoheptanoic acid (Ahe) for Met, Len and other aliphatic amino acids; 2-aminoisobutyric acid (Aib) for Gly; cyclohexylalanine (Cha) for Val, and Leu and Ile; homoarginine (Har) for Arg and Lys; 2,3-diaminopropionic acid (Dpr) for Lys, Arg and His; N-ethylglycine (EtGly) for Gly, Pro, and Ala; N-ethylglycine (EtGly) for Gly, Pro, and Ala; N-ethylasparigine (EtAsn) for Asn, and Gin; Hydroxyllysine (Hyl) for Lys; allohydroxyllysine (AHyl) for Lys; 3-(and 4) hydroxyproline (3Hyp, 4Hyp) for Pro, Ser, and Thr; allo-isoleucine (AIle) for Ile, Leu, and Val; -amidinophenylalanine for Ala; N-methylglycine (MeGly, sarcosine) for Gly, Pro, and Ala; N-methylisoleucine (MeIle) for Ile; Norvaline (Nva) for Met and other aliphatic amino acids; Norleucine (Nle) for Met and other aliphatic amino acids; Ornithine (Orn or Or) for Lys, Arg and His; Citrulline (Cit) and methionine sulfoxide (MSO) for Thr, Asn and Gln; -methylphenylalanine (MePhe), trimethylphenylalanine, halo (F, Cl, Br, and I)phenylalanine, triflourylphenylalanine, for Phe.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244: 1081-1085 (1989)]. Alanine is also typically preferred because it is the most common amino acid. Further, it is frequently found in both buried and exposed positions [Creighton, The Proteins, (W.H. Freeman & Co., N.Y.); Chothia, J. Mol. Biol., 150:1 (1976)]. If alanine substitution does not yield adequate amounts of variant, an isoteric amino acid can be used.

The term “detecting” is intended to include determining the presence or absence of a molecule or determining qualitatively or quantitatively the amount of a molecule. The term thus refers to the use of the materials, compositions, and methods of the present invention for qualitative and quantitative determinations. In general, the particular technique used for detection is not critical for practice of the invention.

For example, “detecting” according to the invention may include detecting: the presence or absence of a molecule, number of cells expressing the polypeptide, a change in the levels of the molecule or amount of the molecule bound to a target or target bound to the molecule; a change in biological function/activity of a molecule (e.g., ligand or receptor binding activity, intracellular signaling (such as NF-kB activation), tumor cell proliferation, B cell proliferation, or survival, etc.), e.g., using methods that are known in the art. In some embodiments, “detecting” may include detecting wild type levels of the molecule (e.g., mRNA or polypeptide levels). Detecting may include quantifying a change (increase or decrease) of any value between 10% and 90%, or of any value between 30% and 60%, or over 100%, when compared to a control. Detecting may include quantifying a change of any value between 2-fold to 10-fold, inclusive, or more e.g., 100-fold. Thus, for example, referral to a BR3 molecule can refer to its mRNA or protein, etc.

As used herein a “BR3 molecule” as used herein refers to a molecule substantially identical to: a BR3 polypeptide; a nucleic acid molecule encoding a BR3 polypeptide; as well as isoforms, fragments, analogs, or variants of the polypeptide or the nucleic acid molecule. For example, a BR3 molecule can include an isoform, fragment, analog, or variant of a BR3 polypeptide derived from a mammal, which BR3 molecule has the ability to bind BAFF.

As used herein a “BAFF molecule” as used herein refers to a molecule substantially identical to: a BAFF polypeptide; a nucleic acid molecule encoding a BAFF polypeptide; as well as isoforms, fragments, analogs, or variants of the polypeptide or the nucleic acid molecule. For example, a BAFF molecule can include an isoform, fragment, analog, or variant of a BAFF polypeptide derived from a mammal, which BAFF molecule that has the ability to bind BR3.

As used herein, a subject to be treated is a mammal (e.g., human, non-human primate, rat, mouse, cow, horse, pig, sheep, goat, dog, cat, etc.). The subject may be a clinical patient, a clinical trial volunteer, an experimental animal, etc. The subject may be suspected of having or at risk for having a cancer or immune disease, be diagnosed with a cancer or immune disease, or be a control subject that is confirmed to not have a cancer. Many diagnostic methods for cancer and immune disease and the clinical delineation of cancer or immune diagnoses are known in the art. According to one preferred embodiment, the subject to be treated according to this invention is a human.

“Treating” or “treatment” or “alleviation” refers to measures, wherein the object is to prevent or slow down (lessen) the targeted pathologic condition or disorder or relieve some of the symptoms of the disorder. Those in need of treatment include can include those already with the disorder as well as those prone to have the disorder or those in whom the disorder is to be prevented. A subject or mammal is successfully “treated” for a cancer if, after receiving a therapeutic amount of a polypeptide or an antibody of the present invention, the patient shows observable and/or measurable reduction in or absence of one or more of the following: reduction in the number of cancer cells or absence of the cancer cells; reduction in the tumor size; inhibition (i.e., slow to some extent and preferably stop) of cancer cell infiltration into peripheral organs including the spread of cancer into soft tissue and bone; inhibition (i.e., slow to some extent and preferably stop) of tumor metastasis; inhibition, to some extent, of tumor growth; and/or relief to some extent, one or more of the symptoms associated with the specific cancer; reduced morbidity and mortality, and improvement in quality of life issues. To the extent the polypeptides of this invention can prevent growth and/or kill existing cancer cells, it can be cytostatic and/or cytotoxic. Reduction of these signs or symptoms may also be felt by the patient.

The term “therapeutically effective amount” refers to an amount of a polypeptide of this invention effective to “alleviate” or “treat” a disease or disorder in a subject. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin can be obtained from any immunoglobulin, such as IgG-1, IgG-2, IgG-3, or IgG-4 subtypes, IgA (including IgA-1 and IgA-2), IgE, IgD or IgM. For example, useful immunoadhesins according to this invention can be polypeptides that comprise the BAFF binding portions of a polypeptide or BR3 binding portions of a polypeptide (e.g., a portion of a BAFF receptor excluding the transmembrane or cytoplasmic sequences fused to an Fc region, TACI receptor extracellular domain-Fc or BCMA extracellular domain-Fc or BR3 extracellular domain-Fc). In one embodiment, a polypeptide sequence of this invention is fused to a constant domain of an immunoglobulin sequence.

An “immunodeficiency disease” is a disorder or condition where the immune response is reduced (e.g., severe combined immunodeficiency (SCID)-X linked, SCID-autosomal, adenosine deaminase deficiency (ADA deficiency), X-linked agammaglobulinemia (XLA). Bruton's disease, congenital agammaglobulinemia, X-linked infantile agammaglobulinemia, acquired agammaglobulinemia, adult onset agammaglobulinemia, late-onset agammaglobulinemia, dysgammaglobulinemia, hypogammaglobulinemia, transient hypogammaglobulinemia of infancy, unspecified hypogammaglobulinemia, agammaglobulinemia, common variable immunodeficiency (CVID) (acquired), Wiskott-Aldrich Syndrome (WAS), X-linked immunodeficiency with hyper IgM, non X-linked immunodeficiency with hyper IgM, selective IgA deficiency, IgG subclass deficiency (with or without IgA deficiency), antibody deficiency with normal or elevated Igs, immunodeficiency with thymoma, Ig heavy chain deletions, kappa chain deficiency, B cell lymphoproliferative disorder (BLPD), selective IgM immunodeficiency, recessive agammaglobulinemia (Swiss type), reticular dysgenesis, neonatal neutropenia, severe congenital leukopenia, thymic alymphoplasia-aplasia or dysplasia with immunodeficiency, ataxia-telangiectasia telangiectasia (cerebellar ataxia, oculocutaneous telangiectasia and immunodeficiency), short limbed dwarfism, X-linked lymphoproliferative syndrome (XLP), Nezelof syndrome-cumbined immunodeficiency with Igs, purine nucleotide phosphorylase deficiency (PNP), MHC Class II deficiency (Bare Lymphocyte Syndrome) and severe combined immunodeficiency,) or conditions associated with an immunodeficiency, Janus Associated Kinase 3 (JAK3) deficiency, DiGeorge's syndrome (isolated T cell deficiency) and Associated syndromes e.g., Down syndrome, chronic mucocutaneous candidiasis, hyper-IgE syndrome, chronic granulomatous disease, partial albinism and WHIM syndrome (warts, hypogammaglobulinemia, infection, and myelokathexis [retention of leukocytes in a hypercellular marrow]).

An “autoimmune disease” herein is a disease or disorder arising from and directed against an individual's own tissues or a co-segregate or manifestation thereof or resulting condition therefrom. Examples of autoimmune diseases or disorders include, but are not limited to arthritis (rheumatoid arthritis such as acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, dermatitis including contact dermatitis, chronic contact dermatitis, allergic dermatitis, allergic contact dermatitis, dermatitis herpetiformis, and atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, and ataxic sclerosis, inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), pyoderma gangrenosum, erythema nodosum, primary sclerosing cholangitis, episcleritis), respiratory distress syndrome, including adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, allergic conditions, allergic reaction, eczema including allergic or atopic eczema, asthma such as asthma bronchiale, bronchial asthma, and auto-immune asthma, conditions involving infiltration of T cells and chronic inflammatory responses, chronic pulmonary inflammatory disease, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis including lymphomatoid granulomatosis, Wegener's granulomatosis, agranulocytosis, vasculitides, including vasculitis (including large vessel vasculitis (including polymyalgia rheumatica and giant cell (Takayasu's) arteritis), medium vessel vasculitis (including Kawasaki's disease and polyarteritis nodosa), microscopic polyarteritis, CNS vasculitis, necrotizing, cutaneous, or hypersensitivity vasculitis, systemic necrotizing vasculitis, and ANCA-associated vasculitis, such as Churg-Strauss vasculitis or syndrome (CSS)), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, immune complex nephritis, antibody-mediated nephritis, neuromyelitis optica, polyneuropathies, chronic neuropathy such as IgM polyneuropathies or IgM-mediated neuropathy, thrombocytopenia (as developed by myocardial infarction patients, for example), including thrombotic thrombocytopenic purpura (TTP) and autoimmune or immune-mediated thrombocytopenia such as idiopathic thrombocytopenic purpura (ITP) including chronic or acute ITP, autoimmune disease of the testis and ovary including autoimune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis); or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis, bronchiolitis obliterans (non-transplant) vs NSIP, Guillain-Barré syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AGED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis (e.g., benign monoclonal gammopathy and monoclonal garnmopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases, diabetic nephropathy, Dressler's syndrome, alopecia greata, CREST syndrome (calcinosis, Raynaud's phenomenon, esophageal dysmotility, sclerodactyl), and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, infertility due to antispermatozoan antobodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis.

As used herein, “B cell depletion” refers to a reduction in B cell levels in an animal or human after drug or antibody treatment, as compared to the B cell level before treatment. B cell levels are measurable using well known assays such as those described in the Experimental Examples. B cell depletion can be complete or partial. In one embodiment, the depletion of BR3 expressing B cells is at least 25%. Not to be limited by any one mechanism, possible mechanisms of B-cell depletion include ADCC, CDC, apoptosis, modulation of calcium flux or a combination of two or more of the preceding.

A “B cell surface marker” or “B cell surface antigen” herein is an antigen expressed on the surface of a B cells.

“B cell depletion agents” refers to agents that reduce peripheral B cells by at least 25%. In another embodiment, the depletion of peripheral B cells is at least 30%, 40%, 50%, 60%, 70%, 80% or 90%. In one preferred embodiment, the B cell depletion agent specifically binds to a white blood cell and not other cells types. In another embodiment, the B cell depletion agent specifically binds to a B cell and not other cell types. In one embodiment, the B cell depletion agent is an antibody. In one preferred embodiment, the antibody is a monoclonal antibody. In another embodiment, the antibody is conjugated to a chemotherapeutic agent or a cytotoxic agent. Specific examples of B cell depletion agents include, but are not limited to, the aforementioned anti-CD20 antibodies.

The B cell neoplasms include Hodgkin's disease including lymphocyte predominant Hodgkin's disease (LPHD); non-Hodgkin's lymphoma (NHL); follicular center cell (FCC) lymphomas; acute lymphocytic leukemia (ALL); chronic lymphocytic leukemia (CLL); Hairy cell leukemia and BR3-positive neoplasms. The non-Hodgkins lymphoma include low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, plasmacytoid lymphocytic lymphoma, mantle cell lymphoma, AIDS—related lymphoma and Waldenstrom's macroglobulinemia. Treatment of relapses of these cancers are also contemplated. LPHD is a type of Hodgkin's disease that tends to relapse frequently despite radiation or chemotherapy treatment and can be characterized by BR3-positive malignant cells. CLL is one of four major types of leukemia. A cancer of mature B-cells called lymphocytes, CLL is manifested by progressive accumulation of cells in blood, bone marrow and lymphatic tissues. Indolent lymphoma is a slow-growing, incurable disease in which the average patient survives between six and 10 years following numerous periods of remission and relapse.

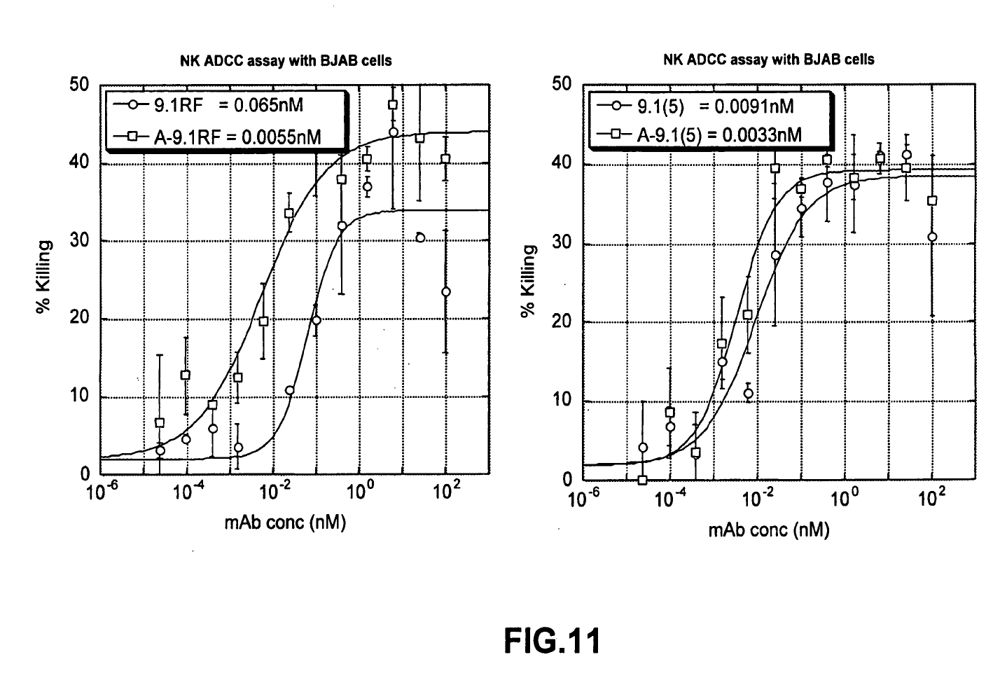

The term “non-Hodgkin's lymphoma” or “NHL”, as used herein, refers to a cancer of the lymphatic system other than Hodgkin's lymphomas. Hodgkin's lymphomas can generally be distinguished from non-Hodgkin's lymphomas by the presence of Reed-Sternberg cells in Hodgkin's lymphomas and the absence of said cells in non-Hodgkin's lymphomas. Examples of non-Hodgkin's lymphomas encompassed by the term as used herein include any that would be identified as such by one skilled in the art (e.g., an oncologist or pathologist) in accordance with classification schemes known in the art, such as the Revised European-American Lymphoma (REAL) scheme as described in Color Atlas of Clinical Hematology, Third Edition; A. Victor Hofibrand and John E. Pettit (eds.) (Harcourt Publishers Limited 2000) (see, in particular FIG. 11.57, 11.58 and/or 11.59). More specific examples include, but are not limited to, relapsed or refractory NHL, front line low grade NHL, Stage III/IV NHL, chemotherapy resistant NHL, precursor B lymphoblastic leukemia and/or lymphoma, small lymphocytic lymphoma, B cell chronic lymphacytic leukemia and/or prolymphocytic leukemia and/or small lymphocytic lymphoma, B-cell prolymphocytic lymphoma, immunocytoma and/or lymphoplasmacytic lymphoma, marginal zone B cell lymphoma, splenic marginal zone lymphoma, extranodal marginal zone-MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), intermediate grade diffuse NHL, diffuse large B-cell lymphoma, aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) T-cell lymphoblastic leukemia and/or lymphoma, adult T-cell lymphoma and/or leukemia, T cell chronic lymphocytic leukemia and/or prolymphacytic leukemia, large granular lymphocytic leukemia, mycosis fungoides and/or Sezary syndrome, extranodal natural killer/T-cell (nasal type) lymphoma, enteropathy type T-cell lymphoma, hepatosplenic T-cell lymphoma, subcutaneous panniculitis like T-cell lymphoma, skin (cutaneous) lymphomas, anaplastic large cell lymphoma, angiocentric lymphoma, intestinal T cell lymphoma, peripheral T-cell (not otherwise specified) lymphoma and angioimmunoblastic T-cell lymphoma.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; multiple myeloma and post-transplant lymphoproliferative disorder (PTLD). According to one preferred embodiment, the cancer comprises a tumor that expresses a BR3 polypeptide on its surface (BR3-positive). According to another embodiment, the BR3-expressing cancer is a CLL cancer.

In specific embodiments, the anti-BR3 antibodies and polypeptides of this invention are used to treat any one or more of the diseases selected from the group consisting of non-Hodgkin's lymphoma (NHL), lymphocyte predominant Hodgkin's disease (LPHD), chronic lymphocytic leukemia (CLL), acute lymphocytic leukemia (ALL), small lymphocytic lymphoma (SLL), diffuse large B cell lymphoma (DLBCL), follicular lymphoma, which are types of non-Hodgkin's lymphoma (NHL), rheumatoid arthritis and juvenile rheumatoid arthritis, systemic lupus erythematosus (SLE) including lupus nephritis, Wegener's disease, inflammatory bowel disease, idiopathic thrombocytopenic purpura (ITP), thrombotic throbocytopenic purpura (TTP), autoimmune thrombocytopenia, multiple sclerosis, psoriasis, IgA nephropathy, IgM polyneuropathies, myasthenia gravis, vasculitis, diabetes mellitus, Reynaud's syndrome, Sjorgen's syndrome, glomerulonephritis and multiple myeloma.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², Bi²¹³, P³² and radioactive isotopes of Lu), chemotherapeutic agents, and toxins such as small molecule toxins or enzymatically active toxins of bacterial, fungal, plant or animal origin, including fragments and/or variants thereof. According to one embodiment, the cytotoxic agent is capable of being internalized. According to another embodiment, the active portion of the cytotoxic agent is 1100 kD or less. According to one embodiment the chemotherapeutic agent is selected from the group consisting of methotrexate, adriamicin, vinca alkaloids (vincristine, vinblastine, etoposide), doxorubicin, melphalan, mitomycin C, chlorambucil, daunorubicin, or other intercalating agents, enzymes and fragments thereof such as nucleolytic enzymes, antibiotics, and toxins such as small molecule toxins or enzymatically active toxins of bacterial, fungal, plant or animal origin, (e.g., monomethylauristatin (MMAE) including fragments and/or variants thereof, and the various antitumor or anticancer agents or grow inhibitory agents disclosed below. Other cytotoxic agents are described below.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Illinois), and TAXOTERE® doxetaxel (Rhône-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluoromethylomithine (DMFO); retinoids such as retinoic acid; capecitabine; and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

A “growth inhibitory agent” when used herein refers to a compound or composition which inhibits growth of a cell in vitro and/or in vivo. Thus, the growth inhibitory agent may be one that significantly reduces the percentage of cells in S phase. Examples of growth inhibitory agents include agents that block cell cycle progression (at a place other than S phase), such as agents that induce GI arrest and M-phase arrest. Classical M-phase blockers include the vincas (vincristine and vinblastine), TAXOL® paclitaxel, and topo II inhibitors such as doxorubicin, epirubicin, daunorubicin, etoposide, and bleomycin. Those agents that arrest GI also spill over into S-phase arrest, for example, DNA alkylating agents such as tanoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antieioplastic drugs” by Murakaini et al. (W B Saunders: Philadelphia, 1995), especially p. 13.

An antibody that “induces cell death” is one that causes a viable cell to become nonviable. The cell is generally one that expresses the antigen to which the antibody binds, especially where the cell overexpresses the antigen. Preferably, the cell is a cancer cell, e.g., a breast, ovarian, stomach, endometrial, salivary gland, lung, kidney, colon, thyroid, pancreatic or bladder cell. In vitro, the cell may be a SKBR3, BT474, Calu 3, MDA-MB-453, MDA-MB-361 or SKOV3 cell. Cell death in vitro may be determined in the absence of complement and immune effector cells to distinguish cell death induced by antibody dependent cell-mediated cytotoxicity (ADCC) or complement dependent cytotoxicity (CDC). Thus, the assay for cell death may be performed using heat inactivated serum (i.e. in the absence of complement) and in the absence of immune effector cells. To determine whether the antibody is able to induce cell death, loss of membrane integrity as evaluated by uptake of propidium iodide (PI), trypan blue (see Moore et al. Cytotechnology, 17:1-11 (1995)) or 7AAD can be assessed relative to untreated cells.

An antibody that “induces apoptosis” is one which induces programmed cell death as determined by binding of annexin V, fragmentation of DNA, cell shrinkage, dilation of endoplasmic reticulum, cell fragmentation, and/or formation of membrane vesicles (called apoptotic bodies). The cell is one which expresses the antigen to which the antibody binds and may be one that overexpresses the antigen. The cell may be a tumor cell, e.g., a breast, ovarian, stomach, endometrial, salivary gland, lung, kidney, colon, thyroid, pancreatic or bladder cell. In vitro, the cell may be a SKBR3, BT474, Calu 3 cell, MDA-MB-453, MDA-MB-361 or SKOV3 cell. Various methods are available for evaluating the cellular events associated with apoptosis. For example, phosphatidyl serine (PS) translocation can be measured by annexin binding; DNA fragmentation can be evaluated through DNA laddering as disclosed in the example herein; and nuclear/chromatin condensation along with DNA fragmentation can be evaluated by any increase in hypodiploid cells. Preferably, the antibody that induces apoptosis is one which results in about 2 to 50 fold, preferably about 5 to 50 fold, and most preferably about 10 to 50 fold, induction of annexin binding relative to untreated cell in an annexin binding assay using cells expressing the antigen to which the antibody binds.

Examples of antibodies that induce apoptosis include the anti-DRS antibodies 3F1 1.39.7 (ATCC HB-12456); 3H3.14.5 (ATCC HB-12534); 3D5.1.10 (ATCC HB-12536); and 3H3.14.5 (ATCC HB-12534), including humanized and/or affinity-matured variants thereof; the human anti-DR5 receptor antibodies 16E2 and 20E6, including affinity-matured variants thereof (WO98/5 1793, expressly incorporated herein by reference); the anti-DR4 antibodies 4E7.24.3 (ATCC HB-12454); 4H6.17.8 (ATCC HB-12455); 1H5.25.9 (ATCC HB-12695); 4G7.18.8 (ATCC PTA-99); and 5GI 1.17.1 (ATCC HB-12694), including humanized and/or affinity-matured variants thereof.

In order to screen for antibodies which bind to an epitope on an antigen bound by an antibody of interest, a routine cross-blocking assay such as that described in Antibodies, A Laboratory Manual, eds. Harlow and Lane (New York: Cold Spring Harbor Laboratory, 1988) can be performed.

A “conjugate” refers to any hybrid molecule, including fusion proteins and as well as molecules that contain both amino acid or protein portions and non-protein portions (e.g., toxin-antibody conjugates, or pegylated-antibody conjugates). Conjugates may be synthesized or engineered by a variety of techniques known in the art including, for example, recombinant DNA techniques, solid phase synthesis, solution phase synthesis, organic chemical synthetic techniques or a combination of these techniques. The choice of synthesis will depend upon the particular molecule to be generated. For example, a hybrid molecule not entirely “protein” in nature may be synthesized by a combination of recombinant techniques and solution phase techniques.

According to one embodiment, the conjugate is an antibody or polypeptide of interest covalently linked to a salvage receptor binding epitope (especially an antibody fragment), as described, e.g., in U.S. Pat. No. 5,739,277. For example, a nucleic acid molecule encoding the salvage receptor binding epitope can be linked in frame to a nucleic acid encoding a polypeptide sequence of this invention so that the fusion protein expressed by the engineered nucleic acid molecule comprises the salvage receptor binding epitope and a polypeptide sequence of this invention. As used herein, the term “salvage receptor binding epitope” refers to an epitope of the Fc region of an IgG molecule (e.g., IgG₁, IgG₂, IgG₃, or IgG₄) that is useful for increasing the in vivo serum half-life of the IgG molecule (e.g., Ghetie, V et al., (2000) Ann. Rev. Immunol. 18:739-766, Table 1).

In another embodiment, the conjugate can be formed, by linkage (especially an antibody fragment) to serum albumin or a portion of serum albumin that binds to the FcRn receptor or a serum albumin-binding peptide or to a non-protein polymer (e.g., a polyethylene glycol moiety). Such polypeptide sequences are disclosed, for example, in WO01/45746. In one preferred embodiment, the serum albumin peptide to be attached comprises an amino acid sequence of DICLPRWGCLW. In another embodiment, the half-life of a Fab according to this invention is increased by these methods. See also, Dennis, M. S., et al., (2002) JBC 277(38):35035-35043 for serum albumin binding peptide sequences.

The word “label” when used herein refers to a detectable compound or composition which is conjugated directly or indirectly to the antibody. The label may itself be detectable by itself (e.g., radioisotope labels or fluorescent labels) or, in the case of an enzymatic label, may catalyze chemical alteration of a substrate compound or composition which is detectable.

### A. Compositions and Methods of the Invention

The invention provides antibodies that bind human BR3, and optionally other primate BR3 as well. According to one embodiment, the H chain has at least one, two or all of the H chain CDRs of a non-human species anti-human BR3 antibody (donor antibody), and substantially all of the framework residues of a human consensus antibody as the recipient antibody. The donor antibody can be from various non-human species including mouse, rat, guinea pig, goat, rabbit, horse, primate but typically will be a murine antibody. “Substantially all” in this context is meant that the recipient FR regions in the humanized antibody may include one or more amino acid substitutions not originally present in the human consensus FR sequence. These FR changes may comprise residues not found in the recipient or the donor antibody.

In one embodiment, the donor antibody is the murine 9.1 antibody, the V region including the CDR and FR sequences of each of the VH and VL chains of which are shown in SEQ ID NO:19 and SEQ ID NO:20. In one embodiment, the residues for the human Fab framework correspond to or were derived from the consensus sequence of a human Vκ subgroup I and of a V_{H} subgroup III. According to one embodiment, a humanized BR3 antibody of the invention has at least one of the CDRs in the H chain of the murine donor antibody. In one embodiment, the humanized BR3 antibody that binds human BR3 comprises the heavy chain CDRs of the H chain of the donor antibody.

In a full length antibody, the humanized BR3 binding antibody of the invention will comprise a V domain joined to a C domain of a human immunoglobulin, e.g., SEQ ID NO:132. In a preferred embodiment, the H chain C region is from human IgG, such as IgG1 or IgG3. According to one embodiment, the L chain C domain is from a human κ chain. According to another embodiment, the Fc sequence of a full length BR3 binding antibody is SEQ ID NO:134, wherein X is selected from the group consisting of N, A, Y, F and H.

The BR3 binding antibodies will bind at least human BR3. According to one embodiment, the BR3-binding antibody will bind other primate BR3 such as that of monkeys including cynomolgus and rhesus monkeys, and chimpanzees. According to another embodiment, the BR3 binding antibody or polypeptide binds a rodent BR3 protein and a human BR3 protein. In another embodiment, the BR3 polypeptide binds a mouse BR3 polypeptide sequence and a human BR3 polypeptide sequence.

According to one embodiment, the biological activity of an antagonist BR3 binding antibodies is any one, any combination or all of the activities selected from the group consisting of (1) binds to a human BR3 extracellular domain sequence with an apparent Kd value of 500 nM or less, 100 nM or less, 50 nM or less, 10 nM or less, 5 nM or less or 1 nM or less; (2) binds to a human BR3 extracellular domain sequence and binds to a mouse BR3 extracellular domain sequence with an apparent Kd value of 500 nM or less, 100 nM or less, 50nM or less, 10 nM or less, 5 nM or less or 1 nM or less; (3) has a functional epitope on human BR3-comprising residues F25, V33 and A34, wherein the monoclonal antibody; (4) inhibits human BAFF and human BR3 binding; (5) has antibody dependent cellular cytotoxicity (ADCC) in the presence of human effector cells or has increased ADCC in the presence of human effector cells; (6) binds the human Fc neonatal receptor (FcRn) with a higher affinity than a polypeptide or parent polypeptide having wild type or native sequence IgG Fc; (9) kills or depletes B cells in vitro or in vivo, preferably by at least 20% when compared to the baseline level or appropriate negative control which is not treated with such antibody; (10) inhibits B cell proliferation in vitro or in vivo and (11) inhibits B cell survival in vitro or in vivo. According to one embodiment of the polypeptides or antibodies of this invention, the functional epitope further comprises residue R30. According to yet another embodiment of this invention, the functional epitope further comprises residues L28 and V29.

In one embodiment, compared to treatment with a control antibody that does not bind a B cell surface antigen or as compared to the baseline level before treatment, the variable domain of an antibody of this invention fused to an Fc region of an mIgG2A can deplete at least 20% of the B cells in any one, any combination or all of following population of cells in mice: (1) B cells in blood, (2) B cells in the lymph nodes, (3) follicular B cells in the spleen and (4) marginal zone B cells in the spleen. In other embodiments, B cell depletion is 25%, 30%, 40%, 50%, 60%, 70%, 80% or greater. In one preferred embodiment, the depletion is measured at day 15 post treatment with antibody. In another preferred embodiment, the depletion assay is carried out as described in Example 18 or 19 herein. In another preferred embodiment, the depletion is measured by the population of peripheral B cells in a mouse day 15 post-treatment.

The desired level of B cell depletion will depend on the disease. For the treatment of a BR3 positive cancer, it may be desirable to maximize the depletion of the B cells which are the target of the anti-BR3 antibodies and polypeptides of the invention. Thus, for the treatment of a BR3 positive B cell neoplasm, it is desirable that the B cell depletion be sufficient to at least prevent progression of the disease which can be assessed by the physician of skill in the art, e.g., by monitoring tumor growth (size), proliferation of the cancerous cell type, metastasis, other signs and symptoms of the particular cancer. According to one preferred embodiment, the B cell depletion is sufficient to prevent progression of disease for at least 2 months, more preferably 3 months, even more preferably 4 months, more preferably 5 months, even more preferably 6 or more months. In even more preferred embodiments, the B cell depletion is sufficient to increase the time in remission by at least 6 months, more preferably 9 months, more preferably one year, more preferably 2 years, more preferably 3 years, even more preferably 5 or more years. In a most preferred embodiment, the B cell depletion is sufficient to cure the disease. In preferred embodiments, the B cell depletion in a cancer patient is at least about 75% and more preferably, 80%, 85%, 90%, 95%, 99% and even 100% of the baseline level before treatment.

For treatment of an autoimmune disease, it can be desirable to modulate the extent of B cell depletion depending on the disease and/or the severity of the condition in the individual patient, by adjusting the dosage of BR3 binding antibody or polypeptide. Thus, B cell depletion can but does not have to be complete. Total B cell depletion may be desired in initial treatment but in subsequent treatments, the dosage may be adjusted to achieve only partial depletion. In one embodiment, the B cell depletion is at least 20%, i.e., 80% or less of BR3 positive B cells remain as compared to the baseline level before treatment. In other embodiments, B cell depletion is 25%, 30%, 40%, 50%, 60%, 70%, 80% or greater. According to one preferred embodiment, the B cell depletion is sufficient to halt progression of the disease, more preferably to alleviate the signs and symptoms of the particular disease under treatment, even more preferably to cure the disease.

The invention also provides bispecific BR3 binding antibodies wherein one arm of the antibody has a humanized H and L chain of the BR3 binding antibody of the invention, and the other arm has V region binding specificity for a second antigen. In specific embodiments, the second antigen is selected from the group consisting of CD3, CD64, CD32A, CD16, NKG2D or other NK activating ligands.

Any cysteine residue not involved in maintaining the proper conformation of the anti-BR3 antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

A particularly preferred type of substitutional variant involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and human BR3. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the anti-BR3 antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the anti-BR3 antibody.

It may be desirable to modify the antibody of the invention with respect to effector function, e.g. so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered which has dual Fc regions and may thereby have enhanced complement mediated lysis and ADCC capabilities. See Stevenson et al. Anti-Cancer Drug Design 3:219-230 (1989).

To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

Other Antibody Modifications

Other modifications of the antibody are contemplated herein. For example, the antibody may be linked to one of a variety of nonproteinaceous polymers, e.g., polyethylene glycol, polypropylene glycol, polyoxyalkylenes, or copolymers of polyethylene glycol and polypropylene glycol. The antibody also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

Screening for Antibodies with the Desired Properties

Antibodies with certain biological characteristics may be selected as described in the Experimental Examples. For example, antibodies that bind BR3 can be selected by binding to BR3 in ELISA assays or, more preferably, by binding to BR3 expressed on the surface of cells (e.g., BJAB cell line). See, e.g., Example 2.

The growth inhibitory effects of an anti-BR3 antibody of the invention may be assessed by the Examples or methods known in the art, e.g., using cells which express BR3 either endogenously or following transfection with the BR3 gene. For example, in one preferred embodiment, primary B cells expressing BR3 can be used in proliferation and survival assays (e.g., Example 7). In another example, tumor cell lines and BR3-transfected cells may treated with an anti-BR3 monoclonal antibody of the invention at various concentrations for a few days (e.g., 2-7) days and stained with crystal violet or MTT or analyzed by some other colorimetric assay. Another method of measuring proliferation would be by comparing ³H-thymidine uptake by the cells treated in the presence or absence an anti-BR3 antibody of the invention. After antibody treatment, the cells are harvested and the amount of radioactivity incorporated into the DNA quantitated in a scintillation counter. Appropriate positive controls include treatment of a selected cell line with a growth inhibitory antibody known to inhibit growth of that cell line.

To select for antibodies which induce cell death, loss of membrane integrity as indicated by, e.g., propidium iodide (PI), trypan blue or 7AAD uptake may be assessed relative to control. A PI uptake assay can be performed in the absence of complement and immune effector cells. BR3-expressing tumor cells are incubated with medium alone or medium containing of the appropriate monoclonal antibody at e.g, about 10 μg/ml. The cells are incubated for a 3 day time period. Following each treatment, cells are washed and aliquoted into 35 mm strainer-capped 12×75 tubes (1 ml per tube, 3 tubes per treatment group) for removal of cell clumps. Tubes then receive PI (10 μg/ml). Samples may be analyzed using a FACSCAN™ flow cytometer and FACSCONVERT™ CellQuest software (Becton Dickinson). Those antibodies which induce statistically significant levels of cell death as determined by PI uptake may be selected as cell death-inducing antibodies.

To screen for antibodies which bind to an epitope on BR3 bound by an antibody of interest, a routine cross-blocking assay such as that described in Antibodies, A Laboratory Manual, Cold Spring Harbor Laboratory, Ed Harlow and David Lane (1988), can be performed. This assay can be used to determine if a test antibody binds the same site or epitope as an anti-BR3 antibody of the invention. Alternatively, or additionally, epitope mapping can be performed by methods known in the art. For example, the antibody sequence can be mutagenized such as by alanine scanning, to identify contact residues. The mutant antibody is initailly tested for binding with polyclonal antibody to ensure proper folding. In a different method, peptides corresponding to different regions of BR3 can be used in competition assays with the test antibodies or with a test antibody and an antibody with a characterized or known epitope.

### Examples of Specific Anti-BR3Antibodies

Antibodies of this invention specifically include antibodies comprising the variable heavy chain sequence of any one of the antibodies disclosed in Table 2 (below), and BR3-binding fragments thereof that has not been produced by a hybridoma cell. Antibodies of this invention specifically include antibodies comprising a variable heavy chain sequence comprising the sequence of any one of SEQ ID NO: 4-13, 15-18, 22, 24, 26-73, 75-76, 78, 80-85, 87-96, 98, 100, 102, 104, 106-107, 109-110, 112, 114, 116, 118, 120, 122, 124, 126 and 127, and BR3-binding fragments thereof. According to a further embodiment, an antibody of this invention comprises the variable heavy and the variable light chain region of any one of the antibodies disclosed in Table 2, and BR3-binding fragments thereof. According to one embodiment, the antibody further comprises an Fc region comprising the sequence of SEQ ID NO:134, wherein X is an amino acid selected from the group consisting of N, A, W, Y, F and H. According to another embodiment, the antibody comprises the sequence of SEQ ID NO:76 or SEQ 131 NO:131, wherein X is an amino acid selected from the group consisting of N, A, W, Y, F and H.

TABLE 2
Examples of Antibody Sequences

| | SEQ ID | SEQ ID | |
|---|---|---|---|
| ANTIBODY | NO: | NO: | FRAMEWORK |
| 2.1 | 1 (VL) | 2 (VH) | Mouse |
| hu2.1-Graft | 3 (VL) | 4 (VH) | R71A/N73T/L78A |
| Hu2.1-RL | 3 (VL) | 5 (VH) | RL |
| Hu2.1-RE | 3 (VL) | 6 (VH) | RF |
| Hu2.1-40 | 3 (VL) | 7 (VH) | RF |
| Hu2.1-46 | 3 (VL) | 8 (VH) | RF |
| Hu2.1-30 | 3 (VL) | 9 (VH) | RF |
| Hu2.1-93 | 3 (VL) | 10 (VH) | RL |
| Hu2.1-94 | 3 (VL) | 11 (VH) | RL |
| Hu2.1-40L | 3 (VL) | 12 (VH) | RL |
| Hu2.1-89 | 3 (VL) | 13 (VH) | RL |
| Hu2.1-46.DANA- | 14 (LC) | 15 (HC) | RF |
| IgG | | | |
| Hu2.1-27 | 3 (VL) | 16 (VH) | RF |
| Hu2.1-36 | 3 (VL) | 17 (VH) | RF |
| Hu2.1-31 | 3 (VL) | 18 (VH) | RF |
| 9.1 | 19 (VL) | 20 (VH) | Mouse |
| Hu9.1-graft | 21 (VL) | 22 (VH) | R71A/N73T/L78A |
| Hu9.1-73 | 23 (VL) | 24 (VH) | R71A/N73T/L78A |
| Hu9.1-70 | 25 (VL) | 26 (VH) | R71A/N73T/L78A |
| Hu9.1-56 | 21 (VL) | 27 (VH) | R71A/N73T/L78A |
| Hu9.1-51 | 21 (VL) | 28 (VH) | R71A/N73T/L78A |
| Hu9.1-59 | 21 (VL) | 29 (VH) | R71A/N73T/L78A |
| Hu9.1-61 | 21 (VL) | 30 (VH) | R71A/N73T/L78A |
| Hu9.1-A | 21 (VL) | 31 (VH) | R71A/N73T/L78A |
| Hu9.1-B | 21 (VL) | 32 (VH) | R71A/N73T/L78A |
| Hu9.1-C | 21 (VL) | 33 (VH) | R71A/N73T/L78A |
| Hu9.1-66 | 21 (VL) | 34 (VH) | R71A/N73T/L78A |
| Hu9.1-RF | 21 (VL) | 35 (VH) | RF |
| Hu9.1-48 | 21 (VL) | 36 (VH) | RF |
| Hu9.1-RL | 21 (VL) | 37 (VH) | RL |
| Hu9.1-91 | 21 (VL) | 38 (VH) | RL |
| Hu9A-90 | 21 (VL) | 39 (VH) | RL |
| Hu9.1-75 | 21 (VL) | 40 (VH) | RL |
| Hu9.1-88 | 21 (VL) | 41 (VH) | RL |
| Hu9.1RL-9 | 21 (VL) | 42 (VH) | RL |
| Hu9.1RL-44 | 21 (VL) | 43 (VH) | RL |
| Hu9.1RL-13 | 21 (VL) | 44 (VH) | RL |
| Hu9.1RL-47 | 21 (VL) | 45 (VH) | RL |
| Hu9.1RL-28 | 21 (VL) | 46 (VH) | RL |
| Hu9.1RL-43 | 21 (VL) | 47 (VH) | RL |
| Hu9.1RL-16 | 21 (VL) | 48 (VH) | RL |
| Hu9.1RL-70 | 21 (VL) | 49 (VH) | RL |
| Hu9.1RL-30 | 21 (VL) | 50 (VH) | RL |
| Hu9.1RL-32 | 21 (VL) | 51 (VH) | RL |
| Hu9.1RL-37 | 21 (VL) | 52 (VH) | RL |
| Hu9.1RL-29 | 21 (VL) | 53 (VH) | RL |
| Hu9.1RL-10 | 21 (VL) | 54 (VH) | RL |
| Hu9.1RL-24 | 21 (VL) | 55 (VH) | RL |
| Hu9.1RL-39 | 21 (VL) | 56 (VH) | RL |
| Hu9.1RL-31 | 21 (VL) | 57 (VH) | RL |
| Hu9.1RL-18 | 21 (VL) | 58 (VH) | RL |
| Hu9.1RL-23 | 21 (VL) | 59 (VH) | RL |
| Hu9.1RL-41 | 21 (VL) | 60 (VH) | RL |
| Hu9.1RL-95 | 21 (VL) | 61 (VH) | RL |
| Hu9.1RL-14 | 21 (VL) | 62 (VH) | RL |
| Hu9.1RL-57 | 21 (VL) | 63 (VH) | RL |
| Hu9.1RL-15 | 21 (VL) | 64 (VH) | RL |
| Hu9.1RL-54 | 21 (VL) | 65 (VH) | RL |
| Hu9.1RL-12 | 21 (VL) | 66 (VH) | RL |
| Hu9.1RL-34 | 21 (VL) | 67 (VH) | RL |
| Hu9.1RL-25 | 21 (VL) | 68 (VH) | RL |
| Hu9.1RL-71 | 21 (VL) | 69 (VH) | RL |
| Hu9.1RL-5 | 21 (VL) | 70 (VH) | RL |
| Hu9.1RL-79 | 21 (VL) | 71 (VH) | RL |
| Hu9.1RL-66 | 21 (VL) | 72 (VH) | RL |
| Hu9.1RL-69 | 21 (VL) | 73 (VH) | RL |
| 9.1RF-IgG | 74 (LC) | 75 (HC) | RF |
| 9.1RF-IgG | 74 (LC) | 76 (HC) | RF |
| (N434X) | | | |
| 11G9 | 77 (VL) | 78 (VH) | Mouse |
| Hu11G9-graft | 79 (VL) | 80 (VH) | R71A/N73T/L78A |
| Hu11G9-RF | 79 (VL) | 81 (VH) | RF |
| Hu11G9-36 | 79 (VL) | 82 (VH) | RF |
| Hu11G9-46 | 79 (VL) | 83 (VH) | RF |
| Hu11G9-35 | 79 (VL) | 84 (VH) | RF |
| Hu11G9-29 | 79 (VL) | 85 (VH) | RF |
| V3-Fab | 86 (LC) | 87 (HC) | |
| V24 | 86 (VL) | 88 (VH) | |
| V44 | 86 (VL) | 89 (VH) | |
| V89 | 86 (VL) | 90 (VH) | |
| V96 | 86 (VL) | 91 (VH) | |
| V46 | 86 (VL) | 92 (VH) | |
| V51 | 86 (VL) | 93 (VH) | |
| V75 | 86 (VL) | 94 (VH) | |
| V58 | 86 (VL) | 95 (VH) | |
| V60 | 86 (VL) | 96 (VH) | |
| V3-1 | 97 (VL) | 98 (VH) | |
| V3-11 | 99 (VL) | 100 (VH) | |
| V3-12 | 101 (VL) | 102 (VH) | |
| V3-13 | 103 (VL) | 104 (VH) | |
| V3-3 | 105 (VL) | 106 (VH) | |
| V3-5 | 97 (VL) | 107 (VH) | |
| V3-9 | 108 (VL) | 98 (VH) | |
| V3-16 | 97 (VL) | 109 (VH) | |
| V3-19 | 97 (VL) | 110 (VH) | |
| V3-24 | 111 (VL) | 112 (VH) | |
| V3-27 | 113 (VL) | 114 (VH) | |
| V3-34 | 115 (VL) | 116 (VH) | |
| V3-35 | 117 (VL) | 118 (VH) | |
| V3-37 | 119 (VL) | 120 (VH) | |
| V3-41 | 121 (VL) | 122 (VH) | |
| V3-46 | 123 (VL) | 124 (VH) | |
| V3-46a | 123 (VL) | 125 (VH) | |
| V3-46q | 123 (VL) | 126 (VH) | |
| V3-46s | 123 (VL) | 127 (VH) | |
| V3-46sFab | 128 (LC) | 129 (VH) | |
| V3-46s IgG | 128 (LC) | 130 (VH) | |
| V3-46s IgG | 128 (LC) | 131 (VH) | |
| (N434X) | | | |
| V3-46s-1 | 194 (LC) | 127 (VH) | |
| V3-46s-7 | 195 (LC) | 127 (VH) | |
| V3-46s-9 | 196 (LC) | 127 (VH) | |
| V3-46s-10 | 197 (LC) | 127 (VH) | |
| V3-46s-12 | 198 (LC) | 193 (VH) | |
| V3-46s-13 | 199 (LC) | 127 (VH) | |
| V3-46s-29 | 200 (LC) | 127 (VH) | |
| V3-46s-31 | 201 (LC) | 127 (VH) | |
| V3-46s-33 | 202 (LC) | 127 (VH) | |
| V3-46s-34 | 203 (LC) | 127 (VH) | |
| V3-46s-37 | 204 (LC) | 127 (VH) | |
| V3-46s-40 | 205 (LC) | 127 (VH) | |
| V3-46s-42 | 206 (LC) | 127 (VH) | |
| V3-46s-45 | 207 (LC) | 127 (VH) | |

Antibodies of this invention include BR3-binding antibodies having an H3 sequence that is at least about 70% amino acid sequence identity, alternatively at least about 71%, 72%, 73%, 74%, 75%, 76%, 77%, 78%, 79%, 80%, 81%, 82%, 83%, 84%, 85%, 86%, 87%, 88%, 89%, 89%, 90%, 91%, 92%, 93%, 94%, 95%, 96%, 97%, 98%, or 99% amino acid sequence identity, to the H3 sequence of any one of the sequences of SEQ ID NO:s: 4-13, 15-18, 22, 24, 26-73, 75-76, 78, 80-85, 87-96, 98, 100, 102, 104, 106-107, 109-110, 112, 114, 116, 118, 120, 122, 124, 126 and 127, and BR3 binding fragments of those antibodies.

Antibodies of this invention include BR3-binding antibodies having H1, H2 and H3 sequences that are at least 70% identical to the underlined portions of any one of the antibodies sequences described in the Figures or to the CDRs of hypervariable regions described in the Sequence Listing, or alternatively at least about 71%, 72%, 73%, 74%, 75%, 76%, 77%, 78%, 79%, 80%, 81%, 82%, 83%, 84%, 85%, 86%, 87%, 88%, 89%, 89%, 90%, 91%, 92%, 93%, 94%, 95%, 96%, 97%, 98%, or 99% amino acid sequence identical.

Antibodies of this invention include BR3-binding antibodies having L1, L2 and L3 sequences that are at least 70% identical to the underlined portions of any one of the antibodies sequences described in the Figures or to the CDRs or hypervariable regions described in the Sequence Listing, or alternatively at least about 71%, 72%, 73%, 74%, 75%, 76%, 77%, 78%, 79%, 80%, 81%, 82%, 83%, 84%, 85%, 86%, 87%, 88%, 89%, 89%, 90%, 91%, 92%, 93%, 94%, 95%, 96%, 97%, 98%, or 99% amino acid sequence identical.

Antibodies of this invention include BR3-binding antibodies having a VH domain with at least 70% homology to a VH domain of any one of the antibodies of Table 2, or alternatively at least about 71%, 72%, 73%, 74%, 75%, 76%, 77%, 78%, 79%, 80%, 81%, 82%, 83%, 84%, 85%, 86%, 87%, 88%, 89%, 89%, 90%, 91%, 92%, 93%, 94%, 95%, 96%, 97%, 98%, or 99% amino acid sequence identical.

Antibodies of this invention include any BR3-binding antibody comprising a heavy chain CDR3 sequence of an antibody sequence of Table 2 that has not been produced by a hybridoma cell. Antibodies of this invention include any BR3-binding antibody comprising a heavy chain CDR3 sequence of any one of SEQ ID NO:s:7-13, 15-18, 36, 38-73, 78, 82-85, 87-96, 98, 100, 102, 104, 106-107, 109-110, 112, 114, 116, 118, 120, 122, 124, 126 and 127, or comprising a H3 sequence that is derived a H3 sequence of any one of SEQ ID NO:s:7-13, 15-18, 36, 38-73, 78, 82-85, 87-96, 98, 100, 102, 104, 106-107, 109-110, 112, 114, 116, 118, 120, 122, 124, 126 and 127. In another embodiment, an antibody of this invention includes any BR3-binding antibody comprising a CDR-H1, CDR-H2 and CDR-H3 of any one of the sequences selected from the group consisting of SEQ ID NOs:7-13, 15-18, 36, 38-73, 78, 82-85, 87-96, 98, 100, 102, 104, 106-107, 109-110, 112, 114, 116, 118, 120, 122, 124, 126 and 127 or is derived from an antibody comprising the CDR-H1, CDR-H2 and CDR-H3 sequences. Antibodies of this invention include any BR3-binding antibody comprising a heavy chain H1, H2 and 1-13 sequence of an antibody of Table 2 that has not been produced by a hybridoma cell.

Antibodies of this invention include the antibodies comprising a polypeptide sequence encoded by the Hu9.1-RF-H-IgG nucleic acid sequence deposited as ATCC deposit number PTA-6315 on Nov. 17, 2004 and anti-BR3 binding antibodies that comprise an amino acid sequence that is at least 70% identical, alternatively at least about 71%, 72%, 73%, 74%, 75%, 76%, 77%, 78%, 79%, 80%, 81%, 82%, 83%, 84%, 85%, 86%, 87%, 88%, 89%, 89%, 90%, 91%, 92%, 93%, 94%, 95%, 96%, 97%, 98%, or 99% amino acid sequence identical, to any one of the variable regions sequence of the Hu9.1-RF-H-IgG polypeptide sequence. Antibodies of this invention include the antibodies comprising a polypeptide sequence encoded by the Hu9.1-RF-L-IgG nucleic acid sequence deposited as ATCC deposit number PTA-6316 on Nov. 17, 2004 and anti-BR3 binding antibodies that comprise an amino acid sequence that is at least 70% identical, alternatively at least about 71%, 72%, 73%, 74%, 75%, 76%, 77%, 78%, 79%, 80%, 81%, 82%, 83%, 84%, 85%, 86%, 87%, 88%, 89%, 89%, 90%, 91%, 92%, 93%, 94%, 95%, 96%, 97%, 98%, or 99% amino acid sequence identical, to the variable region sequence of the Hu9.1-RF-L-IgG polypeptide sequence.

Antibodies of this invention include the antibodies comprising a polypeptide sequence encoded by the Hu2.1-46. DANA-H-IgG nucleic acid sequence deposited as ATCC deposit number PTA-6313 on Nov. 17, 2004 and anti-BR3 binding antibodies that comprise an amino acid sequence that is at least 70% identical, alternatively at least about 71%, 72%, 73%, 74%, 75%, 76%, 77%, 78%, 79%, 80%, 81%, 82%, 83%, 84%, 85%, 86%, 87%, 88%, 89%, 89%, 90%, 91%, 92%, 93%, 94%, 95%, 96%, 97%, 98%, or 99% amino acid sequence identical, to the variable region sequence of the Hu2.1-46. DANA-H-IgG polypeptide sequence. Antibodies of this invention include the antibodies comprising a polypeptide sequence encoded by the Hu2.1-46. DANA-L-IgG nucleic acid sequence deposited as ATCC deposit number PTA-6314 on Nov. 17, 2004 and anti-BR3 binding antibodies that comprise an amino acid sequence that is at least 70% identical, alternatively at least about 71%, 72%, 73%, 74%, 75%, 76%, 77%, 78%, 79%, 80%, 81%, 82%, 83%, 84%, 85%, 86%, 87%, 88%, 89%, 89%, 90%, 91%, 92%, 93%, 94%, 95%, 96%, 97%, 98%, or 99% amino acid sequence identical, to the variable region sequence of the Hu2.1-46. DANA-L-IgG polypeptide sequence.

Antibodies of this invention include the antibodies comprising a polypeptide sequence encoded by the HuV3-46s-H-IgG nucleic acid sequence deposited as ATCC deposit number PTA-6317 on Nov. 17, 2004 and anti-BR3 binding antibodies that comprise an amino acid sequence that is at least 70% identical, alternatively at least about 71%, 72%, 73%, 74%, 75%, 76%, 77%, 78%, 79%, 80%, 81%, 82%, 83%; 84%, 85%, 86%, 87%, 88%, 89%, 89%, 90%, 91%, 92%, 93%, 94%, 95%, 96%, 97%, 98%, or 99% amino acid sequence identical, to the variable region sequence of the HuV3-46s-H-IgG polypeptide sequence. Antibodies of this invention include the antibodies comprising a polypeptide sequence encoded by the HuV3-46s-L-IgG nucleic acid sequence deposited as ATCC deposit number PTA-6318 on Nov. 17, 2004 and anti-BR3 binding antibodies that comprise an amino acid sequence that is at least 70% identical, alternatively at least about 71%, 72%, 73%, 74%, 75%, 76%, 77%, 78%, 79%, 80%, 81%, 82%, 83%, 84%, 85%, 86%, 87%, 88%, 89%, 89%, 90%, 91%, 92%, 93%, 94%, 95%, 96%, 97%, 98%, or 99% amino acid sequence identical, to the variable region sequence of the HuV3-46s-L-IgG polypeptide sequence.

Antibodies of this invention include the Hu9.1-RF-IgG antibody comprising the heavy chain sequence of ATCC deposit no. PTA-6315 and the light chain sequence of ATCC deposit no. PTA-6316. Antibodies of this invention include the Hu2.1-46. DANA-IgG antibody comprising the heavy sequence of ATCC deposit no. PTA-6313 and the light chain sequence of ATCC deposit no. PTA-6314. Antibodies of this invention include the HuV3-46s-IgG antibody comprising the heavy sequence of ATCC deposit no. PTA-6317 and the light chain sequence of ATCC deposit no. PTA-6318.

According to one preferred embodiment, the antibodies of this invention specifically bind to a sequence of a native human BR3 polypeptide. According to yet another embodiment, an antibody of this invention has improved binding to the FcRn receptor at pH 6.0 compared to the antibody known as 9.1-RF Ig. According to yet another embodiment, an antibody of this invention has improved ADCC function in the presence of human effector cells compared to the antibody known as 9.1-RF Ig. According to yet another embodiment, an antibody of this invention has decreased ADCC function in the presence of human effector cells compared to the antibody known as 9.1-RF Ig.

It is understood that all antibodies of this invention include antibodies lacking a signal sequence and antibodies lacking the K447 residue of the Fc region.

### Vectors, Host Cells and Recombinant Methods

The invention also provides an isolated nucleic acid encoding a BR3 binding antibody or BR3 binding polypeptide, vectors and host cells comprising the nucleic acid, and recombinant techniques for the production of the antibody.

For recombinant production of the BR3 binding antibodies and polypeptides, the nucleic acid encoding it is isolated and inserted into a replicable vector for further cloning (amplification of the DNA) or for expression. DNA encoding the monoclonal antibody or polypeptide is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence.

(i) Signal Sequence Component

The antibody or polypeptide of this invention may be produced recombinantly not only directly, but also as a fusion polypeptide with a heterologous polypeptide, which is preferably a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. For prokaryotic host cells that do not recognize and process the native BR3 binding antibody signal sequence, the signal sequence is substituted by a prokaryotic signal sequence selected, for example, from the group of the alkaline phosphatase, penicillinase, 1 pp, or heat-stable enterotoxin II leaders. For yeast secretion the native signal sequence may be substituted by, e.g., the yeast invertase leader, a factor leader (including Saccharomyces and Kluyveromyces α-factor leaders), or acid phosphatase leader, the C. albicans glucoamylase leader, or the signal described in WO 90/13646. In mammalian cell expression, mammalian signal sequences as well as viral secretory leaders, for example, the herpes simplex gD signal, are available.

The DNA for such precursor region is ligated in reading frame to DNA encoding the BR3 binding antibody.

(ii) Origin of Replication

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

(iii) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the BR3 binding antibody nucleic acid, such as DHFR, thymidine kinase, metallothionein-I and -II, preferably primate metallothionein genes, adenosine deaminase, ornithine decarboxylase, etc.

For example, cells transformed with the DHFR selection gene are first identified by culturing all of the transformants in a culture medium that contains methotrexate (Mix), a competitive antagonist of DHFR. An appropriate host cell when wild-type DHFR is employed is the Chinese hamster ovary (CHO) cell line deficient in DHFR activity (e.g., ATCC CRL-9096).

Alternatively, host cells (particularly wild-type hosts that contain endogenous DHFR) transformed or co-transformed with DNA sequences encoding BR3 binding antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trp1 gene present in the yeast plasmid YRp7 (Stinchcomb et al., Nature, 282:39 (1979)). The trp1 gene provides a selection marker for a mutant strain of yeast lacking the ability to grow in tryptophan, for example, ATCC No. 44076 or PEP4-1. Jones, Genetics, 85:12 (1977). The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

In addition, vectors derived from the 1.6 μm circular plasmid pKD1 can be used for transformation of Kluyveromyces yeasts. Alternatively, an expression system for large-scale production of recombinant calf chymosin was reported for K. lactis. Van den Berg, Bio/Technology, 8:135 (1990). Stable multi-copy expression vectors for secretion of mature recombinant human serum albumin by industrial strains of Kluyveromyces have also been disclosed. Fleer et al., Bio/Technology, 9:968-975 (1991).

(iv) Promoter Component

Expression and cloning vectors usually contain a promoter that is recognized by the host organism and is operably linked to the nucleic acid encoding the BR3 binding antibody. Promoters suitable for use with prokaryotic hosts include the phoA promoter, β-lactamase and lactose promoter systems, alkaline phosphatase promoter, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the BR3 binding antibody.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoter sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

Antibody transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus, Simian Virus 40 (SV40), or from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human β-interferon cDNA in mouse cells under the control of a thymidine kinase promoter from herpes simplex virus. Alternatively, the Rous Sarcoma Virus long terminal repeat can be used as the promoter.

(v) Enhancer Element Component

Transcription of a DNA encoding an antibody of this invention by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270); the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the antibody-encoding sequence, but is preferably located at a site 5′ from the promoter.

(vi) Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding antibody. One useful transcription termination component is the bovine growth hormone polyadenylation region. See WO94/11026 and the expression vector disclosed therein.

(vii) Selection and transformation of host cells Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

Full length antibody, antibody fragments, and antibody fusion proteins can be produced in bacteria, in particular when glycosylation and Fc effector function are not needed, such as when the therapeutic antibody is conjugated to a cytotoxic agent (e.g., a toxin) and the immunoconjugate by itself shows effectiveness in tumor cell destruction. Full length antibodies have greater half life in circulation. Production in E. coli is faster and more cost efficient. For expression of antibody fragments and polypeptides in bacteria, see, e.g., U.S. Pat. No. 5,648,237 (Carter et. al.), U.S. Pat. No. 5,789,199 (Joly et al.), and U.S. Pat. No. 5,840,523 (Simmons et al.) which describes translation initiation region (TIR) and signal sequences for optimizing expression and secretion, these patents incorporated herein by reference. After expression, the antibody is isolated from the E. coli cell paste in a soluble fraction and can be purified through, e.g., a protein A or G column depending on the isotype. Final purification can be carried out similar to the process for purifying antibody expressed e.g., in CHO cells.

In addition to prokaryotes, eukaryotic microbes such as filamentous fungi or yeast are suitable cloning or expression hosts for BR3 binding antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated BR3 binding antibody are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for antibody production and cultured in conventional nutrient media modified as appropriate for inducing promoters, selecting transformants, or amplifying the genes encoding the desired sequences.

(viii) Culturing the Host Cells

The host cells used to produce an antibody of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. No. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

(ix) Purification of Antibody

When using recombinant techniques, the antibody can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, hydrophobic interaction chromatography, gel electrophoresis, dialysis, and affinity chromatography, with affinity chromatography being among one of the typically preferred purification steps. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### Antibody Conjugates

The antibody may be conjugated to a cytotoxic agent, such as a chemotherapeutic agent, toxin (e.g., an enzymatically active toxin of bacterial, fungal, plant, or animal origin, or fragments thereof), or a radioactive isotope (i.e., a radioconjugate). In certain embodiments, the toxin is calicheamicin, a maytansinoid, a dolastatin, auristatin E and analogs or derivatives thereof, are preferable.

Chemotherapeutic agents useful in the generation of such immunoconjugates have been described herein. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

Preferred drugs/toxins include DNA damaging agents, inhibitors of microtubule polymerization or depolymerization and antimetabolites. Preferred classes of cytotoxic agents include, for example, the enzyme inhibitors such as dihydrofolate reductase inhibitors, and thymidylate synthase inhibitors, DNA intercalators, DNA cleavers, topoisomerase inhibitors, the anthracycline family of drugs, the vinca drugs, the mitomycins, the bleomycins, the cytotoxic nucleosides, the pteridine family of drugs, diynenes, the podophyllotoxins and differentiation inducers. Particularly useful members of those classes include, for example, methotrexate, methopterin, dichloromethotrexate, 5-fluorouracil, 6-mercaptopurine, cytosine arabinoside, melphalan, leurosine, leurosideine, actinomycin, daunorubicin, doxorubicin, N-(5,5-diacetoxypentyl)doxorubicin, morpholino-doxorubicin, 1-(2-choroehthyl)-1,2-dimethanesulfonyl hydrazide, N⁸-acetyl spermidine, aminopterin methopterin, esperamicin, mitomycin C, mitomycin A, actinomycin, bleomycin, caminomycin, aminopterin, tallysomycin, podophyllotoxin and podophyllotoxin derivatives such as etoposide or etoposide phosphate, vinblastine, vincristine, vindesine, taxol, taxotere, retinoic acid, butyric acid, N⁸⁻acetyl spermidine, camptothecin, calicheamicin, bryostatins, cephalostatins, ansamitocin, actosin, maytansinoids such as DM-1, maytansine, maytansinol, N-desmethyl-4,5-desepoxymaytansinol, C-19-dechloromaytansinol, C-20-hydroxymaytansinol, C-20-demethoxymaytansinol, C-9-SH maytansinol, C-14-alkoxymethylmaytansinol, C-14-hydroxy or acetyloxymethlmaytansinol, C-15-hydroxy/acetyloxymaytansinol, C-15-methoxymaytansinol, C-18-N-demethylmaytansinol and 4,5-deoxymaytansinol, auristatins such as auristatin E, M, PHE and PE; dolostatins such as dolostatin A, dolostatin B, dolostatin C, dolostatin D, dolostatin E (20-epi and 11-epi), dolostatin G, dolostatin H, dolostatin I, dolostatin 1, dolostatin 2, dolostatin 3, dolostatin 4, dolostatin 5, dolostatin 6, dolostatin 7, dolostatin 8, dolostatin 9, dolostatin 10, deo-dolostatin 10, dolostatin 11, dolostatin 12, dolostatin 13, dolostatin 14, dolostatin 15, dolostatin 16, dolostatin 17, and dolostatin 18; cephalostatins such as cephalostatin 1, cephalostatin 2, cephalostatin 3, cephalostatin 4, cephalostatin 5, cephalostatin 6, cephalostatin 7, 25′-epi-cephalostatin 7,20-epi-cephalostatin 7, cephalostatin 8, cephalostatin 9, cephalostatin 10, cephalostatin 11, cephalostatin 12, cephalostatin 13, cephalostatin 14, cephalostatin 15, cephalostatin 16, cephalostatin 17, cephalostatin 18, and cephalostatin 19.

Maytansinoids are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533, the disclosures of which are hereby expressly incorporated by reference.

Maytansine and maytansinoids have been conjugated to antibodies specifically binding to tumor cell antigens. Immunoconjugates containing maytansinoids and their therapeutic use are disclosed, for example, in U.S. Pat. Nos. 5,208,020, 5,416,064 and European Patent EP 0 425 235 B1, the disclosures of which are hereby expressly incorporated by reference. Liu et al., Proc. Natl. Acad. Sci. USA 93:8618-8623 (1996) described immunoconjugates comprising a maytansinoid designated DM1 linked to the monoclonal antibody C242 directed against human colorectal cancer. The conjugate was found to be highly cytotoxic towards cultured colon cancer cells, and showed antitumor activity in an in vivo tumor growth assay. Chari et al. Cancer Research 52:127-131 (1992) describe immunoconjugates in which a maytansinoid was conjugated via a disulfide linker to the murine antibody A7 binding to an antigen on human colon cancer cell lines, or to another murine monoclonal antibody TA.1 that binds the HER-2/neu oncogene.

There are many linking groups known in the art for making antibody-maytansinoid conjugates, including, for example, those disclosed in U.S. Pat. No. 5,208,020 or EP Patent 0 425 235 B1, and Chari et at Cancer Research 52: 127-131 (1992). The linking groups include disufide groups, thioether groups, acid labile groups, photolabile groups, peptidase labile groups, or esterase labile groups, as disclosed in the above-identified patents, disulfide and thioether groups being preferred.

Conjugates of the antibody and maytansinoid may be made using a variety of bifunctional protein coupling agents such as N-succinimidyl-3-(2-pyridyldithio) propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Particularly preferred coupling agents include N-succinimidyl-3-(2-pyridyldithio) propionate (SPDP) (Carlsson et al., Biochem. J. 173:723-737 [1978]) and N-succinimidyl-4-(2-pyridylthio)pentanoate (SPP) to provide for a disulfide linkage.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hyrdoxymethyl, the C-15 position modified with a hydroxyl group, and the C-20 position having a hydroxyl group. In a preferred embodiment, the linkage is formed at the C-3 position of maytansinol or a maytansinol analogue.

Calicheamicin

Another immunoconjugate of interest comprises an BR3 binding antibody conjugated to one or more calicheamicin molecules. The calicheamicin family of antibiotics are capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, 5,877,296 (all to American Cyanamid Company). Structural analogues of calicheamicin which may be used include, but are not limited to, γ₁^{I}, α₂^{I}, α₃^{I}, N-acetyl-γ₁^{I}, PSAG and θ¹₁ (Hinman et al. Cancer Research 53: 3336-3342 (1993), Lode et al. Cancer Research 58: 2925-2928 (1998) and the aforementioned U.S. patents to American Cyanamid). Another anti-tumor drug that the antibody can be conjugated is QFA which is an antifolate. Both calicheamicin and QFA have intracellular sites of action and do not readily cross the plasma membrane. Therefore, cellular uptake of these agents through antibody mediated internalization greatly enhances their cytotoxic effects.

Radioactive Isotopes

For selective destruction of the tumor, the antibody may comprise a highly radioactive atom. A variety of radioactive isotopes are available for the production of radioconjugated anti-BR3 antibodies. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the conjugate is used for diagnosis, it may comprise a radioactive atom for scintigraphic studies, for example tc^{99m} or I¹²³, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as tc^{99m} or I¹²³, Re¹⁸⁶, Re¹⁸⁸ and In¹¹¹ can be attached via a cysteine residue in the peptide. Yttrium-90 can be attached via a lysine residue. The IODOGEN method (Fraker et al (1978) Biochem. Biophys. Res. Commun. 80: 49-57 can be used to incorporate iodine-123. “Monoclonal Antibodies in Immunoscintigraphy” (Chatal, CRC Press 1989) describes other methods in detail.

Conjugates of the antibody and cytotoxic agent may be made using a variety of bifunctional protein coupling agents such as N-succinimidyl-3-(2-pyridyldithio) propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al. Cancer Research 52: 127-131 (1992); U.S. Pat. No. 5,208,020) may be used.

In another embodiment, the antibody can be conjugated to a “receptor” (such as streptavidin) for utilization in tumor pretargeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

### Therapeutic Uses of the BR3 Binding Antibodies

The BR3 binding antibodies of the invention are useful to treat a number of malignant and non-malignant diseases including autoimmune diseases and related conditions, and cancers, including BR3 positive cancers including B cell lymphomas and leukemias. Stem cells (B-cell progenitors) in bone marrow lack the BR3 antigen, allowing healthy B-cells to regenerate after treatment and return to normal levels within several months.

Autoimmune diseases or autoimmune related conditions include arthritis (rheumatoid arthritis, juvenile rheumatoid arthritis, osteoarthritis, psoriatic arthritis), psoriasis, dermatitis including atopic dermatitis; chronic autoimmune urticaria, polymyositis/dermatomyositis, toxic epidermal necrolysis, systemic scleroderma and sclerosis, responses associated with inflammatory bowel disease (IBD) (Crohn's disease, ulcerative colitis), respiratory distress syndrome, adult respiratory distress syndrome (ARDS), meningitis, allergic rhinitis, encephalitis, uveitis, colitis, glomerulonephritis, allergic conditions, eczema, asthma, conditions involving infiltration of T cells and chronic inflammatory responses, atherosclerosis, autoimmune myocarditis, leukocyte adhesion deficiency, systemic lupus erythematosus (SLE), lupus (including nephritis, non-renal, discoid, alopecia), juvenile onset diabetes, multiple sclerosis, allergic encephalomyelitis, immune responses associated with acute and delayed hypersensitivity mediated by cytokines and T-lymphocytes, tuberculosis, sarcoidosis, granulomatosis including Wegener's granulomatosis, agranulocytosis, vasculitis (including ANCA), aplastic anemia, Coombs positive anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome, myasthenia gravis, antigen-antibody complex mediated diseases, anti-glomerular basement membrane disease, anti-phospholipid antibody syndrome, allergic neuritis, Bechet disease, Castleman's syndrome, Goodpasture's Syndrome, Lambert-Eaton Myasthenic Syndrome, Reynaud's syndrome, Sjorgen's syndrome, Stevens-Johnson syndrome, solid organ transplant rejection (including pretreatment for high panel reactive antibody titers, IgA deposit in tissues, etc), graft versus host disease (GVHD), pemphigoid bullous, pemphigus (all including vulgaris, foliatis), autoimmune polyendocrinopathies, Reiter's disease, stiff-man syndrome, giant cell arteritis, immune complex nephritis, IgA nephropathy, IgM polyneuropathies or IgM mediated neuropathy, idiopathic thrombocytopenic purpura (ITP), thrombotic throbocytopenic purpura (TTP), autoimmune thrombocytopenia, autoimmune disease of the testis and ovary including autoimune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes also referred to as insulin-dependent diabetes mellitus (IDDM) and Sheehan's syndrome; autoimmune hepatitis, Lymphoid interstitial pneumonitis (HIV), bronchiolitis obliterans (non-transplant) vs NSIP, Guillain-Barre' Syndrome, Large Vessel Vasculitis (including Polymyalgia Rheumatica and Giant Cell (Takayasu's) Arteritis), Medium Vessel Vasculitis (including Kawasaki's Disease and Polyarteritis Nodosa), ankylosing spondylitis, Berger's Disease (IgA nephropathy), Rapidly Progressive Glomerulonephritis, Primary biliary cirrhosis, Celiac sprue (gluten enteropathy), Cryoglobulinemia, ALS, coronary artery disease.

BR3 positive cancers are those comprising abnormal proliferation of cells that express BR3 on the cell surface. The BR3 positive B cell neoplasms include BR3-positive Hodgkin's disease including lymphocyte predominant Hodgkin's disease (LPHD); non-Hodgkin's lymphoma (NHL); follicular center cell (FCC) lymphomas; acute lymphocytic leukemia (ALL); chronic lymphocytic leukemia (CLL); Hairy cell leukemia. The non-Hodgkins lymphoma include low grade/follicular non-Hodgkin's lymphoma (NHL), small lymphocytic lymphoma (SLL), intermediate grade/follicular NHL, intermediate grade diffuse NHL, high grade immunoblastic NHL, high grade lymphoblastic NHL, high grade small non-cleaved cell NHL, bulky disease NHL, plasmacytoid lymphocytic lymphoma, mantle cell lymphoma, AIDS—related lymphoma and Waldenstrom's macroglobulinemia. Treatment of relapses of these cancers are also contemplated. LPHD is a type of Hodgkin's disease that tends to relapse frequently despite radiation or chemotherapy treatment and is characterized by BR3-positive malignant cells. CLL is one of four major types of leukemia. A cancer of mature B-cells called lymphocytes, CLL is manifested by progressive accumulation of cells in blood, bone marrow and lymphatic tissues.

In specific embodiments, the BR3 binding antibodies and functional fragments thereof are used to treat non-Hodgkin's lymphoma (NHL), lymphocyte predominant Hodgkin's disease (LPHD), small lymphocytic lymphoma (SLL), chronic lymphocytic leukemia, rheumatoid arthritis and juvenile rheumatoid arthritis, systemic lupus erythematosus (SLE) including lupus nephritis, Wegener's disease, inflammatory bowel disease, idiopathic thrombocytopenic purpura (ITP), thrombotic throbocytopenic purpura (TTP), autoimmune thrombocytopenia, multiple sclerosis, psoriasis, IgA nephropathy, IgM polyneuropathies, myasthenia gravis, vasculitis, diabetes mellitus, Reynaud's syndrome, Sjorgen's syndrome and glomerulonephritis.

The BR3 binding antibodies or functional fragments thereof are useful as a single-agent treatment in, e.g., for relapsed or refractory low-grade or follicular, BR3-positive, B-cell NHL, or can be administered to patients in conjunction with other drugs in a multi drug regimen.

Indolent lymphoma is a slow-growing, incurable disease in which the average patient survives between six and 10 years following numerous periods of remission and relapse. In one embodiment, the humanized BR3 binding antibodies or functional fragments thereof are used to treat indolent NHL.

The parameters for assessing efficacy or success of treatment of the neoplasm will be known to the physician of skill in the appropriate disease. Generally, the physician of skill will look for reduction in the signs and symptoms of the specific disease. Parameters can include median time to disease progression, time in remission, stable disease.

The following references describe lymphomas and CLL, their diagnoses, treatment and standard medical procedures for measuring treatment efficacy.

The following references describe lymphomas and CLL, their diagnoses, treatment and standard medical procedures for measuring treatment efficacy. Canellos G P, Lister, T A, Sklar J L: The Lymphomas. W.B. Saunders Company, Philadelphia, 1998; van Besien K and Cabanillas, F: Clinical Manifestations, Staging and Treatment of Non-Hodgkin's Lymphoma, Chap. 70, pp 1293-1338, in: Hematology, Basic Principles and Practice, 3rd ed. Hoffman et al. (editors). Churchill Livingstone, Philadelphia, 2000; and Rai, K and Patel, D:Chronic Lymphocytic Leukemia, Chap. 72, pp 1350-1362; in: Hematology, Basic Principles and Practice, 3rd ed. Hoffman et al. (editors). Churchill Livingstone, Philadelphia, 2000.

The parameters for assessing efficacy or success of treatment of an autoimmune or autoimmune related disease will be known to the physician of skill in the appropriate disease. Generally, the physician of skill will look for reduction in the signs and symptoms of the specific disease. The following are by way of examples.

In one embodiment, the antibodies of the invention are useful to treat rheumatoid arthritis. RA is characterized by inflammation of multiple joints, cartilage loss and bone erosion that leads to joint destruction and ultimately reduced joint function. Additionally, since RA is a systemic disease, it can have effects in other tissues such as the lungs, eyes and bone marrow. Fewer than 50 percent of patients who have had RA for more than 10 years can continue to work or function normally on a day-to-day basis.

The antibodies can be used as first-line therapy in patients with early RA (i.e., methotrexate (MTX) naive) and as monotherapy, or in combination with, e.g., MTX or cyclophosphamide. Or, the antibodies can be used in treatment as second-line therapy for patients who were DMARD and/or MTX refractory, and as monotherapy or in combination with, e.g., MTX. The humanized BR3 binding antibodies are useful to prevent and control joint damage, delay structural damage, decrease pain associated with inflammation in RA, and generally reduce the signs and symptoms in moderate to severe RA. The RA patient can be treated with the humanized BR3 antibody prior to, after or together with treatment with other drugs used in treating RA (see combination therapy below). In one embodiment, patients who had previously failed disease-modifying antirheumatic drugs and/or had an inadequate response to methotrexate alone are treated with a humanized BR3 binding antibody of the invention. In one embodiment of this treatment, the patients are in a 17-day treatment regimen receiving humanized BR3 binding antibody alone (1g iv infusions on days 1 and 15); BR3 binding antibody plus cyclophosphamide (750 mg iv infusion days 3 and 17); or BR3 binding antibody plus methotrexate.

One method of evaluating treatment efficacy in RA is based on American College of Rheumatology (ACR) criteria, which measures the percentage of improvement in tender and swollen joints, among other things. The RA patient can be scored at for example, ACR 20 (20 percent improvement) compared with no antibody treatment (e.g baseline before treatment) or treatment with placebo. Other ways of evaluating the efficacy of antibody treatment include X-ray scoring such as the Sharp X-ray score used to score structural damage such as bone erosion and joint space narrowing. Patients can also be evaluated for the prevention of or improvement in disability based on Health Assessment Questionnaire [HAQ] score, AIMS score, SF-36 at time periods during or after treatment. The ACR 20 criteria may include 20% improvement in both tender (painful) joint count and swollen joint count plus a 20% improvement in at least 3 of 5 additional measures:
1. patient's pain assessment by visual analog scale (VAS),
2. patient's global assessment of disease activity (VAS),
3. physician's global assessment of disease activity (VAS),
4. patient's self-assessed disability measured by the Health Assessment Questionnaire, and
5. acute phase reactants, CRP or ESR.
   The ACR 50 and 70 are defined analogously. Preferably, the patient is administered an amount of a BR3 binding antibody of the invention effective to achieve at least a score of ACR 20, preferably at least ACR 30, more preferably at least ACR50, even more preferably at least ACR70, most preferably at least ACR 75 and higher.

Psoriatic arthritis has unique and distinct radiographic features. For psoriatic arthritis, joint erosion and joint space narrowing can be evaluated by the Sharp score as well. The humanized BR3 binding antibodies of the invention can be used to prevent the joint damage as well as reduce disease signs and symptoms of the disorder.

Yet another aspect of the invention is a method of treating Lupus or SLE by administering to the patient suffering from SLE, a therapeutically effective amount of a BR3 binding antibody of the invention. SLEDAI scores provide a numerical quantitation of disease activity. The SLEDAI is a weighted index of 24 clinical and laboratory parameters known to correlate with disease activity, with a numerical range of 0-103. see Bryan Gescuk & John Davis, “Novel therapeutic agent for systemic lupus erythematosus” in Current Opinion in Rheumatology 2002, 14:515-521. Antibodies to double-stranded DNA are believed to cause renal flares and other manifestations of lupus. Patients undergoing antibody treatment can be monitored for time to renal flare, which is defined as a significant, reproducible increase in serum creatinine, urine protein or blood in the urine. Alternatively or in addition, patients can be monitored for levels of antinuclear antibodies and antibodies to double-stranded DNA. Treatments for SLE include high-dose corticosteroids and/or cyclophosphamide (HDCC).

Spondyloarthropathies are a group of disorders of the joints, including ankylosing spondylitis, psoriatic arthritis and Crohn's disease. Treatment success can be determined by validated patient and physician global assessment measuring tools.

Various medications are used to treat psoriasis; treatment differs directly in relation to disease severity. Patients with a more mild form of psoriasis typically utilize topical treatments, such as topical steroids, anthralin, calcipotriene, clobetasol, and tazarotene, to manage the disease while patients with moderate and severe psoriasis are more likely to employ systemic (methotrexate, retinoids, cyclosporine, PUVA and UVB) therapies. Tars are also used. These therapies have a combination of safety concerns, time consuming regimens, or inconvenient processes of treatment. Furthermore, some require expensive equipment and dedicated space in the office setting. Systemic medications can produce serious side effects, including hypertension, hyperlipidemia, bone marrow suppression, liver disease, kidney disease and gastrointestinal upset. Also, the use of phototherapy can increase the incidence of skin cancers. In addition to the inconvenience and discomfort associated with the use of topical therapies, phototherapy and systemic treatments require cycling patients on and off therapy and monitoring lifetime exposure due to their side effects.

Treatment efficacy for psoriasis is assessed by monitoring changes in clinical signs and symptoms of the disease including Physician's Global Assessment (PGA) changes and Psoriasis Area and Severity Index (PASI) scores, Psoriasis Symptom Assessment (PSA), compared with the baseline condition. The patient can be measured periodically throughout treatment on the Visual analog scale used to indicate the degree of itching experienced at specific time points.

Patients may experience an infusion reaction or infusion-related symptoms with their first infusion of a therapeutic antibody. These symptoms vary in severity and generally are reversible with medical intervention. These symptoms include but are not limited to, flu-like fever, chills/rigors, nausea, urticaria, headache, bronchospasm, angioedema. It would be desirable for the disease treatment methods of the present invention to minimize infusion reactions. Thus, another aspect of the invention is a method of treating the diseases disclosed by administering a BR3 binding antibody wherein the antibody has reduced or no complement dependent cytotoxicity.

Dosage

Depending on the indication to be treated and factors relevant to the dosing that a physician of skill in the field would be familiar with, the antibodies of the invention will be administered at a dosage that is efficacious for the treatment of that indication while minimizing toxicity and side effects. For the treatment of a cancer, an autoimmune disease or an immunodeficiency disease, the therapeutically effective dosage can be in the range of 50 mg/dose to 2.5 g/m². In one embodiment, the dosage administered is about 250 mg/m² to about 400 mg/m² or 500 mg/m². In another embodiment, the dosage is about 250-375 mg/m². In yet another embodiment, the dosage range is 275-375 mg/m².

In one embodiment of the treatment of a BR3 positive B cell neoplasm described herein (e.g., chronic lymphocytic leukemia (CLL), non-Hodgkins lymphoma (NHL), follicular lymphoma (FL) or multiple myeloma), the antibody is administered at a range of 50 mg/dose to 2.5 g/m². For the treatment of patients suffering from B-cell lymphoma such as non-Hodgkins lymphoma, in a specific embodiment, the anti-BR3 antibodies and humanized anti-BR3 antibodies of the invention will be administered to a human patient at a dosage of 10 mg/kg or 375 mg/m². For treating NHL, one dosing regimen would be to administer one dose of the antibody composition a dosage of 10 mg/kg in the first week of treatment, followed by a 2 week interval, then a second dose of the same amount of antibody is administered. Generally, NHL patients can receive such treatment once during a year but upon recurrence of the lymphoma, such treatment can be repeated. In another dosing regimen, patients treated with low-grade NHL receive four weeks of an anti-BR3 antibody (375 mg/m2 weekly) followed at week five by three additional courses of the antibody plus standard CHOP (cyclophosphamide, doxorubicin, vincristine and prednisone) or CVP (cyclophosphamide, vincristine, prednisone) chemotherapy, which was given every three weeks for three cycles.

For treating rheumatoid arthritis, in one embodiment, the dosage range for the anti-BR3 antibody is 125 mg/m² (equivalent to about 200 mg/dose) to 600 mg/m², given in two doses, e.g., the first dose of 200 mg is administered on day one followed by a second dose of 200 mg on day 15. In different embodiments, the dosage is selected from the group consisting of 250 mg/dose, 275 mg/dose, 300 mg/dose, 325 mg/dose, 350 mg/dose, 375 mg/dose, 400 mg/dose, 425 mg/dose, 450 mg/dose, 475 mg/dose, 500 mg/dose, 525 mg/dose, 550 mg/dose, 575 mg/dose and 600 mg/dose.

In treating disease, the BR3 binding antibodies of the invention can be administered to the patient chronically or intermittently, as determined by the physician of skill in the disease.

A patient administered a drug by intravenous infusion or subcutaneously may experience adverse events such as fever, chills, burning sensation, asthenia and headache. To alleviate or minimize such adverse events, the patient may receive an initial conditioning dose(s) of the antibody followed by a therapeutic dose. The conditioning dose(s) will be lower than the therapeutic dose to condition the patient to tolerate higher dosages.

It is contemplated that BR3 binding antibodies of this invention that (1) lack ADCC function or have reduced ADCC function compared to an antibody comprising a wild type human IgG Fc; (2) lack the ability to partially or fully inhibit BAFF binding to BR3 or (3) lack ADCC function or have reduced ADCC function compared to an antibody comprising a wild type human IgG Fc and lack the ability to partially or fully inhibit BAFF binding to BR3 will be useful, for example, as in a replacement therapy, alternative therapy or a maintenance therapy for patients that have or are expected to have significantly adverse responses to therapies with anti-BR3 antibodies that inhibit BAFF and BR3 binding and have ADCC function. For example, it is contemplated that a patient can be first treated with anti-BR3 antibodies that inhibit BAFF and BR3 binding and have ADCC function followed by treatments with anti-BR3 antibodies that (1) lack ADCC function or have reduced ADCC function compared to antibodies-comprising wild type human IgG Fc; (2) lack the ability to partially or fully inhibit BAFF binding to BR3 or (3) lack ADCC function or have reduced ADCC function compared to antibodies comprising wild type human IgG Fc and lack the ability to partially or fully inhibit BAFF binding to BR3.

Route of Administration

The BR3 binding antibodies are administered to a human patient in accord with known methods, such as by intravenous administration, e.g., as a bolus or by continuous infusion over a period of time, by subcutaneous, intramuscular, intraperitoneal, intracerobrospinal, intra-articular, intrasynovial, intrathecal, or inhalation routes, generally by intravenous or subcutaneous administration.

In on embodiment, the anti-BR3 antibody is administered by intravenous infusion with 0.9% sodium chloride solution as an infusion vehicle. In another embodiment, the anti-BR3 antibodies are administered with a pre-filled syringe.

Combination Therapy

The BR3-binding antibodies or polypeptides of this invention can be used in combination with a second therapeutic agent to treat the dease. It should be understood that the term second therapeutic agent does not preclude treating the subjects other additional therapies. The reference to a second therapeutic agent is meant to differentiate the agent from the specific BR3-binding antibody or polypeptide also being used. In one embodiment, a patient to be treated with the BR3 binding antibodies or polypeptides for an autoimmune disease or a cancer can be treated concurrently, sequentially (before or after), or alternatingly with a biologic response modifier (BRM) to stimulate or restore the ability of the immune system to fight disease and/or infection in a multidrug regimen. BRMs can include monoclonal antibodies, such as antibodies that target TNF-alpha or IL-1 (e.g., Enbrel®, Remicade®, and Humira®), interferon, interleukins (e.g, IL-2, IL-12) and various types of colony-stimulating factors (CSF, GM-CSF, G-CSF). For example, the BRMs may interfere with inflammatory activity, ultimately decreasing joint damage.

In one embodiment, the second therapeutic is an IAP inhibitor.

In another embodiment, a patient to be treated with the BR3 binding antibodies or polypeptides for an autoimmune disease or a cancer can be treated concurrently, sequentially (before or after), or alternatingly with a B cell depleting agent.

In one embodiment, a patient to be treated with the BR3 binding antibodies for an autoimmune disease or a cancer can be treated concurrently, sequentially (before or after), or alternatingly with a BAFF antagonist.

In another embodiment, the cancers and neoplasms described above, the patient can be treated with the BR3 binding antibodies of the present invention in conjunction with one or more therapeutic agents such as a chemotherapeutic agent in a multidrug regimen. The BR3 binding antibody can be administered concurrently, sequentially (before or after), or alternating with the chemotherapeutic agent, or after non-responsiveness with other therapy. Standard chemotherapy for lymphoma treatment may include cyclophosphamide, cytarabine, melphalan and mitoxantrone plus melphalan. CHOP is one of the most common chemotherapy regimens for treating Non-Hodgkin's lymphoma. The following are the drugs used in the CHOP regimen: cyclophosphamide (brand names cytoxan, neosar); adriamycin (doxorubicin/hydroxydoxorubicin); vincristine (Oncovin); and prednisolone (sometimes called Deltasone or Orasone). In particular embodiments, the BR3 binding antibody is administered to a patient in need thereof in combination with one or more of the following chemotherapeutic agents of doxorubicin, cyclophosphamide, vincristine and prednisolone. In a specific embodiment, a patient suffering from a lymphoma (such as a non-Hodgkin's lymphoma) is treated with an anti-BR3 antibody of the present invention in conjunction with CHOP (cyclophosphamide, doxorubicin, vincristine and prednisone) therapy. In another embodiment, a cancer or neoplasm in a patient can be treated with a BR3 binding antibody of the invention in combination with CVP (cyclophosphamide, vincristine, and prednisone) chemotherapy. In a specific embodiment, the patient suffering from BR3-positive NHL is treated with humanized anti-BR3 antibody in conjunction with CVP. In a specific embodiment of the treatment of chronic lymphocytic leukemia (CLL,) the BR3 binding antibody is administered in conjunction with chemotherapy with one or more nucleoside analogs, such as fludarabine, Cladribine (2-chlorodeoxyadenosine, 2-CdA[Leustatin]), pentostatin (Nipent), with cyclophosphamide.

In treating the autoimmune diseases or autoimmune related conditions described above, the patient can be treated with the BR3 binding antibodies of the present invention in conjunction with a second therapeutic agent, such as an immunosuppressive agent, such as in a multi drug regimen. The BR3 binding antibody can be administered concurrently, sequentially or alternating with the immunosuppressive agent or upon non-responsiveness with other therapy. The immunosuppressive agent can be administered at the same or lesser dosages than as set forth in the art. The preferred adjunct immunosuppressive agent will depend on many factors, including the type of disorder being treated as well as the patient's history.

“Immunosuppressive agent” as used herein for adjunct therapy refers to substances that act to suppress or mask the immune system of a patient. Such agents would include substances that suppress cytokine production, down regulate or suppress self-antigen expression, or mask the MHC antigens. Examples of such agents include steroids such as glucocorticosteroids, e.g., prednisone, methylprednisolone, and dexamethasone; 2-amino-6-aryl-5-substituted pyrimidines (see U.S. Pat. No. 4,665,077), azathioprine (or cyclophosphamide, if there is an adverse reaction to azathioprine); bromocryptine; glutaraldehyde (which masks the MHC antigens, as described in U.S. Pat. No. 4,120,649); anti-idiotypic antibodies for MHC antigens and MHC fragments; cyclosporin A; cytokine or cytokine receptor antagonists including anti-interferon [symbol] , [symbol] , or [symbol] antibodies; anti-tumor necrosis factor-antibodies; anti-tumor necrosis factor-antibodies; anti-interleukin-2 antibodies and anti-IL-2 receptor antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187 published Jul. 26, 1990); streptokinase; TGF [symbol] ; streptodornase; RNA or DNA from the host; FK506; RS-61443; deoxyspergualin; rapamycin; T-cell receptor (U.S. Pat. No. 5,114,721); T-cell receptor fragments (Offner et al., Science 251:430-432 (1991); WO 90/11294; and WO 91/01133); and T cell receptor antibodies (EP 340,109) such as T10B9.

For the treatment of rheumatoid arthritis, the patient can be treated with a BR3 antibody of the invention in conjunction with any one or more of the following drugs: DMARDS (disease-modifying anti-rheumatic drugs (e.g., methotrexate), NSAI or NSAID (non-steroidal anti-inflammatory drugs), HUMIRA® (adalimumab; Abbott Laboratories), ARAVA® (leflunomide), REMICADE® (infliximab; Centocor Inc., of Malvern, Pa.), ENBREL® (etanercept; Immunex, Wash.), COX-2 inhibitors. DMARDs commonly used in RA are hydroxycloroquine, sulfasalazine, methotrexate, leflunomide, etanercept, infliximab, azathioprine, D-penicillamine, Gold (oral), Gold (intramuscular), minocycline, cyclosporine, Staphylococcal protein A immunoadsorption. Adalimumab is a human monoclonal antibody that binds to TNF. Infliximab is a chimeric monoclonal antibody that binds to TNF. Etanercept is an “immunoadhesin” fusion protein consisting of the extracellular ligand binding portion of the human 75 kD (p75) tumor necrosis factor receptor (TNFR) linked to the Fc portion of a human IgG1. For conventional treatment of RA, see, e.g., “Guidelines for the management of rheumatoid arthritis” Arthritis & Rheumatism 46(2): 328-346 (February, 2002). In a specific embodiment, the RA patient is treated with a BR3 antibody of the invention in conjunction with methotrexate (MTX). An exemplary dosage of MTX is about 7.5-25 mg/kg/wk. MTX can be administered orally and subcutaneously.

For the treatment of ankylosing spondylitis, psoriatic arthritis and Crohn's disease, the patient can be treated with a BR3 binding antibody of the invention in conjunction with, for example, Remicade® (infliximab; from Centocor Inc., of Malvern, Pa.), ENBREL® (etanercept; Immunex, Wash.).

Treatments for SLE include high-dose corticosteroids and/or cyclophosphamide (HDCC).

For the treatment of psoriasis, patients can be administered a BR3 binding antibody in conjunction with topical treatments, such as topical steroids, anthralin, calcipotriene, clobetasol, and tazarotene, or with methotrexate, retinoids, cyclosporine, PUVA and UVB therapies. In one embodiment, the psoriasis patient is treated with the BR3 binding antibody sequentially or concurrently with cyclosporine.

### Pharmaceutical Formulations

Therapeutic formulations of the BR3-binding antibodies used in accordance with the present invention are prepared for storage by mixing an antibody having the desired degree of purity with optional pharmaceutically acceptable carriers, excipients or stabilizers (sRemington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Exemplary anti-BR3 antibody formulations are described in WO98/56418, expressly incorporated herein by reference. Another formulation is a liquid multidose formulation comprising the anti-BR3 antibody at 40 mg/mL, 25 mM acetate, 150 mM trehalose, 0.9% benzyl alcohol, 0.02% polysorbate 20 at pH 5.0 that has a minimum shelf life of two years storage at 2-8° C. Another anti-BR3 formulation of interest comprises 10 mg/mL antibody in 9.0 mg/mL sodium chloride, 7.35 mg/mL sodium citrate dihydrate, 0.7 mg/mL polysorbate 80, and Sterile Water for Injection, pH 6.5. Yet another aqueous pharmaceutical formulation comprises 10-30 mM sodium acetate from about pH 4.8 to about pH 5.5, preferably at pH5.5, polysorbate as a surfactant in a an amount of about 0.01-0.1% v/v, trehalose at an amount of about 2-10% w/v, and benzyl alcohol as a preservative (U.S. Pat. No. 6,171,586). Lyophilized formulations adapted for subcutaneous administration are described in WO97/04801. Such lyophilized formulations may be reconstituted with a suitable diluent to a high protein concentration and the reconstituted formulation may be administered subcutaneously to the mammal to be treated herein.

One formulation for the humanized anti-BR3 antibody is antibody at 12-14 mg/mL in 10 mM histidine, 6% sucrose, 0.02% polysorbate 20, pH 5.8.

In a specific embodiment, anti-BR3 antibody and in particular 9.1RF, 9.1RF (N434 mutants), or V3-46s is formulated at 20 mg/mL antibody in 10 mM histidine sulfate, 60 mg/mL sucrose., 0.2 mg/ml polysorbate 20, and Sterile Water for Injection, at pH5.8.

The formulation herein may also contain more than one active compound as necessary for the particular indication being treated, preferably those with complementary activities that do not adversely affect each other. For example, it may be desirable to further provide a cytotoxic agent, chemotherapeutic agent, cytokine or immunosuppressive agent (e.g. one which acts on T cells, such as cyclosporin or an antibody that binds T cells, e.g. one which binds LFA-1). The effective amount of such other agents depends on the amount of antibody present in the formulation, the type of disease or disorder or treatment, and other factors discussed above. These are generally used in the same dosages and with administration routes as described herein or about from 1 to 99% of the heretofore employed dosages.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semi-permeable matrices of solid hydrophobic polymers containing the antagonist, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

### Articles of Manufacture and Kits

Another embodiment of the invention is an article of manufacture containing materials useful for the treatment of autoimmune diseases and related conditions and BR3 positive cancers such as non-Hodgkin's lymphoma. Yet another embodiment of the invention is an article of manufacture containing materials useful for the treatment of immunodeficiency diseases. The article of manufacture comprises a container and a label or package insert on or associated with the container. Suitable containers include, for example, bottles, vials, syringes, etc. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). At least one active agent in the composition is a BR3 binding antibody of the invention. The label or package insert indicates that the composition is used for treating the particular condition. The label or package insert will further comprise instructions for administering the antibody composition to the patient. Articles of manufacture and kits comprising combinatorial therapies described herein are also contemplated.

Package insert refers to instructions customarily included in commercial packages of therapeutic products, that contain information about the indications, usage, dosage, administration, contraindications and/or warnings concerning the use of such therapeutic products. In one embodiment, the package insert indicates that the composition is used for treating non-Hodgkins' lymphoma.

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for B-cell killing assays, as a positive control for apoptosis assays, for purification or immunoprecipitation of BR3 from cells. For isolation and purification of BR3, the kit can contain an anti-BR3 antibody coupled to beads (e.g., sepharose beads). Kits can be provided which contain the antibodies for detection and quantitation of BR3 in vitro, e.g. in an ELISA or a Western blot. As with the article of manufacture, the kit comprises a container and a label or package insert on or associated with the container. The container holds a composition comprising at least one anti-BR3 antibody of the invention. Additional containers may be included that contain, e.g., diluents and buffers, control antibodies. The label or package insert may provide a description of the composition as well as instructions for the intended in vitro or diagnostic use.

Monoclonal Antibodies

Anti-BR3 antibodies can be monoclonal antibodies. Monoclonal antibodies can be prepared, e.g., using hybridoma methods, such as those described by Kohler and Milstein, Nature, 256:495 (1975) or can be made by recombinant DNA methods (U.S. Pat. No. 4,816,567) or can be produced by the methods described herein in the Example section. In a hybridoma method, a mouse, hamster, or other appropriate host animal is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the BR3 polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Goding, Monoclonal Antibodies: Principles and Practice (New York: Academic Press, 1986), pp. 59-103. Immortalized cell lines are usually transformed mammalian cells, particularly myeloma cells of rodent, bovine, and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high-level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies. Kozbor, J. Immunol., 133:3001 (1984); Brodeur et al., Monoclonal Antibody Production Techniques and Applications (Marcel Dekker, Inc.: New York, 1987) pp. 51-63.

The culture medium in which the hybridoma cells are cultured can then be assayed for the presence of monoclonal antibodies directed against the BR3 polypeptide. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones can be subcloned by limiting dilution procedures and grown by standard methods. Goding, supra. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy- and light-chain constant domains in place of the homologous murine sequences (U.S. Pat. No. 4,816,567; Morrison et al., supra) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies can be monovalent antibodies. Methods for preparing monovalent antibodies are known in the art. For example, one method involves recombinant expression of immunoglobulin light chain and modified heavy chain. The heavy chain is truncated generally at any point in the Fc region so as to prevent heavy-chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly Fab fragments, can be accomplished using techniques known in the art.

### Human and Humanized Antibodies

The anti-BR3 antibodies can further comprise humanized antibodies or human antibodies. Humanized forms of non-human (e.g., murine) antibodies are chimeric immunoglobulins, immunoglobulin chains, or fragments thereof (such as Fv, Fab, Fab′, F(ab′)₂, or other antigen-binding subsequences of antibodies) that typically contain minimal sequence derived from non-human immunoglobulin. Humanized antibodies include human immunoglobulins (recipient antibody) in which residues from a CDR of the recipient are replaced by residues from a CDR of a non-human species (donor antibody) such as mouse, rat, or rabbit having the desired specificity, affinity, and capacity. In some instances, Fv framework residues of the human immunoglobulin are replaced by corresponding non-human residues. Humanized antibodies can also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin, and all or substantially all of the FR regions are those of a human immunoglobulin consensus sequence. The humanized antibody preferably also will comprise at least a portion of an immunoglobulin constant region (Fc), typically that of a human immunoglobulin. Jones et al., Nature, 321: 522-525 (1986); Riechmann et al., Nature, 332: 323-329 (1988); Presta, Curr. Op. Struct. Biol., 2:593-596 (1992).

Some methods for humanizing non-human antibodies are described in the art and below in the Examples. Generally, a humanized antibody has one or more amino acid residues introduced into it from a source that is non-human. These non-human amino acid residues are often referred to as “import” residues, which are typically taken from an “import” variable domain. According to one embodiment, humanization can be essentially performed following the method of Winter and co-workers (Jones et al., Nature 321: 522-525 (1986); Riechmann et al., Nature, 332: 323-327 (1988); Verhoeyen et al., Science, 239: 1534-1536 (1988)), by substituting rodent CDRs or CDR sequences for the corresponding sequences of a human antibody. Accordingly, such “humanized” antibodies are antibodies (U.S. Pat. No. 4,816,567), wherein substantially less than an intact human variable domain has been substituted by the corresponding sequence from a non-human species. In practice, humanized antibodies are typically human antibodies in which some-CDR residues and possibly some FR residues are substituted by residues from analogous sites in rodent antibodies.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806, 5,569,825, 5,591,669 (all of GenPharm); 5,545,807; and WO 97/17852. Alternatively, human antibodies can be made by introducing human immunoglobulin loci into transgenic animals, e.g., mice in which the endogenous immunoglobulin genes have been partially or completely inactivated. Upon challenge, human antibody production is observed that closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; and 5,661,016, and in the following scientific publications: Marks et al., Bio/Technology, 10: 779-783 (1992); Lonberg et al., Nature 368: 856-859 (1994); Morrison, Nature, 368: 812-813 (1994); Fishwild et al., Nature Biotechnology, 14: 845-851 (1996); Neuberger, Nature Biotechnology, 14: 826 (1996); Lonberg and Huszar, Intern. Rev. Immunol., 13: 65-93 (1995). Alternatively, phage display technology (McCafferty et al., Nature 348:552-553 [1990]) can be used to produce human antibodies and antibody fragments in vitro, from immunoglobulin variable (V) domain gene repertoires from unimmunized donors. According to one embodiment of this technique, antibody V domain sequences are cloned in-frame into either a major or minor coat protein gene of a filamentous bacteriophage, such as M13 or fd, and displayed as functional antibody fragments on the surface of the phage particle. Phage display can be performed in a variety of formats, e.g., as described below in the Examples section or as reviewed in, e.g., Johnson, Kevin S. and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

Human antibodies can also be produced using various techniques known in the art, including phage display libraries. Hoogenboom and Winter, J. Mol. Biol., 227: 381 (1991); Marks et al., J. Mol. Biol., 222: 581 (1991). The techniques of Cole et al. and Boerner et al. are also available for the preparation of human monoclonal antibodies. Cole et al., Monoclonal Antibodies and Cancer Therapy, Alan R. Liss, p. 77 (1985) and Boerner et al., J. Immunol., 147(1): 86-95 (1991).

### Multi-Specific Anti-BR3 Antibodies

Multi-specific antibodies are monoclonal, preferably human or humanized, antibodies that have binding specificities for two or more different antigens (e.g., bispecific antibodies have binding specificities for at least two antigens). For example, one of the binding specificities can be for the BR3 polypeptide, the other one can be for any other antigen. According to one preferred embodiment, the other antigen is a cell-surface protein or receptor or receptor subunit. For example, the cell-surface protein can be a natural killer (NK) cell receptor. Thus, according to one embodiment, a bispecific antibody of this invention can bind BR3 and bind a NK cell and, optionally, activate the NK cell.

Examples of methods for making bispecific antibodies have been described. Traditionally, the recombinant production of bispecific antibodies is based on the co-expression of two immunoglobulin heavy-chain/light-chain pairs, where the two heavy chains have different specificities. Milstein and Cuello, Nature 305: 537-539 (1983). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10: 3655-3659 (1991).

Antibody variable domains with the desired binding specificities (antibody-antigen combining sites) can be fused to immunoglobulin constant-domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies, see, for example, Suresh et al., Methods in Enzymology, 121: 210 (1986).

Various techniques for making and isolating bispecific antibody fragments directly from recombinant cell culture have also been described. For example, bispecific antibodies have been produced using leucine zippers. Kostelny et al., J. Iimnunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol. 147: 60 (1991).

### Heteroconjugate Antibodies

Heteroconjugate antibodies are composed of two covalently joined antibodies. Such antibodies have, for example, been proposed to target immune-system cells to unwanted cells (U.S. Pat. No. 4,676,980), and for treatment of HIV infection. WO 91/00360; WO 972/200373; EP 03089. It is contemplated that the antibodies can be prepared in vitro using known methods in synthetic protein chemistry, including those involving crosslinking agents. For example, immunotoxins can be constructed using a disulfide-exchange reaction or by forming a thioether bond. Examples of suitable reagents for this purpose include iminothiolate and methyl-4-mercaptobutyrimidate and those disclosed, for example, in U.S. Pat. No. 4,676,980.

### Engineering of Afucosylated Antibodies and Underfucosylated Antibody Compositions

The invention herein relates to a method for making an afucosylated anti-BR3 antibody or an underfucosylated composition of anti-BR3 antibodies that includes a substantially homogeneous preparation of Fc region-containing antibodies, wherein about 20-100% of the antibodies in the composition comprises a mature core carbohydrate lacking fucose, attached to the Fc region of the antibody. The antibodies can be prepared, for example, by (a) use of an engineered or mutant host cell that is deficient in fucose metabolism such that it has a reduced ability (or is unable to) fucosylate proteins expressed therein; (b) culturing cells under conditions which prevent or reduce fucosylation; (c) post-translational removal of fucose (e.g. with a fucosidase enzyme); (d) post-translational addition of the desired carbohydrate, e.g. after recombinant expression of a non-glycosylated antibody; or (e) purification of the antibody so as to select for product which is not fucosylated. The present invention contemplates combining two or more of these exemplary methods (a)-(e).

According to one preferred embodiment, nucleic acid encoding the desired antibody is expressed in a host cell that has a reduced ability (or is unable to) fucosylate proteins expressed therein. For example, the host cell can be a dihydrofolate reductase (DHFR) deficient chinese hamster ovary (CHO) cell, e.g. a Lec13 CHO cell, or e.g., a CHO-K1, DUX-B11, CHO-DP12 or CHO-DG44 CHO host cell which has been modified so that the antibody produced therein is not substantially fucosylated. Thus, the cell may display altered expression or activity for the fucosyltransferase enzyme, or another enzyme or substrate involved in adding fucose to the N-linked oligosaccharide may have diminished activity and/or reduced levels in the host cell. Alternately, the host cell can be transfected with a vector producing an RNAi targeting a protein in the fucosyl pathway.

The core carbohydrate structure is mature, thus, the use of inhibitors, such as castanospermine, which inhibit or interfere with processing of the mature carbohydrate should generally be avoided. According to one embodiment, anywhere from 1-100% of the anti-BR3 antibodies are afucosylated. According to one preferred embodiment of the invention, an underfucosylated composition of antibodies is recovered wherein about 20-100% of the antibody in the composition recovered from the recombinant host cell producing the antibody will have a core carbohydrate structure which lacks fucose attached to the Fc region of the antibody, hereinafter a “fucose-free antibody composition.” By “recovered” here is meant that material obtained directly from the host cell culture without subjecting that material to a purification step which enriches for fucose-free antibody.

However, the present invention does contemplate enriching the amount of fucose-free antibody by various techniques, such as purification using a lectin substrate to remove fucose-containing antibody from the desired composition.

It will be appreciated that the amount of fucose-free antibody from various batches of recombinantly produced antibody may vary. In one preferred embodiment, about 90-99% of the antibody in the composition comprises a mature core carbohydrate structure which lacks fucose attached to the Fc region of the antibody.

Various forms of the carbohydrate structure may exist in the composition (see FIG. 7). For instance, the carbohydrate attached to the antibody may be represented by the following formula:

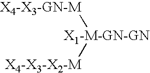

wherein,
M is mannose.

### GN is GlcNAc.

X₁ is an optional bisecting GlcNAc residue, with additional monosaccharide(s) optionally attached to the bisecting GlcNAc.
X₂ is a preferred GlcNAc residue.
X₃ is an optional Gal residue, one Gal residue may be attached to each GN arm.
X₄ is an optional terminal sialic acid residues, one or two sialic acid residues may be attached.

The fucose-free antibody compositions herein display improved binding to one or more FcγRIII receptors, compared to a composition of the same antibody, but where most (e.g. about 50-100%, or about 70-100%) of the antibody in that composition has fucose attached to the mature core carbohydrate structure (hereinafter a “fucose-containing antibody composition). For instance, the fucose-free antibody compositions herein may display 3-1000 fold improved binding to an FcγRIII, such as FcγRIII (F158), when compared to the fucose-containing antibody composition. In that the F158 allotype is less effective in interacting with human IgG than V158, this is thought to provide a significant advantage from a therapeutic perspective, especially in patients who express FcγRIII (F158). Moreover, the fucose-free antibody compositions herein display better ADCC activity compared to their counterpart fucose-containing antibody compositions, e.g. from about 2-20 fold improved ADCC activity.

Aside from the fucose-free mature core carbohydrate structure, additional oligosaccharides may be attached to the core carbohydrate structure. For instance, a bisecting GlcNAc may, or may not be, attached. By the way of example, the host cell may lack the GnTIII enzyme and hence the antibody may be essentially free of bisecting GlcNAc. Alternatively, the antibody may be expressed in a host cell (e.g. a Y0 host or engineered CHO cell) which adds the bisecting GlcNAc. One or more (generally one or two) galactose residues may also be attached to the core carbohydrate structure. Finally, one or more terminal sialic acid residues (usually one or two) may be attached to core carbohydrate structure, e.g. by linkage to galactose residue(s).

The compositions herein are, in the preferred embodiment, prepared and intended for therapeutic use. Hence, the preferred composition is a pharmaceutical preparation comprising the antibody and a pharmaceutically acceptable carrier or diluent such as those exemplified below. Such preparations are usually sterile and may be lyophilized.

### Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See, Caron et al., J. Exp. Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See, Stevenson et al., Anti-Cancer Drug Design, 3: 219-230 (1989).

Mutations or alterations in the Fc region sequences can be made to improve FcR binding (e.g., FcgammaR, FcRn). According to one embodiment, an antibody of this invention has at least one altered effector function selected from the group consisting of ADCC, CDC, and improved FcRn binding compared to a native IgG or a parent antibody. Examples of several useful specific mutations are described in, e.g., Shields, R L et al. (2001) JBC 276(6)6591-6604; Presta, L. G., (2002) Biochemical Society Transactions 30(4):487-490; and WO publication WO00/42072.

According to one embodiment, the Fc receptor mutation is a substitution in at least one position selected from the group consisting of: 238, 239, 246, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 332, 333, 334, 335, 337, 338, 340, 360, 373, 376, 378, 382, 388, 389, 398, 414, 416, 419, 430, 434, 435, 437, 438 or 439 of the Fc region, wherein the numbering of the residues in the Fc region is according to the EU numbering system. According to one specific embodiment, the substitution is a 434 residue substitution selected from the group consisting of N434A, N434F, N4343Y and N434H. According to another embodiment, the substitutions are a D265A/N297A mutation. According to another embodiment, the substitutions are S298A/E333A/K334A or S298A/K326A/E333A/K334A. According to another embodiment, the substitution is K322A.

Examples of native sequence human IgG Fc region sequences, humIgG1 (non-A and A allotypes) (SEQ ID NOs:133 and 135, respectively), humIgG2 (SEQ ID NO:136), humIgG3 (SEQ ID NO:137) and humIgG4 (SEQ ID NO:138) have been described previously. Examples of native sequence murine IgG Fc region sequences, murIgG1 (SEQ ID NO:139), murIgG2A (SEQ ID NO:140), murIgG2B (SEQ ID NO:141) and murIgG3 (SEQ ID NO:142), have also been described previously. Example of other mutations may be found in WO2006053301A2, WO2006047350A2, US20060134105A1, WO2005092925A2, US20050244403A1, WO2005077981A2, US20050249723A1, WO2003074679A2, US20040110226A1, WO2004029207A2 US20040132101A, WO2004099249A2, US20050054832A1, WO2006019447A1, US20060024298A1, US20060121032A1.

In addition, various classes of Fc region variants are described below in Table 3.

TABLE 3
Classes of Fc region variants.

| Class | FcR binding property | Position of Fc region substitution(s) |
|---|---|---|
| 1A | reduced binding to all FcγR | 238, 265, 269, 270, 297*, 327, 329 |
| 1B | reduced binding to both FcγRII and | 239, 294, 295, 303, 338, 373, 376, 416, 435 |
| | FcγRIII | |
| 2 | improved binding to both FcγRII and | 256, 290, 312, 326, 330, 339, 378, 430 |
| | FcγRIII | |
| 3 | improved binding to FcγRII and no effect | 255, 258, 267, 276, 280, 283, 285, 286, 305, |
| | on FcγRIII binding | 307, 309, 315, 320, 331, 337, 398 |
| 4 | improved binding to FcγRII and reduced | 268, 272, 301, 322, 340 |
| | binding to FcγRIII | |
| 5 | reduced binding to FcγRII and no effect | 292, 324, 335, 414, 419, 438, 439 |
| | on FcγRIII binding | |
| 6 | reduced binding to FcγRII and improved | 298, 333 |
| | binding to FcγRIII | |
| 7 | no effect on FcγRII binding and reduced | 248, 249, 252, 254, 278, 289, 293, 296, 338, |
| | binding to FcγRIII | 382, 388, 389, 434, 437 |
| 8 | no effect on FcγRII binding and | 334, 360 |
| | improved binding to FcγRIII | |

*deglycosylated version

### Pharmaceutical Compositions of Antibodies and Polypeptides

Antibodies specifically binding a BR3 polypeptide identified herein, as well as other molecules identified by the screening assays disclosed hereinbefore, can be administered for the treatment of various disorders as noted above and below in the form of pharmaceutical compositions.

Lipofectins or liposomes can be used to deliver the polypeptides and antibodies or compositions of this invention into cells. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See, Gabizon et al., J. National Cancer Inst., 81(19): 1484 (1989).

Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993).

The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences, supra.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they can denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization can be achieved by modifying sulfhydryl residues, lyophilizing from acidic solutions, controlling moisture content, using appropriate additives, and developing specific polymer matrix compositions.

### Diagnostic Use and Imaging

Labeled antibodies, and derivatives and analogs thereof, which specifically bind to a BR3 can be used for diagnostic purposes to detect, diagnose, or monitor diseases and/or disorders associated with the expression, aberrant expression and/or activity of a polypeptide of the invention. According to one preferred embodiment, the anti-BR3 antibodies used in diagnostic assays or imaging assays that involve injection of the anti-BR3 antibody into the subject are antibodies that do not block the interaction between BAFF and BR3 or only partially blocks the interation between BAFF and BR3. The invention provides for the detection of aberrant expression of a BR3 polypeptide, comprising (a) assaying the expression of the BR3 polypeptide in cells or body fluid of an individual using one or more antibodies of this invention and (b) comparing the level of gene expression with a standard gene expression level, whereby an increase or decrease in the assayed gene expression level compared to the standard expression level is indicative of aberrant expression.

The invention provides a diagnostic assay for diagnosing a disorder to be treated with an anti-BR3 antibody or polypeptide of this invention, comprising (a) assaying the expression of BR3 polypeptide in cells or body fluid of an individual using an antibody of this invention, (b) assaying the expression of BAFF polypeptide in cells or body fluid of the individual and (c) comparing the level of BAFF gene expression with a standard gene expression level, whereby an increase or decrease in the assayed BAFF gene expression level compared to the standard expression level and the presence of BR3 polypeptide in the fluid or diseased tissue is indicative of a disorder to be treated with an anti-BR3 antibody or polypeptide. With respect to cancer, the presence of BR3 or a relatively high amount of BR3 transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Antibodies of the invention can be used to assay protein levels in a biological sample using classical immunohistological methods known to those of skill in the art (e.g., see Jalkanen, et al., J. Cell. Biol. 101:976-985 (1985); Jalkanen, et al., J. Cell. Biol. 105:3087-3096 (1987)). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹³¹I, ¹²⁵I, ¹²³I, ¹²¹I), carbon (¹⁴C), sulfur (³⁵S), tritium (³H), indium (^{115m}In, ^{113m}In ¹¹²In, ¹¹¹In), and technetium (⁹⁹Tc, ^{99m}Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y_{,} ⁴⁷Sc, ¹⁸⁶Re, ¹⁸⁸Re, ¹⁴²Pr, ¹⁰⁵Rh, ⁹⁷Ru; lmmol; and fluorescent labels, such as fluorescein and rhodamine, and biotin.

Techniques known in the art may be applied to label antibodies of the invention. Such techniques include, but are not limited to, the use of bifunctional conjugating agents (see e.g., U.S. Pat. Nos. 5,756,065; 5,714,631; 5,696,239; 5,652,361; 5,505,931; 5,489,425; 5,435,990; 5,428,139; 5,342,604; 5,274,119; 4,994,560; and 5,808,003; the contents of each of which are hereby incorporated by reference in its entirety).

Diagnosis of a disease or disorder associated with expression or aberrant expression of a BR3 molecule in an animal, preferably a mammal and most preferably a human can comprise the step of detecting BR3 molecules in the mammal. In one embodiment, diagnosis comprises: (a) administering (for example, parenterally, subcutaneously, or intraperitoneally) to a mammal an effective amount of a labeled anti-BR3 antibody or polypeptide which specifically binds to the BR3 molecule, respectively; (b) waiting for a time interval following the administering for permitting the labeled molecule to preferentially concentrate at sites in the subject where the BR3 molecule is expressed (and for unbound labeled molecule to be cleared to background level); (c) determining background level; and (d) detecting the labeled molecule in the subject, such that detection of labeled molecule above the background level indicates that the subject has a particular disease or disorder associated with expression or aberrant expression of BR3. Background level can be determined by various methods including, comparing the amount of labeled molecule detected to a standard value previously determined for a particular system. According to specific embodiments, the antibodies of the invention are used to quantitate or qualitate concentrations of cells of B cell lineage or cells of monocytic lineage.

According to one specific embodiment, BR3 polypeptide expression or overexpression is determined in a diagnostic or prognostic assay by evaluating levels of BR3 present on the surface of a cell, or secreted by the cell (e.g., via an immunohistochemistry assay using anti-BR3 antibodies or anti-BAFF antibodies; FACS analysis, etc.). Alternatively, or additionally, one can measure levels of BR3 polypeptide-encoding nucleic acid or mRNA in the cell, e.g., via fluorescent in situ hybridization using a nucleic acid based probe corresponding to a BR3-encoding nucleic acid or the complement thereof; (FISH; see WO98/45479 published October, 1998), Southern blotting, Northern blotting, or polymerase chain reaction (PCR) techniques, such as real time quantitative PCR (RT-PCR). One can also study. BR3 molecules or BAFF molecules overexpression by measuring shed antigen in a biological fluid such as serum, e.g., using antibody-based assays (see also, e.g., U.S. Pat. No. 4,933,294 issued Jun. 12, 1990; WO91/05264 published Apr. 18, 1991; U.S. Pat. No. 5,401,638 issued Mar. 28, 1995; and Sias et al., J. Immunol. Methods 132:73-80 (1990)). Aside from the above assays, various in vivo assays are available to the skilled practitioner. For example, one can expose cells within the body of the mammal to an antibody which is optionally labeled with a detectable label, e.g., a radioactive isotope, and binding of the antibody to cells in the mammal can be evaluated, e.g., by external scanning for radioactivity or by analyzing a biopsy taken from a mammal previously exposed to the antibody.

### Assays

All publications (including patents and patent applications) cited herein are hereby incorporated in their entirety by reference, including U.S. Provisional Application No. 60/640,323, filed Dec. 31, 2004.

The following DNA sequences were deposited under the terms of the Budapest Treaty with the American Type Culture Collection (ATCC), 10801 University Blvd., Manassas, Va. 20110-2209, USA as described below:

| Material | Deposit No. | Deposit Date |
|---|---|---|
| Hu9.1-RF-H-IgG | PTA-6315 | Nov. 17, 2004 |
| Hu9.1-RF-L-IgG | PTA-6316 | Nov. 17, 2004 |
| Hu2.1-46.DANA-H-IgG | PTA-6313 | Nov. 17, 2004 |
| Hu2.1-46.DANA-L-IgG | PTA-6314 | Nov. 17, 2004 |
| HuV3-46s-H-IgG | PTA-6317 | Nov. 17, 2004 |
| HuV3-46s-L-IgG | PTA-6318 | Nov. 17, 2004 |
| Murine B Cells: 12B12.1 | PTA-6624 | Apr. 8, 2005 |
| Murine B Cells: 3.1 | PTA-6622 | Apr. 8, 2005 |

The deposits herein were made under the provisions of the Budapest Treaty on the International Recognition of the Deposit of Microorganisms for the Purpose of Patent Procedure and the Regulations thereunder (Budapest Treaty). This assures maintenance of a viable culture of the deposits for 30 years from the date of deposit. The deposits will be made available by ATCC under the terms of the Budapest Treaty, and subject to an agreement between Genentech, Inc. and ATCC, which assures permanent and unrestricted availability of the progeny of the culture of the deposits to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 U.S.C. 122 and the Commissioner's rules pursuant to thereto (including 37 C.F.R. 1.14 with particular reference to 8860G 638).

The assignee of the present application has agreed that if a culture of the materials on deposits should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

Commercially available reagents referred to in the Examples were used according to manufacturer's instructions unless otherwise indicated. The source of those cells identified in the following Examples, and throughout the specification, by ATCC accession numbers is the American. Type Culture Collection, Manassas, Va. Unless otherwise noted, the present invention uses standard procedures of recombinant DNA technology, such as those described hereinabove and in the following textbooks: Sambrook et al., supra-, Ausubel et al., Current Protocols in Molecular Biology (Green Publishing Associates and Wiley Interscience, N.Y., 1989); Innis et al., PCR Protocols: A Guide to Methods and Applications (Academic Press, Inc.: N.Y., 1990); Harlow et al., Antibodies: A Laboratory Manual (Cold Spring Harbor Press: Cold Spring Harbor, 1988); Gait, Oligonucleotide Synthesis (IRL Press: Oxford, 1984); Freshney, Animal Cell Culture, 1987; Coligan et al., Current Protocols in Immunology, 1991.

Throughout this specification and claims, the word “comprise,” or variations such as “comprises” or “comprising,” will be understood to imply the inclusion of a stated integer or group of integers but not the exclusion of any other integer or group of integers.

The foregoing written description is considered to be sufficient to enable one skilled in the art to practice the invention. The following Examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### EXAMPLES

### Example 1

### Materials

Hybridomas producing murine monoclonal antibodies referred to as 2.1 and 9.1, have been previously described (International Patent Application PCT/US01/28006 (WO 02/24909)) and deposited in the American Type Culture Collection (ATCC) as ATCC NO. 3689 and ATCC NO. 3688, respectively (10801 University Blvd., Manassas, Va. 20110-2209, USA). Hybridomas 3.1 and 12B12.1 were deposited as ATCC Deposit PTA-6622 and ATCC Deposit PTA-6624, respectively. Other antibodies were made using phage display techniques. The sequences of those antibodies are provided herein and/or have been deposited as described herein.

### Example 2

### BJAB Cell Binding Assay

BJAB cells, a human Burkitt lymphoma cell line, were cultured in RPMI media supplemented with 10% FBS, penicillin (100 U/ml, Gibco-Invitrogen, Carlsbad, Calif.), streptomycin (100 μg/ml, Gibco), and L-glutamine (10 mM). Analysis of receptor expression by flow cytometry demonstrated that BJAB cells express high levels of BR3 and undetectable levels of BCMA and TACI. For binding assays, cells were washed with cold assay buffer (phosphate buffered saline (PBS), pH 7.4) containing 1% fetal bovine serum (FBS)). The cell density was adjusted to 1.25×10⁶/ml, and 200 μl of cell suspension was aliquoted into the wells of 96 well round-bottom polypropylene plates (NUNC, Neptune, N.J.; 250,000 cells/well). The plates containing the cells were centrifuged at 1200 rpm for 5 min at 4° C., and the supernatant was carefully aspirated away from the cell pellets. V3-1m (or mV3-1) and V3-1h refers to the variable region of the V3-1 antibody fused to the constant regions of mouse IgG2a or human IgG1, respectively. The term chimeric 11G9, chimeric 2.1 or chimeric 9.1 refers to the fusion of the variable regions of 11G9, 2.1 or 9.1, respectively, to the constant regions of a human IgG1. For these experiments, full length antibodies (IgG) were used.

Direct and competitive binding assays were performed as follows. For the direct binding assay, IgG antibody samples were serially diluted in cold assay buffer to concentrations ranging between 300-0.02 nM. Samples (100 μl) were added to the pelleted cells, and the plates were incubated for 45 min on ice. An additional 100 μl assay buffer was then added to each well, and the plates were centrifuged at 1200 rpm for 5 min at 4° C. After carefully aspirating the supernatant, the cells were washed two additional times with 200 μl assay buffer. An anti-mouse IgG Fc-HRP or goat anti-human IgG Fc-HRP, as appropriate, was diluted 1/10,000 in cold assay buffer was added (100 μl/well, Jackson ImmunoResearch, West Grove, Pa.), and the plates were incubated on ice for 45 min. Following two washes with 200 μl cold assay buffer, tetramethyl benzidine (TMB, Kirkegaard & Perry Laboratories, Gaithersburg, Md.) was added, and color was allowed to develop for 10 min. One hundred microliters 1 M H₃PO₄ was added to stop the reaction. The plates were then read on a microplate reader at 450 nm with a 620 nm reference. In the direct binding assay, the indicated concentrations of mAbs were added to BJAB cells and bound mAb was detected.

In the competitive binding assay, the anti-BR3 mAbs compete with biotinylated BAFF for binding to cell surface BR3. Human BAFF expressed and purified at Genentech was biotinylated using NHS-X-biotin (Research Organics, Cleveland, Ohio) as previously described (Rodriguez, C. F., et al., (1998) J. Immunol. Methods 219:45-55). The anti-BR3 antibodies were serially diluted and combined with an equal volume of biotin-BAFF to give final concentrations of 333-0.15 nM mAb and 10 ng/ml biotin-BAFF. The diluted samples were added to the pelleted BJAB cells in 96 well plates as described above. After 45 min incubation on ice, the cells were washed twice with 200 μl cold assay buffer, and streptavidin-HRP (AMDEX, Amersham Biosciences, Piscataway, N.J.) diluted 1/5,000 in assay buffer was added (100 μl/well). The plates were incubated for a final 45 min on ice. After washing twice with cold assay buffer, color was developed using TMB, the reaction was stopped with H₃PO₄, and the plates were read as described above.

Based on the results of the BJAB binding assays, the antibodies could be classified as either blocking or non-blocking. In the competitive assay, four mAbs (11G9, 2.1, 9.1, and V3-1) fully blocked binding of biotin-BAFF while three others (1E9, 7B2, and 8G4) resulted in partial inhibition. MAbs 1A11, 8E4, 10E2, 12B12 and 3.1 were found to be non-blocking. Of these nonblocking antibodies, 1A11 and 8E4 bound relatively poorly to the BJABs in the direct binding assay, while binding of 10E12 and 12B12 gave somewhat higher maximum signal than the other mAbs. Mouse IgG1, IgG2a, and IgG2b isotype controls showed no detectable binding to BJABs, and the HRP-conjugated anti-mouse IgG Fc detection antibody was shown to bind equally to these isotypes. MAbs V3-1m and B9C11 were evaluated in both the BJAB and BHK binding assays. While both of these blocking antibodies bind to murine BR3, only V3-1m binds to human BR3. Results with V3-1h were similar to those observed for V3-1m.

### Example 3

### Epitope Mapping ELISAs

Epitope mapping studies were performed by ELISAs in which dilution curves of unlabeled mAbs competed with biotinylated 2.1, 9.1, 11G9, or 1E9 for binding to vhBR3-Fc. The results for the fully blocking mAbs (11G9, 2.1, and 9.1) suggested that the epitope for 11G9 binding was spatially located between the epitopes for mAbs 2.1 and 9.1 given that both 2.1 and 9.1 effectively displaced binding of biotinylated 1109 but showed only a marginal ability to displace each other. Three mAbs (1E9, 7B2, and 8G4) were characterized as partial blockers in the competitive BJAB binding assay. In the epitope mapping ELISA, these mAbs appeared to bind more peripherally to the central BAFF blocking site given that they only partially inhibited the binding of the 11G9, 2.1, and 9.1. Finally, the non-blocking mAb, 12B12, appeared to bind still further away from the region of the blocking antibodies given that it could be displaced by only 1E9, a partial blocker.

Mapping studies were also performed to evaluate the binding of V3-1m, B9C11, and P1B8 to mouse BR3. The results demonstrated that while the two blocking mAbs (V3-1m and B9C11) were able to cross-compete for binding to mouse BR3, the non-blocking mAb P1B8 appeared to bind to a separate epitope.

### Example 4

### Antagonistic and Agonistic Effects of Anti-BR3 Antibodies on B Cell Proliferation

(a) 2.1, 9.1 and 1109 Inhibit Human B Cell Proliferation

B cells were isolated from peripheral blood mononuclear cells by positive selection using CD19 MACS beads (Miltenyi Biotec). For proliferation assays, B cells were set up cells at 2×10⁵ c/well in flat-bottom 96-well plate in triplicate. Cells were cultured cells for 5 days with anti-IgM (10 mg/ml) (Jackson Immunoresearch), mBAFF (5 μg/ml) and the indicated anti-BR3 antibodies or proteins for 5 days. Antibodies used were chimeric antibodies in an hIgG1 background and purified from tissue culture. The cells were then pulsed with 1 mCi/well tritiated-thymidine for the last 6 hours of culture, harvested onto a filter and counted.

(b) V3-1 Inhibits Murine B Cell Proliferation

Splenic B cells were prepared from C57BL/6 mice or from anti-HEL BCR transgenic mice at the age of 2-4 months, using B cell isolation kit from Miltenyi, according to the manufacture's instruction. B cells with more than 95% purity were consistently obtained. The B cells were cultured in the RPMI-1640 medium, containing 10% heat-inactivated FCS, penicillin/streptomycin, 2 mM L-glutamine and 5×10⁻² μM beta-Mercaptoethanol.

The purified B cells (10⁵B cells at final volume of 200 μl) were cultured with anti-mouse lgM Ab 5 μg/ml (IgG, F(ab′)₂ fragment) (Jackson ImmunoResearch Laboratories) or Hen Egg Lysozyme (Sigma), with or without BAFF (2 ng/ml or 10 ng/ml), in the absence or presence of various concentration of anti-BR3 mAbs. Proliferation was measured by ³H-thymidine uptake (1 uCi/well) for the last 8 hours of 48 hour stimulation. In some experiments, anti-BR3 mAbs as well as BR3-Fc fusion protein were pre-boiled for 5 min using PCR machine to inactivate them (controls).

Both B9C11 and V3-1m can inhibit the BAFF costimulatory activity during anti-IgM mediated primary murine B cell proliferation. Neither B9C11 nor V3-1m showed any direct effect on B cell proliferation in the absence or presence of various doses of anti-IgM antibody (data not shown). Inhibition of proliferation of B cells from anti-HEL BCR transgenic mice with V3-1m and B9C11 (not boiled V3-1m or B9C11) was also observed (data not shown). Both antibodies are not agonistic in that they do not trigger normal murine B cells proliferation on their own.

(a) Other Antibodies

Human B cells were isolated from peripheral blood mononuclear cells by positive selection using CD19 MACS magnetic beads according to the manufacturer's protocol (Miltenyi Biotec, Auburn, Calif.). Cells were either used immediately after isolation or were frozen in liquid nitrogen for later use; fresh and frozen cells performed equivalently in the assay. B cells were cultured at 1×10⁵ cells/well in black 96-well plates with clear, flat-bottomed wells (PE Biosystems, Foster City, Calif.).

For evaluating antagonistic effects of anti-BR3 antibodies, the cells were incubated with soluble recombinant BAFF (10 ng/ml) and a F(ab′)2 goat anti-human IgM (Fc specific) antibody (4 μg/ml) (Jackson ImmunoResearch, West Grove, Pa.) in the presence and absence of various concentrations of anti-BR3 antibody ranging from 100 nM to 1.3 μM (15 μg/ml-1 ng/ml). B cell proliferation was assessed at day 6 by adding Ceiltiter Glo (Promega, Madison, Wis., reconstituted according the manufacturer's instructions) to each assay well. The plates were then read in a luminometer after incubation for 10 minutes at room temperature.

The potential for anti-BR3 antibody agonism to stimulate B cell proliferation was assessed by incubating anti-BR3 antibody (100 nM to 1.3 pM) in the presence of the anti-IgM antibody alone (4 μg/ml) or in the presence of anti-IgM plus a ‘cross-linking’ F(ab′)2 goat anti-human IgG Fc antibody (Pierce, Rockford, Ill., 30 μg/ml) and in the absence of BAFF. Proliferation was assessed at day 6 using Ceiltiter Glo as described above.

9.1-RF blocked BAFF-dependent B cell proliferation and does not agonize. 2.1-46 stimulated B cell proliferation in the presence of anti-IgM, indicating that it can act as an agonist.

### Example 5

### Affinity Measurements Using Biacore

### Materials and Methods

Real-time biospecific interactions were measured by surface plasmon resonance using Pharmacia BIAcore® 3000 (BIAcore AB, Uppsala, Sweden) at room temperature (Karlsson, R., et al. (1994) Methods 6:97-108; Morton, T. A. and Myszka, D. G. (1998) Methods in Enzymology 295: 268-294). Human BR3 ECD or vBR3-Fc was immobilized to the sensor chip (CM5) through primary amine groups. The carboxymethylated sensor chip surface matrix was activated by injecting 20 μl of a mixture of 0.025 M N-hydroxysuccinimide and 0.1 M N-ethyl-N′(dimethylaminopropyl) carbodiimide at 5 μl/min. 5-10 μl of 5 μg/ml solution of BR3 ECD or vBR3-Fc in 10 in/VI sodium acetate, pH 4.5, were injected at 5 μl/min. After coupling, unoccupied sites on the chip were blocked by injecting 20 μl of 1M ethanolamine, pH 8.5. The running buffer was PBS containing 0.05% polysorbate 20. For kinetic measurements, two-fold serial dilutions of anti-BR3 antibodies (6.2-100 nM or 12.5-200 nM) in running buffer were injected over the flow cells for 2 minutes at a flow rate of 30 μl/min and the bound anti-BR3 antibody was allow to dissociate for 20 minutes. The binding surface was regenerated by injecting 20-30 μl of 10 mM glycine•HCl (pH 1.5). Flow cell one, which was activated but did not have BR3 ECD or BR3-Fc immobilized, was used as a reference cell. There was no significant non-specific binding of anti-BR3 antibodies to flow cell one. Data were analyzed using a 1:1 binding model using global fitting. The association and dissociation rate constants were fitted simultaneously (BIAevaluation software). Similar results were obtained whether samples were run in the order of increasing or decreasing concentrations for selected antibodies tested.

Binding kinetics of anti-BR3 antibodies to BR3 ECD or BR3-Fc were measured by BIAcore. BR3 ECD or vBR3-Fc was immobilized on sensor chips, and serial dilutions of antibodies were injected over the flow cells. Alternatively, anti-BR3 antibodies were immobilized on sensor chips, and serial dilutions of BR3 ECD were injected over the flow cells. A high flow rate was used in order to minimize mass transport effects. Results of humanized Fab and humanized IgG antibodies compared side by side. The apparent binding affinities obtained using IgG in solution are higher than those obtained using Fab in solution, likely due to the avidity effects since IgG is bivalent.

### Example 6

### Functional Epitope Mapping

The following assays were used to functionally map the epitopes on BR3 important for anti-BR3 antibody binding.

Library Construction for miniBR3 Shotgun Scanning. Libraries displaying epitope-tagged p-miniBR3 on M13 bacteriophage were constructed by successive mutageneses of phagemid pW1205a as previously described (Weiss, G. A., et al., (2000) Proc Natl Acad Sci USA 97:8950-4; Gordon, N. et al., (2003) Biochemistry 42:5977-83). This phagemid encodes a peptide epitope tag (MADPNRFRGKDLGG) fused to the N-terminus of human growth hormone followed by M13 gene-8 major coat protein. pW1205a was used as a template for Kunkel mutagenesis (Kunkel, J. D., et al., (1987) Methods Enzymol 154:367-82) to generate appropriate templates for miniBR3 shotgun library construction. Oligonucleotides replaced the fragment of pW1205a encoding human growth hormone with DNA fragments encoding a partial sequence of miniBR3 containing TAA stop codons in place of the region to be mutated. The two new templates generated, template 1 (encoding residues 34-42) and template 2 (encoding residues 17-25), were each used to construct a miniBR3 library as previously described (Sidhu, H. et al., Methods Enzymol 328:333-63). Each “partial miniBR3” template was used as the template for Kunkel mutagenesis with mutagenic oligonucleotides designed to replace the template stop codons with the complementary region of miniBR3, while simultaneously introducing mutations at the desired sites. At the sites of mutation, wild-type codons were replaced with the corresponding shotgun alanine codon (Weiss, supra). Each of these two libraries allowed for mutations at 11 residues in miniBR3 with no mutated positions in common between libraries. Library 1 encoded shotgun codons at positions 17, 18, 20-23, 25, 27, 28, 30, and 33, while library 2 encoded shotgun codons at positions 26, 29, 31, 34, and 36-42. Each library contained 2×10⁹ members, allowing for complete representation of the theoretical diversity (>10⁴-fold excess).

Library Sorting and Analysis. Phage from each of the two libraries described above were subjected rounds of binding selection against the neutralizing antibodies 9.1, 2.1, 8G4, 11G9 (functional selection) and V3-1 or an anti-tag antibody (3C8:2 F4, Genentech, Inc.) (display selection) immobilized on 96-well Nunc Maxisorp immunoplates. The display selection was included in order to normalize the anti-BR3 antibody-binding selection for expression differences between library members. Phage eluted from each target were propagated in E. coli XL1-blue; amplified phage were used for selection against the same target as in the previous round. After two rounds of selection, 48 individual clones from each library and selection were grown in a 96-well format in 400 L of 2YT medium supplemented with carbenicillin and KO7 helper phage. Supernatants from these cultures were used directly in phage ELISAs to detect phage-displayed variants of miniBR3 capable of binding the antibody target they were selected against to confirm binding.

Phage ELISA can be performed generally as followed. Maxisorp immunoplates (96-well) were coated with capture target protein (anti-BR3 antibody) for two hours at room temperature (100 μl at 5 μg/ml in 50 mM carbonate buffer (pH 9.6)). The plates were then blocked for one hour with 0.2% BSA in phosphate-buffered saline (PBS) and washed eight times with PBS, 0.05% Tween 20. Phage particles were serially diluted into BSA blocking buffer and 100 μl was transferred to coated wells. After one hour, plates were washed eight times with PBS, 0.05% Tween 20, incubated with 100 μl of 1:3000 horseradish peroxidase/anti-M13 antibody conjugate in BSA blocking buffer for 30 minutes, and then washed eight times with PBS, 0.05% Tween 20 and twice with PBS. Plates were developed using an o-phenylenediamine dihydrochloride/H₂O₂ solution (100 μl), stopped with 2.5 M H₂SO₄ (50 μl), and absorbance measured at 492 nm.

All clones tested were found to be positive in their respective ELISAs and were then sequenced as previously described (Weiss, supra). Sequences of acceptable quality were translated and aligned.

Data for BAFF binding and display selection were previously measured (Gordon, supra). Data for anti-BR3 binding and display selection was similarly calculated. Generally, the occurrence of the wild-type residue (wt) and each ala mutation (mut) found amound sequenced clones following two rounds of selection for binding to anti-BR3 antibody or anti-tag antibody was tabulated. The occurrence of the wild-type residue was divided by that of the mutant to determine a wt/mut ratio for each mutation at each position (not shown).

F-values were calculated as previously described (Weiss, supra-, Gordon, supra). Generally, a normalized frequency ratio (F) was calculated to quantify the effect of each BR3 mutation on BAFF or anti-BR3 antibody-binding while accounting for display efficiencies: i.e., F=[wt/mutant(BAFF or anti-BR3 antibody selection)] divided by [wt/mutant(display selection)]. Deleterious mutations have ratios >1, while advantageous mutations have ratios <1; boldface indicates a >10-fold effect. Mutations that showed a greater than 10-fold effect (i.e., F>10 or F<0.1) were considered particularly significant.

TABLE 4
F values

| Residue | 9.1 | 2.1 | 8G4 | 11G9 | V3-1 | BAFF |
|---|---|---|---|---|---|---|
| T17 | 0.6 | 0.6 | 1.5 | 0.5 | 0.5 | 0.9 |
| P18 | 0.4 | 0.5 | 1.5 | 0.5 | 0.8 | 0.9 |
| C19 | | | | | | |
| V20 | 0.6 | 3 | 2.1 | 1.1 | 0.9 | 1.4 |
| P21 | 1 | 1.9 | 62 | 40 | 0.6 | 0.5 |
| A22 | 0.3 | 3.2 | 69 | 45 | 0.7 | 0.7 |
| E23 | 4.8 | 9.6 | 11 | 6.9 | 2.4 | 5.4 |
| C24 | | | | | | |
| F25 | 81 | 49 | 58 | 38 | 21 | 46 |
| D26 | 8.7 | 6.1 | 6.4 | 8.5 | 8.7 | 17 |
| L27 | 2.1 | 0.8 | 12 | 1.1 | 1.4 | 9.5 |
| L28 | 1.5 | 0.1 | 2.5 | 0.4 | 98 | 210 |
| V29 | 0.3 | 0.5 | 0.8 | 1 | 92 | 57 |
| R30 | 10 | 10 | 11 | 1.7 | 20 | 16 |
| H31 | 0.5 | 0.6 | 3.8 | 2.8 | 0.1 | 0.3 |
| C32 | | | | | | |
| V33 | 10 | 10 | 38 | 24 | 14 | 106 |
| A34 | 14 | 62 | 41 | 32 | 13 | 28 |
| C35 | | | | | | |
| G36 | 1.9 | 14 | 1.7 | 1.8 | 0.7 | 1.3 |
| L37 | 0.7 | 0.1 | 0.8 | 0.7 | 0.7 | 5.4 |
| L38 | 89 | 0.9 | 1 | 0.9 | 1.4 | 47 |
| R39 | 63 | 0.5 | 2.2 | 3.1 | 0.4 | 4.1 |
| T40 | 0.4 | 0.2 | 0.5 | 0.5 | 0.6 | 0.5 |
| P41 | 7.2 | 0.7 | 1.7 | 1.7 | 1.6 | 1.9 |
| R42 | 2.2 | 1.8 | 0.8 | 0.9 | 0.9 | 1.5 |

The data indicates that 11G9, 9.1, and 2.1 exploit regions of sequence variation between human and murine BR3 (Table 4). The functional epitope for V3-1 mimics the functional epitope for BAFF that is highly conserved between human and murine BR3. 11G9, 2.1, 9.1, and V3-1 antibodies do not require BR3 glycosylation for binding. The functional epitope for the 9.1 antibody includes L38 and R39. The functional epitope for 2.1 includes G36. The functional epitope for V3-1 includes L28 and L29. The functional epitope for 11G9 includes P21 and A22. Alanine scanning mutation of residues A34, F25 and V33 also disrupted 9.1, 2.1, 11G9, and V3-1 binding to BR3 in this assay, which residues may be important for maintaining the structural integrity of BR3 in the phage.

### Example 7

### Antibody Dependent Cellular Cytotoxicity

Anti-BR3 chimeric monoclonal antibodies were assayed for their ability to mediate Natural-Killer cell (NK cell) lysis of BJAB cells (ADCC activity), a CD20 expressing Burkitt's lymphoma B-cell line, essentially as described (Shields et al., J. Biol. Chem. 276:6591-6604 (2001)) using a lactate dehydrogenase (LDH) readout. NK cells were prepared from 100 mL of heparinized blood from normal human donors using the RosetteSep® Human NK Cell Enrichment Cocktail (StemCell Technologies, Vancouver, B. C.) according to the manufacturer's protocol. The blood was diluted with an equal volume of phosphate buffered saline, layered over 15 mL of Ficoll-Paque™ (Amersham Biosciences, Uppsala, Sweden), and centrifuged for 20 min at 1450 RPM. White cells at the interface between layers were dispensed to 4 clean 50-mL tubes, which were filled with RPMI medium containing 15% fetal calf serum. Tubes were centrifuged for 5 min at 1450 RPM and the supernatant discarded. NK cells were diluted in assay medium (F12/DMEM 50:50 without glycine, 1 mM HEPES buffer pH 7.2, Penicillin/Streptomycin (100 units/mL; Gibco), glutamine, and 1% heat-inactivated fetal bovine serum) to 2×10⁶ cells/mL.

Serial dilutions of antibody (0.05 mL) in assay medium were added to a 96-well round-bottom tissue culture plate. BJAB cells were diluted in assay buffer to a concentration of 4×10⁵/mL. BJAB cells (0.05 mL per well) were mixed with diluted antibody in the 96-well plate and incubated for 30 min at room temperature to allow binding of antibody to BR3 (opsonization).

The ADCC reaction was initiated by adding 0.05 mL of NK cells to each well. In control wells, 2% Triton X-100 was added. The plate was then incubated for 4 h at 37° C. Levels of LDH released were measured using a cytotoxicity (LDH) detection kit (Kit# 1644793, Roche Diagnostics, Indianapolis, Ind.) following the manufacturers instructions. 0.1 mL of LDH developer was added to each well, followed by mixing for 10s. The plate was then covered with aluminum foil and incubated in the dark at room temperature for 15 min. Optical density at 490 nm was then read and used to calculate % lysis by dividing by the total LDH measured in control wells. Lysis was plotted as a function of antibody concentration, and a 4-parameter curve fit (KaleidaGraph) was used to determine EC₅₀ concentrations.

All humanized anti-BR3 antibodies were strongly active in directing NK cell mediated lysis of BJAB cells (human Burkitt's Lymphoma) with relative potencies less than 1 nM. Similar assays were carried out with Ramos (human Burkitt's lymphoma) and WIL2s cells (human B-cell lymphoma) instead of BJAB cells. ADCC killing of Ramos and WIL2s cells was observed with anti-BR3 antibodies. An anti-Her2 antibody (4D5) was used as a negative control. In general, antibodies with higher affinity for BR3 were more potent in antibody-dependent cell-killing assays.

### Example 8

### Depletion of B Cells with BR3-Fc or Anti-BR3 Antibodies

The ability of anti-BR3 antibodies to deplete B cells was compared with BR3-Fc. Six week old BALB/c mice were treated interperitoneally at day 0 with 500 μg control (mouse IgG2a), mouse BR3-Fc or anti-BR3 (V3-1) antibodies. Mice from each group were sacrificed at day 1, 3, 7 and 15. Flowcytometry analysis of B cells in the blood, lymph nodes and spleen at day 7 of treatment was conducted. The blood, lymph nodes and spleen show fewer B cells (CD21+CD23+ and CD21highCD23low) in V3-1 treated mice than in BR3-Fc and control treated animals. BR3-Fc treatment has previously been shown to significantly reduce the number of B cells compared with control Fc treated animals.

In another experiment under similar conditions, FACS analysis of blood, lymph nodes and spleen generally showed fewer B cells (CD21+CD23+ and CD21highCD23low) in V3-1 treated mice than in BR3-Fc and control treated animals. BR3-Fc significantly reduced the number of B cells compared with control animals particularly at later time points. The absolute number of B cells contained in 1 ml of blood; the % of B cells in lymph nodes and the absolute numbers of follicular (FO—CD21+CD23+) or marginal zone (MZ—CD21high CD23low) in the spleen at days 1, 3, 7 and 15 were detected. Data were expressed as the mean+/−standard error (n=4).

In another experiment under similar conditions, FACS analysis of plasmablasts in the spleen (top row—IgM+Syn+) and germinal center cells (middle row—B220+CD38low) show that anti-BR3 antibodies (V3-1) can deplete some plasmablasts and germinal center cells. BR3-Fc significantly reduced the number of plasmablasts compared with control animals.

The data showed that a greater extent of B cell depletion was observed after treatment with anti-BR3 antibodies than with BR3-Fc, which fusion protein blocks BAFF binding to BR3 but does not have ADCC function.

### Example 9

### Fc-Dependent Cell Killing and BAFF Blockade For Maximal B Cell Reduction

BALB/c mice were treated with a single dose 10 mg/kg of anti-BR3 antibody (mV3-1), mV3-1 with D265A/N297A mutations, a non-BAFF blocking anti-BR3 antibody PIH11 or BR3-Fc. B cells from spleen or peripheral blood were analyzed by flowcytometry at day 6 post treatment. The absolute numbers of peripheral blood B cells (B220+) and splenic follicular B cells (CD21+CD23+) after treatment are detected.

The D265A/N297A Fc mutation abolished binding of FcγRIII in vitro. The results indicate that although both the non-blocking antibody, the anti-BR3 antibodies with defective Fcgamma receptor-binding, and BR3-Fc can reduce B cell populations, the anti-BR3 antibody having both Fc-dependent cell killing activity and BAFF-blocking activity can be a much more potent B cell reducing/depleting agent. This is due to combining both activities, antibody dependent cell cytotoxicity (ADCC) and B cell survival blockade, into one molecule.

### Example 10

### Fcγ Receptor Binding

Human FcγRs (also referred to as hFcgR below) lacking their transmembrane and intracellular domains and comprising His-tagged glutathione S transferase (GST) sequences at their C-terminus were prepared as described previously (Shields, R. L. et al., (2001) JBC 276:6591-6604).

MaxiSorp 96-well microwell plates (Nunc, Roskilde, Denmark) were coated with 2 μg/ml anti-GST (clone 8E2.1.1, Genentech), at 100 μl/well in 50 mM carbonate buffer, pH 9.6, at 4° C. overnight. Plates were washed with PBS containing 0.05% polysorbate, pH 7.4 (wash buffer) and blocked with PBS containing 0.5% BSA, pH 7.4, at 150 μl/well. After an hour incubation at room temperature, plates were washed with wash buffer. Human Fcγ receptor was added to the plates at 0.25 μg/ml, 100 μl/well, in PBS containing 0.5% BSA, 0.05% polysorbate 20, pH 7.4 (assay buffer). The plates were incubated for one hour and washed with wash buffer. For low affinity Fcγ receptors IIa, IIb, III (F158) and high affinity III (V158), antibodies were incubated with goat F(ab′)₂ anti-κ (Cappel, ICN Pharmaceuticals, Inc., Aurora, Ohio) or anti-λ. (BioSource, Camarillo, Calif.) antibody at a 1:2 (w/w) ratio for 1 hour to form antibody complexes. Eleven twofold serial dilutions of complexed IgG antibodies (1.17-50000 ng/ml in threefold serial dilution) in assay buffer were added to the plates. For the high affinity FcγRI, eleven twofold serial dilutions of uncomplexed IgG antibodies (0.017-1000 ng/ml in threefold serial dilution) in assay buffer were added to the plates. After a two-hour incubation, plates were washed with wash buffer. Bound IgG was detected by adding peroxidase labeled goat F(ab′)₂ anti-human IgG F(ab′)₂ (Jackson ImmunoResearch, West Grove, Pa.) at 100 μl/well in assay buffer. After a one-hour incubation, plates were washed with wash buffer and the substrate 3,3′,5,5′-tetramethyl benzidine (TMB) (Kirkegaard & Perry Laboratories) was added at 100 μl/well. The reaction was stopped by adding 1 M phosphoric acid at 100 μl/well. Absorbance was read at 450 nm on a multiskan Ascent reader (Thermo Labsystems, Helsinki, Finland).

The absorbance at the midpoint of the standard curve (mid-OD vs. ng/ml) was calculated. The corresponding concentrations of standard and samples at this mid-OD were determined from the titration curves using a four-parameter nonlinear regression curve-fitting program (KaleidaGraph, Synergy software, Reading, Pa.). The relative activity was calculated by dividing the mid-OD concentration of standard by that of sample. The Herceptin® Ab has previously been shown to bind Fcγ Receptors and was used as a positive control here.

For all FcγR, binding values reported are the binding of each 9.1-RF variant relative to 9.1RF, taken as (A_{450 nm(variant)}/A_{450 nm(9/1RF)}) at 0.33 or 1 μg/ml for FcγRII and FcγRIIIA and 2 μg/ml for FcγRI. A value greater than 1 denotes binding of the variant was improved compared with 9.1RF, whereas a ratio less than 1 denotes reduced binding compared with 9.1RF. The hFcγRIII(F158) and hFcγRIII(V158) refer to hFcγRIII isotypes having lower affinity and higher affinity for human IgG, respectively.

The 9.1 anti-BR3 antibodies bind FcγRs similarly and should promote ADCC.

TABLE 5
Relative binding to Fcγ receptors.

| | | | | hFcgRIII | hFcgRIII |
|---|---|---|---|---|---|
| Antibody | hFcgRI | hFcgRIIa | hFcgRIIb | (F158) | (V158) |
| Herceptin ® | 1.02 | 0.54 | 0.62 | 0.51 | 0.80 |
| Ab | | | | | |
| 9.1-RF | 1.00 | 1.00 | 1.00 | 1.00 | 1.00 |
| 9.1-RF N434A | 0.97 | 0.66 | 0.45 | 0.42 | 0.58 |
| 9.1-RF N434W | 1.00 | 0.64 | 0.40 | 0.24 | 0.51 |

### Example 11

### Anti-BR3 Antibodies with Altered ADCC Activity

(a) FcgammaR Binding

By site-directed mutagenesis, the Fc region of the 9.1RF antibodies were mutated as follows: S298A/K326A/E333A/K334A (“9.1(5)”), S298A/E333A/K334A (“9.1(6)”), 239D/332E (“9.1(7)”) 239D/298A/332E (“9.1(8)”), 239D/268D/298A/332E (“9.1(9)”), 239D/268D/298A/326A/332E (“9.1(10)”), 239D/268D/283L/298A/332E (“9.1(11)”) or 239D/268D/283L/298A/326A/332E (“9.1(12)”). Additionally, by site-directed mutagenesis, the Fc region of the V3-46s antibody was mutated as follows: S298A/K326A/E333A/K334A (“V3(5)”), 239D/332E (“V3(7)”) or 239D/298A/332E (“V3(8)”). Oligonucleotides specifying the amino acid substitutions were chemically synthesized and used for oligonucleotide-directed mutagenesis of plasmid encoding 9.1RF according to the protocol of Kunkel et al. (Methods in Enzymology (1987) 154, 367-382). Variant sequences were confirmed by dideoxynucleotide-based sequencing. Plasmid DNA was purified from 1 L cultures (2YT media containing 50 μg/mL carbenecillin) of E. coli XL-1 Blue (Stratagene, Inc.), transformed with the relevant plasmid and grown at 37° C. with shaking at 200 RPM, by using the gigaprep protocol described by Qiagen, Inc. Proteins were expressed by using the purified plasmid DNA for transient transfection of CHO cells or 293 cells. Antibodies were purified from 1 L of culture supernatant by chromatography on Protein A-Sepharose followed by cation exhange chromatography on SP-Sepharose. The identity of the purified protein was confirmed by SDS-PAGE and amino terminal sequencing. All of the purified antibodies produced a homogeneous peak upon analytical gel filtration chromatography, with a molar mass of 150,000±5000 calculated from static light scattering data, and less than 3% aggregate content. Analysis of N-linked oligosaccharides by MALDI-TOF indicated a carbohydrate composition typical of recombinant antibodies (Table 6).

TABLE 6
MALDI-tof analysis of released N-linked oligosaccharides from 9.1 Fc variants.

| | Oligosaccharide | Oligosaccharide | Oligosaccharide | Oligosaccharide | Oligosaccharide | Oligosaccharide |
|---|---|---|---|---|---|---|
| | area % | area % | area % | area % | area % | area % |
| Variant | Man5 | 1100 | 2000 | 2100 | 2110 | 2120 |
| 9.1(5) | 1 | 2 | 7 | 76 | 13 | 1 |
| 9.1(6) | 2 | 3 | 6 | 77 | 11 | 1 |
| 9.1(7) | 1 | 1 | 2 | 64 | 26 | 4 |
| 9.1(8) | 1 | 1 | 2 | 54 | 35 | 7 |

Binding of the variant antibodies to Fcγ receptors was evaluated using an ELISA-based assay. The extracellular domains of human Fcγ receptors I, IIa, IIb, IIIa(F158), IIIa(V158) and mouse Fcγ receptors I, II, and III, were expressed as His-tagged, GST fusion proteins in CHO cells and purified as described in Shields et al. (J. Biol. Chem. 276:6591-6604 (2001)). For the ELISA assay, the fusion proteins were captured on wells of microtiter plates that had been coated with an anti-GST antibody. Dilutions of the variant antibodies were added and allowed to bind followed by washing of the wells to remove unbound antibody. For the weaker binding-antibodies the samples were complexed with a Fab′2 fragment of an anti-hu γ-chain antibody prior to addition of the samples to the wells. Bound antibody was detected with an HRP-coupled, Fab′2 fragment of a goat anti-huFab′2 antibody. Binding curves were evaluated by using a 4-parameter equation to calculate the ECₛₒ value, the concentration of antibody that gives 50% of the signal observed at saturation. Herceptin® was used as the control antibody in these assays and the fold improvement in binding was calculated from the ratio of the EC₅₀ values (EC₅₀herceptin/EC₅₀sample).

Table 7 shows that the variants had insignificant changes in affinity for human FcγRI. Only 9.1 (7) and 9.1(8) had increased affinity for human FcγRIIb with 9.1(7) being the tightest binder. 9.1(7) and 9.1(8) had increased affinity for all three mouse Fcγ receptors whereas the affinity for 9.1(5) and 9.1(6) was unchanged.

TABLE 7

| | Human | Human | Human | Human | Human | Mouse | Mouse | Mouse |
|---|---|---|---|---|---|---|---|---|
| | Antibody | | | | | | | |
| | | | | IIIa | IIIa | | | |
| | I | IIa | IIb | (F158) | (V158) | I | II | III |
| WT | 1.0 | 2.3 | 0.7 | 2.3 | 1.6 | 1.2 | 0.7 | 0.8 |
| 9.1(5) | 0.6 | 0.2 | 0.7 | 25 | 9.2 | 1.9 | 1.3 | 1.2 |
| 9.1(6) | 0.7 | 0.2 | 0.4 | 18 | 6.9 | 2.5 | 0.4 | 0.6 |
| 9.1(7) | 0.7 | 5.3 | 17 | 110 | 18 | 62 | 19 | 9.9 |
| 9.1(8) | 0.7 | 0.9 | 3.5 | 160 | 34 | 24 | 7.6 | 4.0 |

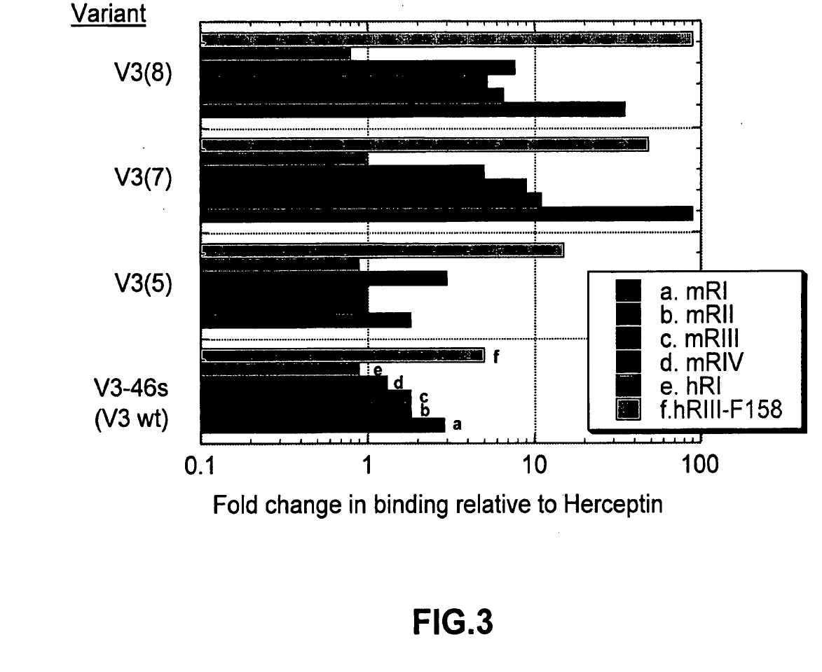

All of the anti-BR3 variants in Table 7 had increased affinity for both the F158 and V158 allotypes of human FcγRIIIa, with 9.1(8) having the greatest affinity for the receptor. FIG. 3 shows the fold change in binding of V3(8), V3(7), V3(5) and V3-46s to mouse FcγR (I, II, III, IV) or human FcγR (I, III-F158) relative to a control antibody (the Herceptin® antibody). Large Increases in binding affinity to mFcγRI and hRIII-F158 were observed for V3(7) and V3(8).

(b) ADCC Activity

The anti-BR3 antibodies were assayed for their ability to mediate Natural-Killer cell (NK cell) lysis of BJAB cells (ADCC activity), a BR3 and CD20 expressing Burkitt's lymphoma B-cell line, essentially as described (Shields et al., J. Biol. Chem. 276:6591-6604 (2001)) using a lactate dehydrogenase (LDH) readout. NK cells isolated from donors heterozygous for the F/V 158 allotype of CD16 were used in the assay at an effector:target ratio of 5:1. NK cells were prepared from 100 mL of heparinized blood using the RosetteSep® Human NK Cell Enrichment Cocktail (StemCell Technologies, Vancouver, B. C.) according to the manufacturer's protocol. The blood was diluted with an equal volume of phosphate buffered saline, layered over 15 mL of Ficoll-Paque™ (Amersham Biosciences, Uppsala, Sweden), and centrifuged for 20 min at 1450 RPM. White cells at the interface between layers were dispensed to 4 clean 50-mL tubes, which were filled with RPMI medium containing 15% fetal calf serum. Tubes were centrifuged for 5 min at 1450 RPM and the supernatant discarded. NK cells were diluted in assay medium (F12/DMEM 50:50 without glycine, 1 mM HEPES buffer pH 7.2, Penicillin/Streptomycin (100 units/mL; Gibco), glutamine, and 1% heat-inactivated fetal bovine serum) to 2×10⁶ cells/mL.

Serial dilutions of antibody (0.05 mL) in assay medium were added to a 96-well round-bottom tissue culture plate. BJAB cells were diluted in assay buffer to a concentration of 4×10⁵/mL. BJAB cells (0.05 mL per well) were mixed with diluted antibody in the 96-well plate and incubated for 30 min at room temperature to allow binding of antibody to BR3 (opsonization).

The ADCC reaction was initiated by adding 0.05 mL of NK cells to each well. In control wells, 2% Triton X-100 was added. The plate was then incubated for 4 h at 37° C. Levels of LDH released were measured using a cytotoxicity (LDH) detection kit (Kit#1644793, Roche Diagnostics, Indianapolis, Ind.) following the manufacturers instructions. 0.1 mL of LDH developer was added to each well, followed by mixing for 10s. The plate was then covered with aluminum foil and incubated in the dark at room temperature for 15 min. Optical density at 490 nm was then read and used to calculate % lysis by dividing by the total LDH measured in control wells. Lysis was plotted as a function of antibody concentration, and a 4-parameter curve fit (KaleidaGraph) was used to determine EC50 concentrations.

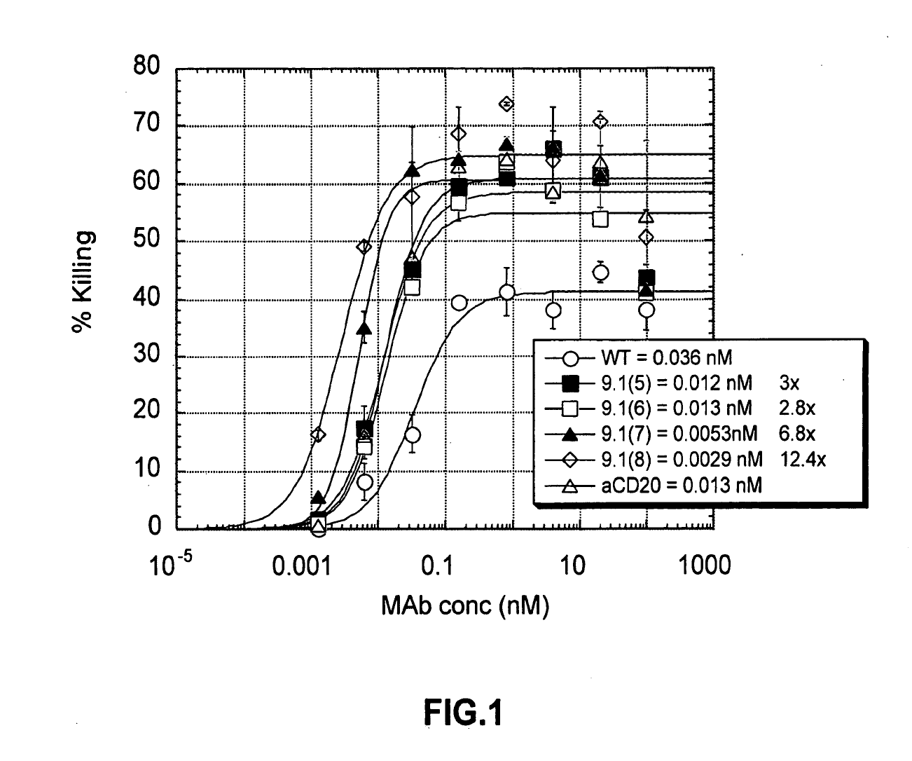

FIG. 1 shows that the anti-BR3 variants were active in the ADCC assay giving EC₅₀ values less than 1 nM (% killing vs antibody concentration). The Fc substitutions led to an increase in potency relative to 9.1RF (data not shown) by the lowering of the EC₅₀ and increase in the maximal % killing. The S298A/K326A/E333A/K334A mutant had a 3 fold higher ADCC activity in this assay relative to 9.1RF (relative EC₅₀ values). The S298A/E333A/1C334A mutant had a 2.8 fold higher ADCC activity in this assay relative to 9.1RF (relative EC₅₀ values). In this comparison, 9.1(8) had the highest activity, about a 12-fold lowering of the EC50 relative to 9.1RF, and both 9.1(7) and 9.1(8) had higher activity than an anti-CD20 antibody, Rituximab® antibody, on this cell line.

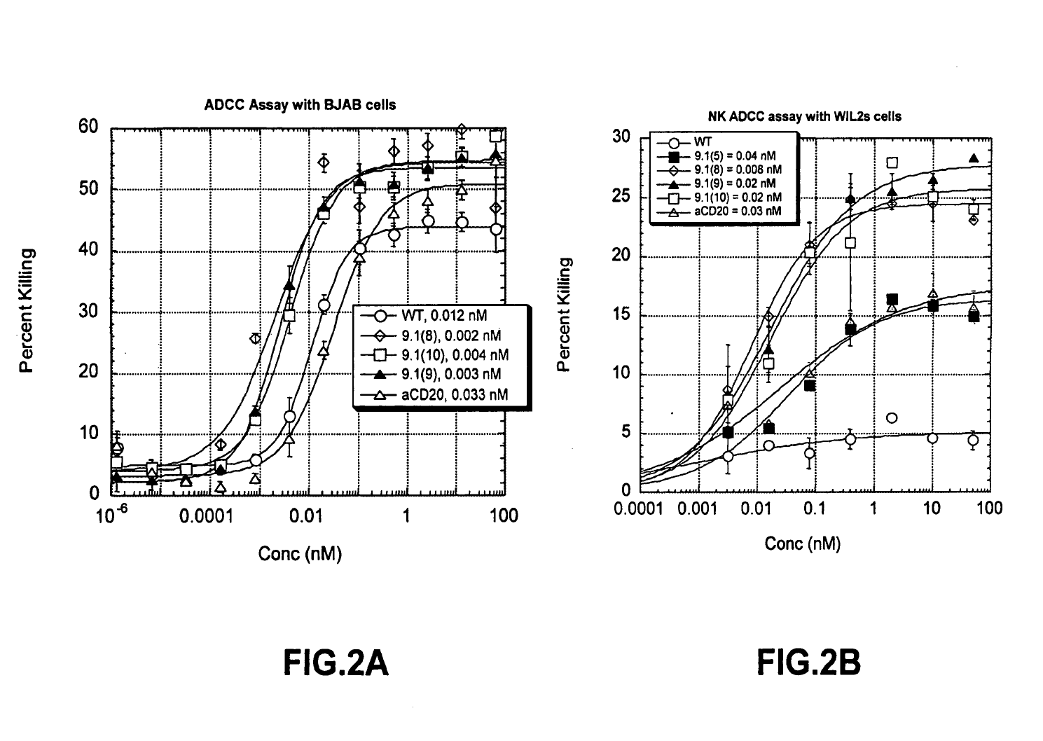

Additional experiments also showed that 9.1(8) was the Fc variant having the most potent ADCC activity on WIL2-S and BJAB cell lines (FIGS. 2A and 2B). Table 8 is a table listing the relative fold increase in ADCC activity of the Fc variants compared to 9.1RF. Studies showed that 9.1(5) and 9.1(8) were not agonistic for B cell proliferation.

TABLE 8

| ABR var. | 239 | 268 | 283 | 298 | 326 | 332 | 333 | 334 | Expression | ADCC |
|---|---|---|---|---|---|---|---|---|---|---|
| WT(9.1RF) | S | H | E | S | K | I | E | K | | |
| 9.1(5) | — | — | — | A | A | — | A | A | CHO | 3 |
| 9.1(6) | — | — | — | A | — | — | A | A | CHO | 3 |
| 9.1(7) | D | — | — | — | — | E | — | — | CHO | 7 |
| 9.1(8) | D | — | — | A | — | E | — | — | CHO | 12 |
| 9.1(9) | D | D | — | A | — | E | — | — | 293 | 10 |
| 9.1(10) | D | D | — | A | A | E | — | — | 293 | 10 |
| 9.1(11) | D | D | L | A | — | E | — | — | 293 | ND |
| 9.1(12) | D | D | L | A | A | E | — | — | 293 | ND |

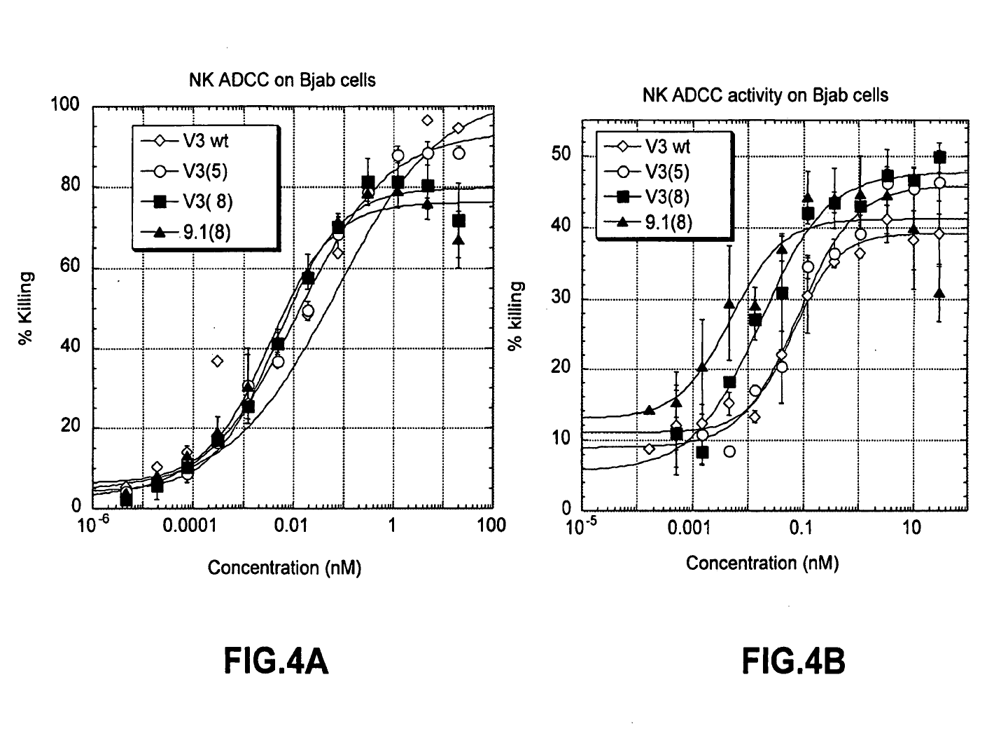

The V3 Fc variants displayed variable, low to substantial increases in ADCC activity (FIGS. 4A and 4B). In these particular experiments, the limits of dynamic range may have been reached and variability in donor natural killer cells may have contributed to the results.

(c) B Cell Depletion with Fc Variants

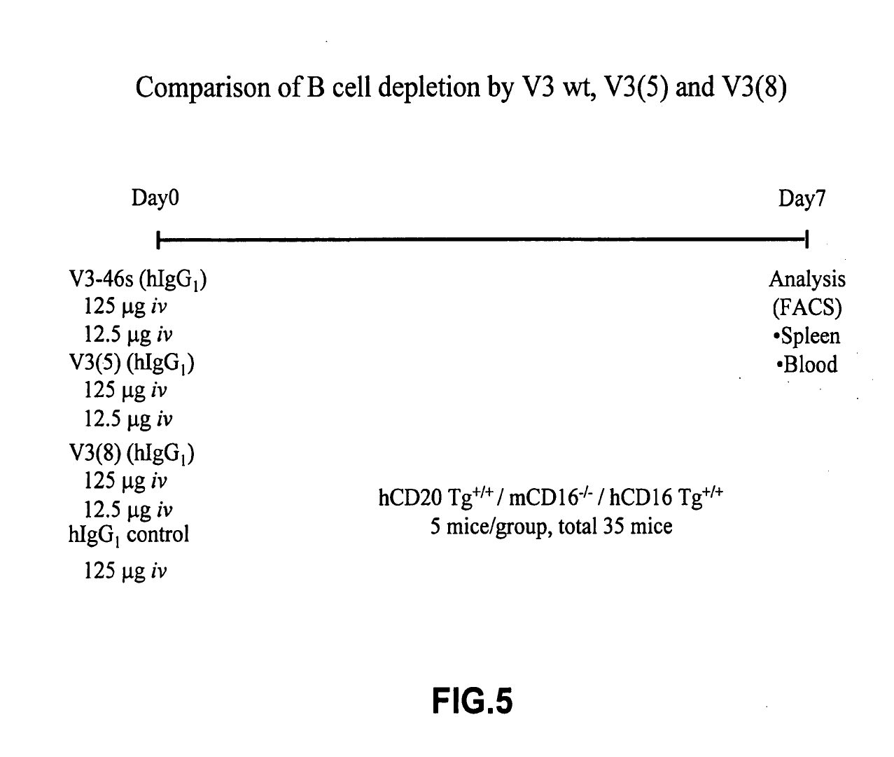

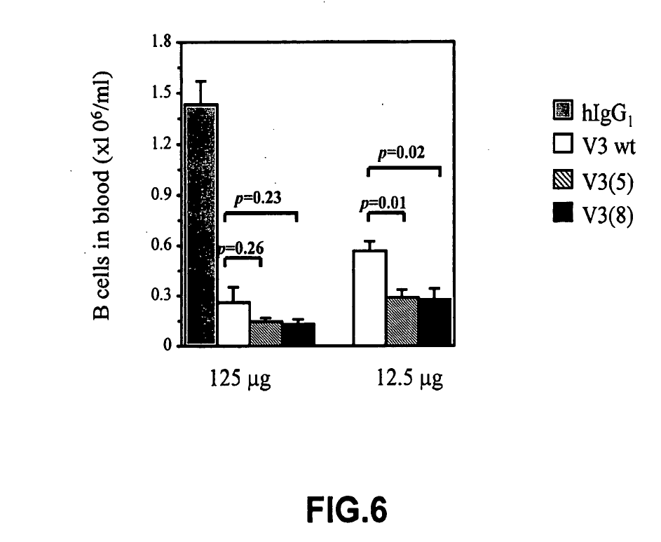

hCD20 Tg+/+mCD16−/− hCD16 Tg+/+ mice were intravenously treated with 125 μg or 12.5 μg V3-46s, V3(5) or V3(8) variants at 125 or 125 μg of a hIgG1 negative control (e.g., Herceptin® antibody) as described in FIG. 5. B cells from the blood and spleen of the mice were assayed by FACS on day 7 after administration. FIG. 6 shows that the lower dose of Fc variant (12.5 μg) resulted in an increased depletion of blood B cells relative to wild-type. At the timepoint assayed, variants V3(3) and V3(8) showed similar results. No significant change for spleen B cells was observed at day 7 (data not shown).

Additional data indicates that anti-BR3 antibody variants of this invention can have potents ADCC activity but have little or no neutrophil killing activity compared to a B cell depleter such as an anti-CD20 antibody (data not shown). These results indicate that anti-BR3 antibody variants of this invention may have less toxicity that an anti-CD20 antibody such as V511 (data not shown).

### Example 12

### Underfucosylated Antibody Compositions

### (a) Conversion of an Existing Cell Line to an Underfucosylated Cell Line.

In order to achieve high yields of non-fucosylated antibodies in mammalian cells, a RNAi approach was employed to knock down the expression of FUT8 gene. A plasmid was used to produce short hairpin siRNA consisting of 19 nt (nucleotide) sense siRNA sequence specific to the gene of FUT8, linked to its reverse complementary antisense siRNA sequence by a short spacer (9 nt hairpin loop), followed by 5-6 U′ s at 3′ end.

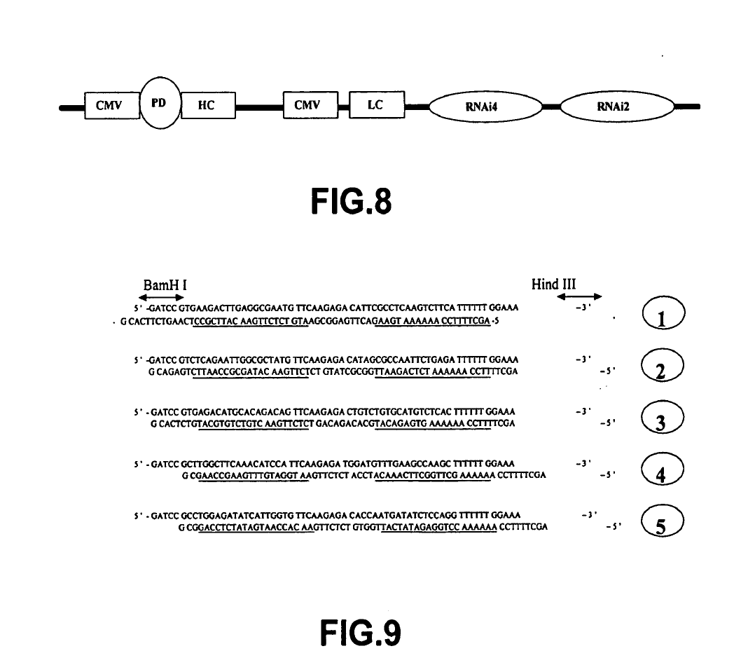

Four different RNAi probes were designed (probe #1-4) to target the different regions based on the available CHO FUT8 DNA sequence (FIGS. 8 and 9).

To test the efficacy of these RNAi probes, a FLAG-tagged FUT8 fusion protein was constructed using the available CHO FUT8 DNA sequence (Genbank accession no. P_AAC63891). RT-PCR was performed with FUT8 primers and the resulting PCR fragment was fused with 5′ FLAG tag sequence. The tagged FUT8 fragment was cloned into an expression vector. The RNAi probe plasmid and flag-tagged FUT8 plasmid were cotransfected into CHO cells. Cell lysate was extracted 24 hours after transfection and the FUT8 fusion protein level was analyzed by anti-flag M2 antibody by immuno blotting. In the presence of RNAi probes, the fusion protein expression was significantly inhibited in four out of the five cases (data not shown).

Probe#2 (RNAi2) and #4 (RNAi4) showed the best inhibitory effect and were chosen for further evaluation.

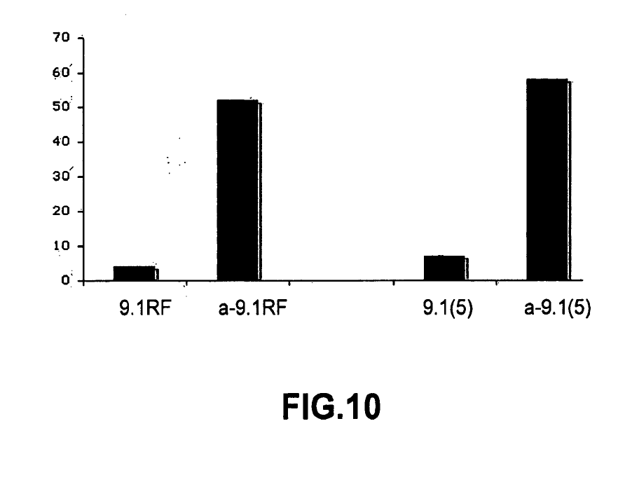

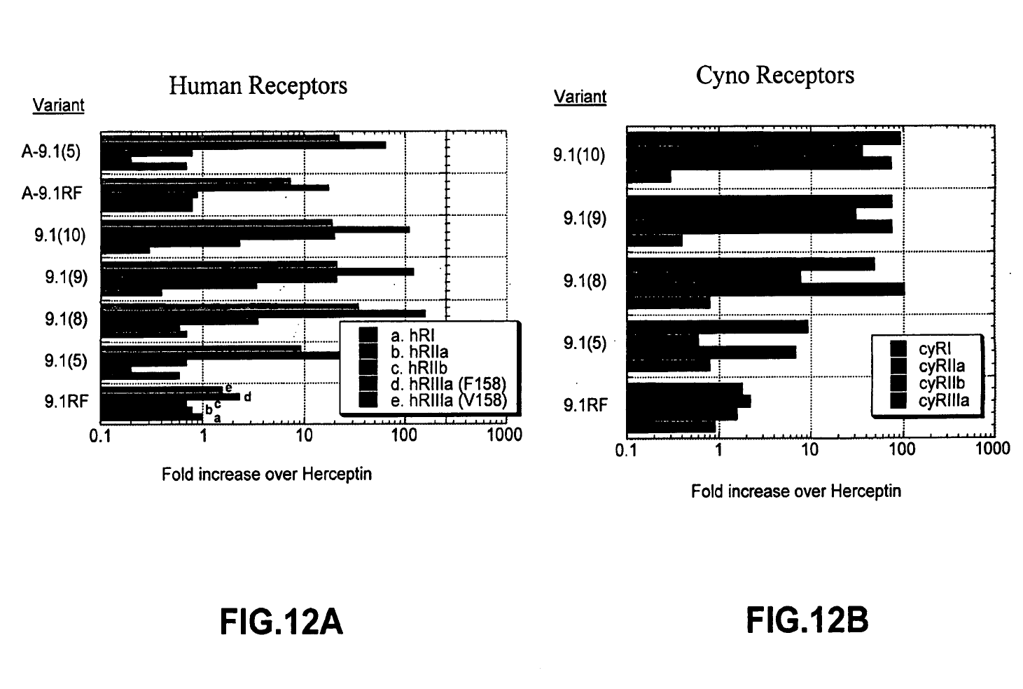

Probes 2 and 4 were transfected into a CHO cell line expressing a antibody described herein. The expressed anti-BR3 antibody was purified by a protein A column and submitted for MALDI-TOF fucose content and FcγR binding assay (described below). The RNAi transiently transfected cells produced about approximately 50%-52% nonfucosylated 9.1RF (“a-9.1RF”) and nonfucosylated 9.1(5) (“a-9.1(5)”) antibody as shown in FIG. 10. In contrast, the 9.1 variant cell lines not transfected with RNAi plasmids had 2-7% nonfucosylated antibodies (see 2000 m/z column). The a-9.1RF antibody pool and a-9.1(5) antibody pool having 50-52% nonfucosylation=showed an approximate 3-10 fold increase in ADCC activity (FIG. 11) and an increase in binding affinity towards FcγRIII (F158 allele) and FcγRIII (V158 allele) (FIGS. 12A and 12B). No effect was seen with hFcγR1, hFcγRIIa and hFcγRIIb (FIGS. 12A and 12B).

To confirm that the RNAi transfected cells do have less FUT8 RNA expression, a Northern blot can be performed using RNA samples extracted from the transfected cells 24 hours after transfection. Total RNA from cells containing a control plasmid (random mouse DNA sequence, no homology to any known mouse proteins) and 2 RNAi plasmids can be purified and hybridized with a 300 by probe. The knock down of endogenous a 1,6-fucosyltransferase RNA can be further confirmed by quantitative PCR (data not shown).

(b) Generation of Stable Cell Line with Simultaneous Knockdown of Fucosylation Level

Materials and Methods

Cell Culture and Transfection

Chinese Hamster Ovary (CHO) cells can be grown in growth medium with 5% FBS (fetal bovine serum) and 1×GHT (glycine, hypoxanthine, and thymidine) at 37° C. For transient transfection, DMRIE-C transfection reagent (Invitrogen) can be used. For stable transfection, Lipofectamine 2000 (Invitrogen) can be used. The cells can be transfected with a plasmid having the configuration of FIG. 8. Alternatively, a stable cells lines having a FUT8 knock-out came be made (e.g., method described in Yamane-Ohnuki, et al., (2004) Biotechnol. Bioeng. 87:614-622).

Selection

After the transfection, cells can be centrifuged to collect the pellet. The pellet can be resuspended in medium containing 25 nM methotrexate (MTX). Medium can be changed every 3 to 4 days. About 2 weeks after transfection, individual clones can be picked and grown in 96-well plates. Usually it takes about 1 week for cells to grow confluent in a 96-well plate.

ELISA Assay

When cells are confluent, the growth medium can be removed and the production medium can be added into each well. The day after adding the production medium, the plate can be incubated at 33° C. for 5-6 days before the ELISA assay. Typically an ELISA is performed with serial dilutions.

RNA Analysis

Total RNA can be purified with Qiagen's RNA purification kit and quantified by Taqman with gene specific primers and probes.

Matrix-Assisted Laser Desorption/Ionization Time-of-flight (MALDI-TOF) Mass Spectral Analysis of Asparagine-Linked Oligosaccharides:

Methods for analyzing the oligosaccharides by MALDI-TOF were conducted generally as follows: N-linked oligosaccharides were released from recombinant glycoproteins using peptide-N-glycosidase-F (PNGase F) procedure of Papac et al., Glycobiology 8, 445-454 (1998). Briefly, the wells of a 96 well PVDF-lined microtitre plate (Millipore, Bedford, Mass.) were conditioned with 100 μl methanol that was drawn through the PDVF membranes by applying vacuum to the Millipore Multiscreen vacuum manifold. The conditioned PVDF membranes were washed with 3×250 μl water. Between all wash steps the wells were drained completely by applying gentle vacuum to the manifold. The membranes were washed with reduction and carboxymethylation buffer (RCM) consisting of 6 M guanidine hydrochloride, 360 mM Tris, 2 mM EDTA, pH 8.6. Glycoprotein samples (50 μg) were applied to individual wells, again drawn through the PVDF membranes by gentle vacuum and the wells were washed with 2×50 μl of RCM buffer. The immobilized samples were reduced by adding 50 μl of a 0.1 M dithiothreitol (DTT) solution to each well and incubating the microtitre plate at 37° C. for 1 hr. DTT was removed by vacuum and the wells were washed 4×250 μl water. Cysteine residues were carboxylmethylated by the addition of 50 μl of a 0.1 M iodoacetic acid (IAA) solution which was freshly prepared in 1 M NaOH and diluted to 0.1 M with RCM buffer. Carboxymethylation was accomplished by incubation for 30 min in the dark at ambient temperature. Vacuum was applied to the plate to remove the IAA solution and the wells were washed with 4×250 μl purified water. The PVDF membranes were blocked by the addition of 100 μl of 1% PVP360 (polyvinylpyrrolidine 360,000 MW) (Sigma) solution and incubation for 30 minutes at ambient temperature. The PVP-360 solution was removed by gentle vacuum and the wells were washed 4×250 μl water. PNGase F (New England Biolabs, Beverly, Mass.) at 25 μl of a 25 Unit/ml solution in 10 mM Tris acetate, pH 8.3, was added to each well and the digest proceeded for 3 hr at 37° C. After digestion, the samples were transferred to 500 μl Eppendorf tubes and 2.5 μl of a 1.5 M acetic acid solution was added to each sample. The acidified samples were incubated for 2 hr at ambient temperature to convert the oligosaccharides from the glycosylamine to the hydroxyl form. Prior to MALDI-TOF mass spectral analysis, the released oligosaccharides were desalted using a 0.7-ml bed of cation exchange resin (AG50W-X8 resin in the hydrogen form) (Bio-Rad, Hercules, Calif.) slurried packed into compact reaction tubes (US Biochemical, Cleveland, Ohio).

For MALDI-TOF mass spectral analysis of the samples in the positive mode, the desalted oligosaccharides (0.5 μl aliquots) were applied to the stainless target with 0.5 μl of the 2,5 dihydroxybenzoic acid matrix (sDHB) that was prepared by dissolving 2 mg 2,5 dihydroxybenzoic acid with 0.1 mg of 5-methoxyslicylic acid in 1 ml of 1 mM NaCl in 25% aqueous ethanol. The sample/matrix mixture was dried by vacuum. The sample/matrix mixture was vacuum dried and then allowed to absorb atmospheric moisture prior to analysis. Released oligosaccharides were analyzed by MALDI-TOF on a PerSeptive BioSystems Voyager-ELITE mass spectrometer. The mass spectrometer was operated in the positive mode at 20 kV with the linear configuration and utilizing delayed extraction. Data were acquired using a laser power of approximately 1100 and in the data summation mode (240 scans) to improve the signal to noise. The instrument was calibrated with a mixture of standard oligosaccharides and the data was smoothed using a 19 point Savitsky-Golay algorithm before the masses were assigned. Integration of the mass spectral data was achieved using Caesar 7.2 data analysis software package (SciBridge Software).

### Example 13

### BJAB B Cell Binding Assay Using Afucosylated HU9.1RF IgG1 Antibody

THE BJAB cell binding assay used to assay the afucosylated anti-BR3 antibody Hu9.1RF IgG1 (comprising the VH and VL sequence of SEQ ID NOs: 35 and 21, respectively (see Table 2)), was essentially as described in Example 2 with the following minor modifications. BJAB cells were cultured in RPMI media supplemented with 10% fetal bovine serum (FBS, Sigma, St. Louis, Mo.), penicillin (100 U/ml, Gibco-Invitrogen, Carlsbad, Calif.), streptomycin (100 μg/ml, Gibco), and L-glutamine (10 mM).

For binding assays, cells were washed with cold assay buffer (phosphate buffered saline [PBS], pH 7.4) containing 1% FBS and 0.5% bovine serum albumin [BSA]). The cell density was adjusted to 1.25×10⁶/ml, and 200 μl of cell suspension was aliquotted into the wells of 96 well round bottom polystyrene plates (NUNC, Neptune, N.J.; 250,000 cells/well). The plates containing the cells were centrifuged at 1500 rpm for 5 minutes at 4′C, and the supernatant was carefully aspirated away from the cell pellets. Direct and competitive binding assays were performed as follows. For the direct binding assay, antibody samples (Hu9.1RF IgG1 and afucosylated Hu9.1RF IgG1) were serially diluted in cold assay buffer to concentrations ranging between 16.7-0.001 nM. Samples (100 μl) were added to the pelleted cells and the plates were shaken briefly on a plate shaker to loosen the cell pellets. The remainder of the assay was exactly as described in Example 2.

In the competitive binding assay, anti-BR3 mAbs compete with FLAG-tagged synthetic murine BAFF (produced at Genentech) for binding to cell surface BR3. The anti-BR3 antibodies (Hu9.1RF IgG1 and afucosylated Hu9.1RF IgG1) were serially diluted and combined with an equal volume of FLAG-BAFF to give final concentrations of 50-0.02 nM mAb and 25 nM FLAG-BAFF. The diluted samples were added to the pelleted BJAB cells in 96 well plates as described above. After a 45 min incubation on ice, the cells were washed three times with 200 μl cold assay buffer, and anti-FLAG-HRP antibody (Sigma) diluted 1/20,000 in assay buffer was added (100 μl/well). The plates were incubated for a final 45 minutes on ice. After three washes with cold assay buffer, color was developed using TMB, the reaction was stopped with H₃PO₄, and the plates were read as described above.

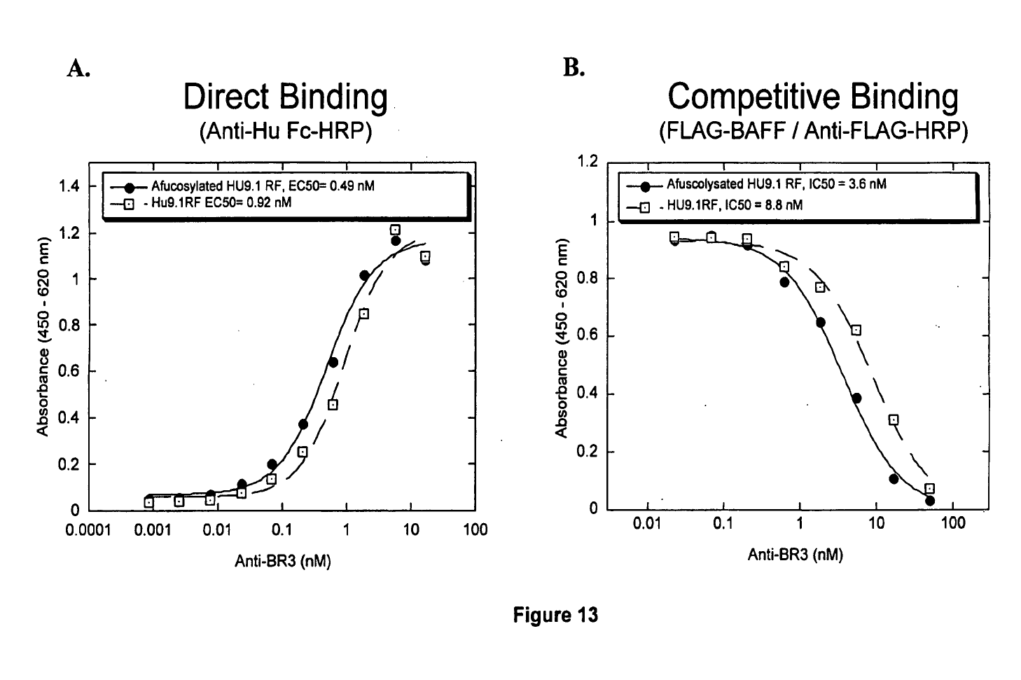

The results of the BJAB binding assays demonstrated that afucosylated Hu9.1RF IgG1 bound to BR3 at least as well as the fucosylated control version of the antibody (FIG. 13A). In the direct binding assay, the EC₅₀ for afucosylated Hu9.1RF IgG1 was 0.49 nM as compared to 0.92 nM for control Hu9.1RF IgG1. Moreover, in the competitive binding assay, afucosylated Hu9.1RF IgG1 displaced BAFF with an IC₅₀ of 3.6 nM as compared to 8.8 nM for the fucosylated form of the antibody (FIG. 13B).

These experiments demonstrate that the afucosylated form of Hu9.1RF IgG1 behaved essentially identically to control Hu9.1RF IgG1 in terms of cell surface BR3 binding and blockade of BAFF binding.

### Example 14

### Antagonistic and Agonistic Effects of Afucosylated HU9.1RF IgG1 Antibody

The B cell proliferation assays were performed essentially as described in Example 4, above with the following modifications. Human B cells isolated from peripheral blood mononuclear cells were either used immediately after isolation or were frozen in liquid nitrogen for later use; fresh and frozen cells performed equivalently in the assay. B cells were cultured at 1×10⁵ cells/well in black 96-well plates with clear, flat-bottomed wells (PE Biosystems, Foster City, Calif.). For evaluating antagonistic effects of antibodies, the cells were incubated with FLAG-BAFF (20 ng/ml) and a F(ab′)₂ goat anti-human IgM (Fc specific) antibody (4 μg/ml) (Jackson ImmunoResearch) in the presence and absence of various concentrations of anti-BR3 antibody ranging from 10 nM to 0.13 pM (1.5 μg/ml-0.02 ng/ml). B cell proliferation was assayed as described in Example 4. The potential for anti-BR3 antibody agonism was assessed by incubating the anti-BR3 antibody (10 nM to 0.13 pM) in the presence of the anti-IgM antibody alone (4 μg/ml). Proliferation was assessed at day 6 using Celltiter Glo as described above. Hu9.1RF IgG1 with an N434W mutation was used as an agonistic positive control and Herceptin was used as a human IgG1 isotype negative control.

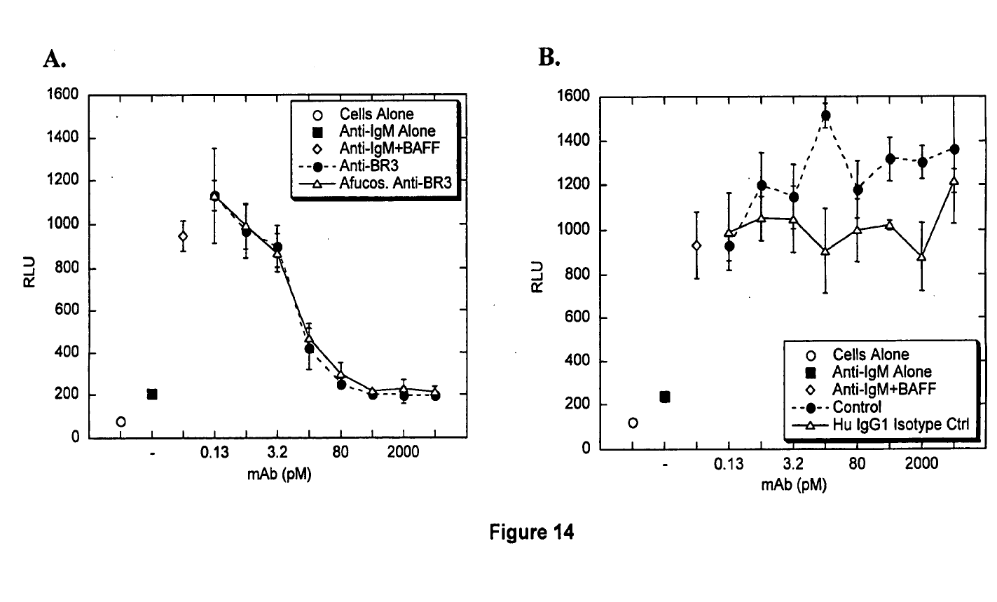

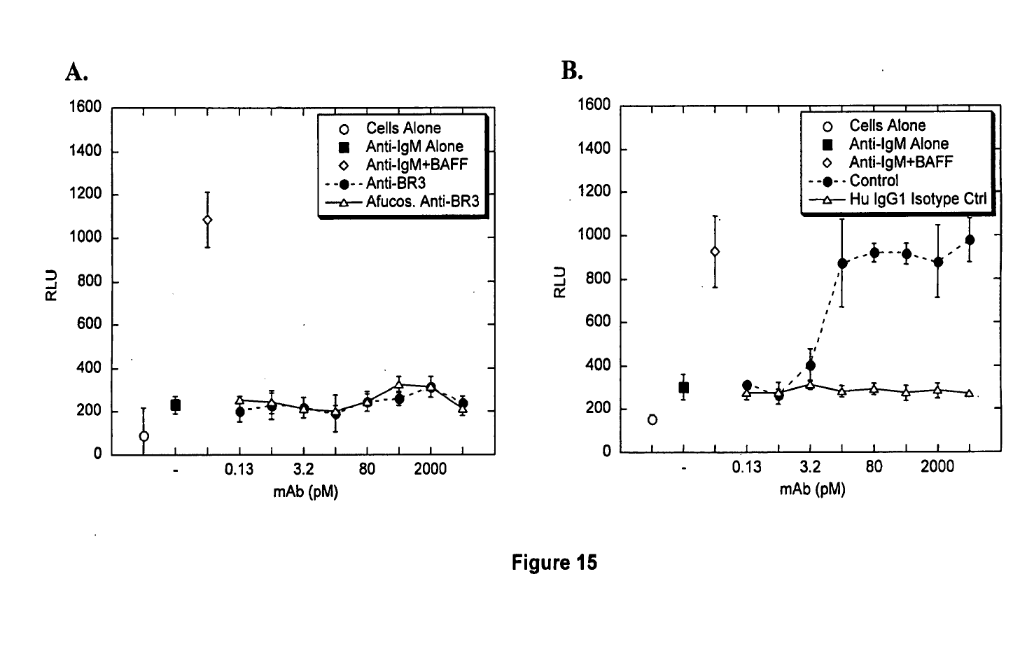

In the primary B cell proliferation assay, afucosylated Hu9.1RF IgG1 (comprising the VH and VL sequence of SEQ ID NOs: 35 and 21, respectively (see Table 2)), inhibited B cell proliferation stimulated by BAFF and anti-IgM essentially identically to control Hu9.1RF IgG1 (FIG. 14). The control antibodies (Hu9.1RF IgG1 with an N434W mutation and the IgG1 isotype control) had no effect on BAFF/anti-IgM stimulated proliferation as expected. In order to assess possible agonistic effects of the antibodies, B cells were stimulated with anti-IgM alone. The results showed that neither the control nor the afucosylated Hu9.1RF IgG1 antibodies stimulated B cell proliferation above the level observed with anti-IgM alone (FIG. 15). The agonistic positive control antibody (Hu9.1RF IgG1 with an N434W mutation) and isotype negative control both behaved as expected, with Hu9.1RF IgG1 with an N434W mutation causing a dose-dependent increase in proliferation in the presence of anti-IgM alone and the IgG1 control having no effect.

These experiments demonstrate that the afucosylated molecule was equally potent to control Hu9.1RF IgG1 in terms of inhibiting BAFF-stimulated primary B cell proliferation and had no evident agonistic effects.

### Example 15

### Affinity Measurements for Afucosylated Hu9.1RF IgG1 Antibody

Real-time biospecific interactions for the anti-BR3 antibodies, including the afucosylated Hu9.1RF IgG1 antibody, were measured by surface plasmon resonance using BIAcore® 3000 (GE Healthcare, Piscataway, N.J.) at room temperature (Karlsson et al., Methods 6, 97-108 (1994); Morton & Myszka, Methods in Enzymology, 295, 268-294 (1998)).

Two formats were used to assess the binding affinities of the anti-BR3 antibodies to BR3 extra cellular domain (BR3 ECD). The first format was similar to the format described in Example 5, above with the following modifications. Human BR3 ECD (52132-5P) was immobilized to a sensor chip (CM5) through primary amine groups and the sensor chip was activated as described above. A total of 70 ml (sequential injections of 5, 15, 30, 10, and 10 ml) of 5 mg/ml solution of BR3 ECD in 10 mM sodium acetate, pH 4.5, was injected at 5 ml/min. For kinetic measurements, two-fold serial dilutions of the anti-BR3 antibodies (1.56-200 nM) in running buffer were injected over the flow cells for 2 minutes at a flow rate of 30 ml/min. The bound anti-BR3 antibodies were allowed to dissociate for 20 minutes before the binding surface was regenerated by injecting 20 ml of 10 mM glycine HCl (pH 1.5). Flow cell one was used as a reference cell as described above. Data were analyzed using the 1:1 Langmuir binding model, as described above, and using the bivalent analyte model.

For the second format, anti-BR3 antibodies Hu9.1RF IgG1 and the afucosylated Hu9.1RF IgG1 were separately immobilized to individual flow cells on a sensor chip (CM5) through primary amine groups. The afucosylated Hu9.1RF IgG1 (Hu9.1RF IgG1-AF) for use in the experiments was generated in a host cell lacking a fucosyltransferase enzyme. The Hu9.1RF IgG1-AF was determined by MALDI-TOF to be 2% G0-F.

The carboxymethylated sensor chip surface matrix was activated by injecting 20 μl of a mixture of 0.025 M N-hydroxysuccinimide and 0.1 M N-ethyl-N′(dimethylaminopropyl) carbodimide at 5 μl/min. The Hu9.1RF IgG1 antibodies were prepared in 10 mM sodium acetate, pH 4.5, at 30 μg/ml for immobilization. 15 μl of the Hu9.1RF IgG1 was injected using three 5 μl injections and 20 μl of the afucosylated. Hu9.1RF IgG1 was injected using sequential 5, 10, and 5 μl injections. After coupling, unoccupied sites on the chip were blocked by injecting 20 μl of 1M ethanolamine, pH 8.5. PBS containing 0.05% polysorbate 20 was used as the running buffer. For kinetic measurements, two-fold serial dilutions of the BR3 ECD (1.95-1000 nM) in running buffer were injected over the flow cells for 2 minutes at a flow rate of 30 μl/min and the bound BR3 ECD was allowed to dissociate for 20 minutes. The binding surface was then regenerated with two injections of 30 μl 10 mM HCl (pH 2.0). Flow cell one, which was activated but did not have Hu9.1RF IgG1 immobilized, was used as a reference cell. There was no significant non-specific binding of BR3 ECD to flow cell one. Data were analyzed using the 1:1 Langmuir binding model and the association and dissociation rate constants were fitted simultaneously with the BIAevaluation software.

Binding kinetics of Hu9.1RF IgG1 and the afucosylated Hu9.1RF IgG1 to BR3 ECD were measured and the apparent kinetic parameters of the Hu9.1RF IgG1 antibodies binding to immobilized BR3 ECD are shown in Tables 9A and 9B.

TABLE 9A
Binding of anti-BR3 antibodies to BR3 ECD using 1 bivalent
analyte model for data analysis.

| Antibody in | | | | |
|---|---|---|---|---|
| solution | Ka (105/Ms) | Kd (10-4/s) | KD (nM) | Rmax (RU) |
| Hu9.1RF IgG1 | 6.58 | 3.39 | 0.52 | 520 |
| Afucosylated | 5.32 | 1.49 | 0.28 | 542 |
| Hu9.1RF IgG1 | | | | |

37 RU of BR3 ECD was immobilized. Samples were analyzed at 1.56-200 nM in 2-fold serial dilution, unless specified otherwise.

TABLE 9B
Binding of anti-BR3 antibodies to BR3 ECD using 1:1 Langmuir
model for data analysis.

| Antibody in | | | | |
|---|---|---|---|---|
| solution | Ka (106/Ms) | Kd (10-5/s) | KD (nM) | Rmax (RU) |
| Hu9.1RF IgG1 | 1.99 | 5.21 | 0.03 | 307 |
| Afucosylated | 1.49 | 5.41 | 0.04 | 312 |
| Hu9.1RF IgG1 | | | | |

The apparent binding affinities obtained using the 1 bivalent analyte model for data analysis are more reliable since the antibodies are bivalent. The apparent binding affinities obtained using the 1:1 Langmuir binding model for data analysis are higher likely due to the avidity effects. The apparent kinetic parameters for BR3 ECD binding to immobilized Hu9.1RF IgG1 antibodies are shown in Table 10.

TABLE 10
Binding of Br3 ECD WT to anti-BR3-1 antibody using
1:1 Langmuir model for data analysis.

| | Amount | | | | |
|---|---|---|---|---|---|
| Antibody on | immobilized | Ka | Kd | KD | Rmax |
| chip | (RU) | (105/Ms) | (10-4/s) | (nM) | (RU) |
| Hu9.1RF IgG1 | 2015 | 9.95 | 2.53 | 0.25 | 155 |
| Afucosylated | 1962 | 8.52 | 1.99 | 0.23 | 150 |
| Hu9.1RF IgG1 | | | | | |

Samples were analyzed at 1.95-1000 nM in 2-fold serial dilution.

In summary, Hu9-1RF IgG1 and afucosylated Hu9.1RF IgG1 gave similar apparent affinities by BIAcore.

### Example 16

### Binding of Afucosylated Hu9.1RF IgG1 Antibody to Fcγ Receptor

Binding of control and test materials to the human Fcγ receptors was assessed using modified versions of procedures originally described by Shields et al. (J Biol Chem 276:6591-604 (2001)). For test materials, varying levels of G0-F content from 2-20% were prepared by mixing 37% G0-F Hu9.1RF IgG1 (generated via RNAi-fucosyltransferase knockdown) with 2% G0-F GLP-Hu9.1RF IgG1. The “37% G0-F is really “52% afucosylated” by adding G1-F content (see FIG. 7). The 37% G0-F starting material included G1-F carbohydrates. The starting material was calculated to be 52% afucosylated if both G1-F and G0-F carbohydrates are included in the calculation. Monomeric IgG is capable of binding to the high-affinity receptor FcγRIA (CD64); however, the low-affinity receptors FcγRIIA (CD32A), FcγRIIB (CD32B), and FcγRIIIA (CD16) require multimeric IgG for binding. Therefore, for the low-affinity receptor binding assays, multimers of the control or test materials were formed by premixing the antibody with F(ab′)₂ fragment goat anti-human κ chain (Cappel, ICN Pharmaceuticals, Inc.; Aurora, Ohio) at a 1:2 (w/w) ratio in assay buffer (0.5% bovine serum albumin [BSA], 0.05% polysorbate 20 in phosphate-buffered saline [PBS]) at pH 7.4. FcγRs were expressed as recombinant fusion proteins of the extracellular domain of the receptor alpha chains with gly/his6/glutathione-s-transferase (GST). Assay plates coated with anti-GST and blocked with BSA were used to capture each FcγR. Plates were washed with wash buffer (0.05% polysorbate 20 in PBS, pH 7.4) after each incubation. Serial dilutions of Hu9.1RF IgG1 or afucosylated Hu9.1RF IgG1 presented as monomers for FcγRIA and as multimers for the low-affinity FcγR, were added to the plates in duplicate and incubated for 2 hours. Bound antibody was detected with the horseradish peroxidase (HRP)-conjugated F(ab′)2 fragment of a goat anti-human IgG F(ab′)₂ (Jackson ImmunoResearch; West Grove, Pa.). Plates were developed with tetramethylbenzidine (TMB) (KPL, Inc.; Gaithersburg, Md.) as the substrate. Absorbance was measured at a wavelength of 450 nm with a reference of 650 nm. Mean absorbance values from duplicate wells were plotted as a function of antibody concentration. Data were fit to a four-parameter equation to determine the concentration that yields 50% of maximum binding to each FcγR (EC₅₀) for each antibody.

As shown in Table 11 and 12 below, no significant difference in binding to FcγR1A, FcγRIIA and FcγRIIB was observed for the Hu9.1RF IgG1 antibodies with variable levels of G0-F. The 100% AF Hu9.1RF IgG1 showed significantly higher binding affinities for FcγRIIIA-F and FcγRIIIA-V.

TABLE 11
Determined G0-F levels.

| Sample | % G0-F |
|---|---|
| (Expected Level of G0-F) | (Determined level) |
| Hu9.1RF IgG1, 2% | 2 |
| Hu9.1RF IgG1, 4% | 3 |
| Hu9.1RF IgG1, 6% | 6 |
| Hu9.1RF IgG1, 10% | 8 |
| Hu9.1RF IgG1, 20% | 23 |
| HV3-46S-Bulk | 4 |

TABLE 12
Average EC₅₀ for Hu9.1RF IgG1 with variable levels of G0-F.

| | | Average_{EC50} | | | | |
|---|---|---|---|---|---|---|
| | | ng/mL | μg/ml | μg/ml | μg/ml | μg/ml |
| | n | FcγR-IA | FcγR-IIA | FcγR-IIB | FcγR-IIIA-F | FcγR-IIIA-V |
| Hu9.1RF IgG1 2% G0-F | 3 | 3.92 | 0.30 | 0.96 | 2.20 | 0.34 |
| Hu9.1RF IgG1 4% G0-F | 3 | 3.73 | 0.26 | 0.95 | 1.07 | 0.21 |
| Hu9.1RF IgG1 6% G0-F | 3 | 4.12 | 0.23 | 0.91 | 0.70 | 0.15 |
| Hu9.1RF IgG1 10% G0-F | 3 | 3.91 | 0.24 | 0.88 | 0.39 | 0.10 |
| Hu9.1RF IgG1 20% G0-F | 3 | 3.74 | 0.17 | 0.82 | 0.16 | 0.06 |
| HV3-46S | 1 | 1.58 | 0.30 | 1.02 | 1.52 | 0.28 |

### Example 17

### Binding of Afucosylated Hu9.1RF IgG1 Antibody to FcRn

As described above, Hu9.1RF IgG1 is a humanized monoclonal IgG1 antibody directed against BR3 and having the VH and VL sequences of SEQ ID NOs: 35 and 21, respectively (see Table 2). Like other IgG, antibodies, the Fc portion of Hu9.1RF IgG1 binds to Fcγ receptors on the surfaces of immune effector cells. This binding initiates cellulariesponses such as antibody-dependent cellular cytotoxicity (ADCC) (Gessner et al., Ann Hematol. 76:231-48 (1998)). Binding of Hu9.1RF IgG1 to the BR3 receptor blocks BAFF-dependent B-cell proliferation, and also induces Fc-mediated cell killing through ADCC, which results in B-cell depletion.

FcRn is the major histocompatibility complex class I-related neonatal Fc receptor and is responsible for the long half-life of circulating IgG. The Fc region of IgG binds to FcRn at acidic pH (pH 6.0-6.5) and releases from the receptor at neutral pH (pH 7.4) (Raghavan et al., Biochemistry 34:14649-57 (1995)). This process protects IgG from degradation and recycles IgG back into circulation. The binding affinity of an IgG for FcRn at acidic pH has been found to correlate with the half-life of the IgG in serum (Ghetie and Ward, Ann. Rev. Immunol. 18:739-66 (2000)). As described above for Example 16, we have generated an afucosylated Hu9.1RF IgG1 (Hu9.1RF IgG1 AF) using a cell line lacking fucosyltransferase. The experiments described below investigated whether afucosylated Hu9.1RF IgG1 altered binding affinity to FcRn compared with Hu9.1RF IgG1 that has normal glycans.

Test material Hu9.1RF IgG1 AF and control antibody Hu9.1RF IgG1 were provided by Genentech, Inc. as clear, colorless liquid solutions. Before their use in the study, all materials were stored in a refrigerator set to maintain a temperature of 2° C.-8° C.

Soluble human FcRn was produced in Chinese hamster ovary cells. Binding of control and test materials to human FcRn was assessed as described by Shields et al. J. Biol. Chem. 277.26733-26740 (2001)). Briefly, assay plates coated with NeutrAvidinä (Pierce Biotechnology; Rockford, Ill.) and blocked with BSA were used to capture biotinylated FcRn in assay buffer at pH 7.4. Plates were washed following each incubation step with wash buffer at pH 7.4. Subsequent incubation and wash steps were carried out at pH 6.0. Serial dilutions of Hu9.1RF IgG1 AF or Hu9.1RF IgG1 control antibody were added to the plates in duplicate and incubated for 2 hours. Bound antibody was detected with the HRP-conjugated F(ab′)₂ fragment of a goat anti-human IgG F(ab′)₂. Plates were developed with TMB as the substrate. Absorbance was measured at a wavelength of 450 nm with a reference of 620 nm.

To evaluate the dissociation of bound IgG from FcRn at pH 7.4, the assay was carried out in a similar manner as previously described, except with the addition of a dissociation step. After the sample-incubation step, the plates were washed with wash buffer at pH 6.0. Assay buffer at pH 6.0 or pH 7.4 was added to the plates and incubated at room temperature for 45 minutes. The assay was then continued as previously described.

Hu9.1RF IgG1 was used as a control for the relative binding affinity analysis for FcRn. The optical density at 450 nm at the midpoint absorbance (mid-OD) of the Hu9.1RF IgG1 titration curve was calculated by first dividing the difference in absorbance at the highest and lowest concentrations by two, and then adding the absorbance reading at the lowest concentration. The corresponding concentrations of control and samples at this mid-OD were determined from the titration curves using a four-parameter regression curve-fitting program (XLfit, ID Business Solutions Ltd.; Guildford, Surrey, UK). The Hu9.1RF IgG1 AF binding affinity relative to Hu9.1RF IgG1 was calculated by dividing the mid-OD concentration of Hu9.1RF IgG1 by that of Hu9.1RF IgG1 AF

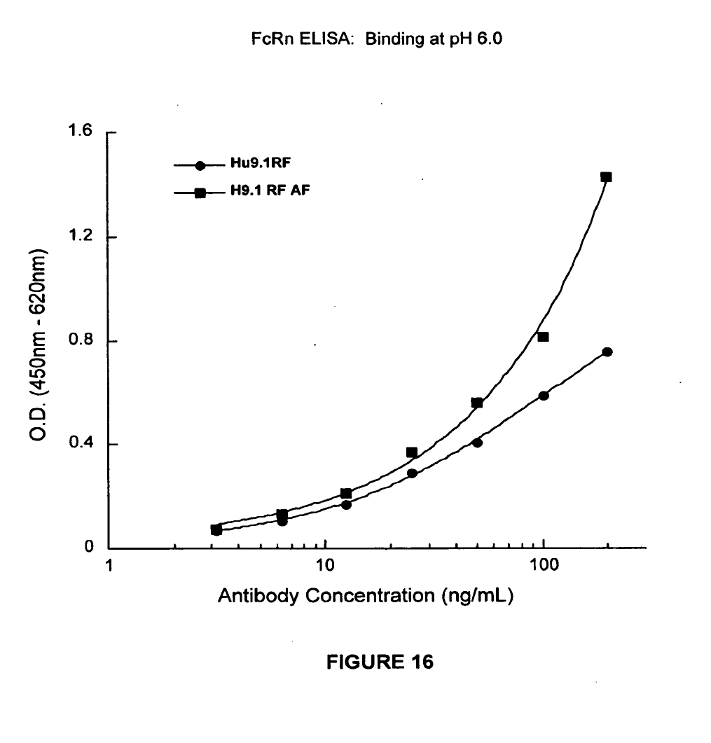

The impact of lack of fucose in the glycans of Hu9.1RF IgG1 AF antibody was characterized by using an ELISA to measure the binding affinity of Hu9.1RF IgG1 AF and Hu9.1RF IgG1 for human FcRn at pH 6.0. Representative binding curves of the two antibodies to human FcRn at pH 6.0 are shown in FIG. 16. Relative to Hu9.1 RF IgG1 that has normal glycans, Hu9.1RF IgG1 AF showed a 1.4-fold higher binding affinity for human FcRn (see Table 13).

We have found that the presence of aggregates in antibody samples may increase the apparent binding affinity for FcRn in this ELISA. The percent aggregate for Hu9.1RF IgG1 AF was determined to be 1.9%, which is higher than that for Hu9.1RF IgG1 (0.2%). The slightly increased binding affinity of HU9.1RF IgG1 AF for FcRn as compared with the wild-type molecule Hu9.1RF IgG1 is likely attributable to the presence of aggregates. Therefore, the absence of fucose did not significantly alter Hu9.1RF IgG1 AF binding affinity to FcRn.

TABLE 13
Summary of Relative Binding Affinities in FcRn Binding Assays

| | Concentration | | |
|---|---|---|---|
| | at Mid-OD | | Relative Affinity to |
| Antibody | ng/mL | nM^{a} | Hu9.1RF IgG1 |
| Hu9.1RF IgG1 | 48.8 | 0.33 | 1.0 |
| Hu9.1RF IgG1 AF | 33.8 | 0.23 | 1.4 |

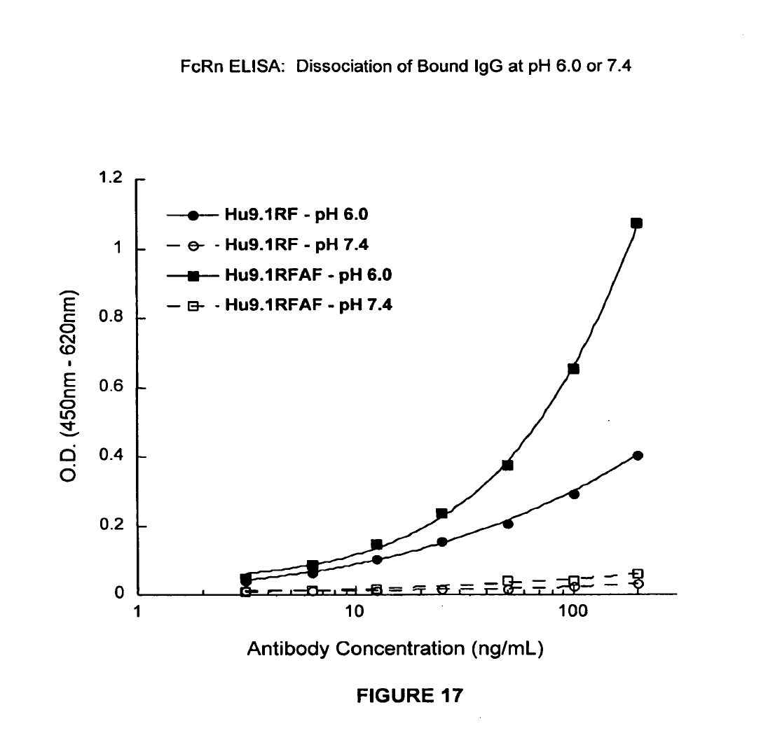

For IgG to recycle back into circulation, it must dissociate from FcRn at physiological pH. The dissociation of Hu9.1RF IgG1 AF and Hu9.1RF IgG1 from FcRn at pH 7.4 was determined. The result indicated that both Hu9.1RF IgG1 AF and Hu9.1RF IgG1 dissociated from FcRn in assay buffer at pH 7.4, whereas the antibodies remained bound at pH 6.0 (see FIG. 17)

Although afucosylated Hu9.1RF IgG1 showed a slightly higher binding affinity for human FcRn at pH 6.0 in the FcRn ELISA, taken into the consideration of aggregate effect on the assay, lack of fucose did not significantly affect Hu9.1RF IgG1 AF binding to FcRn compared with Hu9.1RF IgG1 that has normal fucose content. Both Hu9.1RF IgG1 AF and Hu9.1RF IgG1 antibodies bound to FcRn showed good dissociation at pH-7.4.

### Example 18

### Complement Dependent Cytotoxicity Assay Using Afucosylated Hu9.1RF IgG1 Antibody

The complement dependent cytotoxicity (CDC) assay measures the degree of antibody dependent complement lysis of target cells. Human serum complement protein C1q binds to the Fc domain of an antibody bound to its target cells and triggers the initiation of the complement cascade. This action eventually culminates in the formation of the complement protein membrane attack complex resulting in target cell lysis. The assay is performed in a 96 microwell plate format and in duplicate as follows.

For the experiments described below, AF Hu9.1 RF IgG1 was generated using a cell line lacking fucosyltransferase and is 100% afucosylated. 50 μl of serially diluted (1:3) AF Hu9.1RF IgG1 and controls starting at 300 nM was incubated with 50 μl of B-cell lymphoma cells lines (50,000) BJAB, and WIL2-S (ATCC CRL-8885), along with 50 μl of a 1:4 dilution of normal human serum complement (Quidel, Santa Clara, Calif.). After a 2 hour incubation at 37° C., 50 μl of Alamar Blue (Biosource International, Camarillo, Calif.) was added and incubated for an additional 18 hours at 37° C. The plates were briefly shaken for 15 minutes and then read on a fluorescent plate reader (Ext. 535 nm, Emt 590 nm) to determine the relative fluorescent units (RFU). The RFU value observed was plotted relative to concentration of mAb in KaleidaGraph (Synergy Software, Reading, Pa.). Curves are plotted using a 4-parameter fit.

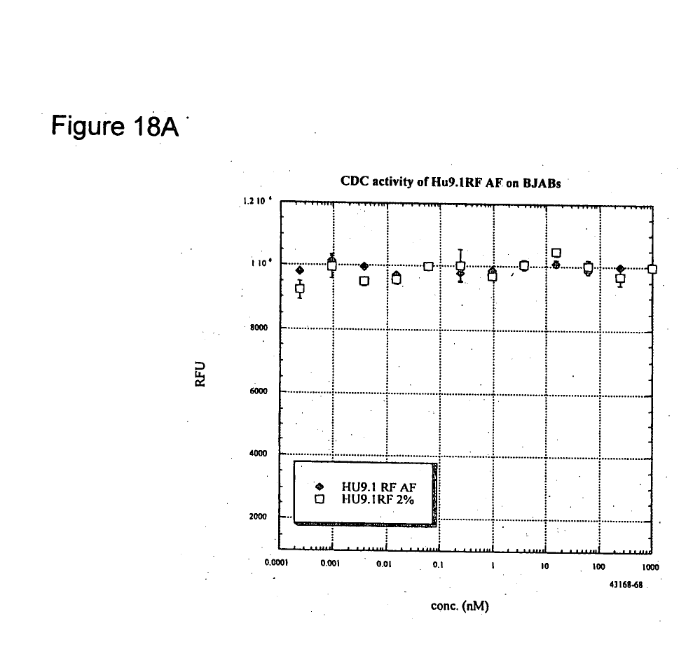

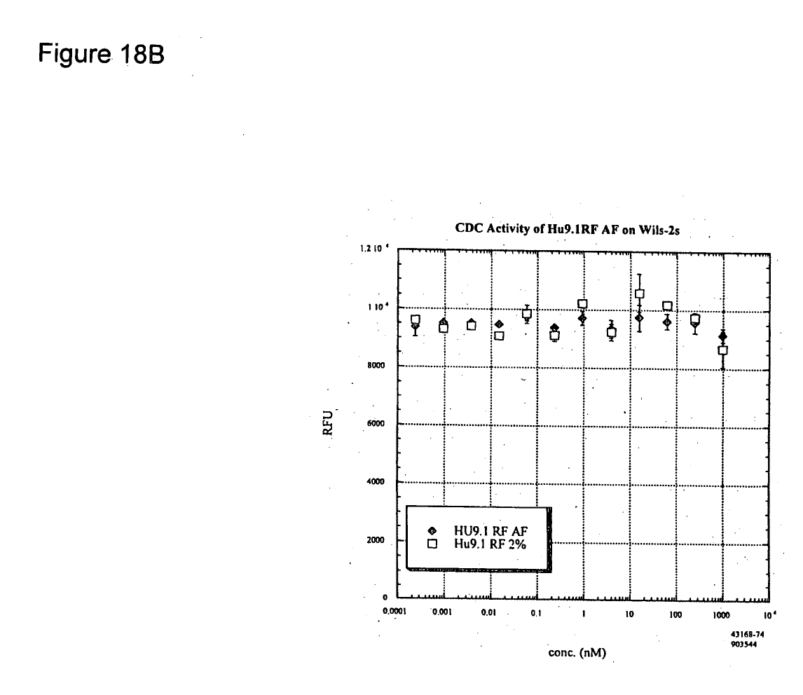

The CDC activity of AF Hu9.1RF IgG1 is shown in FIGS. 18A and 18B. Negative control and positive control monoclonal antibody were tested on BJAB and WIL2s cells. No complement dependent cytotoxicity was observed at any concentration of AF Hu9.1RF IgG1 on BJAB or WIL2-s cells.

### Example 19

### Antibody Dependent Cellular Cytotoxicity Using afucosylated Hu9.1RF IgG1 Antibody

As described in Example 7, above, the antibody dependent cellular cytotoxicity assay (ADCC) measures the degree of antibody dependent, NK cell killing of target cells. NK cells express on their surface the Type III Fc gamma receptor (CD16), a low affinity receptor for the immunoglobulin Fc domain of antibodies. Antibodies that are bound to target cells can bind CD16 with high avidity enabling the recruitment of effector NK cells whereupon NK cell activation leads to lysis of target cells. NK cells are isolated from 100 mL of normal human whole blood using negative selection following the manufacture's (RosetteSep, StemCell Technologies) recommended protocol. Assays were performed in round bottom 96 microwell plates. The assay and all dilutions were in F12/DMEM containing 1% fetal bovine serum. In triplicate, 50 μl of 1:4 serially diluted amounts of anti-BR3 and control monoclonal antibodies, starting at 100 nm, was incubated with 50 μl of target cells. Target cells were BJAB (10,000 per 50 μL), a B-lymphoma cell line. After a 30 minute room temperature incubation with the serially diluted antibodies, 50 μl of effector NK cells (50,000) were added and incubated for an additional 4 hours at 37° C. The effector to target cell ratio was 5:1. The plate(s) were centrifuged at 1500 rpm for 10 minutes and 100 μl of the cell media was removed and assayed. The level of cell lysis was determined by measuring the amount of lactate dehyrogenase (LDH kit, Roche Diagnostics) released from lysed cells. The percent lysis relative to mAb concentration was determined and plotted in KaleidaGraph (Synergy Software, Reading, Pa.) using a 4-parameter curve fit.

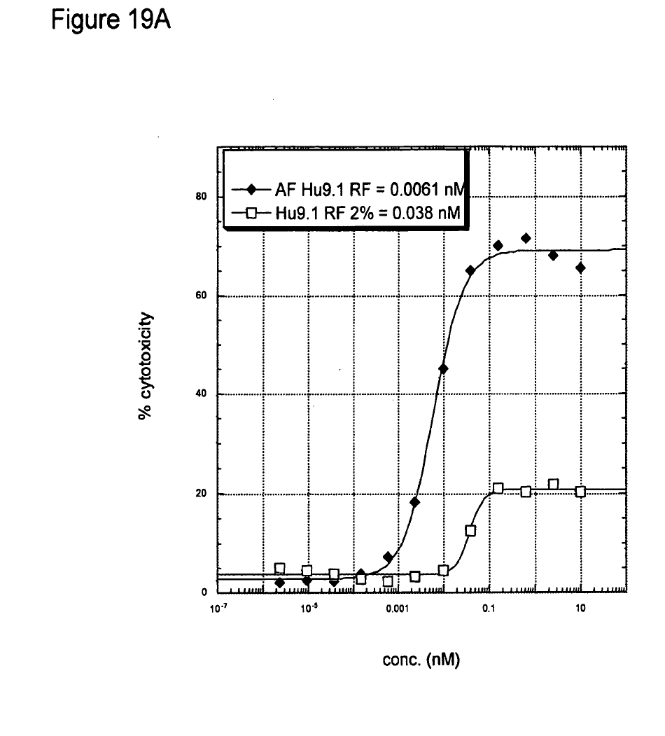

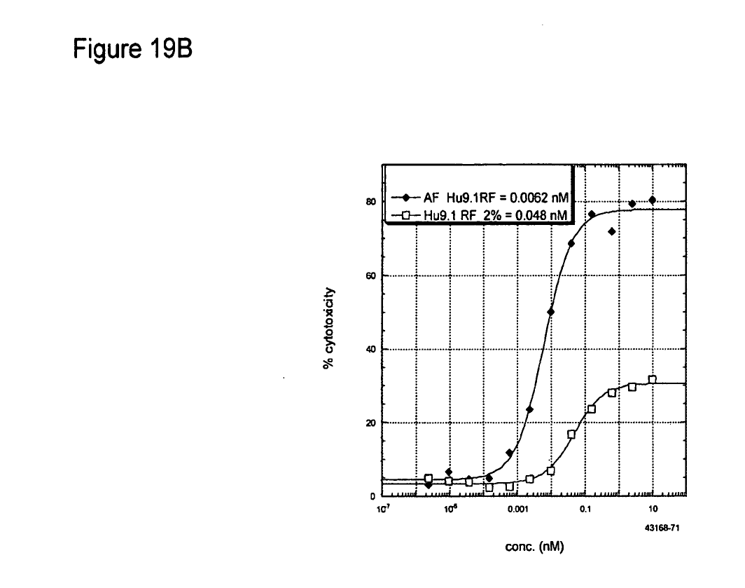

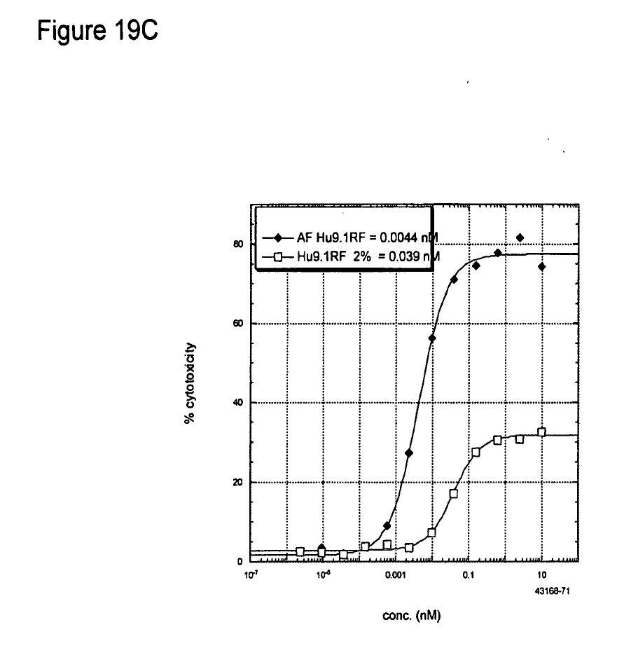

As described in Example 18, AF Hu9.1 RF IgG1 was generated using a cell line lacking fucosyltransferase and is 100% afucosylated. The ADCC activity of AF Hu9.1RF IgG1 was compared with 2% afucosylated Hu9.1RF IgG1 using normal human NK cells from three donors (FIGS. 19A-19C). The ECₛₒ of AF Hu9.1RF IgG1 was greater than 6-fold improved in ADCC activity than 2% afucosylated Hu9.1RF IgG1. In addition, AF Hu9.1RF IgG1 demonstrated a greater than 3-fold increase in percent cytotoxicity over 2% afucosylated Hu9.1RF IgG1. The ADCC activity of Hu9.1RF IgG1 monoclonal antibodies differing in percent afucosylation was also compared using NK cells isolated from three normal donors (FIGS. 20A-20C). The level of ADCC activity increased as the level of afuscoylation increased. This was observed for all three donors.

### Example 20

### Apoptosis Assay

BR3⁺ BJAB lymphoma cells were used to measure the ability of the anti-BR3 monoclonal antibody AF Hu9.1RF IgG1 to induce apoptosis as measured through Annexin V staining and propidium iodide dye exclusion (Cat# V-13241 Molecular Probes, Seattle, Wash.). As described in Example 18, AF Hu9.1 RF IgG1 was generated using a cell line lacking fucosyltransferase and is 100% afucosylated. The BJAB cells were cultured in RPMI 1640 media (Gibco, Rockville, Md.) containing 10% fetal calf serum (Biosource International, Camarillo, Calif.) and 2 mM L-glutamine (Gibco). Prior to being assayed, the cells were washed twice in fresh media and then adjusted to a cell concentration of 2×10⁶ per milliliter. 100 μl of cells were added to 96 well microassays plates (Becton Dickinson, Palo Alto, Calif.) which contained 100 μl of a predetermined amount of AF Hu9.1RF IgG1, negative control IgG and positive control IgG, along with F(ab)′₂ goat anti-human Fc (Cat # 109-006-098 Jackson ImmunoResearch Labs, West Grove, Pa.). The final monoclonal antibody concentrations were 100, 10, 1.0, and 0.1 nM and the F(ab)′2 goat anti-human Fc molecule was set at twice the concentration of the AF Hu9.1RF IgG1 and control antibodies. Each point was performed in duplicate. After a 24-hour incubation period at 37° C., the cells were washed twice with phosphate buffered saline and then stained with Annexin V and propidium iodide according to the manufacture's recommendations. The staining patterns of the BJAB cells were analyzed by flow cytometry using a FACSCalibur Flow Cytometer (Becton Dickinson San Jose, Calif.), and collected for period of 10-20 seconds and by the number of events. The data was reduced using the Cellquest Pro software (Becton Dickinson). BJAB cells that were positive for (1) Annexin V staining, (2) both Annexin V and propiduim iodide double-staining, and (3) the number of unstained live cells were scored and plotted using KaleidaGraph graphing software (Synergy Software, Reading, Pa.).

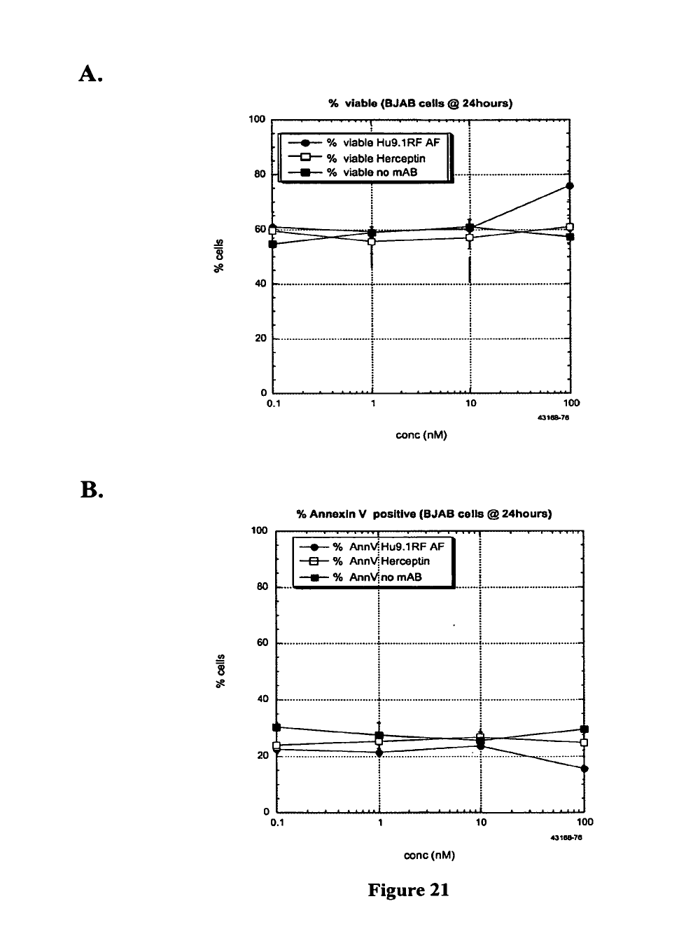

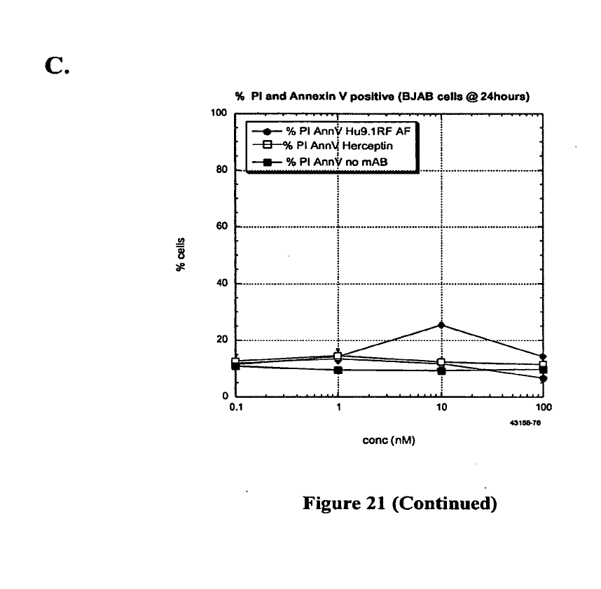

AF Hu9.1RF IgG1, at concentrations ranging from 0.1 to 100 nM and in the presence of an anti-IgG crosslinker, did not increase the level Annexin V staining above background when incubated with BR3 positive BJAB cells for 20 hours (FIGS. 21A-21C). In addition, under the same conditions, there was no observed increase in Annexin V and Propidium Iodide surface staining. These data indicated that crosslinked AF Hu9.1RF IgG1 (0.1 to 100 nM) does not induce apoptosis in BR3 positive BJAB cells.

### Other Embodiments

From the foregoing description, it will be apparent that variations and modifications may be made to the invention described herein to adopt it to various usages and conditions. Such embodiments are also within the scope of the following claims.

All publications, patent applications, and patents mentioned in this specification, including U.S. Provisional Application No. 60/830,969, are herein incorporated by reference to the same extent as if each independent publication, patent, or patent application was specifically and individually indicated to be incorporated by reference.

What is claimed is:

1-35. (canceled)

36. An anti-BR3 antibody having an Fc region wherein said anti-BR3 antibody is afucosylated.

37. An anti-BR3 antibody composition comprising an afucosylated anti-BR3 antibody of claim 36.

38. The anti-BR3 antibody composition of claim 37, wherein said composition comprises at least 2% afucosylated anti-BR3 antibodies.

39. The anti-BR3 antibody composition of claim 39, wherein said composition comprises at least 4% afucosylated-anti-BR3 antibodies.

40. The anti-BR3 antibody composition of claim 39, wherein said composition comprises at least 10% afucosylated anti-BR3 antibodies.

41. The anti-BR3 antibody composition of claim 40, wherein said composition comprises at least 19% afucosylated-anti-BR3 antibodies.

42. The anti-BR3 antibody composition of claim 41, wherein said composition comprises 100% afucosylated anti-BR3 antibodies.

43. The afucosylated anti-BR3 antibody of claim 36, further comprising a variant Fc sequence, wherein the Fc sequence has a substitution at any one or any combination of positions selected from the group consisting of 268D, 326D, 333A/334A, 298A/333A, 298A/334A, 239D/332E, 239D/298A/332E, 239D/268D/298A/332E, 239D/268D/298A/326A/332A, 239D/268D/298A/326A/332E, 239D/268D/283L/298A/332E, 239D/268D/283L/298A/326A/332E, 239D/330L/332E, 272Y/254T/256E, T250Q/M428L, D265A, and N297A, wherein the D265A substitution is in the absence of N297A and the N297A substitution is in the absence of D265A.

44. The afucosylated anti-BR3 antibody of claim 36, further comprising a variant Fc sequence, wherein the antibody comprises an Fc region that has been altered to change the ADCC, CDC and/or pharmacokinetic property of the antibody compared to a wild type IgG Fc sequence by substituting an amino acid at any one or any combination of positions selected from the group consisting of: 238, 239, 246, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 309, 312, 314, 315, 320, 322, 324, 326, 327, 329, 330, 331, 332, 333, 334, 335, 337, 338, 340, 360, 373, 376, 378, 382, 388, 389, 398, 414, 416, 419, 428, 430, 434, 435, 437, 438 and 439 of the Fc region.

45. The afucosylated anti-BR3 antibody of claim 36, wherein the antibody has ADCC activity in the presence of human effector cells or has increased ADCC in the presence of human effector cells compared to an anti-BR3 antibody comprising a human wildtype IgG1 Fc.

46. (canceled)

47. The afucosylated anti-BR3 antibody of claim 36, wherein the antibody can block BAFF (SEQ ID NO:143) from binding to the extracellular domain of BR3 (SEQ ID NO:151).

48. (canceled)

49. The afucosylated anti-BR3 antibody of claim 36, wherein the antibody binds an FcγRIII.

50. The afucosylated anti-BR3 antibody of claim 36, wherein the antibody binds the FcγRIII with better affinity, or mediates antibody-dependent cell-mediated cytotoxicity (ADCC) more effectively, than the glycoprotein with a mature core carbohydrate structure including fucose attached to the Fc region of the glycoprotein.

51-53. (canceled)

54. The afucosylated anti-BR3 antibody of claim 36, wherein the fucosyltransferase gene is the FUT8 gene.

55. The afucosylated anti-BR3 antibody of claim 36, wherein the antibody is essentially free of bisecting N-acetylglucosamine (GlcNAc) attached to the mature core carbohydrate structure.

56. The afucosylated anti-BR3 antibody of claim 36, wherein the antibody has bisecting N-acetylglucosamine (GlcNAc) attached to the mature core carbohydrate structure.

57. The afucosylated anti-BR3 antibody of claim 36, wherein the antibody has one or more galactose residues attached to the mature core carbohydrate structure.

58. The afucosylated anti-BR3 antibody of claim 36, wherein the antibody is essentially free of one or more galactose residues attached to the mature core carbohydrate structure.

59. The afucosylated anti-BR3 antibody of claim 36, wherein the antibody has one or more sialic acid residues attached to the mature core carbohydrate structure.

60. The afucosylated anti-BR3 antibody of claim 36, wherein the antibody is essentially free of one or more sialic acid residues attached to the mature core carbohydrate structure.

61. A pharmaceutical composition comprising the antibody composition of claim 37.

62-63. (canceled)

64. The anti-BR3 antibody of claim 36, wherein the anti-BR3 antibody is a humanized or human antibody that binds to a human BR3 extracellular domain sequence and has an H1, H2 and H3 region with at least 70% homology to the H1, H2 and H3 region, respectively, of any one of the antibodies of Table 2 and has an L1, L2 and L3 region with at least 70% homology to the L1, L2 and L3 region, respectively, of any one of the antibodies of Table 2.

65. The anti-BR3 antibody of claim 36, wherein the anti-BR3 antibody is a human or humanized antibody that binds to a human BR3 extracellular domain sequence and has at least 70% homology to a VH domain of any one of the antibodies of Table 2.

66. The anti-BR3 antibody of claim 36, wherein the anti-BR3 antibody is a humanized antibody that binds to a human BR3 extracellular domain sequence, the antibody comprising an H3 sequence of any one of SEQ ID NOs. 4-13, 15, 16-18, 20, 22, 24, 26, 28-73, 75-76, 78, 80-85, 87-96, 98, 100, 102, 104, 106, 107, 109-110, 112, 116, 118, 120, 122, 124-127 and 129-131 and further comprising the H1 and H2 sequences and the L1, L2, and L3 sequences from any one of the antibodies disclosed in Table 2.

67-69. (canceled)

70. The anti-BR3 antibody of claim 36, wherein the anti-BR3 antibody has been conjugated to a cytotoxic agent or a chemotherapeutic agent.

71. The anti-BR3 antibody of claim 70, wherein the cytotoxic agent is a radioactive isotope or a toxin.

72. The anti-BR3 antibody of claim 36, wherein the antibody is a monoclonal antibody.

73. The anti-BR3 antibody of claim 36, wherein the antibody is a humanized antibody.

74. The anti-BR3 antibody of claim 36, wherein the antibody is derived from a human antibody sequence.

75. The anti-BR3 antibody of claim 36, wherein the antibody is a multi-specific antibody.

76. An isolated nucleic acid molecule that encodes the antibody of claim 36.

77. An expression vector encoding the antibody of claim 36.

78. A host cell comprising a nucleic acid molecule of claim 76.

79. The host cell of claim 78, that produces the antibody of claim 36.

80. The host cell of claim 78, which is a mammalian cell, a yeast cell, or a plant cell.

81. A method of treating a BR3 positive cancer, comprising administering to a patient suffering from the cancer a therapeutically effective amount of a composition of claim 37.

82. A method of treating a B cell neoplasm, comprising administering to a patient suffering from the neoplasm a therapeutically effective amount of a composition of claim 37.

83. A method of treating an autoimmune disease, comprising administering to a patient suffering from the autoimmune disease a therapeutically effective amount of a composition of claim 37.

84. A method of treating a cancer, comprising administering to a patient suffering from the cancer a therapeutically effective amount of a composition of claim 37.

85. A method of depleting B cells from a mixed population of cells comprising contacting the mixed population of cells with a composition of claim 37.

86. The method according to claim 83 further comprising the step of administering a therapeutically effective amount of an anti-CD20 antibody sequentially or concurrently with the anti-BR3 antibody.

87. The method according to claim 86, further comprising the step of contacting the mixed population with an anti-CD20 antibody sequentially or simultaneously with the anti-BR3 antibody.

88. The method of claim 86, wherein the CD20 binding antibody is the rituximab antibody.

89. The method of claim 83, further comprising the sequential or concurrent administration of a therapeutically effective amount of at least one of the group consisting of: a BAFF antagonist, a biologic response modifier, a B cell depletion agent, a cytotoxic agent, a chemotherapeutic agent and an immunosuppressive agent.

90. The method of according to claim 89, wherein the BAFF antagonist is selected from the group consisting of BR3-Fc, TACI-Fc, BCMA-Fc, an anti-BAFF peptibody, an anti-BAFF antibody and an anti-BR3 antibody.

91-92. (canceled)

93. The method of claim 83, wherein the autoimmune disease is selected from the group consisting of rheumatoid arthritis, juvenile rheumatoid arthritis, systemic lupus erythematosus (SLE), Wegener's disease, inflammatory bowel disease, idiopathic thrombocytopenic purpura (ITP), thrombotic thrombocytopenic purpura (TTP), autoimmune thrombocytopenia, multiple sclerosis, psoriasis, IgA nephropathy, IgM polyneuropathies, myasthenia gravis, vasculitis, diabetes mellitus, Reynaud's syndrome, Sjörgen's syndrome and glomerulonephritis.

94. The method of claim 93, wherein the autoimmune disease is rheumatoid arthritis.

95. The method of claim 89, wherein the immunosuppressive agent is methotrexate.

96. The composition of claim 37, wherein about 20-100% of the anti-BR3 antibodies in the composition are afucosylated.
